(19) United States
(12) Patent Application Publication
(10) Pub. No.: US 2025/0353891 A1
(43) Pub. Date: Nov. 20, 2025

(54) GLP-1 AND GLUCAGON DUAL AGONIST PEPTIDES WITH IMPROVED BIOLOGICAL STABILITY

(71) Applicant: MEDIMMUNE LIMITED, Cambridge (GB)

(72) Inventors: Sivaneswary GENAPATHY, Cambridge (GB); Maria Aleksandra BEDNAREK, Cambridge (GB)

(21) Appl. No.: 18/836,305

(22) PCT Filed: Feb. 6, 2023
(86) PCT No.: PCT/EP2023/052778

§ 371 Date: Aug. 6, 2024

Related U.S. Application Data
(60) Provisional application No. 63/307,206, filed on Feb. 7, 2022.

(51) Int. Cl. C07K 14/605; A61K 9/00; A61K 38/00

(52) U.S. Cl. CPC C07K 14/605; A61K 9/0019; A61K 38/00

(57) ABSTRACT

Provided herein are GLP-1 and glucagon dual agonists with improved biological stability (e.g., proteolytic stability) and duration of action. The peptides can be administered, e.g., once weekly.

### BACKGROUND OF THE INVENTION

The incidence of obesity and diabetes have been rising in epidemic proportions. Diabetes is characterized by high levels of blood glucose resulting from defects in insulin production, insulin action, or both. Type 2 diabetes mellitus (T2DM) accounts for some 90 to 95 percent of all diagnosed cases of diabetes, and the risk of type 2 diabetes rises with increasing body weight. The prevalence of type 2 diabetes is three to seven times higher in those who are affected by obesity than in normal weight adults, and is 20 times more likely in those with a body mass index (BMI) greater than 35 kg/m². However, weight-loss can improve control or cure type 2 diabetes.

Glucagon and glucagon-like peptide-1 (GLP-1) derive from pre-proglucagon, a 158 amino acid precursor polypeptide that is processed in different tissues to form a number of different proglucagon-derived peptides, including glucagon, glucagon-like peptide-1 (GLP-1), glucagon-like peptide-2 (GLP-2) and oxyntomodulin (OXM), that are involved in a wide variety of physiological functions, including glucose homeostasis, insulin secretion, gastric emptying, and intestinal growth, as well as the regulation of food intake. Glucagon is a 29-amino acid peptide that corresponds to amino acids 33 through 61 of proglucagon (53 to 81 of preproglucagon), while GLP-1 is produced as a 37-amino acid peptide that corresponds to amino acids 72 through 108 of proglucagon (92 to 128 of preproglucagon). GLP-1 (7-36) amide or GLP-1 (7-37) acid are biologically active forms of GLP-1, that demonstrate essentially equivalent activity at the GLP-1 receptor.

Glucagon is produced by the pancreas and interacts with the glucagon receptor (“glucR”). Glucagon acts in the liver to raise blood glucose via gluconeogenesis and glycogenolysis. When blood glucose begins to fall, glucagon signals the liver to break down glycogen and release glucose, causing blood glucose levels to rise toward a normal level.

GLP-1 has different biological activities compared to glucagon. It is secreted from gut L cells and binds to the GLP-1 receptor. Its activities include stimulation of insulin synthesis and secretion, inhibition of glucagon secretion, and inhibition of food intake.

Both glucagon and GLP-1, acting as agonists at their respective receptors, have been shown to be effective in weight loss. Certain GLP-1 analogs are being sold or are in development for treatment of obesity including, e.g., Liraglutide (Saxenda® from Novo Nordisk) and Semaglutide (Wegovy® from Novo Nordisk). Glucagon/GLP-1 dual agonist peptides such as cotadutide are also known and are in clinical development for treatment of diabetes, obesity, and nonalcoholic steatohepatitis (NASH). However, all of these proposed therapies involve chronic self-medication necessitating patient compliance over an extended period of time. Other peptides, e.g., amylin analogues are also being considered for the treatment of obesity, excess food intake, and diabetes (WO 2018/046719).

Accordingly, there remains a need for therapeutics that can agonize both GLP-1 and glucagon functions to e.g., improve glycemic control, reduce weight, treat type 2 diabetes mellitus, (T2DM) and/or treat NASH, while minimizing burdens associated with administration to improve patient compliance and quality of life.

### BRIEF SUMMARY OF THE INVENTION

Provided herein are GLP-1 and glucagon dual agonists with improved biological stability (e.g., proteolytic stability) and duration of action. The GLP-1 and glucagon dual agonist peptides provided herein can have improved properties, e.g., as compared to semaglutide H(Aib)EGTFTSDVSSYLEGQAAX20EFIAWLVRGRG-acid, wherein X20=Lys[O2Oc-O2Oc-γE-C18diacid] (SEQ ID NO: 539)) and/or cotadutide (HSQGTFTSDX10SEYLDSERARDFVAWLEAGG-acid, wherein X10=Lys[ε-γE-Palmitoyl] (SEQ ID NO: 538)). Accordingly, the GLP-1 and glucagon dual agonist peptides provided herein can be administered once weekly.

Certain aspects of the disclosure are directed to a peptide comprising the sequence: H-X2-X3-G-X5-X6-T-S-D-X10-S-X12-αMethyl-Phenylalanine (αMePhe)-L-X15-X16-X17-X18-A-X20-X21-X22-X23-X24-X25-X26-X27-X28-X29-X30-X31-Z, wherein X2 is Aminoisobutyric acid (Aib), S, or A, X3 is Q, H, or E, X5 is T or S, X6 is F or αMePhe, X10 is V, K or Y, X12 is K, E, or S, X15 is D or E, X16 is T, S, or G, X17 is K, R, E, or Q, X18 is R or A, X20 is R, K, or Q, X21 is D or E, X22 is αMePhe or F, X23 is V or I, X24 is Q or A, X25 is Aib or W, X26 is L or I, X27 is L, A, E, V, or M, X28 is E, N, A, R, or K, X29 is Aib, T, or G, X30 is G, R, or not present, X31 is G or not present, and Z is amide or acid (SEQ ID NO: 540).

In some aspects, X2 is Aib. In some aspects, X3 is Q. In some aspects, X3 is H. In some aspects, X5 is T. In some aspects, X5 is S. In some aspects, X6 is F. In some aspects, X6 is αMePhe. In some aspects, X10 is V. In some aspects, X12 is K. In some aspects, X15 is D. In some aspects, X16 is T. In some aspects, X16 is S. In some aspects, X17 is K. In some aspects, X17 is R. In some aspects, X18 is R. In some aspects, X18 is A. In some aspects, X20 is R. In some aspects, X20 is K. In some aspects, X21 is D. In some aspects, X22 is F. In some aspects, X22 is αMePhe. In some aspects, X23 is V. In some aspects, X24 is Q. In some aspects, X25 is W. In some aspects, X25 is Aib. In some aspects, X26 is L. In some aspects, X26 is I. In some aspects, X27 is L. In some aspects, X27 is A. In some aspects, X28 is E. In some aspects, X28 is N. In some aspects, X29 is Aib. In some aspects, X29 is T. In some aspects, X30 is G. In some aspects, X30 is not present. In some aspects, X31 is not present. In some aspects, Z is amide. In some aspects, Z is acid.

In some aspects, X2 is Aib, X12 is K, and X24 is Q. In some aspects, X16 is T, X17 is K, X27 is L, X28, is E, and X29 is Aib. In some aspects, X3 is Q, X5 is T, X6 is F, X10 is V, X12 is K, X15 is D, X16 is T, X17 is K, X18 is R, X20 is R, X21 is D, X22 is F, X23 is V, X24 is Q, X25 is W, X26 is L, X27 is L, X28 is E, X29 is Aib, X30 is G, X31 is not present, and Z is acid. In some aspects, X3 is H, X5 is S, X6 is αMePhe, X10 is V, X12 is K, X15 is D, X16 is S, X17 is R, X18 is A, X20 is K, X21 is D, X22 is αMePhe, X23 is V, X24 is Q, X25 is Aib, X26 is I, X27 is A, X28 is N, X29 is T, X30 is not present, X31 is not present, and Z is amide.

In some aspects, one or more lysine residues are acylated. In some aspects, the lysine at position 17 is acylated. In some aspects, the lysine at position 20 is acylated.

In some aspects, one or more lysine resides are lipidated. In some aspects, the lysine at position 17 is lipidated. In some aspects, the lysine at position 20 is lipidated.

In some aspects, the lipid is selected from the group consisting of octadecanedioic acid (C18diacid) and icosanedioic acid (C20diacid). In some aspects, the lipid is octadecanedioic acid (C18diacid). In some aspects, the lipid is icosanedioic acid (C20diacid). In some aspects, the lipid is linked to the epsilon amino group of lysine at position 17 or 20 via a linker.

In some aspects, the linker is ((O2Oc)-(O2Oc)-γE) or ((O2Oc)-(O2Oc)-γE-γE) in the C- to N-terminal orientation. In some aspects, the linker is ((O2Oc)-(O2Oc)-γE) in the C- to N-terminal orientation. In some aspects, the linker is ((O2Oc)-(O2Oc)-γE-γE) in the C- to N-terminal orientation. In some aspects, the linker is linked to the epsilon amino group of the residue at position 17 or 20.

Certain aspects of the disclosure are directed to a peptide comprising the sequence of H-Aib-Q-G-T-F-T-S-D-V—S-K-αMePhe-L-D-T-K-R-A-R-D-F-V-Q-W-L-L-E-Aib-G-acid (SEQ ID NO: 541).

In some aspects, the lysine at position 17 is acylated and lipidated, the lipid is linked to the acylated lysine via its epsilon amino group to ((O2Oc)-(O2Oc)-γE) linker in the C to N terminal orientation, and the lipid is octadecanedioic acid (C18diacid). In some aspects, the lysine at position 17 is acylated and lipidated, the lipid is linked to the acylated lysine via its epsilon amino group to ((O2Oc)-(O2Oc)-γE) linker in the C to N terminal orientation, and the lipid is icosanedioic acid (C20diacid).

Certain aspects of the disclosure are directed to a peptide comprising the sequence H-Aib-H-G-S-αMePhe-T-S-D-V—S-K-αMePhe-L-D-S-R-A-A-K (E-(O2Oc)-(O2Oc)-γE-C18diacid) 20-D-αMePhe-V-Q-Aib-I-A-N-T-amide (SEQ ID NO: 228).

Certain aspects of the disclosure are directed to a peptide comprising the sequence H-Aib-H-G-S-αMePhe-T-S-D-V—S-K-αMePhe-L-D-S-R-A-A-K (E-(O2Oc)-(O2Oc)-γE-γE-C20diacid) 20-D-αMePhe-V-Q-Aib-I-A-N-T-amide (SEQ ID NO: 233).

In some aspects, the peptide binds to the GLP-1 receptor (GLP-1R), binds to the glucagon receptor (GCGR), or binds to both a GLP-1 receptor and a glucagon receptor. In some aspects, the GLP-1R is a human GLP-1R. In some aspects, the GCGR is a human GCGR.

In some aspects, the peptide is an agonist of GLP-1 activity, an agonist of glucagon activity, or an agonist of both GLP-1 and glucagon activity. In some aspects, the peptide has increased proteolytic-resistance relative to the natural ligand of the GLP-1R and/or GCGR.

In some aspects, the peptide is isolated.

In some aspects, the peptide has at least 50%, at least 60%, at least 70%, at least 75%, at least 80%, at least 85%, at least 90%, at least 95, or 100% of intact peptide remaining after incubation with a protease at 37° C. for 5 min, 10 min, 15 min, 30 min, 2 hr, 4 hr, or 24 hr. In some aspects, the protease is selected from the group consisting of neprilysin, pepsin, pancreatin, simulated gastric fluid with pepsin, and simulated intestinal fluid with pancreatin.

In some aspects, the peptide has a half-life in cynomolgus monkeys after intravenous administration of at least 45 hours, at least 50 hours, at least 60 hours, at least 70 hours, at least 80 hours, at least 90 hours, at least 100 hours, at least 110 hours, at least 120 hours, or about 130 hours. In some aspects, the peptide has an s.c. bioavaiablity in cynomolgus monkeys of at least 75%, at least 80%, at least 90%, or about 95%.

In some aspects, a pharmaceutical composition comprising the peptide is provided herein. In some aspects, the composition is a solid composition. In some aspects, the composition is a liquid composition.

In some aspects, provided herein is a method of treating or preventing a disease or condition caused or characterized by excess body weight, wherein the method comprises administering to a subject in need of treatment an effective amount of any peptide or composition provide herein. In some aspects, the disease or condition is obesity. In some aspects, the disease or condition is type 2 diabetes.

In some aspects, provided herein is a method of treating or preventing non-alcoholic steatohepatitis (NASH), wherein the method comprises administering to a subject in need of treatment an effective amount of any peptide or composition provide herein.

In some aspects of a method provided herein, the administration is by injection. In some aspects of a method provided herein, the administration is oral. In some aspects of a method provided herein, the administration decreases body weight of the subject, increases insulin secretion in the subject, delays gastric emptying in the subject, decreases food intake in the subject, increases mitochondria function in the subject, inhibits de novo lipogenesis in the subject, decreases HbA1c in the subject, enhances fatty oxidation in the subject, decreases hepatic mitochondrial oxidative stress in the subject, decreases steatosis in the subject, decreases fibrosis in the subject, decreases glycogen synthesis in the subject, increases gluconeogenesis in the subject, halts disease progression in the subject, reverses fibrosis in the subject, and/or reduces risk of death due to cirrhosis, hepatocellular carcinoma, and/or cardiorenal disease in the subject. In some aspects of a method provided herein, the subject is a human. In some aspects of a method provided herein, the peptide is administered about once a week.

### BRIEF DESCRIPTION OF THE DRAWINGS/FIGURES

FIGS. 1A-1G are drawings depicting the exemplary lipids icosanedioic acid (C20diacid) (FIG. 1A), octadecanedioic acid (C18diacid) (FIG. 1B), stearoyl (FIG. 1C), margaroyl (FIG. 1D), palmitoyl (FIG. 1E), myristoyl (FIG. 1F), and lauryl (FIG. 1G).

FIGS. 2A-2F are drawings depicting exemplary linkers attached to lipids depicted in FIGS. 1A-1G 2-(2-(2-aminoethoxy)ethoxy) acetic acid (O2Oc) (FIG. 2A), (O2Oc)-(O2Oc) (FIG. 2B), (O2Oc)-γE-(O2Oc) (FIG. 2C), (PEG)2-(PEG)2-γE-γE (FIG. 2D), (PEG)2-γE-(PEG)2-γE (FIG. 2E), gammaglutamic acid (γE) (FIG. 2F), γE-(O2Oc) (FIG. 2G), γE-(O2Oc)-(O2Oc) (FIG. 2H), γE-(O2Oc)-γE-(O2Oc) (FIG. 2I), γE-(PEG)2-(PEG)2 (FIG. 2J), γE-(PEG)2-γE-(PEG)2 (FIG. 2K), γE-(PEG)4 (FIG. 2L), γE-γE (FIG. 2M), γE-γE-(O2Oc) (FIG. 2N), γE-γE-(O2Oc)-(O2Oc) (FIG. 2O), γE-γE-(PEG)12 (FIG. 2P), γE-γE-(PEG)2-(PEG)2 (FIG. 2Q), γE-γE-(PEG)2-γE-γE (FIG. 2R), γE-γE-(PEG)4 (FIG. 2S), γE-γE-(PEG)8 (FIG. 2T), γE-γE-(O2Oc)-(O2Oc)-γE-γE (FIG. 2U), and (PEG)2-(PEG)2-γE (FIG. 2V). All linkers shown with the N-terminal on the left and C-terminal on right.

FIGS. 3A-3P are drawings depicting the exemplary non-natural amino acids 2-amino-2-methylpropanoic acid (Aib) (FIG. 3A), (S)-2-amino-2-methyl-3-phenylpropanoic acid (αMePhe) (FIG. 3B), (S)-2-amino-3-hydroxy-2-methylpropanoic acid (αMeSer) (FIG. 3C), D-glutamine (dGln) (FIG. 3D), β-dimethylglutamine (β-dimethylGln) (FIG. 3E), (S)-2,5-diamino-2-methyl-5-oxopentanoic acid (αMeGln) (FIG. 3F), D-serine (dSer) (FIG. 3G), methyl-L-gluatmine (N-MeGln) (FIG. 3H), ((1H-imidazol-4-yl)methyl)glycine (NHis) (FIG. 3I), 1-aminocyclopropane-1-carboxylic acid (Acpr) (FIG. 3J), 1-aminocyclobutane-1-carboxylic acid (Acbu) (FIG. 3K), N⁶-acteyl-L-lysine (Ac-Lys) (FIG. 3L), (S)-2-amino-5-guanidino-3,3-dimethylpentaonic acid (β-dimethylArg) (FIG. 3M), (S)-2-amino-3,3-diphenylpropanoic acid (Dip) (FIG. 3N), (S)-2-amino-3-cyclohexylpropanoic acid (Cha) (FIG. 3O), (S)-2-aminohexanoic acid (Nle) (FIG. 3P), (S)-3-([1,1′-biphenyl]-4-yl)-2-aminopropanoic acid (Bip) (FIG. 3Q), 1-methyl-L-tryptophan (1-Methyl-Trp) (FIG. 3R), and(S)-2-amino-3-(5-bromo-1H-indol-3-yl) propanoic acid (5-Br-Trp) (FIG. 3S).

FIGS. 4A-4D are drawings depicting the structure of exemplary peptides: peptide 224 (FIG. 4A), peptide 229 (FIG. 4B), peptide 188 (FIG. 4C), and peptide 195 (FIG. 4D).

### DETAILED DESCRIPTION OF THE INVENTION

### I. Definitions

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

Throughout this disclosure, the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range are also provided.

It is understood that wherever aspects are described herein with the language “comprising,” otherwise analogous aspects described in terms of “consisting of” and/or “consisting essentially of” are also provided. A peptide “comprising” a particular amino acid sequence refers to a peptide containing the amino acid sequence, wherein the peptide may or may not contain additional amino acids or other modifications to the amino acid sequence. A peptide “consisting of” a particular amino acid sequence refers to a peptide containing only the amino acid sequence and no additional amino acids or other modifications to the amino acid sequence. A peptide “comprising” an amino acid sequence “consisting of” a particular amino acid sequence refers to a peptide containing the amino acid sequence and no additional amino acids; however, the peptide may comprise other modifications to the amino acid sequence (e.g., an acyl moiety or a palmitoyl moiety).

As used herein, the term “amino acid” refers to naturally occurring and non-natural amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrrolysine and selenocysteine. Amino acid analogs refers to compounds that have the same basic chemical structure as a naturally occurring amino acid, i.e., an a carbon that is bound to a hydrogen, a carboxyl group, an amino group, and an R group, such as, homoserine, norleucine, methionine sulfoxide, and methionine methyl sulfonium. Such analogs have modified R groups (such as, norleucine) or modified peptide backbones, but retain the same basic chemical structure as a naturally occurring amino acid.

A “non-natural amino acid” refers to an amino acid that is not one of the 20 common amino acids or pyrrolysine or selenocysteine; other terms that may be used synonymously with the term “non-natural amino acid” are “non-naturally encoded amino acid,” “unnatural amino acid,” “non-naturally-occurring amino acid,” and variously hyphenated and non-hyphenated versions thereof. The term “non-natural amino acid” includes, but is not limited to, amino acids that occur naturally by modification of a naturally encoded amino acid (including but not limited to, the 20 common amino acids or pyrrolysine and selenocysteine) but are not themselves incorporated into a growing polypeptide chain by the translation complex.

Examples of naturally-occurring amino acids that are not naturally-encoded include, but are not limited to, ((1H-imidazol-4-yl)methyl)glycine (NHis), S, 1-aminocyclobutane-1-carboxylic acid (1-aminocyclobutane-1-carboxylic acid; Acbu), 1-aminocyclopropane-1-carboxylic acid (1-aminocyclopropane-1-carboxylic acid; Acpr), Aminoisobutyric acid (2-amino-2-methylpropanoic acid; Aib), D-serine (dSer), αMethyl-Serine ((S)-2-amino-3-hydroxy-2-methylpropanoic acid (αMeSer); αMeSer), methyl-L-glutamine (N-MeGln), αMethyl-Glutamine ((S)-2,5-diamino-2-methyl-5-oxopentanoic acid; αMeGln), β-dimethylGln, αMethyl-Phenylalanine(S)-2-amino-2-methyl-3-phenylpropanoic acid; αMePhe), acetylated lysine N⁶-acteyl-L-lysine (Ac-Lys), Diphenylalanine ((S)-2-amino-3,3-diphenylpropanoic acid; Dip), β-dimethylarganine ((S)-2-amino-5-guanidino-3,3-dimethylpentaonic acid; β-dimethylArg), beta-cyclohexyl-L-alanine ((S)-2-amino-3-cyclohexylpropanoic acid; Cha), norleucine ((S)-2-aminohexanoic acid; Nle), D-glutamine (dGln) (S)-3-([1,1′-biphenyl]-4-yl)-2-aminopropanoic acid (Bip), 1-methyl-L-tryptophan (1-Methyl-Trp), and(S)-2-amino-3-(5-bromo-1/-indol-3-yl) propanoic acid (5-Br-Trp).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “polypeptide” is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with any of these terms. The term further includes polypeptides which have undergone post-translational or post-synthesis modifications, for example, conjugation of a palmitoyl group, glycosylation, acetylation, phosphorylation, amidation, derivatization by known protecting/blocking groups, proteolytic cleavage, or modification by non-naturally occurring amino acids.

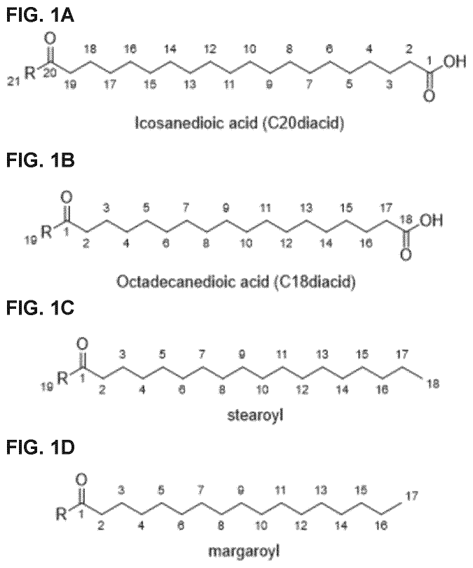

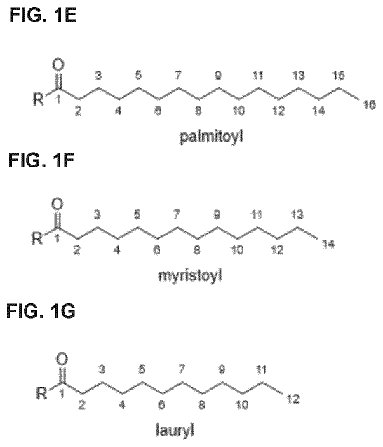

More specifically, the term “peptide” as used herein encompasses full length peptides and fragments, variants or derivatives thereof, e.g., a GLP-1/glucagon agonist peptide (e.g., 29, 30, or 31 amino acids in length). A “peptide” as disclosed herein, e.g., a GLP-1/glucagon agonist peptide, can be part of a fusion polypeptide comprising additional components such as, e.g., an Fc domain or an albumin domain, to increase half-life. A peptide as described herein can also be derivatized in a number of different ways. A peptide described herein can comprise modifications including e.g., conjugation of a lipid including a palmitoyl group, stearoyl group, lauryl group, myristoyl group, margaroyl group, octadecanedioic acid (C18diacid), or icosanedioic acid (C20diacid). Exemplary lipids are provided in FIGS. 1A-1G.

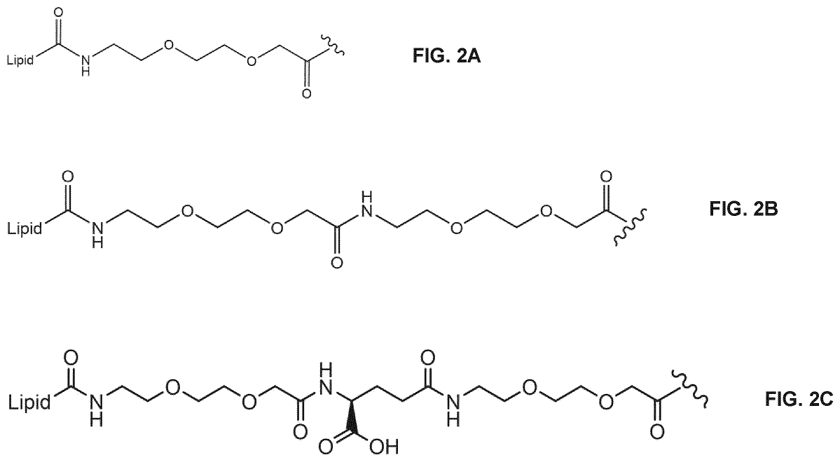

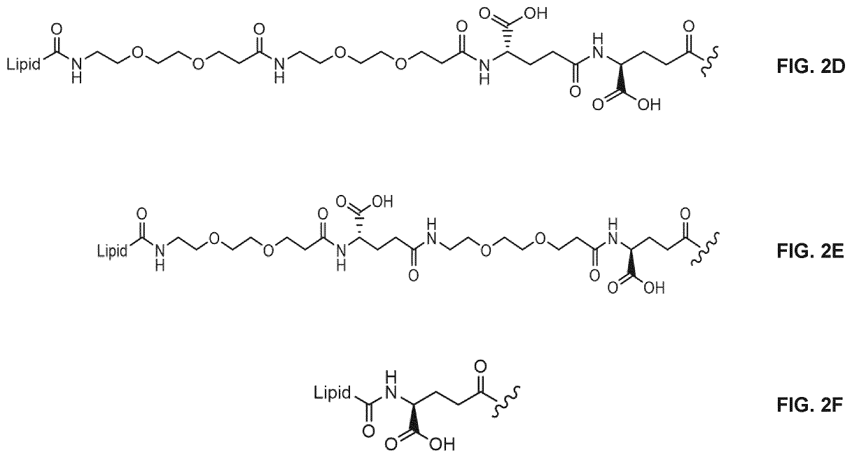

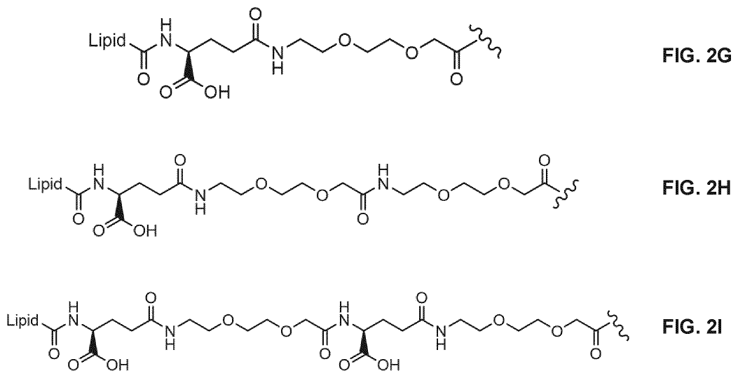

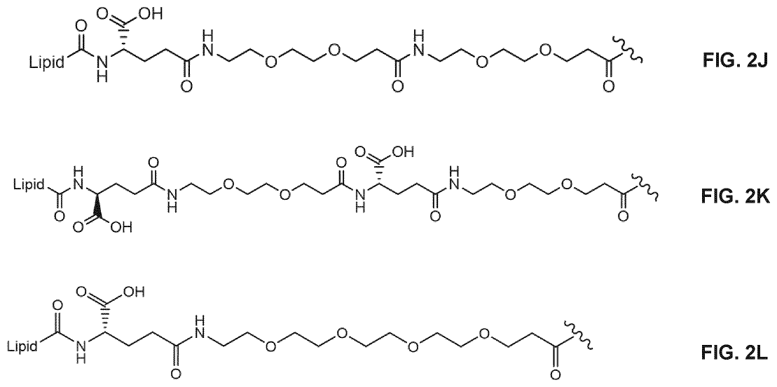

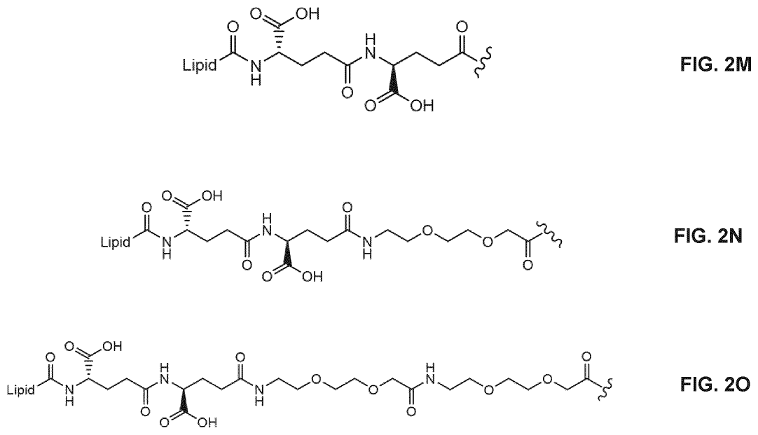

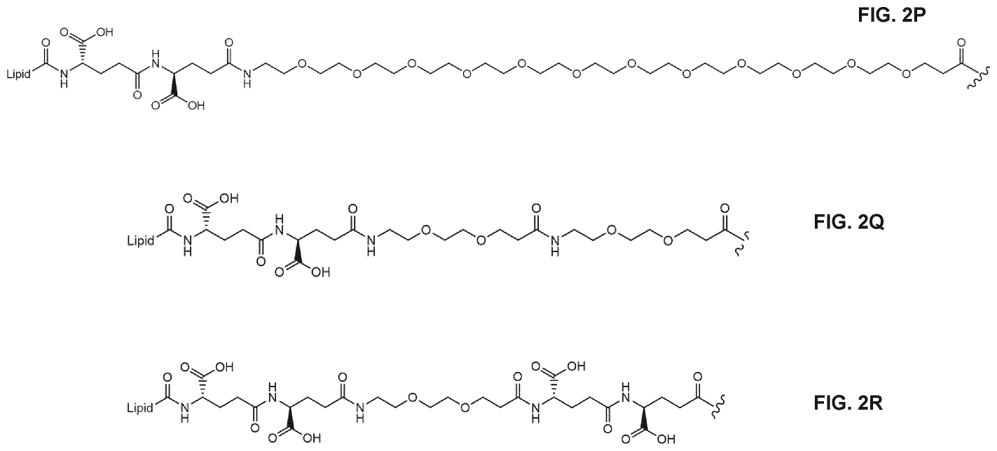

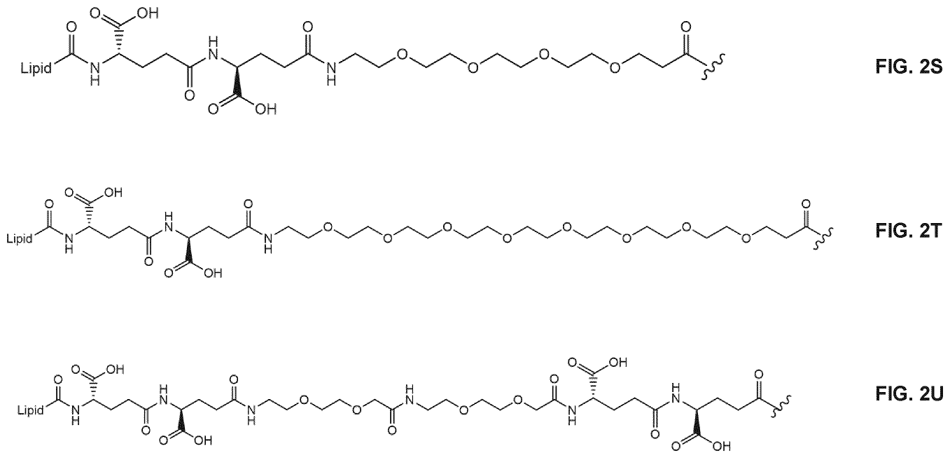

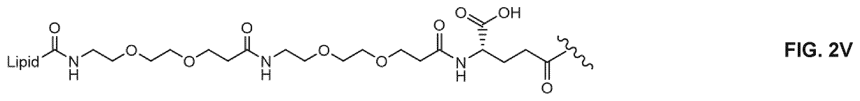

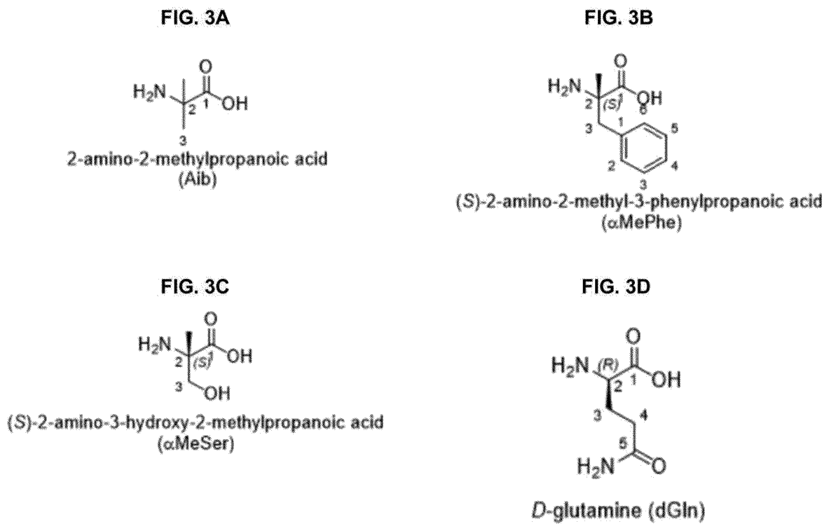

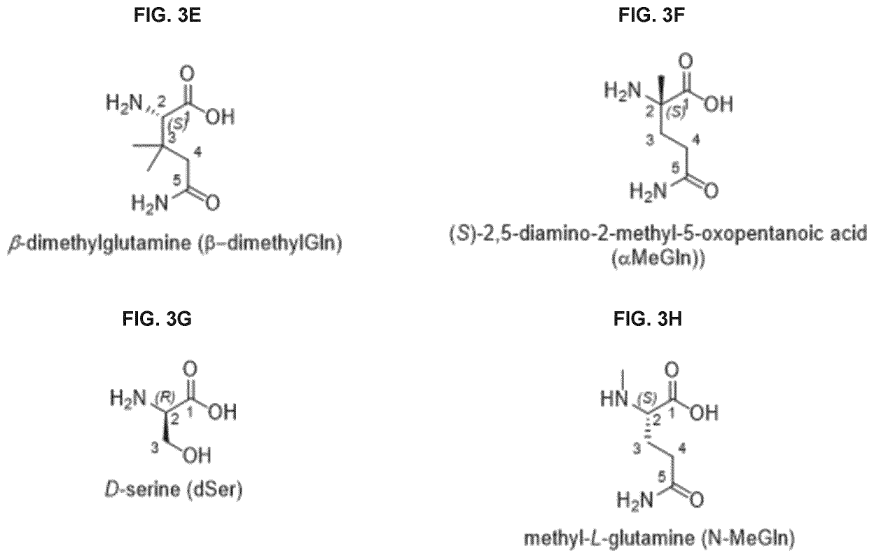

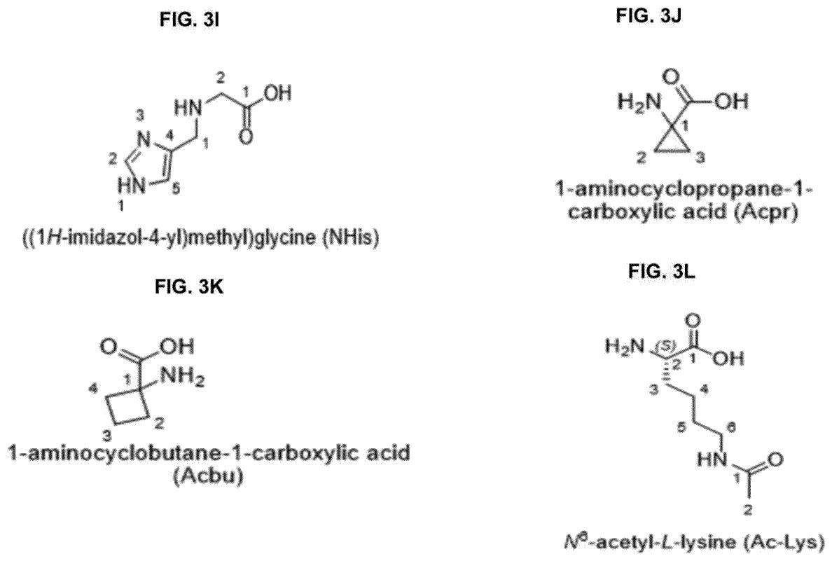

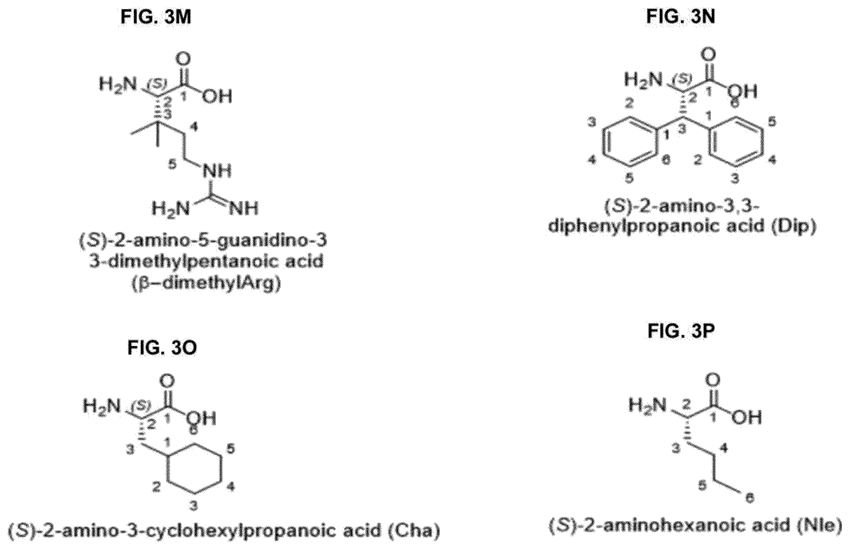

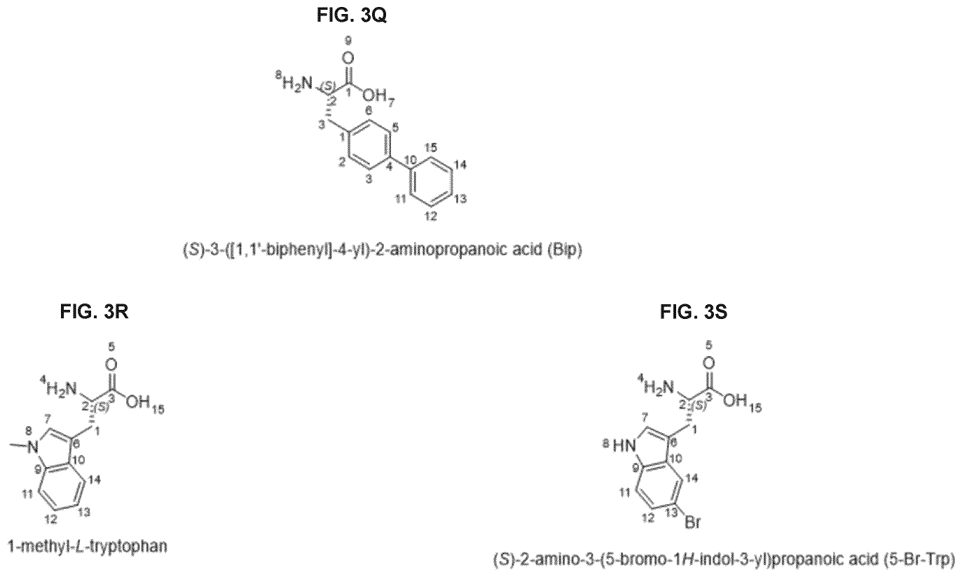

A peptide described herein can comprise modifications including e.g., conjugation of a linker comprising 2-(2-(2-aminoethoxy)ethosy)acetic acid (O2Oc), PEG, and/or gammaglutamic acid (γE). In some aspects, a linker comprises 2-(2-(2-aminoethoxy)ethosy) acetic acid (O2Oc), (O2Oc)-(O2Oc), (O2Oc)-γE-(O2Oc), 3-(2-(2-aminoethoxy)ethoxy)propanoic acid) ((PEG)2), 1-amino-3,6,9,12-tetraoxapentadecan-15-oic acid ((PEG)4), 1-amino-3,6,9,12,15,18,21,14-octaoxaheptacosan-27-oic acid ((PEG)8), 1-amino-3,6,9,12,15,18,21,24,27,30,33,36-dodecaoxanonatriacontan-39-oic acid ((PEG)12), gammaglutamic acid (γE), (PEG)2-(PEG)2-γE-γE, (PEG)2-γE-(PEG)2-γE, γE-(O2Oc), γE-(O2Oc)-(O2Oc), γE-(O2Oc)-γE-(O2Oc), γE-(PEG)2-(PEG)2, γE-(PEG)2-γE-(PEG)2, γE-(PEG)4, γE-γE, γE-γE-(O2Oc), γE-γE-(O2Oc)-(O2Oc), γE-γE-(PEG)12, γE-γE-(PEG)2-(PEG)2, γE-γE-(PEG)2-γE-γE, γE-γE-(PEG)4, γE-γE-(PEG)8, γE-γE-(O2Oc)-(O2Oc)-γE-γE, and (PEG)2-(PEG)2-γE. Exemplary linkers are provided in FIGS. 2A-2V.

The term “isolated” refers to the state in which peptides or nucleic acids, will generally be in accordance with the present disclosure. Isolated peptides and isolated nucleic acids will be free or substantially free of material with which they are naturally associated such as other peptides or nucleic acids with which they are found in their natural environment, or the environment in which they are prepared (e.g. cell culture) when such preparation is by recombinant DNA technology practiced in vitro or in vivo. Peptides and nucleic acid can be formulated with diluents or adjuvants and still for practical purposes be isolated—for example the peptides will normally be mixed with gelatin or other carriers if used to coat microtitre plates for use in immunoassays, or will be mixed with pharmaceutically acceptable carriers or diluents when used in diagnosis or therapy.

A “recombinant” peptide refers to a peptide produced via recombinant DNA technology. Recombinantly produced peptides expressed in host cells are considered isolated for the purpose of the present disclosure, as are native or recombinant polypeptides which have been separated, fractionated, or partially or substantially purified by any suitable technique.

The terms “fragment,” “analog,” “derivative,” or “variant” when referring to a GLP-1/glucagon agonist peptide include any peptide which retains at least some desirable activity, e.g., binding to glucagon receptors and/or GLP-1 receptors. Fragments of GLP-1/glucagon agonist peptides provided herein include proteolytic fragments, deletion fragments which exhibit desirable properties during expression, purification, and or administration to a subject.

The term “variant,” as used herein, refers to a peptide that differs from the recited peptide due to amino acid substitutions, deletions, insertions, and/or modifications. Variants can be produced using art-known mutagenesis techniques. Variants can also, or alternatively, contain other modifications. For example, a peptide can be conjugated or coupled, e.g., fused to a heterologous amino acid sequence or other moiety, e.g., for increasing half-life, solubility, or stability. Examples of moieties to be conjugated or coupled to a peptide provided herein include, but are not limited to, albumin, an immunoglobulin Fc region, polyethylene glycol (PEG), and the like. The peptide can also be conjugated or produced coupled to a linker or other sequence for ease of synthesis, purification or identification of the peptide (e.g., 6-His), or to enhance binding of the polypeptide to a solid support.

The terms “composition” or “pharmaceutical composition” refer to compositions containing a GLP-1/glucagon agonist peptide provided herein, along with e.g., pharmaceutically acceptable carriers, excipients, or diluents for administration to a subject in need of treatment, e.g., a human subject in need of improved glycemic control, weight loss, treatment of Type 2 Diabetes Mellitis, and/or treatment of NASH.

The term “pharmaceutically acceptable” refers to compositions that are, within the scope of sound medical judgment, suitable for contact with the tissues of human beings and animals without excessive toxicity or other complications commensurate with a reasonable benefit/risk ratio.

An “effective amount” is that amount of an agent provided herein (e.g., a GLP-1/glucagon agonist peptide), the administration of which to a subject, either in a single dose or as part of a series, is effective for treatment, e.g., for improved glycemic control, weight loss, treatment of Type 2 Diabetes Mellitis, and/or treatment of NASH.

As used herein, the terms “subject” and “patient” are used interchangeably. The subject can be an animal. In some aspects of the present disclosure, the subject is a mammal such as a non-human animal (e.g., cow, pig, horse, cat, dog, rat, mouse, monkey or other primate, etc.). In some aspects of the present disclosure, the subject is a cynomolgus monkey. In some aspects of the present disclosure, the subject is a human.

As used herein, a “subject in need thereof” or a “patient in need thereof” refers to an individual for whom it is desirable to treat, e.g., a subject in need of improved glycemic control, weight loss, treatment of Type 2 Diabetes Mellitis, and/or treatment of NASH.

Terms such as “treating” or “treatment” or “to treat” refer to therapeutic measures that cure and/or halt progression of a diagnosed pathologic condition or disorder. Terms such as “preventing” refer to prophylactic or preventative measures that prevent and/or slow the development of a targeted pathologic condition or disorder. Thus, those in need of treatment include those already with the disease or condition. Those in need of prevention include those prone to have the disease or condition and those in whom the disease or condition is to be prevented.

Terms such as “decreasing the severity” refer to therapeutic measures that slow down or lessen the symptoms of a diagnosed pathologic condition or disorder.

As used herein, a “GLP-1 agonist peptide” is a peptide that is not native GLP-1 but exhibits activity at the GLP-1 receptor of about at least 1% or more relative to native GLP-1, under the conditions of the CAMP assay (see Example 2). In some aspects, a GLP-1 agonist peptide exhibits activity at the GLP-1 receptor of at least 5%, 10%, 20%, 30%, 40%, 50%, 60%, 70%, 80%, 90%, 95%, or more relative to native GLP-1, under the conditions of the CAMP assay (see Example 2).

As used herein, a “glucagon agonist peptide” is a peptide that is not native glucagon but exhibits activity at the glucagon receptor of at least 1%, or more relative to native glucagon, under the conditions of the CAMP assay (see Example 2). In some aspects, a glucagon agonist peptide exhibits activity at the glucagon receptor of at least 5%, 10%, 20%, 30%, 40%, 50%, 60%, 70%, 80%, 90%, 95%, or more relative to native GLP-1, under the conditions of the CAMP assay (see Example 2).

As used herein a “GLP-1/glucagon agonist peptide,” “GLP-1/glucagon coagonist peptide,” “GLP-1 and glucagon dual agonist peptide” or “GLP-1 and glucagon dual coagonist peptide” is a peptide that it not native GLP-1 and is not native glucagon that exhibits activity at the glucagon receptor of at least 1% or more relative to native glucagon and also exhibits activity at the GLP-1 receptor of about at least 1% or more relative to native GLP-1, under the conditions of the CAMP assay (see Example 2). In some aspects, a “GLP-1/glucagon agonist peptide” or a “GLP-1 and glucagon dual agonist peptide” exhibits activity at the glucagon receptor of at least 5%, 10%, 20%, 30%, 40%, 50%, 60%, 70%, 80%, 90%, 95%, or more relative to native glucagon and also exhibits activity at the GLP-1 receptor of about at least 5%, 10%, 20%, 30%, 40%, 50%, 60%, 70%, 80%, 90%, 95%, or more relative to native GLP-1, under the conditions of the CAMP assay (see Example 2).

As used herein, term “relative potency ratio” refers to the % GLP-IR activity relative to GLP-1/% GlucR activity relative to glucagon.

As used herein the term “native glucagon” refers to naturally-occurring glucagon, e.g., human glucagon, comprising the sequence of SEQ ID NO: 1. The term “native GLP-1” refers to naturally-occurring GLP-1, e.g., human GLP-1, and is a generic term that encompasses, e.g., GLP-1 (7-36) amide (SEQ ID NO: 2), GLP-1 (7-37) acid (SEQ ID NO: 3), or a mixture of those two compounds. As used herein, a general reference to “glucagon” or “GLP-1” in the absence of any further designation is intended to mean native human glucagon or native human GLP-1, respectively. Unless otherwise indicated, “glucagon” refers to human glucagon, and “GLP-1” refers to human GLP-1.

The term “sequence identity” as used herein refers to a relationship between two or more polynucleotide sequences or between two or more polypeptide sequences. When a position in one sequence is occupied by the same nucleic acid base or amino acid in the corresponding position of the comparator sequence, the sequences are said to be “identical” at that position. The percentage “sequence identity” is calculated by determining the number of positions at which the identical nucleic acid base or amino acid occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window. In order to optimally align sequences for comparison, the portion of a polynucleotide or polypeptide sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which was available from the National Center for Biotechnology Information as of Sep. 1, 2004, which program incorporates the programs BLASTN (for nucleotide sequence comparison) and BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” parameters that were default parameters as of Sep. 1, 2004, can be used for word size (3), open gap penalty (11), extension gap penalty (1), gap drop-off (50), expect value (10), and any other required parameter including but not limited to matrix option.

Glucagon
(SEQ ID NO: 1)
HSQGTFTSDYSKYLDSRRAQDFVQWLMNT-acid
GLP-1(7-36) amide
(SEQ ID NO: 2)
HAEGTFTSDVSSYLEGQAAKEFIAWLVKGR-amide
GLP-1(7-37) acid
(SEQ ID NO: 3)
HAEGTFTSDVSSYLEGQAAKEFIAWLVKGRG-acid

### II. GLP-1/Glucacon Agonist Peptides

Provided herein are peptides that bind both to a glucagon receptor and to a GLP-1 receptor. In some aspects, the peptides provided herein are co-agonists (dual agonists) of glucagon and GLP-1 activity. Such peptides are referred to herein as GLP-1/glucagon agonist peptides. In some aspects, GLP-1/glucagon agonist peptides as provided herein are active at the human GLP1 and human glucagon receptors.

In certain aspects, GLP-1/glucagon agonist peptides provided herein exhibit in vitro potencies at the GLP-1 receptor as shown by an EC50 in the CAMP assay (see Example 2) of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, or less than 2 pM.

In certain aspects, GLP-1/glucagon agonist peptides provided herein exhibit in vitro potencies at the glucagon receptor as shown by an EC50 in the CAMP assay (see Example 2) of less than 10,000 pM, less than 5000 pM, less than 2500 pM, less than 1000 pM, less than 900 pM, less than 800 pM, less than 700 pM, less than 600 pM, less than 500 pM, less than 400 pM, less than 300 pM, less than 200 pM, less than 100 pM, less than 50 pM, less than 25 pM, less than 20 pM, less than 15 pM, less than 10 pM, less than 5 pM, less than 4 pM, less than 3 pM, or less than 2 pM.

In certain aspects, GLP-1/glucagon agonist peptides provided herein have a hGLP-1R/hGCGR relative potency ratio of about 1 to about 25. In certain aspects, GLP-1/glucagon agonist peptides provided herein have a hGLP-1R/hGCGR relative potency ratio of about 1 to about 20. In certain aspects, GLP-1/glucagon agonist peptides provided herein have a hGLP-1R/hGCGR relative potency ratio of about 1 to about 15. In certain aspects, GLP-1/glucagon agonist peptides provided herein have a hGLP-1R/hGCGR relative potency ratio of about 1 to about 10.

In certain aspects, GLP-1/glucagon agonist peptides provided herein have a hGLP-1R/hGCGR relative potency ratio of about 2 to about 25. In certain aspects, GLP-1/glucagon agonist peptides provided herein have a hGLP-1R/hGCGR relative potency ratio of about 2 to about 20. In certain aspects, GLP-1/glucagon agonist peptides provided herein have a hGLP-1R/hGCGR relative potency ratio of about 2 to about 15. In certain aspects, GLP-1/glucagon agonist peptides provided herein have a hGLP-1R/hGCGR relative potency ratio of about 2 to about 10.

In certain aspects, GLP-1/glucagon agonist peptides provided herein, when administered to mice at 10 nmol/kg (as performed in Example 3) reduce 24-hour food intake by at least 10% relative to mice treated with a vehicle control. In certain aspects, GLP-1/glucagon agonist peptides provided herein, when administered to mice at 10 nmol/kg (as performed in Example 3) reduce 24-hour food intake by at least 20% relative to mice treated with a vehicle control. In certain aspects, GLP-1/glucagon agonist peptides provided herein, when administered to mice at 10 nmol/kg (as performed in Example 3) reduce 24-hour food intake by at least 30% relative to mice treated with a vehicle control.

In certain aspects, GLP-1/glucagon agonist peptides provided herein, when administered to mice at 10 nmol/kg (as performed in Example 3) reduce 24-hour food intake by at 10-70% relative to mice treated with a vehicle control. In certain aspects, GLP-1/glucagon agonist peptides provided herein, when administered to mice at 10 nmol/kg (as performed in Example 3) reduce 24-hour food intake by at least 20% or by 20-70% relative to mice treated with a vehicle control. In certain aspects, GLP-1/glucagon agonist peptides provided herein, when administered to mice at 10 nmol/kg (as performed in Example 3) reduce 24-hour food intake by at least 30% or by 30-70% relative to mice treated with a vehicle control.

In certain aspects, GLP-1/glucagon agonist peptides provided herein are stable. For example, in certain aspects, GLP-1/glucagon agonist peptides provided herein stable in the presence of FasSSGF (fasted state simulated gastric fluid) such that at least 25% of the GLP-1/glucagon agonist peptide remains intact after incubation with FasSSGF for 30 minutes (as performed in Example 4.) In certain aspects, GLP-1/glucagon agonist peptides provided herein stable in the presence of FasSSGF such that at least 40% of the GLP-1/glucagon agonist peptide remains intact after incubation with FasSSGF for 30 minutes (as performed in Example 4.) In certain aspects, GLP-1/glucagon agonist peptides provided herein stable in the presence of FasSSGF such that at least 50% of the GLP-1/glucagon agonist peptide remains intact after incubation with FasSSGF for 30 minutes (as performed in Example 4.) In certain aspects, GLP-1/glucagon agonist peptides provided herein stable in the presence of FasSSGF such that at least 60% of the GLP-1/glucagon agonist peptide remains intact after incubation with FasSSGF for 30 minutes (as performed in Example 4.) In certain aspects, GLP-1/glucagon agonist peptides provided herein stable in the presence of FasSSGF such that at least 70% of the GLP-1/glucagon agonist peptide remains intact after incubation with FasSSGF for 30 minutes (as performed in Example 4.)

In certain aspects, GLP-1/glucagon agonist peptides provided herein stable in the presence of neprilysin such that at least 55% of the GLP-1/glucagon agonist peptide remains intact after incubation with neprilysin for 24 hours (as performed in Example 5.) In certain aspects, GLP-1/glucagon agonist peptides provided herein stable in the presence of neprilysin such that at least 60% of the GLP-1/glucagon agonist peptide remains intact after incubation with neprilysin for 24 hours (as performed in Example 5.) In certain aspects, GLP-1/glucagon agonist peptides provided herein stable in the presence of neprilysin such that at least 65% of the GLP-1/glucagon agonist peptide remains intact after incubation with neprilysin for 24 hours (as performed in Example 5.) In certain aspects, GLP-1/glucagon agonist peptides provided herein stable in the presence of neprilysin such that at least 70% of the GLP-1/glucagon agonist peptide remains intact after incubation with neprilysin for 24 hours (as performed in Example 5.) In certain aspects, GLP-1/glucagon agonist peptides provided herein stable in the presence of neprilysin such that at least 75% of the GLP-1/glucagon agonist peptide remains intact after incubation with neprilysin for 24 hours (as performed in Example 5.) In certain aspects, GLP-1/glucagon agonist peptides provided herein stable in the presence of neprilysin such that at least 80% of the GLP-1/glucagon agonist peptide remains intact after incubation with neprilysin for 24 hours (as performed in Example 5.) In certain aspects, GLP-1/glucagon agonist peptides provided herein stable in the presence of neprilysin such that at least 85% of the GLP-1/glucagon agonist peptide remains intact after incubation with neprilysin for 24 hours (as performed in Example 5.) In certain aspects, GLP-1/glucagon agonist peptides provided herein stable in the presence of neprilysin such that at least 90% of the GLP-1/glucagon agonist peptide remains intact after incubation with neprilysin for 24 hours (as performed in Example 5.) In certain aspects, GLP-1/glucagon agonist peptides provided herein stable in the presence of neprilysin such that at least 95% of the GLP-1/glucagon agonist peptide remains intact after incubation with neprilysin for 24 hours (as performed in Example 5.)

In certain aspects, GLP-1/glucagon agonist peptides provided herein are stable. For example, in certain aspects, GLP-1/glucagon agonist peptides provided herein stable in the presence of FasSSIF (fasted state simulated intestinal fluid)/Pancreatin such that at least 10% of the GLP-1/glucagon agonist peptide remains intact after incubation with FasSSIF/Pancreatin for 30 minutes (as performed in Example 6.) In certain aspects, GLP-1/glucagon agonist peptides provided herein are stable. For example, in certain aspects, GLP-1/glucagon agonist peptides provided herein stable in the presence of FasSSIF/Pancreatin such that at least 20% of the GLP-1/glucagon agonist peptide remains intact after incubation with FasSSIF/Pancreatin for 30 minutes (as performed in Example 6.) In certain aspects, GLP-1/glucagon agonist peptides provided herein are stable. For example, in certain aspects, GLP-1/glucagon agonist peptides provided herein stable in the presence of FasSSIF/Pancreatin such that at least 25% of the GLP-1/glucagon agonist peptide remains intact after incubation with FasSSIF/Pancreatin for 30 minutes (as performed in Example 6.) In certain aspects, GLP-1/glucagon agonist peptides provided herein are stable. For example, in certain aspects, GLP-1/glucagon agonist peptides provided herein stable in the presence of FaSSSIF/Pancreatin such that at least 50% of the GLP-1/glucagon agonist peptide remains intact after incubation with FasSSSIF/Pancreatin for 30 minutes (as performed in Example 6.) In certain aspects, GLP-1/glucagon agonist peptides provided herein are stable. For example, in certain aspects, GLP-1/glucagon agonist peptides provided herein stable in the presence of FasSSIF/Pancreatin such that at least 75% of the GLP-1/glucagon agonist peptide remains intact after incubation with FasSSIF/Pancreatin for 30 minutes (as performed in Example 6.) In certain aspects, GLP-1/glucagon agonist peptides provided herein are stable. For example, in certain aspects, GLP-1/glucagon agonist peptides provided herein stable in the presence of FasSSIF/Pancreatin such that at least 80% of the GLP-1/glucagon agonist peptide remains intact after incubation with FasSSIF/Pancreatin for 30 minutes (as performed in Example 6.) In certain aspects, GLP-1/glucagon agonist peptides provided herein are stable. For example, in certain aspects, GLP-1/glucagon agonist peptides provided herein stable in the presence of FasSSIF/Pancreatin such that at least 90% of the GLP-1/glucagon agonist peptide remains intact after incubation with FasSSIF/Pancreatin for 30 minutes (as performed in Example 6.)

A GLP-1/glucagon agonist peptide as disclosed herein can comprise a heterologous moiety, e.g., to extend half-life. The heterologous moiety can be a protein, a peptide, a protein domain, a linker, an organic polymer, an inorganic polymer, a polyethylene glycol (PEG), biotin, an albumin, a human serum albumin (HSA), a HSA FcRn binding portion, an antibody, a domain of an antibody, an antibody fragment, a single chain antibody, a domain antibody, an albumin binding domain, an enzyme, a ligand, a receptor, a binding peptide, a non-FnIII scaffold, an epitope tag, a recombinant polypeptide polymer, a cytokine, and a combination of two or more of such moieties.

In some aspects, a GLP-1/glucagon agonist peptide as disclosed herein binds to a GLP-1 receptor (GLP-1R), binds to a glucagon receptor (GCGR), or binds to both a GLP-1R and a GCGR. In some aspects, the GLP-1R is human GLP-1R. In some aspects, the GCGR is human GCGR. In some aspects, the peptide is an agonist of GLP-1 activity, an agonist of glucagon activity, or an agonist of both GLP-1 and glucagon activity.

In some aspects, a GLP-1/glucagon agonist peptide as disclosed herein has increased proteolytic-resistance relative to the natural ligand of the GLP-1R and/or GCGR. In some aspects, a GLP-1/glucagon agonist peptide as disclosed herein has increased proteolytic-resistance relative to cotadutide (SEQ ID NO: 358). In some aspects, a GLP-1/glucagon agonist peptide as disclosed herein has increased proteolytic-resistance relative to semaglutide (SEQ ID NO: 359). In some aspects, a GLP-1/glucagon agonist peptide as disclosed herein has increased proteolytic-resistance relative to cotadutide (SEQ ID NO: 358) and semaglutide (SEQ ID NO: 359).

In some aspects, a GLP-1/glucagon agonist peptide provided herein comprises the sequence: X1-X2-X3-G-X5-X6-T-S-D-X10-S-X12-X13-L-X15-X16-X17-X18-X19-X20-X21-X22-X23-X24-X25-X26-X27-X28-X29-X30-X31-Z, wherein X1 is H or NHis, X2 is S, 1-aminocyclobutane-1-carboxylic acid (Acbu), 1-aminocyclopropane-1-carboxylic acid (Acpr), Aminoisobutyric acid (Aib), D-serine (dSer), or Methyl-Serine (αMeSer), X3 is Q, H, I, D-glutamine (dGln), methyl-L-glutamine (N-MeGln), α-Methyl-glutamine (αMeGln), or β-dimethylglutamine (β-dimethylGln), X5 is T or S, X6 is F or αMethyl-Phenylalanine (αMePhe), X10 is Y, K, or V, wherein the K can comprise an acyl moiety and/or can be lipidated, X12 is K, acetylated lysine (Ac-Lys), E, or R, X13 is Y, Aib, αMethyl-Phenylalanine (αMePhe), Diphenylalanine (Dip), I, or K, wherein the K can comprise an acyl moiety and/or can be lipidated, X15 is D or E, X16 is S, Aib, E, T, R, A, K, L, or V, X17 is R, E, K, Q, or β-dimethylarganine (β-dimethylArg), wherein the K can comprise an acyl moiety and/or can be lipidated, X18 is R, A, Aib, Q, S, or β-dimethylArg, X19 is A or V, X20 is Q, Aib, E, K, L, or R, wherein the K can comprise an acyl moiety and/or can be lipidated, X21 is D, E, or L, X22 is F, I, or αMePhe, X23 is V or I, X24 is Q, A, E, K, L, or R, wherein the K can comprise an acyl moiety and/or can be lipidated, X25 is W, Aib, Dip, H, I, S, biphenyl-alanine (Bip), 1-methyl tryptophan (1-Methyl-Trp), 5-Bromo trptophan (5-BrTrp), or αMePhe, X26 is L, beta-cyclohexyl-L-alanine (Cha), I, or V, X27 is M, A, E, I, L, norleucine (Nle), S, K, or V, X28 is N, (PEG)4, A, Aib, E, G, R, S, or not present, X29 is T, Aib, E, G, A, R, or not present, X30 is not present, E, A, Aib, K, T, or G, X31 is not present, I, or G, and Z is amide or acid (SEQ ID NO: 4), wherein the peptide does not comprise SEQ ID NO: 1 and does not comprise HSQGTFTSDX10SEYLDSERARDFVAWLEAGG-acid, wherein X10-Lys[ε-γE-Palmitoyl] (SEQ ID NO: 538). In some aspects, X2 is Aib and/or X10 is V. In some aspects, X3 is Q, X15 is D, X18 is R, X20 is R, X21 is D, X23 is V, and/or X30 is G. In some aspects, X13 is αMePhe, X16 is T, X17 is K, X27 is L, X28 is E, and/or X29 is Aib.

In some aspects, the residue at position 10, 13, 17, 20, or 24 is acylated. In some aspects, the residue at position 10, 13, 17, 20, or 24 is lipidated. In some aspects, the lipid is selected from the group consisting of a palmitoyl group, stearoyl group, lauryl group, myristoyl group, margaroyl group, arachidoyl group, octadecanedioic acid (C18diacid), and icosanedioic acid (C20diacid).

In some aspects, the lipid attached to the residue at position 10, 13, 17, 20, or 24 is attached via a linker. In some aspects, the linker is selected from the group consisting of (O2Oc), (O2Oc)-(O2Oc), (O2Oc)-γE-(O2Oc), (PEG)2-(PEG)2-γE-γE, (PEG)2-γE-(PEG)2-γE, γE, γE-(O2Oc), γE-(O2Oc)-(O2Oc), γE-(O2Oc)-γE-(O2Oc), γE-(PEG)2-(PEG)2, γE-(PEG)2-γE-(PEG)2, γE-(PEG)4, γE-γE, γE-γE-(O2Oc), γE-γE-(O2Oc)-(O2Oc), γE-γE-(PEG)12, γE-γE-(PEG)2-(PEG)2, γE-γE-(PEG)2-γE-γE, γE-γE-(PEG)4, (PEG)2-(PEG)2-γE, γE-γE-(O2Oc)-(O2Oc)-γE-γE, and γE-γE-(PEG)8.

In some aspects, the linker is linked to the epsilon amino group of the residue at position 10, 13, 17, 20, and/or 24.

In some aspects, the peptide comprises any one of SEQ ID NOs: 6-411 and 418-537. In some aspects, the peptide comprises SEQ ID NO: 99. In some aspects, the peptide comprises SEQ ID NO: 106. In some aspects, the peptide comprises SEQ ID NO: 228. In some aspects, the peptide comprises SEQ ID NO: 233.

In some aspects, the peptide comprises the sequence: H-X2-X3-G-X5-X6-T-S-D-X10-S-X12-X13-L-X15-X16-X17-X18-A-X20-D-X22-X23-X24-X25-X26-X27-X28-X29-X30-X31-Z, wherein X2 is S, Aminoisobutyric acid (Aib), or αMethyl-Serine (αMeSer), X3 is Q or H, X5 is T or S, X6 is F or αMethyl-Phenylalanine (αMePhe), X10 is Y or V, X12 is K or acetylated lysine (Ac-Lys), X13 is Y, αMePhe, Aib, Diphenylalanine (Dip), or I, X15 is D or E, X16 is S, T, A, E, K, L, R, or V, X17 is R or K, wherein the K can comprise an acyl moiety and/or can be lipidated, X18 is R, A, Q, or β-dimethylarganine (β-dimethylArg), X20 is Q, R, Aib, L, or E, X22 is F, I, or αMePhe, X23 is V or I, X24 is Q, E, A, L, or R, X25 is W, Aib, S, Dip, I, H, biphenyl-alanine (Bip), 1-methyl tryptophan (1-Methyl-Trp), 5-Bromo trptophan (5-BrTrp), or αMePhe, X26 is L, I, or beta-cyclohexyl-L-alanine (Cha) or V, X27 is M, A, L, E, V, I, K, norleucine (Nle), or S, X28 is N, Aib, E, (PEG)4, A, S, or G, X29 is T, not present, Aib, G, A, R, or E, X30 is not present, G, A, Aib, K, or E, X31 is not present, and Z is amide or acid (SEQ ID NO: 5), wherein the peptide does not comprise SEQ ID NO: 1.

In some aspects, the residue at position 17 is acylated. In some aspects, the residue at position 17 is lipidated. In some aspects, the lipid is selected from the group consisting of a palmitoyl group, stearoyl group, lauryl group, myristoyl group, margaroyl group, arachidoyl group, octadecanedioic acid (C18diacid), and icosanedioic acid (C20diacid).

In some aspects, the lipid attached to the residue at position 17 is attached via a linker. In some aspects, the linker is selected from the group consisting of (O2Oc), (O2Oc)-(O2Oc), (O2Oc)-γE-(O2Oc), (PEG)2-(PEG)2-γE-γE, (PEG)2-γE-(PEG)2-γE, γE, γE-(O2Oc), γE-(O2Oc)-(O2Oc), γE-(O2Oc)-γE-(O2Oc), γE-(PEG)2-(PEG)2, γE-(PEG)2-γE-(PEG)2, γE-(PEG)4, γE-γE, γE-γE-(O2Oc), γE-γE-(O2Oc)-(O2Oc), γE-γE-(PEG)12, γE-γE-(PEG)2-(PEG)2, γE-γE-(PEG)2-γE-γE, γE-γE-(PEG)4, and γE-γE-(PEG)8. In some aspects, the linker is selected from the group consisting of γE-γE-(O2Oc)-(O2Oc)-γE-γE, (O2Oc)-(O2Oc), γE, γE-(O2Oc)-(O2Oc), γE-γE-(O2Oc)-(O2Oc), γE-γE-(PEG)4, γE-γE-(PEG)2-(PEG)2, γE-γE-(PEG)8, γE-γE-(PEG)12, (PEG)2-(PEG)2-γE-γE, (PEG)2-γE-(PEG)2-γE, γE-(PEG)2-γE-(PEG)2, γE-(PEG)2-(PEG)2, (PEG)2-(PEG)2-γE, and γE-(PEG)4.

In some aspects, the linker is linked to the epsilon amino group of the residue at position 17.

In some aspects, the peptide comprises the sequence: H-Aminoisobutyric acid (Aib)-Q-G-T-X6-T-S-D-V-S-K-αMethyl-Phenylalanine (αMePhe)-L-X15-X16-K-X18-A-X20-X21-X22-X23-X24-W-X26-X27-X28-X29-X30-X31-Z, wherein X6 is F or αMethyl-Phenylalanine (αMePhe), X15 is E or D, X16 is T, S, K, E, A, L, or R, X18 is R or A, X20 is R, Q, or L, X21 is D or E, X22 is F or αMePhe, X23 is V or I, X24 is R, A, Q, or L, X25 is W, αMePhe biphenyl-alanine (Bip), 1-methyl tryptophan (1-Methyl-Trp), 5-Bromo trptophan (5-BrTrp), or Aib, X26 is L, I, or V, X27 is L, A, E, V, I, or K, X28 is E, S, A, Aib, not present, or R, X29 is G, Aib, R, T, E, A, or not present, X30 is G, Aib, E, A, K, or not present, X31 is I or not present, and or G, and Z is amide or acid (SEQ ID NO: 412).

In some aspects, the lysine at position 17 is acylated. In some aspects, the lysine at position 17 is lipidated. In some aspects, the lipid is selected from the group consisting of a palmitoyl group, stearoyl group, lauryl group, myristoyl group, margaroyl group, arachidoyl group, octadecanedioic acid (C18diacid), and icosanedioic acid (C20diacid). In some aspects, the lipid is selected from the group consisting of octadecanedioic acid (C18diacid) and icosanedioic acid (C20diacid). In some aspects, the lipid is a octadecanedioic acid (C18diacid). In some aspects, the lipid is an icosanedioic acid (C20diacid).

In some aspects, the lipid is linked to the lysine at position 17 via a linker. In some aspects, the linker is selected from the group consisting of (O2Oc), (O2Oc)-(O2Oc), (O2Oc)-YE-(O2Oc), (PEG)2-(PEG)2-γE-γE, (PEG)2-γE-(PEG)2-γE, γE, γE-(O2Oc), γE-(O2Oc)-(O2Oc), γE-(O2Oc)-γE-(O2Oc), γE-(PEG)2-(PEG)2, γE-(PEG)2-γE-(PEG)2, γE-(PEG)4, γE-γE, γE-γE-(O2Oc), γE-γE-(O2Oc)-(O2Oc), γE-γE-(PEG)12, γE-γE-(PEG)2-(PEG)2, γE-γE-(PEG)2-γE-γE, γE-γE-(PEG)4, PEG)2-(PEG)2-γE, γE-γE-(O2Oc)-(O2Oc), and γE-γE-(PEG)8. In some aspects, the peptide of claim X, wherein the linker is selected from the group consisting of (PEG)2-(PEG)2-γE, (O2Oc)-(O2Oc), γE, γE-(O2Oc)-(O2Oc), γE-γE-(O2Oc)-(O2Oc), and γE-γE (O2Oc)-(O2Oc)-γE-γE. In some aspects, the linker is γE-(O2Oc)-(O2Oc).

In some aspects, the linker is linked to the epsilon amino group of the residue at position 17.

In certain aspects, GLP-1/glucagon agonist peptides as disclosed have desirable potencies at the glucagon and GLP-1 receptors, and have desirable relative potencies for promoting weight loss.

In some aspects, the peptide has the sequence of any one of SEQ ID NOs: 6-206 and 418-531. In some aspects, the peptide comprises SEQ ID NO: 99. In some aspects, the peptide comprises SEQ ID NO: 106.

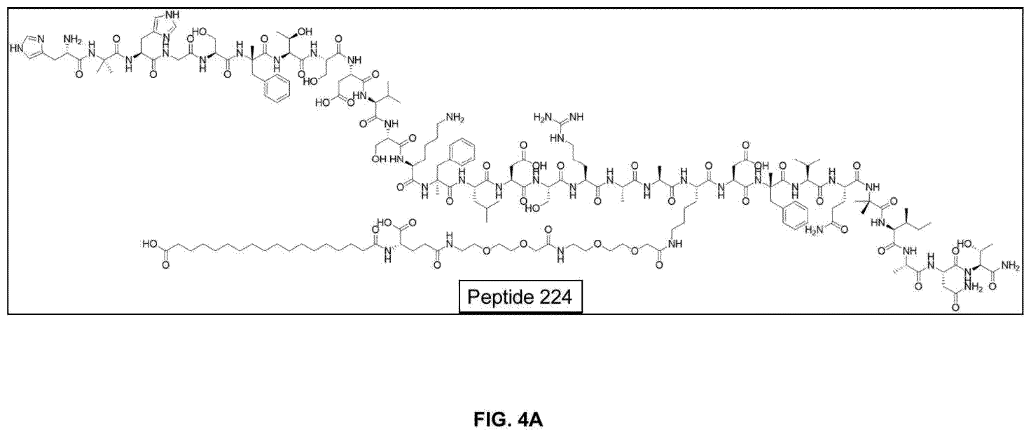

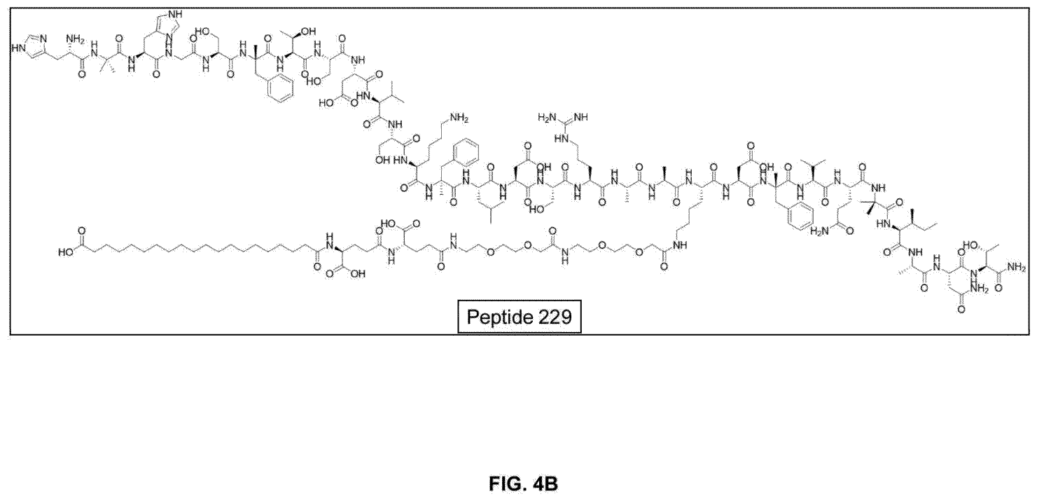

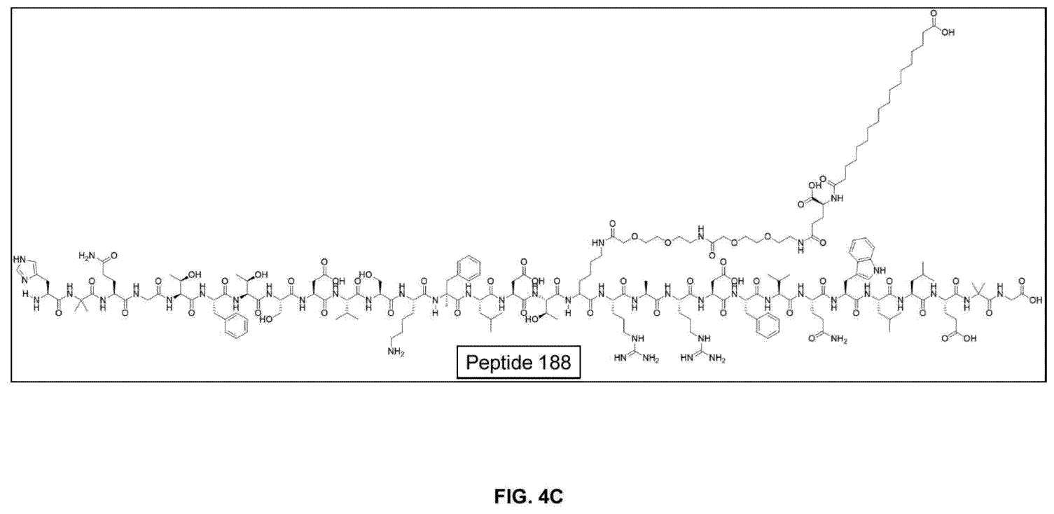

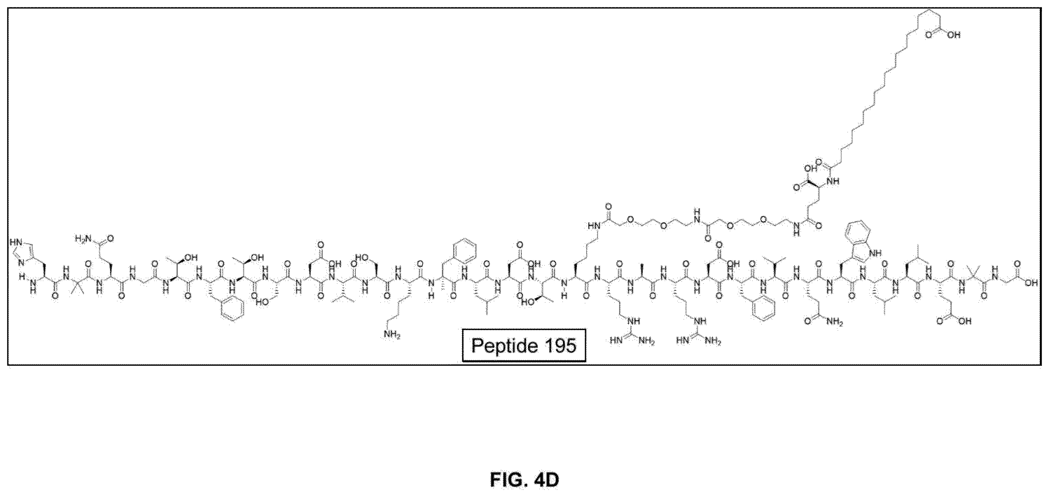

In some aspects, the peptide has the structure of any one of the structures depicted in FIGS. 4C-4D. In some aspects, the peptide has the structure of FIG. 4C. In some aspects, the peptide has the structure of FIG. 4D.

In some aspects, the peptide is any one of the peptides in Table 1.

TABLE 1
Peptides Modified at Position 17

| | | | | Linker | | | Sequence |
|---|---|---|---|---|---|---|---|
| | | SEQ | Albumin | (described | Acyl- | | modification |
| | | ID | binding | N→C | ation | C- | with respect |
| Peptide | Sequence | NO | moiety | term.) | site | term. | to glucagon. |
| Peptide | HSQGS(αMePhe)TSDVS | 6 | C18diacid | γE- | 17 | Amide | 5S, |
| 1 | K(Aib)LDSK(O2Oc- | | | (O2Oc)- | | | 6αMePhe, |
| | O2Oc-γE- | | | (O2Oc) | | | 10V, 13Aib, |
| | C18diacid)17AAQD | | | | | | 17K, 18A, |
| | (αMePhe)VQ(Aib)IAN- | | | | | | 22αMePhe, |
| | amide | | | | | | 25Aib, 26I, |
| | | | | | | | 27A, des29T |
| Peptide | HSQGS(αMePhe)TSDVS | 7 | C20diacid | γE- | 17 | Amide | 5S, |
| 2 | K(αMePhe)LDSK(O2Oc- | | | (O2Oc)- | | | 6αMePhe, |
| | O2Oc-γE- | | | (O2Oc) | | | 10V, |
| | C20diacid)17AAQD | | | | | | 13αMePhe, |
| | (αMePhe)VQWIANT-amide | | | | | | 17K, 18A, |
| | | | | | | | 22αMePhe, |
| | | | | | | | 26I, 27A |
| Peptide | HSQGS(αMePhe)TSDVS | 8 | C18diacid | γE | 17 | Amide | 5S, |
| 3 | K(αMePhe)LDSK(γΕ- | | | | | | 6αMePhe, |
| | C18diacid)17AAQDFVQ | | | | | | 10V, |
| | WIANT-amide | | | | | | 13αMePhe, |
| | | | | | | | 17K, 18A, |
| | | | | | | | 26I, 27A |
| Peptide | HSQGS(αMePhe)TSDVS | 9 | C18diacid | γE- | 17 | Amide | 5S, |
| 4 | K(αMePhe)LDSK(O2Oc- | | | (O2Oc)- | | | 6αMePhe, |
| | O2Oc-γE- | | | (O2Oc) | | | 10V, |
| | C18diacid)17AAQDFVQ | | | | | | 13αMePhe, |
| | WIANT-amide | | | | | | 17K, 18A, |
| | | | | | | | 26I, 27A |
| Peptide | H(Aib)QGS(αMePhe)TS | 10 | C18diacid | γE-γE- | 17 | Amide | 2Aib, 5S, |
| 5 | DVSK(αMePhe)LDSK | | | (O2Oc)- | | | 6αMePhe, |
| | (O2Oc-O2Oc-γE-yE- | | | (O2Oc) | | | 10V, |
| | C18diacid)17AAQD | | | | | | 13αMePhe, |
| | (αMePhe)VQ(Aib)IAN- | | | | | | 17K, 18A, |
| | amide | | | | | | 22αMePhe, |
| | | | | | | | 25Aib, 26I, |
| | | | | | | | 27A, des29T |
| Peptide | H(Aib)HGS(αMePhe)TS | 11 | C18diacid | γE- | 17 | Amide | 2Aib, 3H, 5S, |
| 6 | DVSK(αMePhe)LDSK | | | (O2Oc)- | | | 6αMePhe, |
| | (O2Oc-O2Oc-γE- | | | (O2Oc) | | | 10V |
| | C18diacid)17AAQD | | | | | | 13αMePhe, |
| | (αMePhe)VEWIAN-amide | | | | | | 17K, 18A, |
| | | | | | | | 22αMePhe, |
| | | | | | | | 24E, 26I, |
| | | | | | | | 27A, des29T |
| Peptide | H(Aib)HGS(αMePhe)TS | 12 | C18diacid | γE | 17 | Amide | 2Aib, 3H, 5S, |
| 7 | DVSK(αMePhe)LDSK(γE- | | | | | | 6αMePhe, |
| | C18diacid)17AAQD | | | | | | 10V, |
| | (αMePhe)VEWIANT-amide | | | | | | 13αMePhe, |
| | | | | | | | 17K, 18A, |
| | | | | | | | 22αMePhe, |
| | | | | | | | 24E, 26I, 27A |
| Peptide | H(Aib)HGS(αMePhe)TS | 13 | C18diacid | γE- | 17 | Amide | 2Aib, 3H, 5S, |
| 8 | DVSK(αMePhe)LDSK | | | (O2Oc)- | | | 6αMePhe, |
| | (O2Oc-O2Oc-γE- | | | (O2Oc) | | | 10V, |
| | C18diacid)17AAQDFVQ | | | | | | 13αMePhe, |
| | WIANT-amide | | | | | | 17K, 18A, |
| | | | | | | | 26I, 27A |
| Peptide | H(Aib)HGS(αMePhe)TS | 14 | C18diacid | γE- | 17 | Amide | 2Aib, 3H, 5S, |
| 9 | DVSK(αMePhe)LDSK | | | (O2Oc)- | | | 6αMePhe, |
| | (O2Oc-O2Oc-γE- | | | (O2Oc) | | | 10V, |
| | C18diacid)17AARDFVQ | | | | | | 13αMePhe, |
| | WIANT-amide | | | | | | 17K, 18A, |
| | | | | | | | 20R, 26I, |
| | | | | | | | 27A |
| Peptide | H(Aib)HGS(αMePhe)TS | 15 | C18diacid | γE- | 17 | Amide | 2Aib, 3H, 5S, |
| 10 | DVSK(αMePhe)LDSK | | | (O2Oc)- | | | 6αMePhe, |
| | (O2Oc-O2Oc-γE- | | | (O2Oc) | | | 10V, |
| | C18diacid)17AARDFVE | | | | | | 13αMePhe, |
| | WIANT-amide | | | | | | 17K, 18A, |
| | | | | | | | 20R, 24E, |
| | | | | | | | 26I, 27A |
| Peptide | H(Aib)HGS(αMePhe)TS | 16 | C18diacid | γE- | 17 | Amide | 2Aib, 3H, 5S, |
| 11 | DVSK(αMePhe)LDSK | | | (O2Oc)- | | | 6αMePhe, |
| | (O2Oc-O2Oc-γE- | | | (O2Oc) | | | 10V, |
| | C18diacid)17AAQDFVQ | | | | | | 13αMePhe, |
| | (Aib)IANT-amide | | | | | | 17K, 18A, |
| | | | | | | | 25Aib, 26I, |
| | | | | | | | 27A |
| Peptide | H(Aib)HGS(αMePhe)TS | 17 | C18diacid | γE- | 17 | Amide | 2Aib, 3H, 5S, |
| 12 | DVSK(αMePhe)LDSK | | | (O2Oc)- | | | 6αMePhe, |
| | (O2Oc-O2Oc-yE- | | | (O2Oc) | | | 10V, |
| | C18diacid)17AARDFVQ | | | | | | 13αMePhe, |
| | (Aib)IANT-amide | | | | | | 17K, 18A, |
| | | | | | | | 20R, 25Aib, |
| | | | | | | | 26I, 27A |
| Peptide | H(Aib)HGS(αMePhe)TS | 18 | C18diacid | γE- | 17 | Amide | 2Aib, 3H, 5S, |
| 13 | DVSK(αMePhe)LDSK | | | (O2Oc)- | | | 6αMePhe, |
| | (O2Oc-O2Oc-γE- | | | (O2Oc) | | | 10V, |
| | C18diacid)17AARD | | | | | | 13αMePhe, |
| | (αMePhe)VQ(Aib)IANT- | | | | | | 17K, 18A, |
| | amide | | | | | | 20R, |
| | | | | | | | 22αMePhe, |
| | | | | | | | 25Aib, 26I, |
| | | | | | | | 27A |
| Peptide | H(Aib)HGS(αMePhe)TS | 19 | C20diacid | γE-γE- | 17 | Amide | 2Aib, 3H, 5S, |
| 14 | DVSK(αMePhe)LDSK | | | (O2Oc)- | | | 6αMePhe, |
| | (O2Oc2-yE2- | | | (O2Oc) | | | 10V, |
| | C20diacid)17AARD | | | | | | 13αMePhe, |
| | (αMePhe)VQ(Aib)IANT- | | | | | | 17K, 18A, |
| | amide | | | | | | 20R, |
| | | | | | | | 22αMePhe, |
| | | | | | | | 25Aib, 26I, |
| | | | | | | | 27A |
| Peptide | H(Aib)HGS(αMePhe)TS | 20 | C18diacid | γE- | 17 | Amide | 2Aib, 3H, 5S, |
| 15 | DVSK(αMePhe)LDSK | | | (O2Oc)- | | | 6αMePhe, |
| | (O2Oc-O2Oc-γE- | | | (O2Oc) | | | 10V, |
| | C18diacid)17AARD | | | | | | 13αMePhe, |
| | (αMePhe)VE(Aib)IANT- | | | | | | 17K, 18A, |
| | amide | | | | | | 20R, |
| | | | | | | | 22αMePhe, |
| | | | | | | | 24E, 25Aib, |
| | | | | | | | 26I, 27A |
| Peptide | H(Aib)HGS(αMePhe)TS | 21 | C18diacid | γE- | 17 | Amide | 2Aib, 3H, 5S, |
| 16 | DVSK(αMePhe)LDSK | | | (O2Oc)- | | | 6αMePhe, |
| | (O2Oc-O2Oc-γE- | | | (O2Oc) | | | 10V, |
| | C18diacid)17AARD | | | | | | 13αMePhe, |
| | (αMePhe)IA(Aib)IANT- | | | | | | 17K, 18A, |
| | amide | | | | | | 20R, |
| | | | | | | | 22αMePhe, |
| | | | | | | | 231, 24A, |
| | | | | | | | 25Aib, 26I, |
| | | | | | | | 27A |
| Peptide | H(Aib)HGS(αMePhe)TS | 22 | C18diacid | γE- | 17 | Amide | 2Aib, 3H, 5S, |
| 17 | DVSK(αMePhe)LDSK | | | (O2Oc)- | | | 6αMePhe, |
| | (O2Oc-O2Oc-γE- | | | (O2Oc) | | | 10V, |
| | C18diacid)17AARD | | | | | | 13αMePhe, |
| | (αMePhe)VESIANT-amide | | | | | | 17K, 18A, |
| | | | | | | | 20R, |
| | | | | | | | 22αMePhe, |
| | | | | | | | 24E, 25S, |
| | | | | | | | 26I, 27A |
| Peptide | H(Aib)HGS(αMePhe)TS | 23 | C18diacid | γE- | 17 | Amide | 2Aib, 3H, 5S, |
| 18 | DVSK(αMePhe)LDSK | | | (O2Oc)- | | | 6αMePhe, |
| | (O2Oc-O2Oc-γE- | | | (O2Oc) | | | 10V, |
| | C18diacid)17AARD | | | | | | 13αMePhe, |
| | (αMePhe)VQSIANT-amide | | | | | | 17K, 18A, |
| | | | | | | | 20R, |
| | | | | | | | 22αMePhe, |
| | | | | | | | 25S, 26I, 27A |
| Peptide | H(Aib)HGS(αMePhe)TS | 24 | C18diacid | γE-γE- | 17 | Amide | 2Aib, 3H, 5S, |
| 19 | DVSK(αMePhe)LDSK | | | (O2Oc)- | | | 6αMePhe, |
| | (O2Oc2-γE2- | | | (O2Oc) | | | 10V, |
| | C18diacid)17AARD | | | | | | 13αMePhe, |
| | (αMePhe)VQ(Aib)IANT- | | | | | | 17K, 18A, |
| | amide | | | | | | 20R, |
| | | | | | | | 22αMePhe, |
| | | | | | | | 25Aib, 26I, |
| | | | | | | | 27A |
| Peptide | H(Aib)HGS(αMePhe)TS | 25 | C18diacid | γE-γE- | 17 | Amide | 2Aib, 3H, 5S, |
| 20 | DVSK(αMePhe)LDSK | | | (O2Oc)- | | | 6αMePhe, |
| | (O2Oc2-γE2- | | | (O2Oc) | | | 10V, |
| | C18diacid)17AARD | | | | | | 13αMePhe, |
| | (αMePhe)VE(Aib)IANT- | | | | | | 17K, 18A, |
| | amide | | | | | | 20R, |
| | | | | | | | 22αMePhe, |
| | | | | | | | 24E, 25Aib, |
| | | | | | | | 26I, 27A |
| Peptide | H(Aib)HGS(αMePhe)TS | 26 | C18diacid | γE | 17 | Amide | 2Aib, 3H, 5S, |
| 21 | DVSK(αMePhe)LDSK(γE- | | | | | | 6αMePhe, |
| | C18diacid)17AAQD | | | | | | 10V, |
| | (αMePhe)VESIANT-amide | | | | | | 13αMePhe, |
| | | | | | | | 17K, 18A, |
| | | | | | | | 22αMePhe, |
| | | | | | | | 24E, 25S, |
| | | | | | | | 26I, 27A |
| Peptide | H(Aib)HGS(αMePhe)TS | 27 | C20diacid | γE-γE- | 17 | Amide | 2Aib, 3H, 5S, |
| 22 | DVSK(αMePhe)LDSK | | | (O2Oc)- | | | 6αMePhe, |
| | (O2Oc-O2Oc-γE-γE- | | | (O2Oc) | | | 10V, |
| | C20diacid)17AAQD | | | | | | 13αMePhe, |
| | (αMePhe)VESIANT-amide | | | | | | 17K, 18A, |
| | | | | | | | 22αMePhe, |
| | | | | | | | 24E, 25S, |
| | | | | | | | 26I, 27A |
| Peptide | H(Aib)HGS(αMePhe)TS | 28 | C18diacid | γE- | 17 | Amide | 2Aib, 3H, 5S, |
| 23 | DVSK(αMePhe)LDSK | | | (O2Oc)- | | | 6αMePhe, |
| | (O2Oc-O2Oc-γE- | | | (O2Oc) | | | 10V, |
| | C18diacid)17AAQD | | | | | | 13αMePhe, |
| | (αMePhe)VESIANT-amide | | | | | | 17K, 18A, |
| | | | | | | | 22αMePhe, |
| | | | | | | | 24E, 25S, |
| | | | | | | | 26I, 27A |
| Peptide | H(Aib)HGS(αMePhe)TS | 29 | C18diacid | γE- | 17 | Amide | 2Aib, 3H, 5S, |
| 24 | DVSK(αMePhe)LDSK | | | (O2Oc)- | | | 6αMePhe, |
| | (O2Oc-O2Oc-γE- | | | (O2Oc) | | | 10V, |
| | C18diacid)17AAQD | | | | | | 13αMePhe, |
| | (αMePhe)VQ(Aib)IANT- | | | | | | 17K, 18A, |
| | amide | | | | | | 22αMePhe, |
| | | | | | | | 25Aib, 26I, |
| | | | | | | | 27A |
| Peptide | H(Aib)HGS(αMePhe)TS | 30 | C20diacid | γE-yE- | 17 | Amide | 2Aib, 3H, 5S, |
| 25 | DVSK(αMePhe)LDSK | | | (O2Oc)- | | | 6αMePhe, |
| | (O2Oc-O2Oc-γE-γE- | | | (O2Oc) | | | 10V, |
| | C20diacid)17AAQD | | | | | | 13αMePhe, |
| | (αMePhe)VQ(Aib)IANT- | | | | | | 17K, 18A, |
| | amide | | | | | | 22αMePhe, |
| | | | | | | | 25Aib, 26I, |
| | | | | | | | 27A |
| Peptide | H(Aib)QGS(αMePhe)TS | 31 | C18diacid | γE- | 17 | Amide | 2Aib, 5S, |
| 26 | DVSK(αMePhe)LDSK | | | (O2Oc)- | | | 6αMePhe, |
| | (O2Oc-O2Oc-γE- | | | (O2Oc) | | | 10V, |
| | C18diacid)17AAQD | | | | | | 13αMePhe, |
| | (αMePhe)VQWIAN-amide | | | | | | 17K, 18A, |
| | | | | | | | 22αMePhe, |
| | | | | | | | 26I, 27A, |
| | | | | | | | des29T |
| Peptide | H(Aib)QGS(αMePhe)TS | 32 | C18diacid | γE- | 17 | Amide | 2Aib, 5S, |
| 27 | DVSK(αMePhe)LDSK | | | (O2Oc)- | | | 6αMePhe, |
| | (O2Oc-O2Oc-γE- | | | (O2Oc) | | | 10V, |
| | C18diacid)17AAQD | | | | | | 13αMePhe, |
| | (αMePhe)VEWIAN-amide | | | | | | 17K, 18A, |
| | | | | | | | 22αMePhe, |
| | | | | | | | 24E, 26I, |
| | | | | | | | 27A, des29T |
| Peptide | H(Aib)QGTFTSDVSK | 33 | C18diacid | γE- | 17 | Acid | 2Aib, 10V, |
| 28 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27L, 28Aib |
| | WLL(Aib)T-acid | | | | | | |
| Peptide | H(Aib)QGTFTSDVSK | 34 | C18diacid | γE- | 17 | Acid | 2Aib, 10V, |
| 29 | (αMePhe)LDSK(O2Oc- | | | (O2Oc)- | | | 13αMePhe, |
| | O2Oc-γE- | | | (O2Oc) | | | 17K, 20Aib, |
| | C18diacid)17RA(Aib)DF | | | | | | 27L, 28Aib |
| | VQWLL(Aib)T-acid | | | | | | |
| Peptide | H(Aib)QGTFTSDVSK | 35 | C18diacid | γE- | 17 | Acid | 2Aib, 10V, |
| 30 | (αMePhe)LDSK(02Oc- | | | (O2Oc)- | | | 13αMePhe, |
| | O2Oc-yE- | | | (O2Oc) | | | 17K, 24R, |
| | C18diacid)17RAQDFVR | | | | | | 27L, 28Aib, |
| | WLL(Aib)TG-acid | | | | | | 30G |
| Peptide | H(Aib)QGTFTSDVSK | 36 | C18diacid | γE- | 17 | Acid | 2Aib, 10V, |
| 31 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27L, 28E, |
| | WLLE(Aib)G-acid | | | | | | 29Aib, 30G |
| Peptide | H(Aib)QGTFTSDVSK | 37 | C18diacid | γE | 17 | Amide | 2Aib, 10V, |
| 32 | (αMePhe)LDSK(γE- | | | | | | 13αMePhe, |
| | C18diacid)17RA(Aib)DF | | | | | | 17K, 20Aib, |
| | VQWIANT-amide | | | | | | 26I, 27A |
| Peptide | H(Aib)QGTFTSDVSK | 38 | C18diacid | γE- | 17 | Acid | 2Aib, 10V, |
| 33 | (αMePhe)LDSK(O2Oc- | | | (O2Oc)- | | | 13αMePhe, |
| | O2Oc-γE- | | | (O2Oc) | | | 17K, 27E, |
| | C18diacid)17RAQDFVQ | | | | | | 28Aib |
| | WLE(Aib)T-acid | | | | | | |
| Peptide | H(Aib)QGTFTSDVSK | 39 | C18diacid | γE- | 17 | Acid | 2Aib, 10V, |
| 34 | (αMePhe)LDSK(O2Oc- | | | (O2Oc)- | | | 13αMePhe, |
| | O2Oc-γE- | | | (O2Oc) | | | 17K, 27L, |
| | C18diacid)17RAQDFVQ | | | | | | 28Aib, 29E |
| | WLL(Aib)E-acid | | | | | | |
| Peptide | H(Aib)QGTFTSDVSK | 40 | C18diacid | γE- | 17 | Acid | 2Aib, 10V, |
| 35 | (αMePhe)LDTK(O2Oc- | | | (O2Oc)- | | | 13αMePhe, |
| | O2Oc-γE- | | | (O2Oc) | | | 16T, 17K, |
| | C18diacid)17RAQDFVQ | | | | | | 27L, 28Aib |
| | WLL(Aib)T-acid | | | | | | |
| Peptide | H(Aib)QGTFTSDVSK | 41 | C18diacid | γE- | 17 | Acid | 2Aib, 10V, |
| 36 | (αMePhe)LDSK(O2Oc- | | | (O2Oc)- | | | 13αMePhe, |
| | O2Oc-γE- | | | (O2Oc) | | | 17K, 20R, |
| | C18diacid)17RARDFVQ | | | | | | 27L, 28Aib |
| | WLL(Aib)T-acid | | | | | | |
| Peptide | H(Aib)QGTFTSDVSK | 42 | C18diacid | γE- | 17 | Acid | 2Aib, 10V, |
| 37 | (αMePhe)LDSK(O2Oc- | | | (O2Oc)- | | | 13αMePhe, |
| | O2Oc-γE- | | | (O2Oc) | | | 17K, 20E, |
| | C18diacid)17RAEDFVQ | | | | | | 27L, 28Aib |
| | WLL(Aib)T-acid | | | | | | |
| Peptide | H(Aib)QGTFTSDVSK | 43 | C18diacid | γE- | 17 | Acid | 2Aib, 10V, |
| 38 | (αMePhe)LDSK(02Oc- | | | (O2Oc)- | | | 13αMePhe, |
| | O2Oc-γE- | | | (O2Oc) | | | 17K, 24E, |
| | C18diacid)17RAQDFVE | | | | | | 27L, 28Aib |
| | WLL(Aib)T-acid | | | | | | |
| Peptide | H(Aib)QGTFTSDVSK | 44 | C18diacid | γE- | 17 | Acid | 2Aib, 10V, |
| 39 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27L, 28Aib, |
| | WLL(Aib)TE-acid | | | | | | 30E |
| Peptide | H(Aib)QGTFTSDVSK | 45 | C18diacid | γE- | 17 | Acid | 2Aib, 10V, |
| 40 | (αMePhe)LDSK(O2Oc- | | | (O2Oc)- | | | 13αMePhe, |
| | O2Oc-γE- | | | (O2Oc) | | | 17K, 27L, |
| | C18diacid)17RAQDFVQ | | | | | | 28Aib, |
| | WLL(Aib)T-acid | | | | | | |
| Peptide | H(Aib)QGTFTSDYSK | 46 | C18diacid | γE- | 17 | Amide | 2Aib, |
| 41 | (αMePhe)LDSK(O2Oc- | | | (O2Oc)- | | | 13αMePhe, |
| | O2Oc-γE- | | | (O2Oc) | | | 17K, 27V, |
| | C18diacid)17RAQDFVQ | | | | | | 28(PEG)4, |
| | WLV(PEG)4-amide | | | | | | des29 |
| Peptide | H(Aib)QGTFTSDVSK | 47 | C18diacid | γE- | 17 | Acid | 2Aib, 10V, |
| 42 | (αMePhe)LDSK(O2Oc- | | | (O2Oc)- | | | 13αMePhe, |
| | O2Oc-γE- | | | (O2Oc) | | | 17K, 25Aib, |
| | C18diacid)17RAQDFVQ | | | | | | 27V, 28A |
| | (Aib)LVAT-acid | | | | | | |
| Peptide | H(Aib)QGS(αMePhe)TS | 114 | Palmitoyl | γE-γE- | 17 | Amide | 2Aib, 5S, |
| 43 | DVSK(Dip)LDSK((PEG)4- | | | (PEG)4 | | | 6αMePhe, |
| | γE-γE- | | | | | | 10V, 13Dip, |
| | Palmitoyl)17RAQD | | | | | | 17K, |
| | (αMePhe)VE(Aib)LEAGG- | | | | | | 22αMePhe, |
| | amide | | | | | | 24E, 25Aib, |
| | | | | | | | 27E, 28A, |
| | | | | | | | 29G, 30G |
| Peptide | H(Aib)QGS(αMePhe)TS | 115 | Stearoyl | γE-yE- | 17 | Amide | 2Aib, 5S, |
| 44 | DVSK(Dip)LDSK((PEG)2- | | | (PEG)2- | | | 6αMePhe, |
| | (PEG)2-γE-γE- | | | (PEG)2 | | | 10V, 13Dip, |
| | Stearoyl)17AAQD | | | | | | 17K, |
| | (αMePhe)VE(Aib)LEAGG- | | | | | | 22αMePhe, |
| | amide | | | | | | 24E, 25Aib, |
| | | | | | | | 27E, 28A, |
| | | | | | | | 29G, 30G |
| Peptide | H(Aib)QGS(αMePhe)TS | 116 | Stearoyl | γE-γE- | 17 | Amide | 2Aib, 5S, |
| 45 | DVSK(Dip)LDSK((PEG)2- | | | (PEG)2- | | | 6αMePhe, |
| | (PEG)2-γE-γE- | | | (PEG)2 | | | 10V, 13Dip, |
| | Stearoyl)17AAQD | | | | | | 17K, |
| | (αMePhe)VE(Aib)LANT- | | | | | | 22αMePhe, |
| | amide | | | | | | 24E, 25Aib, |
| | | | | | | | 27A |
| Peptide | HSQGS(αMePhe)TSDVS | 117 | Stearoyl | γE-γE- | 17 | Amide | 5S, |
| 46 | K(Dip)LDSK((PEG)2- | | | (PEG)2- | | | 6αMePhe, |
| | (PEG)2-γE-γE- | | | (PEG)2 | | | 10V, 13Dip, |
| | Stearoyl)17AAQD | | | | | | 17K, 18A, |
| | (αMePhe)VE(Aib)LEAGG- | | | | | | 22αMePhe, |
| | amide | | | | | | 24E, 25Aib, |
| | | | | | | | 27E, 28A, |
| | | | | | | | 29G, 30G |
| Peptide | HSQGS(αMePhe)TSDVS | 118 | Stearoyl | γE-γE- | 17 | Amide | 5S, |
| 47 | K(Dip)LDSK((PEG)2- | | | (PEG)2- | | | 6αMePhe, |
| | (PEG)2-γE-γE- | | | (PEG)2 | | | 10V, 13Dip, |
| | Stearoyl)17AAQD | | | | | | 17K, 18A, |
| | Phe)VE(Aib)LANT- | | | | | | 22αMePhe, |
| | (αMeamide | | | | | | 24E, 25Aib, |
| | | | | | | | 27A |
| Peptide | HSQGS(αMePhe)TSDVS | 119 | Stearoyl | γE-γE- | 17 | Amide | 5S, |
| 48 | K(Dip)LDSK((PEG)2- | | | (PEG)2- | | | 6αMePhe, |
| | (PEG)2-γE-γE- | | | (PEG)2 | | | 10V, 13Dip, |
| | Stearoyl)17AAQD | | | | | | 17K, 18A, |
| | (αMePhe)VE(Aib)LISG- | | | | | | 22αMePhe, |
| | amide | | | | | | 24E, 25Aib, |
| | | | | | | | 27I, 28S, 29G |
| Peptide | HSQGS(αMePhe)TSDVS | 120 | Stearoyl | γE-γE- | 17 | Amide | 5S, |
| 49 | K(Dip)LDSK((PEG)2- | | | (PEG)2- | | | 6αMePhe, |
| | (PEG)2-γE-γE- | | | (PEG)2 | | | 10V, 13Dip, |
| | Stearoyl)17AAQD | | | | | | 17K, 18A, |
| | (αMePhe)VE(Aib)IANT- | | | | | | 22αMePhe, |
| | amide | | | | | | 24E, 25Aib, |
| | | | | | | | 26I, 27A |
| Peptide | HSQGS(αMePhe)TSDVS | 121 | Stearoyl | γE-γE- | 17 | Amide | 5S, |
| 50 | K(Dip)LDSK((PEG)2- | | | (PEG)2- | | | 6αMePhe, |
| | (PEG)2-γE-γE- | | | (PEG)2 | | | 10V, 13Dip, |
| | Stearoyl)17AAQD | | | | | | 17K, 18A, |
| | (αMePhe)VE(Aib)IINT- | | | | | | 22αMePhe, |
| | amide | | | | | | 24E, 25Aib, |
| | | | | | | | 26I, 27I |
| Peptide | HSQGS(αMePhe)TSDVS | 122 | Stearoyl | γE-γE- | 17 | Amide | 5S, |
| 51 | K(Dip)LDSK((PEG)2- | | | (PEG)2- | | | 6αMePhe, |
| | (PEG)2-γE-γE- | | | (PEG)2 | | | 10V, 13Dip, |
| | Stearoyl)17AAQD | | | | | | 17K, 18A, |
| | (αMePhe)VE(Aib)LLNT- | | | | | | 22αMePhe, |
| | amide | | | | | | 24E, 25Aib, |
| | | | | | | | 27L |
| Peptide | HSQGS(αMePhe)TSDVS | 123 | Stearoyl | γE-γE- | 17 | Amide | 5S, |
| 52 | K(Dip)LDSK((PEG)2- | | | (PEG)2- | | | 6αMePhe, |
| | (PEG)2-γE-γE- | | | (PEG)2 | | | 10V, 13Dip, |
| | Stearoyl)17AAQD | | | | | | 17K, 18A, |
| | (αMePhe)VE(Aib)ChaANT- | | | | | | 22αMePhe, |
| | amide | | | | | | 24E, 25Aib, |
| | | | | | | | 26Cha, 27A |
| Peptide | HSQGS(αMePhe)TSDVS | 124 | Stearoyl | γE-γE- | 17 | Amide | 5S, |
| 53 | K(Dip)LDSK((PEG)2- | | | (PEG)2- | | | 6αMePhe, |
| | (PEG)2-γE-γE- | | | (PEG)2 | | | 10V, 13Dip, |
| | Stearoyl)17AAQD | | | | | | 17K, 18A, |
| | Phe)VE(Aib)VVEGG- | | | | | | 22αMePhe, |
| | (αMeamide | | | | | | 24E, 25Aib, |
| | | | | | | | 26V, 27V, |
| | | | | | | | 28E, 29G, |
| | | | | | | | 30G |
| Peptide | HSQGS(αMePhe)TSDVS | 125 | Stearoyl | γE-yE- | 17 | Amide | 5S, |
| 54 | K(Dip)LDSK((PEG)2- | | | (PEG)2- | | | 6αMePhe, |
| | (PEG)2-γE-γE- | | | (PEG)2 | | | 10V, 13Dip, |
| | Stearoyl)17AAQD | | | | | | 17K, 18A, |
| | (αMePhe)VQ(Aib)IAN- | | | | | | 22αMePhe, |
| | amide | | | | | | 25Aib, 26I, |
| | | | | | | | 27A, des29T |
| Peptide | HSQGS(αMePhe)TSDVS | 126 | Stearoyl | γE-γE- | 17 | Amide | 5S, |
| 55 | K(Dip)LDSK((PEG)2- | | | (PEG)2- | | | 6αMePhe, |
| | (PEG)2-γE-γE- | | | (PEG)2 | | | 10V, 13Dip, |
| | Stearoyl)17AAQD | | | | | | 17K, 18A, |
| | (αMePhe)VQ(Dip)LEA- | | | | | | 22αMePhe, |
| | amide | | | | | | 25Dip, 27E, |
| | | | | | | | 28A, des29T |
| Peptide | HSQGS(αMePhe)TSDVS | 127 | Stearoyl | γE-γE- | 17 | Amide | 5S, |
| 56 | K(Dip)LDSK((PEG)4-γE- | | | (PEG)4 | | | 6αMePhe, |
| | γE- | | | | | | 10V, 13Dip, |
| | Stearoyl)17AAQD | | | | | | 17K, 18A, |
| | (αMePhe)VE(Aib)IINT- | | | | | | 22αMePhe, |
| | amide | | | | | | 24E, 25Aib, |
| | | | | | | | 26I, 271 |
| Peptide | HSQGS(αMePhe)TSDVS | 128 | Stearoyl | γE-γE- | 17 | Amide | 5S, |
| 57 | K(Dip)LDSK((PEG)8-γE- | | | (PEG)8 | | | 6αMePhe, |
| | γE- | | | | | | 10V, 13Dip, |
| | Stearoyl)17AAQD | | | | | | 17K, 18A, |
| | (αMePhe)VE(Aib)IINT- | | | | | | 22αMePhe, |
| | amide | | | | | | 24E, 25Aib, |
| | | | | | | | 26I, 27I |
| Peptide | HSQGS(αMePhe)TSDVS | 129 | Stearoyl | γE-γE- | 17 | Amide | 5S, |
| 58 | K(Dip)LDSK((PEG)12- | | | (PEG)12 | | | 6αMePhe, |
| | γE-γE- | | | | | | 10V, 13Dip, |
| | Stearoyl)17AAQD | | | | | | 17K, 18A, |
| | (αMePhe)VE(Aib)IINT- | | | | | | 22αMePhe, |
| | amide | | | | | | 24E, 25Aib, |
| | | | | | | | 26I, 27I |
| Peptide | HSQGS(αMePhe)TSDVS | 130 | Stearoyl | (PEG)2- | 17 | Amide | 5S, |
| 59 | K(Dip)LDSK(γE-γE- | | | (PEG)2- | | | 6αMePhe, |
| | (PEG)2-(PEG)2- | | | γE-γE | | | 10V, 13Dip, |
| | Stearoyl)17AAQD | | | | | | 17K, 18A, |
| | (αMePhe)VE(Aib)IINT- | | | | | | 22αMePhe, |
| | amide | | | | | | 24E, 25Aib, |
| | | | | | | | 26I, 27I |
| Peptide | HSQGS(αMePhe)TSDVS | 131 | Stearoyl | (PEG)2- | 17 | Amide | 5S, |
| 60 | K(Dip)LDSK(γE-(PEG)2- | | | γE- | | | 6αMePhe, |
| | γE-(PEG)2- | | | (PEG)2- | | | 10V, 13Dip, |
| | Stearoyl)17AAQD | | | γE | | | 17K, 18A, |
| | (αMePhe)VE(Aib)IINT- | | | | | | 22αMePhe, |
| | amide | | | | | | 24E, 25Aib, |
| | | | | | | | 26I, 27I |
| Peptide | HSQGS(αMePhe)TSDVS | 132 | Stearoyl | γE- | 17 | Amide | 5S, |
| 61 | K(Dip)LDSK((PEG)2-γE- | | | (PEG)2- | | | 6αMePhe, |
| | (PEG)2-γE- | | | γE- | | | 10V, 13Dip, |
| | Stearoyl)17AAQD | | | (PEG)2 | | | 17K, 18A, |
| | (αMePhe)VE(Aib)IINT- | | | | | | 22αMePhe, |
| | amide | | | | | | 24E, 25Aib, |
| | | | | | | | 26I, 27I |
| Peptide | HSQGS(αMePhe)TSDVS | 133 | Lauryl | γE-γE- | 17 | Amide | 5S, |
| 62 | K(Dip)LDSK((PEG)2- | | | (PEG)2- | | | 6αMePhe, |
| | (PEG)2-γE-γE- | | | (PEG)2 | | | 10V, 13Dip, |
| | Lauryl)17AAQD | | | | | | 17K, 18A, |
| | (αMePhe)VE(Aib)IINT- | | | | | | 22αMePhe, |
| | amide | | | | | | 24E, 25Aib, |
| | | | | | | | 26I, 27I |
| Peptide | HSQGS(αMePhe)TSDVS | 134 | Myristoyl | γE-γE- | 17 | Amide | 5S, |
| 63 | K(Dip)LDSK((PEG)2- | | | (PEG)2- | | | 6αMePhe, |
| | (PEG)2-γE-γE- | | | (PEG)2 | | | 10V, 13Dip, |
| | Myristyl)17AAQD | | | | | | 17K, 18A, |
| | (αMePhe)VE(Aib)IINT- | | | | | | 22αMePhe, |
| | amide | | | | | | 24E, 25Aib, |
| | | | | | | | 26I, 27I |
| Peptide | HSQGS(αMePhe)TSDVS | 135 | Palmitoyl | γE-yE- | 17 | Amide | 5S, |
| 64 | K(Dip)LDSK((PEG)2- | | | (PEG)2- | | | 6αMePhe, |
| | (PEG)2-γE-γE- | | | (PEG)2 | | | 10V, 13Dip, |
| | Palmitoyl)17AAQD | | | | | | 17K, 18A, |
| | (αMePhe)VE(Aib)IINT- | | | | | | 22αMePhe, |
| | amide | | | | | | 24E, 25Aib, |
| | | | | | | | 26I, 27I |
| Peptide | HSQGS(αMePhe)TSDVS | 136 | Stearoyl | γE-γE- | 17 | Amide | 5S, |
| 65 | K(Dip)LDSK((PEG)2- | | | (PEG)2- | | | 6αMePhe, |
| | (PEG)2-γE-γE- | | | (PEG)2 | | | 10V, 13Dip, |
| | C18diacid)17AAQD | | | | | | 17K, 18A, |
| | (αMePhe)VE(Aib)IINT- | | | | | | 22αMePhe, |
| | amide | | | | | | 24E, 25Aib, |
| | | | | | | | 26I, 27I |
| Peptide | H(Aib)QGS(αMePhe)TS | 137 | Stearoyl | γE-γE- | 17 | Amide | 2Aib, 5S, |
| 66 | DVSK(Dip)LDSK(ϵ- | | | (PEG)2- | | | 6αMePhe, |
| | ((PEG)2-(PEG)2-γE-γE- | | | (PEG)2 | | | 10V, 13Dip, |
| | Stearyl)17AAQD | | | | | | 17K, 18A, |
| | (αMePhe)VQ(Aib)IAN- | | | | | | 22αMePhe, |
| | amide | | | | | | 25Aib, 26I, |
| | | | | | | | 27A, des29T |
| Peptide | H(Aib)QGS(αMePhe)TS | 138 | Palmitoyl | γE- | 17 | Amide | 2Aib, 5S, |
| 67 | DVSK(Dip)LDSK(ϵ- | | | (PEG)2- | | | 6αMePhe, |
| | ((PEG)2-(PEG)2-γE- | | | (PEG)2 | | | 10V, 13Dip, |
| | Palmitoyl)17AAQD | | | | | | 17K, 18A, |
| | (αMePhe)VQ(Aib)IAN- | | | | | | 22αMePhe, |
| | amide | | | | | | 25Aib, 26I, |
| | | | | | | | 27A, des29T |
| Peptide | H(Aib)QGTFTSDVSK | 139 | Palmitoyl | γE- | 17 | Amide | 2Aib, 10V, |
| 68 | (αMePhe)LDVK((PEG)4- | | | (PEG)4 | | | 13αMePhe, |
| | γE- | | | | | | 16V, 17K, |
| | Palmitoyl)17RAQD | | | | | | 22αMePhe, |
| | (αMePhe)VE(Aib) | | | | | | 24E, 25Aib, |
| | LNleET-amide | | | | | | 27Nle, 28E |
| Peptide | H(Aib)QGTFTSDVSK | 140 | Stearoyl | γE-γE- | 17 | Amide | 2Aib, 10V, |
| 69 | (αMePhe)LDSK((PEG)2- | | | (PEG)2- | | | 13αMePhe, |
| | (PEG)2-γE-γE- | | | (PEG)2 | | | 16V, 17K, |
| | Stearyl)17RAQD(αMeP | | | | | | 22αMePhe, |
| | he)VQ(Aib)LNleET- | | | | | | 24E, 25Aib, |
| | amide | | | | | | 27Nle, 28E |
| Peptide | H(Aib)QGS(αMePhe)TS | 141 | Stearoyl | γE-γE- | 17 | Amide | 2Aib, 5S, |
| 70 | DVSK(αMePhe)LDSK | | | (PEG)4 | | | 6αMePhe, |
| | ((PEG)4-γE-γE- | | | | | | 10V, |
| | Stearoyl)17RAQD | | | | | | 13αMePhe, |
| | (αMePhe)VE(Aib)LEAGG- | | | | | | 17K, |
| | amide | | | | | | 22αMePhe, |
| | | | | | | | 24E, 25Aib, |
| | | | | | | | 27E, 28A, |
| | | | | | | | 29G, 30G |
| Peptide | H(Aib)QGS(αMePhe)TS | 142 | Stearoyl | γE-γE- | 17 | Amide | 2Aib, 5S, |
| 71 | DVSK(αMePhe)LDSK | | | (PEG)4 | | | 6αMePhe, |
| | ((PEG)4-γE-γE- | | | | | | 10V, |
| | Stearoyl)17AAQD | | | | | | 13αMePhe, |
| | (αMePhe)VE(Aib)LEAGG- | | | | | | 17K, 18A, |
| | amide | | | | | | 22αMePhe, |
| | | | | | | | 24E, 25Aib, |
| | | | | | | | 27E, 28A, |
| | | | | | | | 29G, 30G |
| Peptide | H(Aib)QGS(αMePhe)TS | 143 | Palmitoyl | γE-γE- | 17 | Amide | 2Aib, 5S, |
| 72 | DVSK(αMePhe)LDSK | | | (PEG)4 | | | 6αMePhe, |
| | ((PEG)4-γE-γE- | | | | | | 10V, |
| | Palmitoyl)17AAQD | | | | | | 13αMePhe, |
| | (αMePhe)VE(Aib)LEAGG- | | | | | | 17K, 18A, |
| | amide | | | | | | 22αMePhe, |
| | | | | | | | 24E, 25Aib, |
| | | | | | | | 27E, 28A, |
| | | | | | | | 29G, 30G |
| Peptide | H(Aib)QGS(αMePhe)TS | 144 | Palmitoyl | γE-γE- | 17 | Amide | 2Aib, 5S, |
| 73 | DVSK(αMePhe)LDSK | | | (PEG)2- | | | 6αMePhe, |
| | ((PEG)2-(PEG)2-γE-γE- | | | (PEG)2 | | | 10V, |
| | Palmitoyl)17AAQD | | | | | | 13αMePhe, |
| | (αMePhe)VQ(Aib)IAN- | | | | | | 17K, 18A, |
| | amide | | | | | | 22αMePhe, |
| | | | | | | | 25Aib, 26I, |
| | | | | | | | 27A, des29T |
| Peptide | H(Aib)QGS(αMePhe)TS | 145 | Stearoyl | γE-γE- | 17 | Amide | 2Aib, 5S, |
| 74 | DVSK(αMePhe)LDSK | | | (PEG)2- | | | 6αMePhe, |
| | ((PEG)2-(PEG)2-γE-γE- | | | (PEG)2 | | | 10V, |
| | Stearoyl)17(β- | | | | | | 13αMePhe, |
| | dimethylGln)AQD | | | | | | 17K, 18β- |
| | (αMePhe)VE(Aib)LEAGG- | | | | | | dimethyl-R, |
| | amide | | | | | | 22αMePhe, |
| | | | | | | | 24E, 25Aib, |
| | | | | | | | 27E, 28A, |
| | | | | | | | 29G, 30G |
| Peptide | H(Aib)QGS(αMePhe)TS | 146 | Palmitoyl | γE-γE- | 17 | Amide | 2Aib, 5S, |
| 75 | DVSK(αMePhe)LDSK | | | (PEG)2- | | | 6αMePhe, 10V, |
| | ((PEG)2-(PEG)2-γE-γE- | | | (PEG)2 | | | 13αMePhe, |
| | Palmitoyl)17AAQDIVQII | | | | | | 17K, 18A, |
| | AN-amide | | | | | | 22I, 251, |
| | | | | | | | 26I, |
| | | | | | | | 27A, des29T |
| Peptide | H(Aib)QGS(αMePhe)TS | 147 | Stearoyl | γE-γE- | 17 | Amide | 2Aib, 5S, |
| 76 | DVSK(αMePhe)LDSK | | | (PEG)2- | | | 6αMePhe, |
| | ((PEG)2-(PEG)2-yE-γE- | | | (PEG)2 | | | 10V, |
| | Stearoyl)17AAQD | | | | | | 13αMePhe, |
| | (αMePhe)VQ(Aib)VVEGG- | | | | | | 17K, 18A, |
| | amide | | | | | | 22αMePhe, |
| | | | | | | | 25Aib, 26V, |
| | | | | | | | 27V, 28E, |
| | | | | | | | 29G, 30G |
| Peptide | H(Aib)QGS(αMePhe)TS | 148 | Stearoyl | γE-γE- | 17 | Amide | 2Aib, 5S, |
| 77 | DVSK(αMePhe)LDSK | | | (PEG)2- | | | 6αMePhe, |
| | ((PEG)2-(PEG)2-γE-γE- | | | (PEG)2 | | | 10V |
| | Stearoyl)17AAQD | | | | | | 13αMePhe, |
| | (αMePhe)VQ(Aib)LISG- | | | | | | 17K, 18A, |
| | amide | | | | | | 22αMePhe, |
| | | | | | | | 25Aib, 27I, |
| | | | | | | | 28S, 29G |
| Peptide | HSQGS(αMePhe)TSDVS | 149 | Stearoyl | γE-γE- | 17 | Amide | 5S, |
| 78 | K(αMePhe)LDSK((PEG) | | | (PEG)2- | | | 6αMePhe, |
| | 2-(PEG)2-γE-γE- | | | (PEG)2 | | | 10V, |
| | Stearoyl)17AAQD | | | | | | 13αMePhe, |
| | (αMePhe)VQ(Aib)LEAGG- | | | | | | 17K, 18A, |
| | amide | | | | | | 22αMePhe, |
| | | | | | | | 25Aib, 27E, |
| | | | | | | | 28A, 29G, |
| | | | | | | | 30G |
| Peptide | HSQGS(αMePhe)TSDVS | 150 | Palmitoyl | γE-γE- | 17 | Amide | 5S, |
| 79 | K(αMePhe)LDSK((PEG) | | | (PEG)2- | | | 6αMePhe, |
| | 2-(PEG)2-γE-γE- | | | (PEG)2 | | | 10V, |
| | Palmitoyl)17AAQD | | | | | | 13αMePhe, |
| | (αMePhe)VQ(Aib)LEAGG- | | | | | | 17K, 18A, |
| | amide | | | | | | 22αMePhe, |
| | | | | | | | 25Aib, 27E, |
| | | | | | | | 28A, 29G, |
| | | | | | | | 30G |
| Peptide | HSQGS(αMePhe)TSDVS | 151 | Palmitoyl | γE-γE- | 17 | Amide | 5S, |
| 80 | K(αMePhe)LDSK((PEG) | | | (PEG)2- | | | 6αMePhe, |
| | 2-(PEG)2-γE-γE- | | | (PEG)2 | | | 10V, |
| | Margaroyl)17AAQD | | | | | | 13αMePhe, |
| | (αMePhe)VQ(Aib)LEAGG- | | | | | | 17K, 18A, |
| | amide | | | | | | 22αMePhe, |
| | | | | | | | 25Aib, 27E, |
| | | | | | | | 28A, 29G, |
| | | | | | | | 30G |
| Peptide | HSQGS(αMePhe)TSDVS | 152 | Stearoyl | γE-γE- | 17 | Amide | 5S, |
| 81 | K(αMePhe)LDSK((PEG) | | | (PEG)2- | | | 6αMePhe, |
| | 2-(PEG)2-γE-γE- | | | (PEG)2 | | | 10V, |
| | Stearoyl)17AAQD | | | | | | 13αMePhe, |
| | (αMePhe)VE(Aib)LEAGG- | | | | | | 17K, 18A, |
| | amide | | | | | | 22αMePhe, |
| | | | | | | | 25Aib, 27E, |
| | | | | | | | 28A, 29G, |
| | | | | | | | 30G |
| Peptide | HSQGS(αMePhe)TSDVS | 153 | Stearoyl | γE-γE- | 17 | Amide | 5S, |
| 82 | K(αMePhe)LDSK((PEG) | | | (PEG)2- | | | 6αMePhe, |
| | 2-(PEG)2-γE-γE- | | | (PEG)2 | | | 10V, |
| | Stearoyl)17AAQD | | | | | | 13αMePhe, |
| | (αMePhe)VQ(Aib)IANT- | | | | | | 17K, 18A, |
| | amide | | | | | | 22αMePhe, |
| | | | | | | | 25Aib, 26I, |
| | | | | | | | 27A |
| Peptide | HSQGS(αMePhe)TSDVS | 154 | Stearoyl | γE-γE- | 17 | Amide | 5S, |
| 83 | K(αMePhe)LDSK((PEG) | | | (PEG)2- | | | 6αMePhe, |
| | 2-(PEG)2-γE-γE- | | | (PEG)2 | | | 10V, |
| | Stearoyl)17AAQD | | | | | | 13αMePhe, |
| | (αMePhe)VQ(Aib)IINT- | | | | | | 17K, 18A, |
| | amide | | | | | | 22αMePhe, |
| | | | | | | | 25Aib, 26I, |
| | | | | | | | 27I |
| Peptide | HSQGS(αMePhe)TSDVS | 155 | Stearoyl | γE-γE- | 17 | Amide | 5S, |
| 84 | K(αMePhe)LDSK((PEG) | | | (PEG)2- | | | 6αMePhe, |
| | 2-(PEG)2-γE-γE- | | | (PEG)2 | | | 10V, |
| | Stearoyl)17AAQD | | | | | | 13αMePhe, |
| | (αMePhe)VQ(Aib)LLNT- | | | | | | 17K, 18A, |
| | amide | | | | | | 22αMePhe, |
| | | | | | | | 25Aib, 27L |
| Peptide | HSQGS(αMePhe)TSDVS | 156 | Stearoyl | γE-γE- | 17 | Amide | 5S, |
| 85 | K(αMePhe)LDSK((PEG) | | | (PEG)2- | | | 6αMePhe, |
| | 2-(PEG)2-γE-γE- | | | (PEG)2 | | | 10V, |
| | Stearoyl)17AAQD | | | | | | 13αMePhe, |
| | (αMePhe)VQ(Aib)VVEGG- | | | | | | 17K, 18A, |
| | amide | | | | | | 22αMePhe, |
| | | | | | | | 25Aib, 26V, |
| | | | | | | | 27V, 28E, |
| | | | | | | | 29G, 30G |
| Peptide | HSQGS(αMePhe)TSDVS | 157 | Stearoyl | γE-γE- | 17 | Amide | 5S, |
| 86 | K(αMePhe)LDSK((PEG) | | | (PEG)2- | | | 6αMePhe, |
| | 2-(PEG)2-γE-γE- | | | (PEG)2 | | | 10V, |
| | Stearoyl)17AAQD | | | | | | 13αMePhe, |
| | (αMePhe)VQ(Aib)IAN- | | | | | | 17K, 18A, |
| | amide | | | | | | 22αMePhe, |
| | | | | | | | 25Aib, 26I, |
| | | | | | | | 27A, des29T |
| Peptide | HSQGS(αMePhe)TSDVS | 158 | Palmitoyl | γE-yE- | 17 | Amide | 5S, |
| 87 | K(αMePhe)LDSK((PEG) | | | (PEG)2- | | | 6αMePhe, |
| | 2-(PEG)2-γE-γE- | | | (PEG)2 | | | 10V, |
| | Palmitoyl)17AAQD | | | | | | 13αMePhe, |
| | (αMePhe)VQ(Aib)IAN- | | | | | | 17K, 18A, |
| | amide | | | | | | 22αMePhe, |
| | | | | | | | 25Aib, 26I, |
| | | | | | | | 27A, des29T |
| Peptide | HSQGS(αMePhe)TSDVS | 159 | Margaroyl | γE-γE- | 17 | Amide | 5S, |
| 88 | K(αMePhe)LDSK((PEG) | | | (PEG)2- | | | 6αMePhe, |
| | 2-(PEG)2-γE-γE- | | | (PEG)2 | | | 10V, |
| | Margaroyl)17AAQD | | | | | | 13αMePhe, |
| | (αMePhe)VQ(Aib)IAN- | | | | | | 17K, 18A, |
| | amide | | | | | | 22αMePhe, |
| | | | | | | | 25Aib, 26I, |
| | | | | | | | 27A, des29T |
| Peptide | HSQGS(αMePhe)TSDVS | 160 | Palmitoyl | γE-γE- | 17 | Amide | 5S, |
| 89 | K(αMePhe)LDSK((PEG) | | | (PEG)2- | | | 6αMePhe, |
| | 2-(PEG)2-γE-γE- | | | (PEG)2 | | | 10V, |
| | Palmitoyl)17AAQD | | | | | | 13αMePhe, |
| | (αMePhe)VQIIAN-amide | | | | | | 17K, 18A, |
| | | | | | | | 22αMePhe, |
| | | | | | | | 25Aib, 26I, |
| | | | | | | | 27A, des29T |
| Peptide | HSQGS(αMePhe)TSDVS | 161 | Palmitoyl | γE-γE- | 17 | Amide | 5S, |
| 90 | K(αMePhe)LDSK((PEG) | | | (PEG)2- | | | 6αMePhe, |
| | 2-(PEG)2-γE-γE- | | | (PEG)2 | | | 10V, |
| | Palmitoyl)17AAQD | | | | | | 13αMePhe, |
| | (αMePhe)VE(Aib)IAN- | | | | | | 17K, 18A, |
| | amide | | | | | | 22αMePhe, |
| | | | | | | | 24E, 25Aib, |
| | | | | | | | 26I, 27A, |
| | | | | | | | des29T |
| Peptide | HSQGS(αMePhe)TSDVS | 162 | Palmitoyl | γE-γE- | 17 | Amide | 5S, |
| 91 | K(αMePhe)LDSK((PEG) | | | (PEG)2- | | | 6αMePhe, |
| | 2-(PEG)2-γE-γE- | | | (PEG)2 | | | 10V, |
| | Palmitoyl)17AAQD | | | | | | 13αMePhe, |
| | (αMePhe)VQ(Aib)IAG- | | | | | | 17K, 18A, |
| | amide | | | | | | 22αMePhe, |
| | | | | | | | 25Aib, 26I, |
| | | | | | | | 27A, 28A, |
| | | | | | | | des29T |
| Peptide | HSQGS(αMePhe)TSDVS | 163 | Stearoyl | γE-γE- | 17 | Amide | 5S, |
| 92 | K(Aib)LDSK((PEG)2- | | | (PEG)2- | | | 6αMePhe, |
| | (PEG)2-γE-γE- | | | (PEG)2 | | | 10V, 13Aib, |
| | Stearoyl)17AAQD | | | | | | 17K, 18A, |
| | (αMePhe)VQ(Aib)LEA- | | | | | | 22αMePhe, |
| | amide | | | | | | 25Aib, 27E, |
| | | | | | | | 28A, des29T |
| Peptide | HSQGS(αMePhe)TSDVS | 164 | Stearoyl | γE-γE- | 17 | Amide | 5S, |
| 93 | K(Aib)LDSK((PEG)2- | | | (PEG)2- | | | 6αMePhe, |
| | (PEG)2-γE-γE- | | | (PEG)2 | | | 10V, 13Aib, |
| | Stearoyl)17AAQD | | | | | | 17K, 18A, |
| | (αMePhe)VQ(Aib)LAN- | | | | | | 22αMePhe, |
| | amide | | | | | | 25Aib, 27A, |
| | | | | | | | des29T |
| Peptide | HSQGS(αMePhe)TSDVS | 165 | Stearoyl | γE-γE- | 17 | Amide | 5S, |
| 94 | K(Aib)LDSK((PEG)2- | | | (PEG)2- | | | 6αMePhe, |
| | (PEG)2-γE-γE- | | | (PEG)2 | | | 10V, 13Aib, |
| | Stearoyl)17AAQD | | | | | | 17K, 18A, |
| | (αMePhe)VQ(Aib)LSE- | | | | | | 22αMePhe, |
| | amide | | | | | | 25Aib, 27S, |
| | | | | | | | 28E, des29T |
| Peptide | HSQGS(αMePhe)TSDVS | 166 | Stearoyl | γE-γE- | 17 | Amide | 5S, |
| 95 | K(Aib)LDSK((PEG)2- | | | (PEG)2- | | | 6αMePhe, |
| | (PEG)2-γE-γE- | | | (PEG)2 | | | 10V, 13Aib, |
| | Stearoyl)17AAQD | | | | | | 17K, 18A, |
| | (αMePhe)VQ(Aib)LANT- | | | | | | 22αMePhe, |
| | amide | | | | | | 25Aib, 27A |
| Peptide | HSQGS(αMePhe)TSDVS | 167 | Stearoyl | γE-γE- | 17 | Amide | 5S, |
| 96 | K(Aib)LDSK((PEG)2- | | | (PEG)2- | | | 6αMePhe, |
| | (PEG)2-γE-γE- | | | (PEG)2 | | | 10V, 13Aib, |
| | Stearoyl)17AAQD | | | | | | 17K, 18A, |
| | (αMePhe)VQ(Aib)LEAGG- | | | | | | 22αMePhe, |
| | amide | | | | | | 25Aib, 27E, |
| | | | | | | | 28A, 29G, |
| | | | | | | | 30G |
| Peptide | HSQGS(αMePhe)TSDVS | 168 | Stearoyl | γE-γE- | 17 | Amide | 5S, |
| 97 | K(Aib)LDSK((PEG)2- | | | (PEG)2- | | | 6αMePhe, |
| | (PEG)2-γE-γE- | | | (PEG)2 | | | 10V, 13Aib, |
| | Stearoyl)17AAQD | | | | | | 17K, 18A, |
| | (αMePhe)VQ(Aib)IAN- | | | | | | 22αMePhe, |
| | amide | | | | | | 25Aib, 26I, |
| | | | | | | | 27A, des29T |
| Peptide | HSQGS(αMePhe)TSDVS | 169 | Stearoyl | γE-γE- | 17 | Amide | 5S, |
| 98 | K(Aib)LDSK((PEG)2- | | | (PEG)2- | | | 6αMePhe, |
| | (PEG)2-γE-γE- | | | (PEG)2 | | | 10V, 13Aib, |
| | Stearoyl)17AAQD | | | | | | 17K, 18A, |
| | (αMePhe)VQ(Aib)IIE- | | | | | | 22αMePhe, |
| | amide | | | | | | 25Aib, 26I, |
| | | | | | | | 27I, 28E, |
| | | | | | | | des29T |
| Peptide | HSQGS(αMePhe)TSDVS | 170 | Palmitoyl | γE-γE- | 17 | Amide | 5S, |
| 99 | K(Aib)LDSK((PEG)2- | | | (PEG)2- | | | 6αMePhe, |
| | (PEG)2-γE-γE- | | | (PEG)2 | | | 10V, 13Aib, |
| | Palmitoyl)17AAQD | | | | | | 17K, 18A, |
| | (αMePhe)VQ(Aib)IAN- | | | | | | 22αMePhe, |
| | amide | | | | | | 25Aib, 26I, |
| | | | | | | | 27A, des29T |
| Peptide | H(Aib)QGS(αMePhe)TS | 171 | Stearoyl | γE-γE- | 17 | Amide | 2Aib, 5S, |
| 100 | DVSK(Aib)LDSK((PEG)2- | | | (PEG)2- | | | 6αMePhe, |
| | (PEG)2-γE-γE- | | | (PEG)2 | | | 10V, 13Aib, |
| | Stearyl)17AAQD | | | | | | 17K, 18A, |
| | (αMePhe)VQ(Aib)IAN- | | | | | | 22αMePhe, |
| | amide | | | | | | 25Aib, 26I, |
| | | | | | | | 27A, des29T |
| Peptide | H(Aib)QGS(αMePhe)TS | 172 | Stearoyl | γE-γE- | 17 | Amide | 2Aib, 5S, |
| 101 | DVSK(Aib)LDSK((PEG)2- | | | (PEG)2- | | | 6αMePhe, |
| | (PEG)2-γE-γE- | | | (PEG)2 | | | 10V, 13Aib, |
| | Stearyl)17AAQD | | | | | | 17K, 18A, |
| | (αMePhe)VQ(Aib)IAN- | | | | | | 22αMePhe, |
| | acid | | | | | | 25Aib, 26I, |
| | | | | | | | 27A, des29T |
| Peptide | H(Aib)QGS(αMePhe)TS | 173 | Stearoyl | γE-γE- | 17 | Amide | 2Aib, 5S, |
| 102 | DVSK(Aib)LDSK((PEG)2- | | | (PEG)2- | | | 6αMePhe, |
| | (PEG)2-γE-γE- | | | (PEG)2 | | | 10V, 13Aib, |
| | Stearyl)17RAQD | | | | | | 17K, 18R, |
| | (αMePhe)VQ(Aib)IAN- | | | | | | 22αMePhe, |
| | amide | | | | | | 25Aib, 26I, |
| | | | | | | | 27A, des29T |
| Peptide | H(Aib)QGS(αMePhe)TS | 174 | Stearoyl | γE-γE- | 17 | Amide | 2Aib, 5S, |
| 103 | DVSK(Aib)LDSK((PEG)2- | | | (PEG)2- | | | 6αMePhe, |
| | (PEG)2-γE-γE- | | | (PEG)2 | | | 10V, 13Aib, |
| | Stearyl)17AAQD | | | | | | 17K, 18A, |
| | (αMePhe)VQWIAN- | | | | | | 22αMePhe, |
| | amide | | | | | | 26I, 27A, |
| | | | | | | | des29T |
| Peptide | HTQGS(αMePhe)TSDVS | 175 | Stearoyl | γE-γE- | 17 | Amide | 2T, 5S, |
| 104 | K(Aib)LDSK((PEG)2- | | | (PEG)2- | | | 6αMePhe, |
| | (PEG)2-γE-γE- | | | (PEG)2 | | | 10V, 13Aib, |
| | Stearyl)17AAQD | | | | | | 17K, 18A, |
| | (αMePhe)VQ(Aib)IAN- | | | | | | 22αMePhe, |
| | amide | | | | | | 25Aib, 26I, |
| | | | | | | | 27A, des29T |
| Peptide | HSQGS(αMePhe)TSDVS | 176 | Palmitoyl | γE-γE- | 17 | Amide | 5S, |
| 105 | K(Aib)LDSK((PEG)2- | | | (PEG)2- | | | 6αMePhe, |
| | (PEG)2-γE-yE- | | | (PEG)2 | | | 10V, 13Aib, |
| | Palmitoyl)17QAQD | | | | | | 17K, 18Q, |
| | (αMePhe)VQ(Aib)IAN- | | | | | | 22αMePhe, |
| | amide | | | | | | 25Aib, 26I, |
| | | | | | | | 27A, des29T |
| Peptide | HSQGS(αMePhe)TSDVS | 177 | Palmitoyl | γE-γE- | 17 | Amide | 5S, |
| 106 | K(Aib)LDSK((PEG)2- | | | (PEG)2- | | | 6αMePhe, |
| | (PEG)2-γE-γE- | | | (PEG)2 | | | 10V, 13Aib, |
| | Palmitoyl)17AAQD | | | | | | 17K, 18A, |
| | (αMePhe)VQHIAN-amide | | | | | | 22αMePhe, |
| | | | | | | | 25H, 26I, |
| | | | | | | | 27A, des29T |
| Peptide | HSQGS(αMePhe)TSDVS | 178 | Palmitoyl | γE-γE- | 17 | Amide | 5S, |
| 107 | K(Aib)LDSK((PEG)2- | | | (PEG)2- | | | 6αMePhe, |
| | (PEG)2-γE-γE- | | | (PEG)2 | | | 10V, 13Aib, |
| | Palmitoyl)17QAQD | | | | | | 17K, 18Q, |
| | (αMePhe)VQHIAN-amide | | | | | | 22αMePhe, |
| | | | | | | | 25H, 26I, |
| | | | | | | | 27A, des29T |
| Peptide | HSQGS(αMePhe)TSDVS | 179 | C18diacid | γE- | 17 | Amide | 5S, |
| 108 | K(Aib)LDSK(O2Oc- | | | (O2Oc)- | | | 6αMePhe, |
| | O2Oc-γE- | | | (O2Oc) | | | 10V, 13Aib, |
| | C18diacid)17AAQD | | | | | | 17K, 18A, |
| | (αMePhe)VQ(Aib)IAN- | | | | | | 22αMePhe, |
| | amide | | | | | | 25Aib, 26I, |
| | | | | | | | 27A, des29T |
| Peptide | HSQGS(αMePhe)TSDVS | 180 | C18diacid | γE- | 17 | Amide | 5S, |
| 109 | (Ac- | | | (O2Oc)- | | | 6αMePhe, |
| | Lys)(Aib)LDSK(O2Oc- | | | (O2Oc) | | | 10V, 12Ac- |
| | O2Oc-γE- | | | | | | Lys, 13Aib, |
| | C18diacid)17AAQD | | | | | | 17K, 18A, |
| | (αMePhe)VQ(Aib)IAN- | | | | | | 22αMePhe, |
| | amide | | | | | | 25Aib, 26I, |
| | | | | | | | 27A, des29T |
| Peptide | HSQGS(αMePhe)TSDVS | 181 | Stearoyl | γE-γE- | 17 | Amide | 5S, |
| 110 | (Ac- | | | (PEG)2- | | | 6αMePhe, |
| | Lys)(Aib)LDSK((PEG)2- | | | (PEG)2 | | | 10V, 12Ac- |
| | (PEG)2-γE-yE- | | | | | | Lys, 13Aib, |
| | Stearyl)17AAQD | | | | | | 17K, 18A, |
| | (αMePhe)VQ(Aib)IAN- | | | | | | 22αMePhe, |
| | amide | | | | | | 25Aib, 26I, |
| | | | | | | | 27A, des29T |
| Peptide | HSQGS(αMePhe)TSDVS | 182 | Palmitoyl | γE-γE- | 17 | Amide | 5S, |
| 111 | KILDSK((PEG)2-(PEG)2- | | | (PEG)2- | | | 6αMePhe, |
| | γE-yE- | | | (PEG)2 | | | 10V, 13I, |
| | Palmitoyl)17AAQD | | | | | | 17K, 18A, |
| | (αMePhe)VQ(Aib)IAN- | | | | | | 22αMePhe, |
| | amide | | | | | | 25Aib, 26I, |
| | | | | | | | 27A, des29T |
| Peptide | HSQGS(αMePhe)TSDVS | 183 | Palmitoyl | γE-γE- | 17 | Amide | 5S, |
| 112 | KILDSK((PEG)2-(PEG)2- | | | (PEG)2- | | | 6αMePhe, |
| | γE-γE- | | | (PEG)2 | | | 10V, 13I, |
| | Palmitoyl)17AAQD | | | | | | 17K, 18A, |
| | (αMePhe)VQIIAN-amide | | | | | | 22αMePhe, |
| | | | | | | | 25I, 26I, |
| | | | | | | | 27A, des29T |
| Peptide | HSQGS(αMePhe)TSDVS | 184 | Palmitoyl | γE-yE- | 17 | Amide | 5S, |
| 113 | KILDSK((PEG)2-(PEG)2- | | | (PEG)2- | | | 6αMePhe, |
| | γE-γE- | | | (PEG)2 | | | 10V, 13I, |
| | Palmitoyl)17AAQD | | | | | | 17K, 18A, |
| | (αMePhe)VQIIAG-amide | | | | | | 22αMePhe, |
| | | | | | | | 25I, 26I, |
| | | | | | | | 27A28G, |
| | | | | | | | des29T |
| Peptide | HSQGS(αMePhe)TSDVS | 185 | Palmitoyl | γE-γE- | 17 | Amide | 5S, |
| 114 | KILDSK((PEG)2-(PEG)2- | | | (PEG)2- | | | 6αMePhe, |
| | γE-γE- | | | (PEG)2 | | | 10V, 13I, |
| | Palmitoyl)17AAQD | | | | | | 17K, 18A, |
| | (αMePhe)VEIIAN-amide | | | | | | 22αMePhe, |
| | | | | | | | 24E, 25I, |
| | | | | | | | 26I, 27A, |
| | | | | | | | des29T |
| Peptide | HSQGS(αMePhe)TSDVS | 186 | Palmitoyl | γE-yE- | 17 | Amide | 5S, |
| 115 | KILDSK((PEG)2-(PEG)2- | | | (PEG)2- | | | 6αMePhe, |
| | γE-γE- | | | (PEG)2 | | | 10V, 13I, |
| | Palmitoyl)17AAQD | | | | | | 17K, 18A, |
| | (αMePhe)VQ(αMePhe)IAN- | | | | | | 22αMePhe, |
| | amide | | | | | | 25αMePhe, |
| | | | | | | | 26I, 27A, |
| | | | | | | | des29T |
| Peptide | HSQGS(αMePhe)TSDVS | 187 | Palmitoyl | γE-γE- | 17 | Amide | 5S, |
| 116 | KILDSK((PEG)2-(PEG)2- | | | (PEG)2- | | | 6αMePhe, |
| | γE-γE- | | | (PEG)2 | | | 10V, 13I, |
| | Palmitoyl)17AAQD | | | | | | 17K, 18A, |
| | (αMePhe)VEWIINT-amide | | | | | | 22MePhe, |
| | | | | | | | 24E, 26I, |
| | | | | | | | 27I |
| Peptide | HSQGS(αMePhe)TSDVS | 188 | Stearoyl | γE-γE- | 17 | Amide | 5S, |
| 117 | KILDSK((PEG)2-(PEG)2- | | | (PEG)2- | | | 6αMePhe, |
| | γE-γE- | | | (PEG)2 | | | 10V, 13I, |
| | Stearoyl)17AAQD | | | | | | 17K, 18A, |
| | (αMePhe)VQ(Aib)IINT- | | | | | | 22αMePhe, |
| | amide | | | | | | 25Aib, 26I, |
| | | | | | | | 27I |
| Peptide | HSQGS(αMePhe)TSDVS | 189 | Palmitoyl | γE-γE- | 17 | Amide | 5S, |
| 118 | KILDSK((PEG)2-(PEG)2- | | | (PEG)2- | | | 6αMePhe, |
| | γE-γE- | | | (PEG)2 | | | 10V, 13I, |
| | Palmitoyl)17AAQD | | | | | | 17K, 18A, |
| | (αMePhe)VQ(Aib)IINT- | | | | | | 22αMePhe, |
| | amide | | | | | | 25Aib, 26I, |
| | | | | | | | 27I |
| Peptide | HSQGS(αMePhe)TSDVS | 190 | Myristoyl | γE-γE- | 17 | Amide | 5S, |
| 119 | KILDSK((PEG)2-(PEG)2- | | | (PEG)2- | | | 6αMePhe, |
| | γE-γE- | | | (PEG)2 | | | 10V, 13I, |
| | Myristoyl)17AAQD | | | | | | 17K, 18A, |
| | (αMePhe)VQ(Aib)IINT- | | | | | | 22αMePhe, |
| | amide | | | | | | 25Aib, 26I, |
| | | | | | | | 27I |
| Peptide | HSQGS(αMePhe)TSDVS | 191 | Stearoyl | γE- | 17 | Amide | 5S, |
| 120 | KILDSK((PEG)2-(PEG)2- | | | (PEG)2- | | | 6αMePhe, |
| | γE- | | | (PEG)2 | | | 10V, 13I, |
| | Stearoyl)17AAQD | | | | | | 17K, 18A, |
| | (αMePhe)VQ(Aib)IINT- | | | | | | 22αMePhe, |
| | amide | | | | | | 25Aib, 26I, |
| | | | | | | | 27I |
| Peptide | HSQGS(αMePhe)TSDVS | 192 | Palmitoyl | γE- | 17 | Amide | 5S, |
| 121 | KILDSK((PEG)2-(PEG)2- | | | (PEG)2- | | | 6αMePhe, |
| | γE- | | | (PEG)2 | | | 10V, 13I, |
| | Palmitoyl)17AAQD | | | | | | 17K, 18A, |
| | ePhe)VQ(Aib)IINT- | | | | | | 22αMePhe, |
| | (αMamide | | | | | | 25Aib, 26I, |
| | | | | | | | 27I |
| Peptide | HSQGS(αMePhe)TSDVS | 193 | Myristoyl | γE- | 17 | Amide | 5S, |
| 122 | KILDSK((PEG)2-(PEG)2- | | | (PEG)2- | | | 6αMePhe, |
| | γE- | | | (PEG)2 | | | 10V, 13I, |
| | Myristoyl)17AAQD | | | | | | 17K, 18A, |
| | (αMePhe)VQ(Aib)IINT- | | | | | | 22αMePhe, |
| | amide | | | | | | 25Aib, 26I, |
| | | | | | | | 27I |
| Peptide | H(Aib)QGS(αMePhe)TS | 194 | Palmitoyl | γE-γE- | 17 | Amide | 2Aib, 5S, |
| 123 | DVSKILDSK((PEG)2- | | | (PEG)2- | | | 6αMePhe, |
| | (PEG)2-γE-γE- | | | (PEG)2 | | | 10V, 13I, |
| | Palmitoyl)17AAQD | | | | | | 17K, 18A, |
| | (αMePhe)VQIIAN-amide | | | | | | 22αMePhe, |
| | | | | | | | 251, 26I, |
| | | | | | | | 27A, des29T |
| Peptide | H(Aib)QGS(αMePhe)TS | 195 | Stearoyl | γE-γE- | 17 | Amide | 2Aib, 5S, |
| 124 | DVSKILDSK((PEG)2- | | | (PEG)2- | | | 6αMePhe, |
| | (PEG)2-γE-γE- | | | (PEG)2 | | | 10V, 13I, |
| | Stearoyl)17AAQD | | | | | | 17K, 18A, |
| | (αMePhe)VQ(Aib)IINT- | | | | | | 22αMePhe, |
| | amide | | | | | | 25Aib, 26I, |
| | | | | | | | 27I |
| Peptide | H(Aib)QGS(αMePhe)TS | 196 | Palmitoyl | γE-γE- | 17 | Amide | 2Aib, 5S, |
| 125 | DVSKILDSK((PEG)2- | | | (PEG)2- | | | 6αMePhe, |
| | (PEG)2-γE-γE- | | | (PEG)2 | | | 10V, 13I, |
| | Palmitoyl)17AAQD | | | | | | 17K, 18A, |
| | (αMePhe)VQ(Aib)IINT- | | | | | | 22αMePhe, |
| | amide | | | | | | 25Aib, 26I, |
| | | | | | | | 27I |
| Peptide | H(Aib)QGS(αMePhe)TS | 197 | Myristoyl | γE-γE- | 17 | Amide | 2Aib, 5S, |
| 126 | DVSKILDSK((PEG)2- | | | (PEG)2- | | | 6αMePhe, |
| | (PEG)2-γE-γE- | | | (PEG)2 | | | 10V, 13I, |
| | Myristoyl)17AAQD | | | | | | 17K, 18A, |
| | (αMePhe)VQ(Aib)IINT- | | | | | | 22αMePhe, |
| | amide | | | | | | 25Aib, 26I, |
| | | | | | | | 27I |
| Peptide | H(Aib)QGS(αMePhe)TS | 198 | Stearoyl | γE- | 17 | Amide | 2Aib, 5S, |
| 127 | DVSKILDSK((PEG)2- | | | (PEG)2- | | | 6αMePhe, |
| | (PEG)2-γE- | | | (PEG)2 | | | 10V, 13I, |
| | Stearoyl)17AAQD | | | | | | 17K, 18A, |
| | Phe)VQ(Aib)IINT- | | | | | | 22αMePhe, |
| | (αMeamide | | | | | | 25Aib, 26I, |
| | | | | | | | 27I |
| Peptide | H(Aib)QGS(αMePhe)TS | 199 | Palmitoyl | γE- | 17 | Amide | 2Aib, 5S, |
| 128 | DVSKILDSK((PEG)2- | | | (PEG)2- | | | 6αMePhe, |
| | (PEG)2-γE- | | | (PEG)2 | | | 10V, 13I, |
| | Palmitoyl)17AAQD | | | | | | 17K, 18A, |
| | (αMePhe)VQ(Aib)IINT- | | | | | | 22αMePhe, |
| | amide | | | | | | 25Aib, 26I, |
| | | | | | | | 27I |
| Peptide | H(Aib)QGS(αMePhe)TS | 200 | Myristoyl | γE- | 17 | Amide | 2Aib, 5S, |
| 129 | DVSKILDSK((PEG)2- | | | (PEG)2- | | | 6αMePhe, |
| | (PEG)2-γE- | | | (PEG)2 | | | 10V, 13I, |
| | Myristoyl)17AAQD | | | | | | 17K, 18A, |
| | (αMePhe)VQ(Aib)IINT- | | | | | | 22αMePhe, |
| | amide | | | | | | 25Aib, 26I, |
| | | | | | | | 27I |
| Peptide | H(αMeSer)QGS(αMePh | 201 | Stearoyl | γE-γE- | 17 | Amide | 2αMeSer, |
| 130 | e)TSDVSKILDSK((PEG)2- | | | (PEG)2- | | | 5S, |
| | (PEG)2-γE-γE- | | | (PEG)2 | | | 6αMePhe, |
| | Stearoyl)17AAQD | | | | | | 10V, 13I, |
| | (αMePhe)VQ(Aib)IINT- | | | | | | 17K, 18A, |
| | amide | | | | | | 22αMePhe, |
| | | | | | | | 25Aib, 26I, |
| | | | | | | | 27I |
| Peptide | H(αMeSer)QGS(αMePh | 202 | Palmitoyl | γE-γE- | 17 | Amide | 2αMeSer, |
| 131 | e)TSDVSKILDSK((PEG)2- | | | (PEG)2- | | | 5S, |
| | (PEG)2-γE-γE- | | | (PEG)2 | | | 6αMePhe, |
| | Palmitoyl)17AAQD | | | | | | 10V, 13I, |
| | (αMePhe)VQ(Aib)IINT- | | | | | | 17K, 18A, |
| | amide | | | | | | 22αMePhe, |
| | | | | | | | 25Aib, 26I, |
| | | | | | | | 27I |
| Peptide | H(αMeSer)QGS(αMePh | 203 | Myristoyl | γE-γE- | 17 | Amide | 2αMeSer, |
| 132 | e)TSDVSKILDSK((PEG)2- | | | (PEG)2- | | | 5S, |
| | (PEG)2-γE-γE- | | | (PEG)2 | | | 6αMePhe, |
| | Myristoyl)17AAQD | | | | | | 10V, 13I, |
| | (αMePhe)VQ(Aib)IINT- | | | | | | 17K, 18A, |
| | amide | | | | | | 22αMePhe, |
| | | | | | | | 25Aib, 26I, |
| | | | | | | | 27I |
| Peptide | H(αMeSer)QGS(αMePh | 204 | Stearoyl | γE- | 17 | Amide | 2αMeSer, |
| 133 | e)TSDVSKILDSK((PEG)2- | | | (PEG)2- | | | 5S, |
| | (PEG)2-γE- | | | (PEG)2 | | | 6αMePhe, |
| | Stearoyl)17AAQD | | | | | | 10V, 13I, |
| | (αMePhe)VQ(Aib)IINT- | | | | | | 17K, 18A, |
| | amide | | | | | | 22αMePhe, |
| | | | | | | | 25Aib, 26I, |
| | | | | | | | 27I |
| Peptide | H(αMeSer)QGS(αMePh | 205 | Palmitoyl | γE- | 17 | Amide | 2αMeSer, |
| 134 | e)TSDVSKILDSK((PEG)2- | | | (PEG)2- | | | 5S, |
| | (PEG)2-γE- | | | (PEG)2 | | | 6αMePhe, |
| | Palmitoyl)17AAQD | | | | | | 10V, 13I, |
| | (αMePhe)VQ(Aib)IINT- | | | | | | 17K, 18A, |
| | amide | | | | | | 22αMePhe, |
| | | | | | | | 25Aib, 26I, |
| | | | | | | | 27I |
| Peptide | H(αMeSer)QGS | 206 | Myristoyl | γE- | 17 | Amide | 2αMeSer, |
| 135 | (αMePhe)TSDVSKILDSK | | | (PEG)2- | | | 5S, |
| | ((PEG)2-(PEG)2-γE- | | | (PEG)2 | | | 6αMePhe, |
| | Myristoyl)17AAQD | | | | | | 10V, 13I, |
| | (αMePhe)VQ(Aib)IINT- | | | | | | 17K, 18A, |
| | amide | | | | | | 22αMePhe, |
| | | | | | | | 25Aib, 26I, |
| | | | | | | | 27I |
| Peptide | H(Aib)QGTFTSDVSK | 48 | C18diacid | γE- | 17 | Acid | 2Aib, 10V, |
| 136 | (αMePhe)LDSK(γE-γE- | | | γE(O2Oc)- | | | 13αMePhe, |
| | O2Oc-O2Oc-γE-γE- | | | (O2Oc)- | | | 17K, 20R, |
| | C18diacid)17RARDFVR | | | γE-γE | | | 24R, 27L, |
| | WLLE(Aib)G-acid | | | | | | 28E, 29Aib, |
| | | | | | | | 30G |
| Peptide | H(Aib)QGTFTSDVSK | 49 | C18diacid | γE- | 17 | Acid | 2Aib, 10V, |
| 137 | (αMePhe)LDSK(γE-γE- | | | γE(O2Oc)- | | | 13αMePhe, |
| | O2Oc-O2Oc-γE-γE- | | | (O2Oc)- | | | 17K, 24R, |
| | C18diacid)17RAQDFVR | | | γE-γE | | | 27L, 28E, |
| | WLLEG(Aib)-acid | | | | | | 29G, 30Aib |
| Peptide | H(Aib)QGTFTSDVSK | 50 | C18diacid | γE- | 17 | Acid | 2Aib, 10V, |
| 138 | (αMePhe)LDSK(γE-γE- | | | γE(O2Oc)- | | | 13αMePhe, |
| | O2Oc-O2Oc-γE-yE- | | | (O2Oc)- | | | 17K, 24R, |
| | C18diacid)17RAQDFVR | | | γE-γE | | | 27L, 28E, |
| | WLLE(Aib)G-acid | | | | | | 29Aib, 30G |
| Peptide | H(Aib)QGTFTSDVSK | 51 | C20diacid | γE- | 17 | Acid | 2Aib, 10V, |
| 139 | (αMePhe)LDSK(γE-γE- | | | γE(O2Oc)- | | | 13αMePhe, |
| | O2Oc-O2Oc-γE-γE- | | | (O2Oc)- | | | 17K, 24R, |
| | C20diacid)17RAQDFVR | | | γE-γE | | | 27L, 28E, |
| | WLLE(Aib)G-acid | | | | | | 29Aib, 30G |
| Peptide | H(Aib)QGTFTSDVSK | 52 | C18diacid | γE- | 17 | Acid | 2Aib, 10V, |
| 140 | (αMePhe)LDTK(O2Oc- | | | (O2Oc)- | | | 13αMePhe, |
| | O2Oc-γE- | | | (O2Oc) | | | 16T, 17K, |
| | C18diacid)17RAQDFVR | | | | | | 24R, 27L, |
| | WLLE(Aib)G-acid | | | | | | 28E, 29Aib, |
| | | | | | | | 30G |
| Peptide | H(Aib)QGTFTSDVSK | 53 | C18diacid | γE- | 17 | Acid | 2Aib, 10V, |
| 141 | (αMePhe)LDSK(O2Oc- | | | (O2Oc)- | | | 13αMePhe, |
| | O2Oc-γE- | | | (O2Oc) | | | 17K, 24A, |
| | C18diacid)17RAQDFVA | | | | | | 27L, 28E, |
| | WLLE(Aib)G-acid | | | | | | 29Aib, 30G |
| Peptide | H(Aib)QGTFTSDVSK | 54 | C18diacid | γE- | 17 | Acid | 2Aib, 10V, |
| 142 | (αMePhe)LDSK(O2Oc- | | | (O2Oc)- | | | 13αMePhe, |
| | O2Oc-γE- | | | (O2Oc) | | | 17K, 20R, |
| | C18diacid)17RARDFVA | | | | | | 24A, 27L, |
| | WLLE(Aib)G-acid | | | | | | 28E, 29Aib, |
| | | | | | | | 30G |
| Peptide | H(Aib)QGTFTSDVSK | 55 | C18diacid | γE- | 17 | Acid | 2Aib, 10V, |
| 143 | (αMePhe)LDSK(O2Oc- | | | (O2Oc)- | | | 13αMePhe, |
| | O2Oc-γE- | | | (O2Oc) | | | 17K, 20R, |
| | C18diacid)17RARDFVQ | | | | | | 27L, 28E, |
| | WLLE(Aib)G-acid | | | | | | 29Aib, 30G |
| Peptide | H(Aib)QGTFTSDVSK | 56 | C18diacid | γE- | 17 | Acid | 2Aib, 10V, |
| 144 | (αMePhe)LDSK(γE-γE- | | | γE(O2Oc)- | | | 13αMePhe, |
| | O2Oc-O2Oc-γE-γE- | | | (O2Oc)- | | | 17K, 20R, |
| | C18diacid)17RARDFVR | | | γE-γE | | | 24R, 27L, |
| | WLLE(Aib)G-acid | | | | | | 28E, 29Aib, |
| | | | | | | | 30G |
| Peptide | H(Aib)QGTFTSDVSK | 57 | C18diacid | γE- | 17 | Acid | 2Aib, 10V, |
| 145 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27A, 28S, |
| | WLASRGI-acid | | | | | | 29R, 30G, |
| | | | | | | | 31I |
| Peptide | H(Aib)QGTFTSDVSK | 58 | C18diacid | γE- | 17 | Acid | 2Aib, 10V, |
| 146 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27A, 28S, |
| | WLASR-acid | | | | | | 29R |
| Peptide | H(Aib)QGTFTSDVSK | 59 | C18diacid | γE- | 17 | Acid | 2Aib, 10V, |
| 147 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27E, 28A, |
| | WLEA(Aib)G-acid | | | | | | 29Aib, 30G |
| Peptide | H(Aib)QGTFTSDVSK | 60 | C18diacid | γE- | 17 | Acid | 2Aib, 10V, |
| 148 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 26I, 27A, |
| | WIAE(Aib)G-acid | | | | | | 28E, 29Aib, |
| | | | | | | | 30G |
| Peptide | H(Aib)QGTFTSDVSK | 61 | C18diacid | γE- | 17 | Acid | 2Aib, 10V, |
| 149 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 26V, 27V, |
| | WVVE(Aib)G-acid | | | | | | 28E, 29Aib, |
| | | | | | | | 30G |
| Peptide | H(Aib)QGTFTSDVSK | 62 | C18diacid | γE- | 17 | Acid | 2Aib, 10V, |
| 150 | (αMePhe)LDSK(O2Oc- | | | (O2Oc)- | | | 13αMePhe, |
| | O2Oc-γE- | | | (O2Oc) | | | 17K, 27L, |
| | C18diacid)17RAQDFVQ | | | | | | 28Aib, 30E |
| | WLL(Aib)TE-acid | | | | | | |
| Peptide | H(Aib)QGTFTSDVSK | 63 | C20diacid | γE- | 17 | Acid | 2Aib, 10V, |
| 151 | (αMePhe)LDSK(O2Oc- | | | (O2Oc)- | | | 13αMePhe, |
| | O2Oc-γE- | | | (O2Oc) | | | 17K, 24R, |
| | C20diacid)17RAQDFVR | | | | | | 27L, 28Aib, |
| | WLL(Aib)TE-acid | | | | | | 30E |
| Peptide | H(Aib)QGTFTSDVSK | 64 | C18diacid | γE- | 17 | Acid | 2Aib, 10V, |
| 152 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27L, 28Aib, |
| | WLL(Aib)E-acid | | | | | | 29E |
| Peptide | H(Aib)QGTFTSDVSK | 65 | C20diacid | γE- | 17 | Acid | 2Aib, 10V, |
| 153 | (αMePhe)LDSK(O2Oc- | | | (O2Oc)- | | | 13αMePhe, |
| | O2Oc-yE- | | | (O2Oc) | | | 17K, 24R, |
| | C20diacid)17RAQDFVR | | | | | | 27L, 28Aib, |
| | WLL(Aib)E-acid | | | | | | 29E |
| Peptide | H(Aib)QGTFTSDVSK | 65 | C18diacid | γE | 17 | Acid | 2Aib, 10V, |
| 154 | (αMePhe)LDSK(γE- | | | | | | 13αMePhe, |
| | C18diacid)17RAQDFVQ | | | | | | 17K, 27L, |
| | WLL(Aib)T-acid | | | | | | 28Aib |
| Peptide | H(Aib)QGTFTSDVSK | 66 | C18diacid | γE | 17 | Acid | 2Aib, 10V, |
| 155 | (αMePhe)LDSK(γE- | | | | | | 13αMePhe, |
| | C18diacid)17RAQDFVR | | | | | | 17K, 24R, |
| | WLLE(Aib)G-acid | | | | | | 27L, 28E, |
| | | | | | | | 29Aib, 30G |
| Peptide | H(Aib)QGTFTSDVSK | 67 | C18diacid | γE- | 17 | Acid | 2Aib, 10V, |
| 156 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27L, 28E, |
| | WLLEG(Aib)-acid | | | | | | 29G, 30Aib |
| Peptide | H(Aib)QGTFTSDVSK | 68 | C18diacid | γE | 17 | Acic | 2Aib, 10V, |
| 157 | (αMePhe)LDSK(γE- | | | | | | 13αMePhe, |
| | C18diacid)17RAQDFVR | | | | | | 17K, 24R, |
| | WLLEG(Aib)-acid | | | | | | 27L, 28E, |
| | | | | | | | 29G, 30Aib |
| Peptide | H(Aib)QGTFTSDVSK | 69 | C20diacid | γE- | 17 | Acid | 2Aib, 10V, |
| 158 | (αMePhe)LDSK(O2Oc- | | | (O2Oc)- | | | 13αMePhe, |
| | O2Oc-γE- | | | (O2Oc) | | | 17K, 24R, |
| | C20diacid)17RAQDFVR | | | | | | 27L, 28E, |
| | WLLE(Aib)G-acid | | | | | | 29Aib, 30G |
| Peptide | H(Aib)QGTFTSDVSK | 70 | C20diacid | γE- | 17 | Acid | 2Aib, 10V, |
| 159 | (αMePhe)LDTK(O2Oc- | | | (O2Oc)- | | | 13αMePhe, |
| | O2Oc-γE- | | | (O2Oc) | | | 16T, 17K, |
| | C20diacid)17RAQDFVR | | | | | | 24R, 27L, |
| | WLLE(Aib)G-acid | | | | | | 28E, 29Aib, |
| | | | | | | | 30G |
| Peptide | H(Aib)QGTFTSDVSK | 71 | C18diacid | γE- | 17 | Acid | 2Aib, 10V, |
| 160 | (αMePhe)LESK(O2Oc- | | | (O2Oc)- | | | 13αMePhe, |
| | O2Oc-γE- | | | (O2Oc) | | | 15E, 17K, |
| | C18diacid)17RAQDFVR | | | | | | 24R, 27L, |
| | WLLE(Aib)G-acid | | | | | | 28E, 29Aib, |
| | | | | | | | 30G |
| Peptide | H(Aib)QGTFTSDVSK | 72 | C18diacid | γE- | 17 | Acid | 2Aib, 10V, |
| 161 | (αMePhe)LEAK(O2Oc- | | | (O2Oc)- | | | 13αMePhe, |
| | O2Oc-γE- | | | (O2Oc) | | | 15E, 16A, |
| | C18diacid)17RARDFVA | | | | | | 17K, 20R, |
| | WLLE(Aib)G-acid | | | | | | 24A, 27L, |
| | | | | | | | 28E, 29Aib, |
| | | | | | | | 30G |
| Peptide | H(Aib)QGTFTSDVSK | 73 | C18diacid | γE- | 17 | Acid | 2Aib, 10V, |
| 162 | (αMePhe)LDSK(O2Oc- | | | (O2Oc)- | | | 13αMePhe, |
| | O2Oc-yE- | | | (O2Oc) | | | 17K, 24R, |
| | C18diacid)17RAQDFVR | | | | | | 26I, 27A, |
| | WIA(Aib)TE-acid | | | | | | 28Aib, 30E |
| Peptide | H(Aib)QGTFTSDVSK | 74 | C18diacid | γE- | 17 | Acid | 2Aib, 10V, |
| 163 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27L, 28E, |
| | WLLE(Aib)A-acid | | | | | | 29Aib, 30A |
| Peptide | H(Aib)QGTFTSDVSK | 75 | C18diacid | γE- | 17 | Acid | 2Aib, 10V, |
| 164 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27L, 28E, |
| | WLLEA(Aib)-acid | | | | | | 29A, 30Aib |
| Peptide | H(Aib)QGTFTSDVSK | 76 | C18diacid | γE- | 17 | Acid | 2Aib, 10V, |
| 165 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27V, 28Aib, |
| | WLV(Aib)TE-acid | | | | | | 30E |
| Peptide | H(Aib)QGTFTSDVSK | 77 | C18diacid | γE- | 17 | Acid | 2Aib, 10V, |
| 166 | (αMePhe)LDSK(O2Oc- | | | (O2Oc)- | | | 13αMePhe, |
| | O2Oc-γE- | | | (O2Oc) | | | 17K, 20R, |
| | C18diacid)17RARDFVR | | | | | | 24R, 27L, |
| | WLL(Aib)TE-acid | | | | | | 28Aib, 30E |
| Peptide | H(Aib)QGTFTSDVSK | 78 | C18diacid | γE- | 17 | Acid | 2Aib, 10V, |
| 167 | (αMePhe)LDSK(O2Oc- | | | (O2Oc)- | | | 13αMePhe, |
| | O2Oc-γE- | | | (O2Oc) | | | 17K, 20R, |
| | C18diacid)17RARDFVA | | | | | | 24A, 27L, |
| | WLL(Aib)TE-acid | | | | | | 28Aib, 30E |
| Peptide | H(Aib)QGTFTSDVSK | 79 | C18diacid | γE- | 17 | Acid | 2Aib, 10V, |
| 168 | (αMePhe)LDSK(O2Oc- | | | (O2Oc)- | | | 13αMePhe, |
| | O2Oc-γE- | | | (O2Oc) | | | 17K, 20L, |
| | C18diacid)17RALDFVR | | | | | | 24A, 27L, |
| | WLL(Aib)TE-acid | | | | | | 28Aib, 30E |
| Peptide | H(Aib)QGTFTSDVSK | 80 | C18diacid | γE- | 17 | Acid | 2Aib, 10V, |
| 169 | (αMePhe)LELK(O2Oc- | | | (O2Oc)- | | | 13αMePhe, |
| | O2Oc-γE- | | | (O2Oc) | | | 15E, 16L, |
| | C18diacid)17RAQDFVR | | | | | | 17K, 24R, |
| | WLL(Aib)TE-acid | | | | | | 27L, 28Aib, |
| | | | | | | | 30E |
| Peptide | H(Aib)QGTFTSDVSK | 81 | C18diacid | γE- | 17 | Acid | 2Aib, 10V, |
| 170 | (αMePhe)LDEK(O2Oc- | | | (O2Oc)- | | | 13αMePhe, |
| | O2Oc-γE- | | | (O2Oc) | | | 16E, 17K, |
| | C18diacid)17RAQDFVR | | | | | | 24R, 27L, |
| | WLL(Aib)TE-acid | | | | | | 28Aib, 30E |
| Peptide | H(Aib)QGTFTSDVSK | 82 | C18diacid | γE- | 17 | Acid | 2Aib, 10V, |
| 171 | (αMePhe)LDSK(O2Oc- | | | (O2Oc)- | | | 13αMePhe, |
| | O2Oc-γE- | | | | | | 17K, 24R, |
| | C18diacid)17RAQDFVR | | | (O2Oc) | | | 27E, 28Aib, |
| | WLE(Aib)TE-acid | | | | | | 30E |
| Peptide | H(Aib)QGTFTSDVSK | 83 | C18diacid | γE- | 17 | Acid | 2Aib, 10V, |
| 172 | (αMePhe)LESK(O2Oc- | | | (O2Oc)- | | | 13αMePhe, |
| | O2Oc-γE- | | | (O2Oc) | | | 15E, 17K, |
| | C18diacid)17RAQDFVR | | | | | | 24R, 27L, |
| | WLL(Aib)TE-acid | | | | | | 28Aib, 30E |
| Peptide | H(Aib)QGTFTSDVSK | 84 | C18diacid | γE- | 17 | Acid | 2Aib, 10V, |
| 173 | (αMePhe)LESK(O2Oc- | | | (O2Oc)- | | | 13αMePhe, |
| | O2Oc-γE- | | | (O2Oc) | | | 15E, 17K, |
| | C18diacid)17RAQDFVR | | | | | | 24R, 27L, |
| | WLL(Aib)E-acid | | | | | | 28Aib, 29E |
| Peptide | H(Aib)QGTFTSDVSK | 85 | C18diacid | γE- | 17 | Acid | 2Aib, 10V, |
| 174 | (αMePhe)LDTK(O2Oc- | | | (O2Oc)- | | | 13αMePhe, |
| | O2Oc-γE- | | | (O2Oc) | | | 16T, 17K, |
| | C18diacid)17RAQDFVR | | | | | | 24R, 27L, |
| | WLL(Aib)TE-acid | | | | | | 28Aib, 30E |
| Peptide | H(Aib)QGTFTSDVSK | 86 | C18diacid | γE- | 17 | Acid | 2Aib, 10V, |
| 175 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27A, 28Aib, |
| | WLA(Aib)TE-acid | | | | | | 30E |
| Peptide | H(Aib)QGTFTSDVSK | 87 | C18diacid | γE- | 17 | Acid | 2Aib, 10V, |
| 176 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27A, 28Aib, |
| | WLL(Aib)TA-acid | | | | | | 30A |
| Peptide | H(Aib)QGTFTSDVSK | 88 | C18diacid | YE- | 17 | Acid | 2Aib, 10V, |
| 177 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27A, 28Aib, |
| | WLL(Aib)TK-acid | | | | | | 30K |
| Peptide | H(Aib)QGTFTSDVSK | 89 | C18diacid | γE- | 17 | Acid | 2Aib, 10V, |
| 178 | (αMePhe)LDKK(O2Oc- | | | (O2Oc)- | | | 13αMePhe, |
| | O2Oc-γE- | | | (O2Oc) | | | 16K, 17K, |
| | C18diacid)17RAQDFVR | | | | | | 24R, 27L, |
| | WLL(Aib)TE-acid | | | | | | 28Aib, 30E |
| Peptide | H(Aib)QGTFTSDVSK | 90 | C18diacid | γE- | 17 | Acid | 2Aib, 10V, |
| 179 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27L, 28E, |
| | WLLEAG-acid | | | | | | 29A, 30G |
| Peptide | H(Aib)QGTFTSDVSK | 91 | C18diacid | γE- | 17 | Acid | 2Aib, 10V, |
| 180 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27L, 28A, |
| | WLLA(Aib)G-acid | | | | | | 29Aib, 30G |
| Peptide | H(Aib)QGTFTSDVSK | 92 | C18diacid | (O2Oc)- | 17 | Acid | 2Aib, 10V, |
| 181 | (αMePhe)LDSK(O2Oc- | | | (O2Oc) | | | 13αMePhe, |
| | O2Oc- | | | | | | 17K, 24R, |
| | C18diacid)17RAQDFVR | | | | | | 27L, 28E, |
| | WLLE(Aib)G-acid | | | | | | 29Aib, 30G |
| Peptide | H(Aib)QGTFTSDVSK | 93 | C18diacid | γE- | 17 | Acid | 2Aib, 10V, |
| 182 | (αMePhe)LDKK(O2Oc- | | | (O2Oc)- | | | 13αMePhe, |
| | O2Oc-γE- | | | (O2Oc) | | | 16K, 17K, |
| | C18diacid)17RAQDFVR | | | | | | 24R, 27L, |
| | WLLE(Aib)G-acid | | | | | | 28E, 29Aib, |
| | | | | | | | 30G |
| Peptide | H(Aib)QGTFTSDVSK | 94 | C18diacid | γE- | 17 | Acid | 2Aib, 10V, |
| 183 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27L, 28E, |
| | WLLE(Aib)K-acid | | | | | | 29Aib, 30K |
| Peptide | H(Aib)QGTFTSDVSK( | 95 | C18diacid | (O2Oc)- | 17 | Acid | 2Aib, 10V, |
| 184 | αMePhe)LDKK(O2Oc- | | | (O2Oc) | | | 13αMePhe, |
| | O2Oc- | | | | | | 16K, 17K, |
| | C18diacid)17RAQDFVR | | | | | | 24R, 27L, |
| | WLLA(Aib)K-acid | | | | | | 28A, 29Aib, |
| | | | | | | | 30K |
| Peptide | H(Aib)QGTFTSDVSK | 96 | C18diacid | γE- | 17 | Amide | 2Aib, 10V, |
| 185 | (αMePhe)LDTK(O2Oc- | | | (O2Oc)- | | | 13αMePhe, |
| | O2Oc-γE- | | | (O2Oc) | | | 16T, 17K, |
| | C18diacid)17RAQDFVR | | | | | | 24R, 27L, |
| | WLLE(Aib)G-amide | | | | | | 28E, 29Aib, |
| | | | | | | | 30G |
| Peptide | H(Aib)QGTFTSDVSK | 97 | C18diacid | γE- | 17 | Acid | 2Aib, 10V, |
| 186 | (αMePhe)LDTK(O2Oc- | | | (O2Oc)- | | | 13αMePhe, |
| | O2Oc-γE- | | | (O2Oc) | | | 16T, 17K, |
| | C18diacid)17RAQDFVR | | | | | | 24R, 27L, |
| | WLLEAG-acid | | | | | | 28E, 29A, |
| | | | | | | | 30G |
| Peptide | H(Aib)QGTFTSDVSK | 98 | C18diacid | γE- | 17 | Acid | 2Aib, 10V, |
| 187 | (αMePhe)LDTK(O2Oc- | | | (O2Oc)- | | | 13αMePhe, |
| | O2Oc-γE- | | | (O2Oc) | | | 16T, 17K, |
| | C18diacid)17RARDFVR | | | | | | 20R, 24R, |
| | WLVE(Aib)G-acid | | | | | | 27V, 28E, |
| | | | | | | | 29Aib, 30G |
| Peptide | H(Aib)QGTFTSDVSK | 99 | C18diacid | γE- | 17 | Acid | 2Aib, 10V, |
| 188 | (αMePhe)LDTK(O2Oc- | | | (O2Oc)- | | | 13αMePhe, |
| | O2Oc-γE- | | | (O2Oc) | | | 16T, 17K, |
| | C18diacid)17RARDFVQ | | | | | | 20R, 27L, |
| | WLLE(Aib)G-acid | | | | | | 28E, 29Aib, |
| | | | | | | | 30G |
| Peptide | H(Aib)QGTFTSDVSK | 100 | C18diacid | γE- | 17 | Acid | 2Aib, 10V, |
| 189 | (αMePhe)LDTK(O2Oc- | | | (O2Oc)- | | | 13αMePhe, |
| | O2Oc-γE- | | | (O2Oc) | | | 16T, 17K, |
| | C18diacid)17RARDFVR | | | | | | 20R, 24R, |
| | WLLE(Aib)G-acid | | | | | | 27L, 28E, |
| | | | | | | | 29Aib, 30G |
| Peptide | H(Aib)QGTFTSDVSK | 101 | C20diacid | γE- | 17 | Acid | 2Aib, 10V, |
| 190 | (αMePhe)LDTK(O2Oc- | | | (O2Oc)- | | | 13αMePhe, |
| | O2Oc-γE- | | | (O2Oc) | | | 16T, 17K, |
| | C20diacid)17RAQDFVR | | | | | | 24R, 27V, |
| | WLVE(Aib)G-acid | | | | | | 28E, 29Aib, |
| | | | | | | | 30G |
| Peptide | H(Aib)QGTFTSDVSK | 102 | C18diacid | γE- | 17 | Acid | 2Aib, 10V, |
| 191 | (αMePhe)LDTK(O2Oc- | | | (O2Oc)- | | | 13αMePhe, |
| | O2Oc-γE- | | | (O2Oc) | | | 16T, 17K, |
| | C18diacid)17RAQDFVR | | | | | | 24R, 27V, |
| | WLVE(Aib)G-acid | | | | | | 28E, 29Aib, |
| | | | | | | | 30G |
| Peptide | H(Aib)QGTFTSDVSK | 103 | C18diacid | γE- | 17 | Acid | 2Aib, 10V, |
| 192 | (αMePhe)LDTK(O2Oc- | | | (O2Oc)- | | | 13αMePhe, |
| | O2Oc-γE- | | | (O2Oc) | | | 16T, 17K, |
| | C18diacid)17RAQDFVR | | | | | | 24R, 27L, |
| | WLL(Aib)E-acid | | | | | | 28Aib, 29E |
| Peptide | H(Aib)QGTFTSDVSK | 104 | C20diacid | γE- | 17 | Acid | 2Aib, 10V, |
| 193 | (αMePhe)LDTK(O2Oc- | | | (O2Oc)- | | | 13αMePhe, |
| | O2Oc-γE- | | | (O2Oc) | | | 16T, 17K, |
| | C20diacid)17RAQDFVR | | | | | | 24R, 27L, |
| | WLL(Aib)E-acid | | | | | | 28Aib, 29E |
| Peptide | H(Aib)QGTFTSDVSK | 105 | C20diacid | γE- | 17 | Acid | 2Aib, 10V, |
| 194 | (αMePhe)LDTK(O2Oc- | | | (O2Oc)- | | | 13αMePhe, |
| | O2Oc-γE-γE- | | | (O2Oc) | | | 16T, 17K, |
| | C20diacid)17RAQDFVR | | | | | | 24R, 27L, |
| | WLL(Aib)E-acid | | | | | | 28Aib, 29E |
| Peptide | H(Aib)QGTFTSDVSK | 106 | C20diacid | γE- | 17 | Acid | 2Aib, 10V, |
| 195 | (αMePhe)LDTK(O2Oc- | | | (O2Oc)- | | | 13αMePhe, |
| | O2Oc-γE- | | | (O2Oc) | | | 16T, 17K, |
| | C20diacid)17RARDFVQ | | | | | | 20R, 27L, |
| | WLLE(Aib)G-acid | | | | | | 28E, 29Aib, |
| | | | | | | | 30G |
| Peptide | H(Aib)QGTFTSDVSK | 107 | C18diacid | γE- | 17 | Acid | 2Aib, 10V, |
| 196 | (αMePhe)LDSK(O2Oc- | | | (O2Oc)- | | | 13αMePhe, |
| | O2Oc-γE- | | | (O2Oc) | | | 17K, 24R, |
| | C18diacid)17RAQDFVR | | | | | | 27I, 28Aib, |
| | WLI(Aib)E-acid | | | | | | 29E |
| Peptide | H(Aib)QGTFTSDVSK | 108 | C18diacid | γE- | 17 | Acid | 2Aib, 10V, |
| 197 | (αMePhe)LDTK(O2Oc- | | | (O2Oc)- | | | 13αMePhe, |
| | O2Oc-γE- | | | (O2Oc) | | | 16T, 17K, |
| | C18diacid)17RARDFVR | | | | | | 20R, 24R, |
| | WLL(Aib)E-acid | | | | | | 27L, 28Aib, |
| | | | | | | | 29E |
| Peptide | H(Aib)QGTFTSDVSK | 109 | C18diacid | γE- | 17 | Acid | 2Aib, 10V, |
| 198 | (αMePhe)LDTK(O2Oc- | | | (O2Oc)- | | | 13αMePhe, |
| | O2Oc-γE- | | | (O2Oc) | | | 16T, 17K, |
| | C18diacid)17RARDFIA | | | | | | 20R, 23I, |
| | WLL(Aib)E-acid | | | | | | 24A, 27L, |
| | | | | | | | 28Aib, 29E |
| Peptide | H(Aib)QGTFTSDVSK(αM | 110 | C18diacid | γE- | 17 | Acid | 2Aib, 10V, |
| 199 | ePhe)LDTK(O2Oc- | | | (O2Oc)- | | | 13αMePhe, |
| | O2Oc-γE- | | | (O2Oc) | | | 16T, 17K, |
| | C18diacid)17RARDFIA | | | | | | 20R, 23I, |
| | WLLE(Aib)G-acid | | | | | | 24A, 27L, |
| | | | | | | | 28E, 29Aib, |
| | | | | | | | 30G |
| Peptide | H(Aib)QGTFTSDVSK | 111 | C18diacid | γE- | 17 | Acid | 2Aib, 10V, |
| 200 | (αMePhe)LDTK(O2Oc- | | | (O2Oc)- | | | 13αMePhe, |
| | O2Oc-γE- | | | (O2Oc) | | | 16T, 17K, |
| | C18diacid)17RARDFVA | | | | | | 20R, |
| | WLLE(Aib)G-acid | | | | | | 24A, 27L, |
| | | | | | | | 28E, |
| | | | | | | | 29Aib, 30G |
| Peptide | H(Aib)QGTFTSDVSK | 112 | C18diacid | γE- | 17 | Acid | 2Aib, 10V, |
| 201 | (αMePhe)LDTK(O2Oc- | | | (O2Oc)- | | | 13αMePhe, |
| | O2Oc-γE- | | | (O2Oc) | | | 16T, 17K, |
| | C18diacid)17RARDFVA | | | | | | 20R, 24A, |
| | WLEA(Aib)G-acid | | | | | | 27E, 28A, |
| | | | | | | | 29Aib, 30G |
| Peptide | H(Aib)QGTFTSDVSK | 113 | C18diacid | γE- | 17 | Acid | 2Aib, 10V, |
| 202 | (αMePhe)LDTK(O2Oc- | | | (O2Oc)- | | | 13αMePhe, |
| | O2Oc-γE- | | | (O2Oc) | | | 16T, 17K, |
| | C18diacid)17RARDFVQ | | | | | | 20R, 27E, |
| | WLEA(Aib)G-acid | | | | | | 28A, 29Aib, |
| | | | | | | | 30G |
| Peptide | H(Aib)QGT(αMFTSDVSK | 418 | C20diacid | γE- | 17 | Acid | 2(Aib), 10V, |
| 407 | ePhe)LDTK(O2Oc- | | | (O2Oc)- | | | 13(αMePhe), |
| | O2Oc-yE- | | | (O2Oc) | | | 16T, 17K, |
| | C20diacid)17RAQDFVR | | | | | | 24R, 27E, |
| | WLEA(Aib)G-acid | | | | | | 28A, 29(Aib), |
| | | | | | | | 30G |
| Peptide | H(Aib)QGTFTSDVSK | 419 | C20diacid | γE-γE- | 17 | Acid | 2(Aib), 10V, |
| 408 | (αMePhe)LDTK(O2Oc- | | | (O2Oc)- | | | 13(αMePhe), |
| | O2Oc-γE-γE- | | | (O2Oc) | | | 16T, 17K, |
| | C20diacid)17RAQDFVR | | | | | | 24R, 27E, |
| | WLEA(Aib)G-acid | | | | | | 28A, 29(Aib), |
| | | | | | | | 30G |
| Peptide | H(Aib)QGTFTSDVSK | 420 | C18diacid | γE- | 17 | Acid | 2(Aib), 10V, |
| 409 | (αMePhe)LDTK(O2Oc- | | | (O2Oc)- | | | 13(αMePhe), |
| | O2Oc-γE- | | | (O2Oc) | | | 16T, 17K, |
| | C18diacid)17RARDFVA | | | | | | 20R, 24A, |
| | WLL(Aib)TE-acid | | | | | | 27L, 28(Aib), |
| | | | | | | | 30E |
| Peptide | H(Aib)QGTFTSDVSK | 421 | C20diacid | γE- | 17 | Acid | 2(Aib), 10V, |
| 410 | (αMePhe)LDTK(O2Oc- | | | (O2Oc)- | | | 13(αMePhe), |
| | O2Oc-γE- | | | (O2Oc) | | | 16T, 17K, |
| | C20diacid)17RARDFVA | | | | | | 20R, 24A, |
| | WLL(Aib)TE-acid | | | | | | 27L, 28(Aib), |
| | | | | | | | 30E |
| Peptide | H(Aib)QGTFTSDVSK | 422 | C18diacid | γE- | 17 | Acid | 2(Aib), 10V, |
| 411 | (αMePhe)LDTK(O2Oc- | | | (O2Oc)- | | | 13(αMePhe), |
| | O2Oc-γE- | | | (O2Oc) | | | 16T, 17K, |
| | C18diacid)17RAQDFVA | | | | | | 24A, 27E, |
| | WLEAGG-acid | | | | | | 28A, 29G, |
| | | | | | | | 30G |
| Peptide | H(Aib)QGTFTSDVSK | 423 | C20diacid | γE- | 17 | Acid | 2(Aib), 10V, |
| 412 | (αMePhe)LDTK(O2Oc- | | | (O2Oc)- | | | 13(αMePhe), |
| | O2Oc-γE- | | | (O2Oc) | | | 16T, 17K, |
| | C20diacid)17RAQDFVA | | | | | | 24A, 27E, |
| | WLEAGG-acid | | | | | | 28A, 29G, |
| | | | | | | | 30G |
| Peptide | H(Aib)QGTFTSDVSK | 424 | C20diacid | γE-γE- | 17 | Acid | 2(Aib), 10V, |
| 413 | (αMePhe)LDTK(O2Oc- | | | (O2Oc)- | | | 13(αMePhe), |
| | O2Oc-γE-γE- | | | (O2Oc) | | | 16T, 17K, |
| | C20diacid)17RAQDFVA | | | | | | 24A, 27E, |
| | WLEAGG-acid | | | | | | 28A, 29G, |
| | | | | | | | 30G |
| Peptide | H(Aib)QGTFTSDVSK | 425 | C18diacid | γE- | 17 | Acid | 2(Aib), 10V, |
| 414 | (αMePhe)LDKK(O2Oc- | | | (O2Oc)- | | | 13(αMePhe), |
| | O2Oc-γE- | | | (O2Oc) | | | 16K, 17K, |
| | C18diacid)17RARDFVR | | | | | | 20R, 24R, |
| | WLL(Aib)E-acid | | | | | | 27L, 28(Aib), |
| | | | | | | | 29E |
| Peptide | H(Aib)QGTFTSDVSK | 426 | C18diacid | γE- | 17 | Acid | 2(Aib), 10V, |
| 415 | (αMePhe)LESK(O2Oc- | | | (O2Oc)- | | | 13(αMePhe), |
| | O2Oc-γE- | | | (O2Oc) | | | 15E, 17K, |
| | C18diacid)17RAQDFVR | | | | | | 24R, 27E, |
| | WLEA(Aib)G-acid | | | | | | 28A, 29(Aib), |
| | | | | | | | 30G |
| Peptide | H(Aib)QGTFTSDVSK | 427 | C20diacid | γE- | 17 | Acid | 2(Aib), 10V, |
| 416 | (αMePhe)LESK(O2Oc- | | | (O2Oc)- | | | 13(αMePhe), |
| | O2Oc-γE- | | | (O2Oc) | | | 15E, 17K, |
| | C20diacid)17RAQDFVR | | | | | | 24R, 27E, |
| | WLEA(Aib)G-acid | | | | | | 28A, 29(Aib), |
| | | | | | | | 30G |
| Peptide | H(Aib)QGTFTSDVSK | 428 | C18diacid | γE-γE- | 17 | Acid | 2(Aib), 10V, |
| 417 | (αMePhe)LDTK(O2Oc- | | | (O2Oc)- | | | 13(αMePhe), |
| | O2Oc-γE-yE- | | | (O2Oc) | | | 16T, 17K, |
| | C18diacid)17RAQDFVA | | | | | | 24A, 27E, |
| | WLEA(Aib)G-acid | | | | | | 28A, 29(Aib), |
| | | | | | | | 30G |
| Peptide | H(Aib)QGTFTSDVSK | 429 | C20diacid | γE-γE- | 17 | Acid | 2(Aib), 10V, |
| 418 | (αMePhe)LDTK(O2Oc- | | | (O2Oc)- | | | 13(αMePhe), |
| | O2Oc-γE-yE- | | | (O2Oc) | | | 16T, 17K, |
| | C20diacid)17RAQDFVA | | | | | | 24A, 27E, |
| | WLEA(Aib)G-acid | | | | | | 28A, 29(Aib), |
| | | | | | | | 30G |
| Peptide | H(Aib)QGTFTSDVSK | 430 | C18diacid | γE- | 17 | Acid | 2(Aib), 10V, |
| 419 | (αMePhe)LDTK(O2Oc- | | | (O2Oc)- | | | 13(αMePhe), |
| | O2Oc-γE- | | | (O2Oc) | | | 16T, 17K, |
| | C18diacid)17RAQDFVR | | | | | | 24R, 27E, |
| | WLE(Aib)E-acid | | | | | | 28(Aib), 29E |
| Peptide | H(Aib)QGTFTSDVSK | 431 | C18diacid | γE- | 17 | Amide | 2(Aib), 10V, |
| 420 | (αMePhe)LDKK(O2Oc- | | | (O2Oc)- | | | 13(αMePhe), |
| | O2Oc-γE- | | | (O2Oc) | | | 16K, 17K, |
| | C18diacid)17RAQDFVR | | | | | | 24R, 27L, |
| | WLL(Aib)E-amide | | | | | | 28(Aib), 29E |
| Peptide | H(Aib)QGTFTSDVSK | 432 | C18diacid | γE- | 17 | Acid | 2(Aib), 10V, |
| 421 | (αMePhe)LDRK(O2Oc- | | | (O2Oc)- | | | 13(αMePhe), |
| | O2Oc-γE- | | | (O2Oc) | | | 16R, 17K, |
| | C18diacid)17RAQDFVR | | | | | | 24R, 27L, |
| | WLL(Aib)TE-acid | | | | | | 28(Aib), 30E |
| Peptide | H(Aib)QGTFTSDVSK | 433 | C18diacid | γE- | 17 | Amide | 2(Aib), 10V, |
| 422 | (αMePhe)LDRK(O2Oc- | | | (O2Oc)- | | | 13(αMePhe), |
| | O2Oc-γE- | | | (O2Oc) | | | 16R, 17K, |
| | C18diacid)17RAQDFVR | | | | | | 24R, 27L, |
| | WLL(Aib)TE-amide | | | | | | 28(Aib), 30E |
| Peptide | H(Aib)QGTFTSDVSK | 434 | C18diacid | γE- | 17 | Acid | 2(Aib), 10V, |
| 423 | (αMePhe)LDKK(O2Oc- | | | (O2Oc)- | | | 13(αMePhe), |
| | O2Oc-γE- | | | (O2Oc) | | | 16K, 17K, |
| | C18diacid)17RAQDFVR | | | | | | 24R, 27K, |
| | WLK(Aib)TE-acid | | | | | | 28(Aib), 30E |
| Peptide | H(Aib)QGTFTSDVSK | 435 | C18diacid | γE- | 17 | Acid | 2(Aib), 10V, |
| 424 | (αMePhe)LDKK(O2Oc- | | | (O2Oc)- | | | 13(αMePhe), |
| | O2Oc-γE- | | | (O2Oc) | | | 16K, 17K, |
| | C18diacid)17RAQDFVR | | | | | | 24R, 27L, |
| | WLL(Aib)TK-acid | | | | | | 28(Aib), 30K |
| Peptide | H(Aib)QGTFTSDVSK | 436 | C18diacid | γE- | 17 | Acid | 2(Aib), 10V, |
| 425 | (αMePhe)LDKK(O2Oc- | | | (O2Oc)- | | | 13(αMePhe), |
| | O2Oc-γE- | | | (O2Oc) | | | 16K, 17K, |
| | C18diacid)17RAQDFVR | | | | | | 24R, 27K, |
| | WLKA(Aib)G-acid | | | | | | 28A, 29(Aib), |
| | | | | | | | 30G |
| Peptide | H(Aib)QGTFTSDVSK | 437 | C18diacid | γE- | 17 | Acid | 2(Aib), 10V, |
| 426 | (αMePhe)LDKK(O2Oc- | | | (O2Oc)- | | | 13(αMePhe), |
| | O2Oc-γE- | | | (O2Oc) | | | 16K, 17K, |
| | C18diacid)17RAQDFVR | | | | | | 24R, 27L, |
| | WLLR(Aib)K-acid | | | | | | 28R, 29(Aib), |
| | | | | | | | 30K |
| Peptide | H(Aib)QGTFTSDVSK | 438 | C18diacid | γE- | 17 | Amide | 2(Aib), 10V, |
| 427 | (αMePhe)LDTK(O2Oc- | | | (O2Oc)- | | | 13(αMePhe), |
| | O2Oc-yE- | | | (O2Oc) | | | 16T, 17K, |
| | C18diacid)17RARDFVQ | | | | | | 20R, 27L, |
| | WLLE(Aib)G-amide | | | | | | 28E, 29(Aib), |
| | | | | | | | 30G |
| Peptide | H(Aib)QGTFTSDVSK | 439 | C18diacid | γE-yE- | 17 | Amide | 2(Aib), 10V, |
| 428 | (αMePhe)LDTK(O2Oc- | | | (O2Oc)- | | | 13(αMePhe), |
| | O2Oc-γE-yE- | | | (O2Oc) | | | 16T, 17K, |
| | C18diacid)17RARDFVQ | | | | | | 20R, 27L, |
| | WLLE(Aib)G-amide | | | | | | 28E, 29(Aib), |
| | | | | | | | 30G |
| Peptide | H(Aib)QGTFTSDVSK | 440 | C18diacid | γE- | 17 | Amide | 2(Aib), 10V, |
| 429 | (αMePhe)LDSK(O2Oc- | | | (O2Oc)- | | | 13(αMePhe), |
| | O2Oc-γE- | | | (O2Oc) | | | 17K, 24R, |
| | C18diacid)17RAQDFVR | | | | | | 27E, 28A, |
| | WLEA(Aib)G-amide | | | | | | 29(Aib), 30G |
| Peptide | H(Aib)QGTFTSDVSK | 441 | C18diacid | γE-γE- | 17 | Amide | 2(Aib), 10V, |
| 430 | (αMePhe)LDSK(O2Oc- | | | (O2Oc)- | | | 13(αMePhe), |
| | O2Oc-γE-γE- | | | (O2Oc) | | | 17K, 24R, |
| | C18diacid)17RAQDFVR | | | | | | 27E, 28A, |
| | WLEA(Aib)G-amide | | | | | | 29(Aib), 30G |
| Peptide | H(Aib)QGTFTSDVSK | 442 | C18diacid | γE-γE- | 17 | Amide | 2(Aib), 10V, |
| 431 | (αMePhe)LDTK(O2Oc- | | | (O2Oc)- | | | 13(αMePhe), |
| | O2Oc-γE-yE- | | | (O2Oc) | | | 16T, 17K, |
| | C18diacid)17RAQDFVA | | | | | | 24A, 27E, |
| | WLEA(Aib)G-amide | | | | | | 28A, 29(Aib), |
| | | | | | | | 30G |
| Peptide | H(Aib)QGTFTSDVSK | 443 | C18diacid | γE- | 17 | Acid | 2(Aib), 10V, |
| 432 | (αMePhe)LDSK(O2Oc- | | | (O2Oc)- | | | 13(αMePhe), |
| | O2Oc-γE- | | | (O2Oc) | | | 17K, 24R, |
| | C18diacid)17RAQDFVR | | | | | | 25(Bip), 27L, |
| | (Bip)LL(Aib)E-acid | | | | | | 28(Aib), 29E |
| Peptide | H(Aib)QGTFTSDVSK | 444 | C18diacid | γE- | 17 | Acid | 2(Aib), 10V, |
| 433 | (αMePhe)LDSK(O2Oc- | | | (O2Oc)- | | | 13(αMePhe), |
| | O2Oc-γE- | | | (O2Oc) | | | 17K, 24R, |
| | C18diacid)17RAQDFVR | | | | | | 25(1-Methyl- |
| | (1-Methyl-Trp)LL | | | | | | Trp), 27L, |
| | (Aib)E-acid | | | | | | 28(Aib), 29E |
| Peptide | H(Aib)QGTFTSDVSK | 445 | C18diacid | γE- | 17 | Acid | 2(Aib), 10V, |
| 434 | (αMePhe)LDSK(O2Oc- | | | (O2Oc)- | | | 13(αMePhe), |
| | O2Oc-γE- | | | (O2Oc) | | | 17K, 24R, |
| | C18diacid)17RAQDFVR | | | | | | 25(5-BrTrp), |
| | (5-BrTrp)LL(Aib)E- | | | | | | 27L, 28(Aib), |
| | acid | | | | | | 29E |
| Peptide | H(Aib)QGTFTSDVSK | 446 | C18diacid | γE- | 17 | Acid | 2(Aib), 10V, |
| 435 | (αMePhe)LDTK(O2Oc- | | | (O2Oc)- | | | 13(αMePhe), |
| | O2Oc-γE- | | | (O2Oc) | | | 16T, 17K, |
| | C18diacid)17RARDFVQ | | | | | | 20R, 25(Aib), |
| | (Aib)LLE(Aib)G-acid | | | | | | 27L, 28E, |
| | | | | | | | 29(Aib), 30G |
| Peptide | H(Aib)QGTFTSDVSK | 447 | C18diacid | γE- | 17 | Amide | 2(Aib), 10V, |
| 436 | (αMePhe)LDRK(O2Oc- | | | (O2Oc)- | | | 13(αMePhe), |
| | O2Oc-γE- | | | (O2Oc) | | | 16R, 17K, |
| | C18diacid)17RAQDFVR | | | | | | 24R, 27V, |
| | WLV(Aib)E-amide | | | | | | 28(Aib), 29E |
| Peptide | H(Aib)QGTFTSDVSK | 448 | C20diacid | γE- | 17 | Amide | 2(Aib), 10V, |
| 437 | (αMePhe)LDRK(O2Oc- | | | (O2Oc)- | | | 13(αMePhe), |
| | O2Oc-γE- | | | (O2Oc) | | | 16R, 17K, |
| | C20diacid)17RAQDFVR | | | | | | 24R, 27V, |
| | WLV(Aib)E-amide | | | | | | 28(Aib), 29E |
| Peptide | H(Aib)QGTFTSDVSK | 449 | C18diacid | γE- | 17 | Amide | 2(Aib), 10V, |
| 438 | (αMePhe)LDKK(O2Oc- | | | (O2Oc)- | | | 13(αMePhe), |
| | O2Oc-γE- | | | (O2Oc) | | | 16K, 17K, |
| | C18diacid)17RAQDFVR | | | | | | 24R, 27E, |
| | WLEA(Aib)G-amide | | | | | | 28A, 29(Aib), |
| | | | | | | | 30G |
| Peptide | H(Aib)QGTFTSDVSK | 450 | C18diacid | γE- | 17 | Amide | 2(Aib), 10V, |
| 439 | (αMePhe)LDRK(O2Oc- | | | (O2Oc)- | | | 13(αMePhe), |
| | O2Oc-γE- | | | (O2Oc) | | | 16R, 17K, |
| | C18diacid)17RAQDFVR | | | | | | 24R, 27L, |
| | WLL(Aib)E-amide | | | | | | 28(Aib), 29E |
| Peptide | H(Aib)QGTFTSDVSK | 451 | C20diacid | γE- | 17 | Acid | 2(Aib), 10V, |
| 440 | (αMePhe)LDKK(O2Oc- | | | (O2Oc)- | | | 13(αMePhe), |
| | O2Oc-γE- | | | (O2Oc) | | | 16K, 17K, |
| | C20diacid)17RARDFVR | | | | | | 20R, 24R, |
| | WLL(Aib)E-acid | | | | | | 27L, 28(Aib), |
| | | | | | | | 29E |
| Peptide | H(Aib)QGTFTSDVSK | 452 | C20diacid | γE- | 17 | Amide | 2(Aib), 10V, |
| 441 | (αMePhe)LDKK(O2Oc- | | | (O2Oc)- | | | 13(αMePhe), |
| | O2Oc-γE- | | | (O2Oc) | | | 16K, 17K, |
| | C20diacid)17RAQDFVR | | | | | | 24R, 27L, |
| | WLL(Aib)E-amide | | | | | | 28(Aib), 29E |
| Peptide | H(Aib)QGTFTSDVSK | 453 | C18diacid | γE- | 17 | Amide | 2(Aib), 10V, |
| 442 | (αMePhe)LDKK(O2Oc- | | | (O2Oc)- | | | 13(αMePhe), |
| | O2Oc-γE- | | | (O2Oc) | | | 16K, 17K, |
| | C18diacid)17RAQDFVR | | | | | | 24R, 27L, |
| | WLLE(Aib)G-amide | | | | | | 28E, 29(Aib), |
| | | | | | | | 30G |
| Peptide | H(Aib)QGTFTSDVSK | 454 | C18diacid | γE- | 17 | Amide | 2(Aib), 10V, |
| 443 | (αMePhe)LDKK(O2Oc- | | | (O2Oc)- | | | 13(αMePhe), |
| | O2Oc-γE- | | | (O2Oc) | | | 16K, 17K, |
| | C18diacid)17RARDFVR | | | | | | 20R, 24R, |
| | WLL(Aib)E-amide | | | | | | 27L, 28(Aib), |
| | | | | | | | 29E |
| Peptide | H(Aib)QGTFTSDVSK | 455 | C18diacid | γE- | 17 | Acid | 2(Aib), 10V, |
| 444 | (αMePhe)LETK(O2Oc- | | | (O2Oc)- | | | 13(αMePhe), |
| | O2Oc-γE- | | | (O2Oc) | | | 15E, 16T, |
| | C20diacid)17RARDFVQ | | | | | | 17K, 20R, |
| | WLLE(Aib)G-acid | | | | | | 27L, 28E, |
| | | | | | | | 29(Aib), 30G |
| Peptide | H(Aib)QGTFTSDVSK | 456 | C18diacid | γE- | 17 | Acid | 2(Aib), 10V, |
| 445 | (αMePhe)LETK(O2Oc- | | | (O2Oc)- | | | 13(αMePhe), |
| | O2Oc-γE- | | | (O2Oc) | | | 15E, 16T, |
| | C18diacid)17RARDFVQ | | | | | | 17K, 20R, |
| | WLLE(Aib)G-acid | | | | | | 27L, 28E, |
| | | | | | | | 29(Aib), 30G |
| Peptide | H(Aib)QGTFTSDVSK | 457 | C18diacid | γE-γE- | 17 | Amide | 2(Aib), 10V, |
| 446 | (αMePhe)LDRK(O2Oc- | | | (O2Oc)- | | | 13(αMePhe), |
| | O2Oc-γE-γE- | | | (O2Oc) | | | 16R, 17K, |
| | C18diacid)17RAQDFVR | | | | | | 24R, 27V, |
| | WLV(Aib)E-amide | | | | | | 28(Aib), 29E |
| Peptide | H(Aib)QGTFTSDVSK | 458 | C20diacid | γE-yE- | 17 | Amide | 2(Aib), 10V, |
| 447 | (αMePhe)LDRK(O2Oc- | | | (O2Oc)- | | | 13(αMePhe), |
| | O2Oc-γE-γE- | | | (O2Oc) | | | 16R, 17K, |
| | C20diacid)17RAQDFVR | | | | | | 24R, 27V, |
| | WLV(Aib)E-amide | | | | | | 28(Aib), 29E |
| Peptide | H(Aib)QGTFTSDVSK | 459 | C18diacid | γE-γE- | 17 | Acid | 2(Aib), 10V, |
| 448 | (αMePhe)LDKK(O2Oc- | | | (O2Oc)- | | | 13(αMePhe), |
| | O2Oc-γE-γE- | | | (O2Oc) | | | 16K, 17K, |
| | C18diacid)17RARDFVR | | | | | | 20R, 24R, |
| | WLL(Aib)E-acid | | | | | | 27L, 28(Aib), |
| | | | | | | | 29E |
| Peptide | H(Aib)QGTFTSDVSK | 460 | C20diacid | γE-γE- | 17 | Acid | 2(Aib), 10V, |
| 449 | (αMePhe)LDTK(O2Oc- | | | (O2Oc)- | | | 13(αMePhe), |
| | O2Oc-γE-yE- | | | (O2Oc) | | | 16T, 17K, |
| | C20diacid)17RARDFVQ | | | | | | 20R, 27L, |
| | WLLE(Aib)G-acid | | | | | | 28E, 29(Aib), |
| | | | | | | | 30G |
| Peptide | H(Aib)QGTFTSDVSK | 461 | C18diacid | γE-γE- | 17 | Acid | 2(Aib), 10V, |
| 450 | (αMePhe)LDKK(O2Oc- | | | (O2Oc)- | | | 13(αMePhe), |
| | O2Oc-γE-γE- | | | (O2Oc) | | | 16K, 17K, |
| | C18diacid)17RAQDFVR | | | | | | 24R, 27L, |
| | WLLE(Aib)G-acid | | | | | | 28E, 29(Aib), |
| | | | | | | | 30G |
| Peptide | H(Aib)QGTFTSDVSK | 462 | C20diacid | γE- | 17 | Amide | 2(Aib), 10V, |
| 451 | (αMePhe)LDKK(O2Oc- | | | (O2Oc)- | | | 13(αMePhe), |
| | O2Oc-γE- | | | (O2Oc) | | | 16K, 17K, |
| | C20diacid)17RARDFVL | | | | | | 20R, 24L, |
| | WLL(Aib)E-amide | | | | | | 27L, 28(Aib), |
| | | | | | | | 29E |
| Peptide | H(Aib)QGTFTSDVSK | 463 | C20diacid | γE- | 17 | Amide | 2(Aib), 10V, |
| 452 | (αMePhe)LDKK(O2Oc- | | | (O2Oc)- | | | 13(αMePhe), |
| | O2Oc-γE- | | | (O2Oc) | | | 16K, 17K, |
| | C20diacid)17RALDFVR | | | | | | 20L, 24R, |
| | WLL(Aib)E-amide | | | | | | 27L, 28(Aib), |
| | | | | | | | 29E |
| Peptide | H(Aib)QGTFTSDVSK | 464 | C20diacid | γE- | 17 | Amide | 2(Aib), 10V, |
| 453 | (αMePhe)LDKK(O2Oc- | | | (O2Oc)- | | | 13(αMePhe), |
| | O2Oc-γE- | | | (O2Oc) | | | 16K, 17K, |
| | C20diacid)17RAQDFVR | | | | | | 24R, 27L, |
| | WLLE(Aib)G-amide | | | | | | 28E, 29(Aib), |
| | | | | | | | 30G |
| Peptide | H(Aib)QGTFTSDVSK | 465 | C20diacid | γE- | 17 | Amide | 2(Aib), 10V, |
| 454 | (αMePhe)LDKK(O2Oc- | | | (O2Oc)- | | | 13(αMePhe), |
| | O2Oc-γE- | | | (O2Oc) | | | 16K, 17K, |
| | C20diacid)17RAQDFVR | | | | | | 24R, 27E, |
| | WLEA(Aib)G-amide | | | | | | 28A, 29(Aib), |
| | | | | | | | 30G |
| Peptide | H(Aib)QGTFTSDVSK | 466 | C20diacid | γE-γE- | 17 | Amide | 2(Aib), 10V, |
| 455 | (αMePhe)LDKK(O2Oc- | | | (O2Oc)- | | | 13(αMePhe), |
| | O2Oc-γE-yE- | | | (O2Oc) | | | 16K, 17K, |
| | C20diacid)17RAQDFVR | | | | | | 24R, 27L, |
| | WLL(Aib)E-amide | | | | | | 28(Aib), 29E |
| Peptide | H(Aib)QGTFTSDVSK | 467 | C20diacid | γE- | 17 | Amide | 2(Aib), 10V, |
| 456 | (αMePhe)LDRK(O2Oc- | | | (O2Oc)- | | | 13(αMePhe), |
| | O2Oc-γE- | | | (O2Oc) | | | 16R, 17K, |
| | C20diacid)17RAQDFVR | | | | | | 24R, 27L, |
| | WLL(Aib)E-amide | | | | | | 28(Aib), 29E |
| Peptide | H(Aib)QGTFTSDVSK | 468 | C20diacid | γE- | 17 | Amide | 2(Aib), 10V, |
| 457 | (αMePhe)LDTK(O2Oc- | | | (O2Oc)- | | | 13(αMePhe), |
| | O2Oc-γE- | | | (O2Oc) | | | 16T, 17K, |
| | C20diacid)17RARDFVQ | | | | | | 20R, 27L, |
| | WLLE(Aib)G-amide | | | | | | 28E, 29(Aib), |
| | | | | | | | 30G |
| Peptide | H(Aib)QGTFTSDVSK | 469 | C20diacid | γE-γE- | 17 | Amide | 2(Aib), 10V, |
| 458 | (αMePhe)LDTK(O2Oc- | | | (O2Oc)- | | | 13(αMePhe), |
| | O2Oc-γE-γE- | | | (O2Oc) | | | 16T, 17K, |
| | C20diacid)17RARDFVQ | | | | | | 20R, 27L, |
| | WLLE(Aib)G-amide | | | | | | 28E, 29(Aib), |
| | | | | | | | 30G |
| Peptide | H(Aib)QGTFTSDVSK | 470 | C18diacid | γE- | 17 | Amide | 2(Aib), 10V, |
| 459 | (αMePhe)LDKK(O2Oc- | | | (O2Oc)- | | | 13(αMePhe), |
| | O2Oc-γE- | | | (O2Oc) | | | 16K, 17K, |
| | C18diacid)17RAQDFVR | | | | | | 24R, 27L, |
| | WLLAE-amide | | | | | | 28A, 29E |
| Peptide | H(Aib)QGTFTSDVSK | 471 | C20diacid | γE- | 17 | Amide | 2(Aib), 10V, |
| 460 | (αMePhe)LDKK(O2Oc- | | | (O2Oc)- | | | 13(αMePhe), |
| | O2Oc-γE- | | | (O2Oc) | | | 16K, 17K, |
| | C20diacid)17RAQDFVR | | | | | | 24R, 27L, |
| | WLLAE-amide | | | | | | 28A, 29E |
| Peptide | H(Aib)QGTFTSDVSK | 472 | C18diacid | γE- | 17 | Amide | 2(Aib), 10V, |
| 461 | (αMePhe)LDRK(O2Oc- | | | (O2Oc)- | | | 13(αMePhe), |
| | O2Oc-γE- | | | (O2Oc) | | | 16R, 17K, |
| | C18diacid)17RAQDFVR | | | | | | 24R, 27L, |
| | WLLAE-amide | | | | | | 28A, 29E |
| Peptide | H(Aib)QGTFTSDVSK | 473 | C18diacid | γE- | 17 | Acid | 2(Aib), 10V, |
| 462 | (αMePhe)LDTK(O2Oc- | | | (O2Oc)- | | | 13(αMePhe), |
| | O2Oc-yE- | | | (O2Oc) | | | 16T, 17K, |
| | C18diacid)17RARDFVQ | | | | | | 20R, 27L, |
| | WLLEAG-acid | | | | | | 28E, 29A, |
| | | | | | | | 30G |
| Peptide | H(Aib)QGTFTSDVSK | 474 | C18diacid | γE-γE- | 17 | Amide | 2(Aib), 10V, |
| 463 | (αMePhe)LDSK(O2Oc- | | | (O2Oc)- | | | 13(αMePhe), |
| | O2Oc-γE-γE- | | | (O2Oc) | | | 17K, 27L, |
| | C18diacid)17RAQDFVQ | | | | | | 28(Aib) |
| | WLL(Aib)T-amide | | | | | | |
| Peptide | H(Aib)QGTFTSDVSK | 475 | C18diacid | γE- | 17 | Acid | 2(Aib), 10V, |
| 464 | (αMePhe)LDTK(O2Oc- | | | (O2Oc)- | | | 13(αMePhe), |
| | O2Oc-γE- | | | (O2Oc) | | | 16T, 17K, |
| | C18diacid)17RAQDFVQ | | | | | | 27L, 28E, |
| | WLLE(Aib)G-acid | | | | | | 29(Aib), 30G |
| Peptide | H(Aib)QGTFTSDVSK | 476 | C18diacid | γE-yE- | 17 | Acid | 2(Aib), 10V, |
| 465 | (αMePhe)LDLK(O2Oc- | | | (O2Oc)- | | | 13(αMePhe), |
| | O2Oc-γE-γE- | | | (O2Oc) | | | 16L, 17K, |
| | C18diacid)17RAQDFVQ | | | | | | 27L, 28(Aib) |
| | WLL(Aib)T-acid | | | | | | |
| Peptide | H(Aib)QGTFTSDVSK | 477 | C18diacid | γE- | 17 | Amide | 2(Aib), 10V, |
| 466 | (αMePhe)LDTK(O2Oc- | | | (O2Oc)- | | | 13(αMePhe), |
| | O2Oc-γE- | | | (O2Oc) | | | 16T, 17K, |
| | C18diacid)17RAQDFVQ | | | | | | 27L, 28E, |
| | WLLE(Aib)G-amide | | | | | | 29(Aib), 30G |
| Peptide | H(Aib)QGTFTSDVSK | 478 | C20diacid | γE- | 17 | Amide | 2(Aib), 10V, |
| 467 | (αMePhe)LDTK(O2Oc- | | | (O2Oc)- | | | 13(αMePhe), |
| | O2Oc-γE- | | | (O2Oc) | | | 16T, 17K, |
| | C20diacid)17RAQDFVQ | | | | | | 27L, 28E, |
| | WLLE(Aib)G-amide | | | | | | 29(Aib), 30G |
| Peptide | H(Aib)QGTFTSDVSK | 479 | C18diacid | γE-γE- | 17 | Amide | 2(Aib), 10V, |
| 468 | (αMePhe)LDTK(O2Oc- | | | (O2Oc)- | | | 13(αMePhe), |
| | O2Oc-γE-yE- | | | (O2Oc) | | | 16T, 17K, |
| | C18diacid)17RAQDFVQ | | | | | | 27L, 28E, |
| | WLLE(Aib)G-amide | | | | | | 29(Aib), 30G |
| Peptide | H(Aib)QGTFTSDVSK | 480 | C20diacid | γE-γE- | 17 | Amide | 2(Aib), 10V, |
| 469 | (αMePhe)LDTK(O2Oc- | | | (O2Oc)- | | | 13(αMePhe), |
| | O2Oc-γE-yE- | | | (O2Oc) | | | 16T, 17K, |
| | C20diacid)17RAQDFVQ | | | | | | 27L, 28E, |
| | WLLE(Aib)G-amide | | | | | | 29(Aib), 30G |
| Peptide | H(Aib)QGTFTSDVSK | 481 | C20diacid | γE- | 17 | Amide | 2(Aib), 10V, |
| 470 | (αMePhe)LDKK(O2Oc- | | | (O2Oc)- | | | 13(αMePhe), |
| | O2Oc-γE- | | | (O2Oc) | | | 16K, 17K, |
| | C20diacid)17RARDFVQ | | | | | | 20R, 27L, |
| | WLL(Aib)E-amide | | | | | | 28(Aib), 29E |
| Peptide | H(Aib)QGTFTSDVSK | 482 | C20diacid | γE- | 17 | Amide | 2(Aib), 10V, |
| 471 | (αMePhe)LDRK(O2Oc- | | | (O2Oc)- | | | 13(αMePhe), |
| | O2Oc-yE- | | | (O2Oc) | | | 16R, 17K, |
| | C20diacid)17RARDFVQ | | | | | | 20R, 27L, |
| | WLL(Aib)E-amide | | | | | | 28(Aib), 29E |
| Peptide | H(Aib)QGTFTSDVSK | 483 | C18diacid | γE- | 17 | Amide | 2(Aib), 10V, |
| 472 | (αMePhe)LDKK(O2Oc- | | | (O2Oc)- | | | 13(αMePhe), |
| | O2Oc-γE- | | | (O2Oc) | | | 16K, 17K, |
| | C18diacid)17RARDFVQ | | | | | | 20R, 27L, |
| | WLL(Aib)E-amide | | | | | | 28(Aib), 29E |
| Peptide | H(Aib)QGTFTSDVSK | 484 | C18diacid | γE- | 17 | Amide | 2(Aib), 10V, |
| 473 | (αMePhe)LDRK(O2Oc- | | | (O2Oc)- | | | 13(αMePhe), |
| | O2Oc-γE- | | | (O2Oc) | | | 16R, 17K, |
| | C18diacid)17RARDFVQ | | | | | | 20R, 27L, |
| | WLL(Aib)E-amide | | | | | | 28(Aib), 29E |
| Peptide | H(Aib)QGTFTSDVSK | 485 | C18diacid | γE- | 17 | Amide | 2(Aib), 10V, |
| 474 | (αMePhe)LDTK(O2Oc- | | | (O2Oc)- | | | 13(αMePhe), |
| | O2Oc-γE- | | | (O2Oc) | | | 16T, 17K, |
| | C18diacid)17RARDFVQ | | | | | | 20R, 27L, |
| | WLLA(Aib)G-amide | | | | | | 28A, 29(Aib), |
| | | | | | | | 30G |
| Peptide | H(Aib)QGTFTSDVSK | 486 | C20diacid | γE- | 17 | Amide | 2(Aib), 10V, |
| 475 | (αMePhe)LDTK(O2Oc- | | | (O2Oc)- | | | 13(αMePhe), |
| | O2Oc-γE- | | | (O2Oc) | | | 16T, 17K, |
| | C20diacid)17RARDFVQ | | | | | | 20R, 27L, |
| | WLLA(Aib)G-amide | | | | | | 28A, 29(Aib), |
| | | | | | | | 30G |
| Peptide | H(Aib)QGTFTSDVSK | 487 | C18diacid | γE- | 17 | Amide | 2(Aib), 10V, |
| 476 | (αMePhe)LDTK(O2Oc- | | | (O2Oc)- | | | 13(αMePhe), |
| | O2Oc-γE- | | | (O2Oc) | | | 16T, 17K, |
| | C18diacid)17RAQDFVR | | | | | | 24R, 27L, |
| | WLLA(Aib)G-amide | | | | | | 28A, 29(Aib), |
| | | | | | | | 30G |
| Peptide | H(Aib)QGTFTSDVSK | 488 | C20diacid | γE- | 17 | Amide | 2(Aib), 10V, |
| 477 | (αMePhe)LDTK(O2Oc- | | | (O2Oc)- | | | 13(αMePhe), |
| | O2Oc-γE- | | | (O2Oc) | | | 16T, 17K, |
| | C20diacid)17RAQDFVR | | | | | | 24R, 27L, |
| | WLLA(Aib)G-amide | | | | | | 28A, 29(Aib), |
| | | | | | | | 30G |
| Peptide | H(Aib)QGTFTSDVSK | 489 | C18diacid | γE- | 17 | Amide | 2(Aib), 10V, |
| 478 | (αMePhe)LDTK(O2Oc- | | | (O2Oc)- | | | 13(αMePhe), |
| | O2Oc-yE- | | | (O2Oc) | | | 16T, 17K, |
| | C18diacid)17RAQDFVR | | | | | | 24R, 27L, |
| | WLLE(Aib)K-amide | | | | | | 28E, 29(Aib), |
| | | | | | | | 30K |
| Peptide | H(Aib)QGTFTSDVSK | 490 | C20diacid | γE- | 17 | Amide | 2(Aib), 10V, |
| 479 | (αMePhe)LDTK(O2Oc- | | | (O2Oc)- | | | 13(αMePhe), |
| | O2Oc-γE- | | | (O2Oc) | | | 16T, 17K, |
| | C20diacid)17RAQDFVR | | | | | | 24R, 27L, |
| | WLLE(Aib)K-amide | | | | | | 28E, 29(Aib), |
| | | | | | | | 30K |
| Peptide | H(Aib)QGTFTSDVSK | 491 | C18diacid | γE- | 17 | Amide | 2(Aib), 10V, |
| 480 | (αMePhe)LDTK(O2Oc- | | | (O2Oc)- | | | 13(αMePhe), |
| | O2Oc-γE- | | | (O2Oc) | | | 16T, 17K, |
| | C18diacid)17RARDFVQ | | | | | | 20R, 27L, |
| | WLLE(Aib)K-amide | | | | | | 28E, 29(Aib), |
| | | | | | | | 30K |
| Peptide | H(Aib)QGTFTSDVSK | 492 | C20diacid | γE- | 17 | Amide | 2(Aib), 10V, |
| 481 | (αMePhe)LDTK(O2Oc- | | | (O2Oc)- | | | 13(αMePhe), |
| | O2Oc-yE- | | | (O2Oc) | | | 16T, 17K, |
| | C20diacid)17RARDFVQ | | | | | | 20R, 27L, |
| | WLLE(Aib)K-amide | | | | | | 28E, 29(Aib), |
| | | | | | | | 30K |
| Peptide | H(Aib)QGTFTSDVSK | 493 | C18diacid | γE- | 17 | Acid | 2(Aib), 10V, |
| 482 | (αMePhe)LESK(O2Oc- | | | (O2Oc)- | | | 13(αMePhe), |
| | O2Oc-γE- | | | (O2Oc) | | | 15E, 17K, |
| | C18diacid)17RAQDFVQ | | | | | | 27L, 28(Aib), |
| | WLL(Aib)E-acid | | | | | | 29E |
| Peptide | H(Aib)QGTFTSDVSK | 494 | C18diacid | γE- | 17 | Acid | 2(Aib), 10V, |
| 483 | (αMePhe)LDTK(O2Oc- | | | (O2Oc)- | | | 13(αMePhe), |
| | O2Oc-γE- | | | (O2Oc) | | | 16T, 17K, |
| | C18diacid)17RAQDFVQ | | | | | | 27L, 28(Aib), |
| | WLL(Aib)E-acid | | | | | | 29E |
| Peptide | H(Aib)QGTFTSDVSK | 495 | C20diacid | γE- | 17 | Acid | 2(Aib), 10V, |
| 484 | (αMePhe)LDTK(O2Oc- | | | (O2Oc)- | | | 13(αMePhe), |
| | O2Oc-yE- | | | (O2Oc) | | | 16T, 17K, |
| | C20diacid)17RAQDFVQ | | | | | | 27L, 28(Aib), |
| | WLL(Aib)E-acid | | | | | | 29E |
| Peptide | H(Aib)QGTFTSDVSK | 496 | C20diacid | γE- | 17 | Amide | 2(Aib), 10V, |
| 485 | (αMePhe)LDTK(O2Oc- | | | (O2Oc)- | | | 13(αMePhe), |
| | O2Oc-γE- | | | (O2Oc) | | | 16T, 17K, |
| | C20diacid)17RAQDFVQ | | | | | | 27L, 28(Aib), |
| | WLL(Aib)E-amide | | | | | | 29E |
| Peptide | H(Aib)QGTFTSDVSK | 497 | C20diacid | γE- | 17 | Acid | 2(Aib), 10V, |
| 486 | (αMePhe)LDLK(O2Oc- | | | (O2Oc)- | | | 13(αMePhe), |
| | O2Oc-γE- | | | (O2Oc) | | | 16L, 17K, |
| | C20diacid)17RAQDFVR | | | | | | 24R, 27L, |
| | WLL(Aib)E-acid | | | | | | 28(Aib), 29E |
| Peptide | H(Aib)QGTFTSDVSK | 498 | C18diacid | γE- | 17 | Acid | 2(Aib), 10V, |
| 487 | (αMePhe)LDTK(O2Oc- | | | (O2Oc)- | | | 13(αMePhe), |
| | O2Oc-γE- | | | (O2Oc) | | | 16T, 17K, |
| | C18diacid)17RARDFVQ | | | | | | 20R, 27L, |
| | WLL(Aib)TE-acid | | | | | | 28(Aib), 30E |
| Peptide | H(Aib)QGTFTSDVSK | 499 | C20diacid | γE- | 17 | Acid | 2(Aib), 10V, |
| 488 | (αMePhe)LDTK(O2Oc- | | | (O2Oc)- | | | 13(αMePhe), |
| | O2Oc-γE- | | | (O2Oc) | | | 16T, 17K, |
| | C20diacid)17RARDFVQ | | | | | | 20R, 27L, |
| | WLL(Aib)TE-acid | | | | | | 28(Aib), 30E |
| Peptide | H(Aib)QGTFTSDVSK | 500 | C18diacid | γE- | 17 | Amide | 2(Aib), 10V, |
| 489 | (αMePhe)LDTK(O2Oc- | | | (O2Oc)- | | | 13(αMePhe), |
| | O2Oc-γE- | | | (O2Oc) | | | 16T, 17K, |
| | C18diacid)17RARDFVQ | | | | | | 20R, 27L, |
| | WLL(Aib)TE-amide | | | | | | 28(Aib), 30E |
| Peptide | H(Aib)QGTFTSDVSK | 501 | C20diacid | γE- | 17 | Amide | 2(Aib), 10V, |
| 490 | (αMePhe)LDTK(O2Oc- | | | (O2Oc)- | | | 13(αMePhe), |
| | O2Oc-γE- | | | (O2Oc) | | | 16T, 17K, |
| | C20diacid)17RARDFVQ | | | | | | 20R, 27L, |
| | WLL(Aib)TE-amide | | | | | | 28(Aib), 30E |
| Peptide | H(Aib)QGTFTSDVSK | 502 | C18diacid | γE- | 17 | Acid | 2(Aib), 10V, |
| 491 | (αMePhe)LDTK(O2Oc- | | | (O2Oc)- | | | 13(αMePhe), |
| | O2Oc-γE- | | | (O2Oc) | | | 16T, 17K, |
| | C18diacid)17RARDFVR | | | | | | 20R, 24R, |
| | WLL(Aib)TE-acid | | | | | | 27L, 28(Aib), |
| | | | | | | | 30E |
| Peptide | H(Aib)QGTFTSDVSK | 503 | C20diacid | γE- | 17 | Acid | 2(Aib), 10V, |
| 492 | (αMePhe)LDTK(O2Oc- | | | (O2Oc)- | | | 13(αMePhe), |
| | O2Oc-yE- | | | (O2Oc) | | | 16T, 17K, |
| | C20diacid)17RARDFVR | | | | | | 20R, 24R, |
| | WLL(Aib)TE-acid | | | | | | 27L, 28(Aib), |
| | | | | | | | 30E |
| Peptide | H(Aib)QGTFTSDVSK | 504 | C18diacid | γE- | 17 | Acid | 2(Aib), 10V, |
| 493 | (αMePhe)LDTK(O2Oc- | | | (O2Oc)- | | | 13(αMePhe), |
| | O2Oc-γE- | | | (O2Oc) | | | 16T, 17K, |
| | C18diacid)17RAQDFVR | | | | | | 24R, 27L, |
| | WLLR(Aib)K-acid | | | | | | 28R, 29(Aib), |
| | | | | | | | 30K |
| Peptide | H(Aib)QGTFTSDVSK | 505 | C20diacid | γE- | 17 | Acid | 2(Aib), 10V, |
| 494 | (αMePhe)LDTK(O2Oc- | | | (O2Oc)- | | | 13(αMePhe), |
| | O2Oc-γE- | | | (O2Oc) | | | 16T, 17K, |
| | C20diacid)17RAQDFVR | | | | | | 24R, 27L, |
| | WLLR(Aib)K-acid | | | | | | 28R, 29(Aib), |
| | | | | | | | 30K |
| Peptide | H(Aib)QGTFTSDVSK | 506 | C18diacid | γE- | 17 | Amide | 2(Aib), 10V, |
| 495 | (αMePhe)LDTK(O2Oc- | | | (O2Oc)- | | | 13(αMePhe), |
| | O2Oc-γE- | | | (O2Oc) | | | 16T, 17K, |
| | C18diacid)17RAQDFVR | | | | | | 24R, 27L, |
| | WLLR(Aib)K-amide | | | | | | 28R, 29(Aib), |
| | | | | | | | 30K |
| Peptide | H(Aib)QGTFTSDVSK | 507 | C20diacid | γE- | 17 | Amide | 2(Aib), 10V, |
| 496 | (αMePhe)LDTK(O2Oc- | | | (O2Oc)- | | | 13(αMePhe), |
| | O2Oc-γE- | | | (O2Oc) | | | 16T, 17K, |
| | C20diacid)17RAQDFVR | | | | | | 24R, 27L, |
| | WLLR(Aib)K-amide | | | | | | 28R, 29(Aib), |
| | | | | | | | 30K |
| Peptide | H(Aib)QGTFTSDVSK | 508 | C18diacid | γE- | 17 | Acid | 2(Aib), 10V, |
| 497 | (αMePhe)LDTK(O2Oc- | | | (O2Oc)- | | | 13(αMePhe), |
| | O2Oc-γE- | | | (O2Oc) | | | 16T, 17K, |
| | C18diacid)17RAQDFVR | | | | | | 24R, 27L, |
| | WLLR(Aib)A-acid | | | | | | 28R, 29(Aib), |
| | | | | | | | 30A |
| Peptide | H(Aib)QGTFTSDVSK | 509 | C20diacid | γE- | 17 | Acid | 2(Aib), 10V, |
| 498 | (αMePhe)LDTK(O2Oc- | | | (O2Oc)- | | | 13(αMePhe), |
| | O2Oc-γE- | | | (O2Oc) | | | 16T, 17K, |
| | C20diacid)17RAQDFVR | | | | | | 24R, 27L, |
| | WLLR(Aib)A-acid | | | | | | 28R, 29(Aib), |
| | | | | | | | 30A |
| Peptide | H(Aib)QGTFTSDVSK | 510 | C18diacid | γE- | 17 | Acid | 2(Aib), 10V, |
| 499 | (αMePhe)LDTK(O2Oc- | | | (O2Oc)- | | | 13(αMePhe), |
| | O2Oc-γE- | | | (O2Oc) | | | 16T, 17K, |
| | C18diacid)17RAQDFVQ | | | | | | 27L, 28R, |
| | WLLR(Aib)A-acid | | | | | | 29(Aib), 30A |
| Peptide | H(Aib)QGTFTSDVSK | 511 | C20diacid | γE- | 17 | Acid | 2(Aib), 10V, |
| 500 | (αMePhe)LDTK(O2Oc- | | | (O2Oc)- | | | 13(αMePhe), |
| | O2Oc-γE- | | | (O2Oc) | | | 16T, 17K, |
| | C20diacid)17RAQDFVQ | | | | | | 27L, 28R, |
| | WLLR(Aib)A-acid | | | | | | 29(Aib), 30A |
| Peptide | H(Aib)QGTFTSDVSK | 512 | C18diacid | γE- | 17 | Acid | 2(Aib), 10V, |
| 501 | (αMePhe)LDTK(O2Oc- | | | (O2Oc)- | | | 13(αMePhe), |
| | O2Oc-γE- | | | (O2Oc) | | | 16T, 17K, |
| | C18diacid)17RARDFVQ | | | | | | 20R, 27L, |
| | WLLR(Aib)A-acid | | | | | | 28R, 29(Aib), |
| | | | | | | | 30A |
| Peptide | H(Aib)QGTFTSDVSK | 513 | C20diacid | γE- | 17 | Acid | 2(Aib), 10V, |
| 502 | (αMePhe)LDTK(O2Oc- | | | (O2Oc)- | | | 13(αMePhe), |
| | O2Oc-γE- | | | (O2Oc) | | | 16T, 17K, |
| | C20diacid)17RARDFVQ | | | | | | 20R, 27L, |
| | WLLR(Aib)A-acid | | | | | | 28R, 29(Aib), |
| | | | | | | | 30A |
| Peptide | H(Aib)QGTFTSDVSK | 514 | C20diacid | γE- | 17 | Acid | 2(Aib), 10V, |
| 503 | (αMePhe)LDTK(O2Oc- | | | (O2Oc)- | | | 13(αMePhe), |
| | O2Oc-γE- | | | (O2Oc) | | | 16T, 17K, |
| | C20diacid)17RAQDFVR | | | | | | 24R, 27L, |
| | WLLE(Aib)K-acid | | | | | | 28E, 29(Aib), |
| | | | | | | | 30K |
| Peptide | H(Aib)QGTFTSDVSK | 515 | C20diacid | γE- | 17 | Acid | 2(Aib), 10V, |
| 504 | (αMePhe)LESK(O2Oc- | | | (O2Oc)- | | | 13(αMePhe), |
| | O2Oc-γE- | | | (O2Oc) | | | 15E, 17K, |
| | C20diacid)17RAQDFVR | | | | | | 24R, 27L, |
| | WLLE(Aib)K-acid | | | | | | 28E, 29(Aib), |
| | | | | | | | 30K |
| Peptide | H(Aib)QGTFTSDVSK | 516 | C20diacid | γE- | 17 | Amide | 2(Aib), 10V, |
| 505 | (αMePhe)LDTK(O2Oc- | | | (O2Oc)- | | | 13(αMePhe), |
| | O2Oc-γE- | | | (O2Oc) | | | 16T, 17K, |
| | C20diacid)17RAQDFVQ | | | | | | 27L, 28E, |
| | WLLE(Aib)K-amide | | | | | | 29(Aib), 30K |
| Peptide | H(Aib)QGTFTSDVSK | 517 | C20diacid | γE- | 17 | Acid | 2(Aib), 10V, |
| 506 | (αMePhe)LDTK(O2Oc- | | | (O2Oc)- | | | 13(αMePhe), |
| | O2Oc-γE- | | | (O2Oc) | | | 16T, 17K, |
| | C20diacid)17RARDFVA | | | | | | 20R, 24A, |
| | WLLE(Aib)G-acid | | | | | | 27L, 28E, |
| | | | | | | | 29(Aib), 30G |
| Peptide | H(Aib)QGTFTSDVSK | 518 | C20diacid | γE- | 17 | Amide | 2(Aib), 10V, |
| 507 | (αMePhe)LDTK(O2Oc- | | | (O2Oc)- | | | 13(αMePhe), |
| | O2Oc-γE- | | | (O2Oc) | | | 16T, 17K, |
| | C20diacid)17RARDFVA | | | | | | 20R, 24A, |
| | WLLE(Aib)G-amide | | | | | | 27L, 28E, |
| | | | | | | | 29(Aib), 30G |
| Peptide | H(Aib)QGTFTSDVSK | 519 | C20diacid | γE- | 17 | Amide | 2(Aib), 10V, |
| 508 | (αMePhe)LESK(O2Oc- | | | (O2Oc)- | | | 13(αMePhe), |
| | O2Oc-γE- | | | (O2Oc) | | | 15E, 17K, |
| | C20diacid)17RAQDFVR | | | | | | 24R, 27V, |
| | WLVE(Aib)G-amide | | | | | | 28E, 29(Aib), |
| | | | | | | | 30G |
| Peptide | H(Aib)QGTFTSDVSK | 520 | C20diacid | γE- | 17 | Acid | 2(Aib), 10V, |
| 509 | (αMePhe)LESK(O2Oc- | | | (O2Oc)- | | | 13(αMePhe), |
| | O2Oc-γE- | | | (O2Oc) | | | 15E, 17K, |
| | C20diacid)17RAQDFVR | | | | | | 24R, 27L, |
| | WLL(Aib)TE-acid | | | | | | 28(Aib), 30E |
| Peptide | H(Aib)QGTFTSDVSK | 521 | C20diacid | γE- | 17 | Acid | 2(Aib), 10V, |
| 510 | (αMePhe)LDTK(O2Oc- | | | (O2Oc)- | | | 13(αMePhe), |
| | O2Oc-γE- | | | (O2Oc) | | | 16T, 17K, |
| | C20diacid)17RARDFVR | | | | | | 20R, 24R, |
| | WLL(Aib)E-acid | | | | | | 27L, 28(Aib), |
| | | | | | | | 29E |
| Peptide | H(Aib)QGTFTSDVSK | 522 | C20diacid | γE- | 17 | Amide | 2(Aib), 10V, |
| 511 | (αMePhe)LDTK(O2Oc- | | | (O2Oc)- | | | 13(αMePhe), |
| | O2Oc-γE- | | | (O2Oc) | | | 16T, 17K, |
| | C20diacid)17RARDFVR | | | | | | 20R, 24R, |
| | WLL(Aib)E-amide | | | | | | 27L, 28(Aib), |
| | | | | | | | 29E |
| Peptide | H(Aib)QGT(αMePhe)TS | 523 | C18diacid | γE- | 17 | Amide | 2(Aib), |
| 512 | DVSK(αMePhe)LDTK | | | (O2Oc)- | | | 6(αMePhe), |
| | (O2Oc-O2Oc-γE- | | | (O2Oc) | | | 10V, |
| | C18diacid)17RAQD | | | | | | 13(αMePhe), |
| | (αMePhe)VRWLLA(Aib)G- | | | | | | 16T, 17K, |
| | amide | | | | | | 22(αMePhe), |
| | | | | | | | 24R, 27L, |
| | | | | | | | 28A, 29(Aib), |
| | | | | | | | 30G |
| Peptide | H(Aib)QGT(αMePhe)TS | 524 | C18diacid | γE- | 17 | Amide | 2(Aib), |
| 513 | DVSK(αMePhe)LDTK | | | (O2Oc)- | | | 6(αMePhe), |
| | (O2Oc-O2Oc-γE- | | | (O2Oc) | | | 10V, |
| | C18diacid)17RARD | | | | | | 13(αMePhe), |
| | (αMePhe)VQWLLA(Aib)G- | | | | | | 16T, 17K, |
| | amide | | | | | | 20R, |
| | | | | | | | 22(αMePhe), |
| | | | | | | | 27L, 28A, |
| | | | | | | | 29(Aib), 30G |
| Peptide | H(Aib)QGTFTSDVSK | 525 | C18diacid | γE- | 17 | Amide | 2(Aib), 10V, |
| 514 | (αMePhe)LDTK(O2Oc- | | | (O2Oc)- | | | 13(αMePhe), |
| | O2Oc-γE- | | | (O2Oc) | | | 16T, 17K, |
| | C18diacid)17AARDFVQ | | | | | | 18A, 20R, |
| | WLLE(Aib)G-amide | | | | | | 27L, 28E, |
| | | | | | | | 29(Aib), 30G |
| Peptide | H(Aib)QGTFTSDVSK | 526 | C20diacid | γE- | 17 | Amide | 2(Aib), 10V, |
| 515 | (αMePhe)LDTK(O2Oc- | | | (O2Oc)- | | | 13(αMePhe), |
| | O2Oc-γE- | | | (O2Oc) | | | 16T, 17K, |
| | C20diacid)17AARDFVQ | | | | | | 18A, 20R, |
| | WLLE(Aib)G-amide | | | | | | 27L, 28E, |
| | | | | | | | 29(Aib), 30G |
| Peptide | H(Aib)QGTFTSDVSK | 527 | C20diacid | γE- | 17 | Amide | 2(Aib), 10V, |
| 516 | (αMePhe)LESK(O2Oc- | | | (O2Oc)- | | | 13(αMePhe), |
| | O2Oc-γE- | | | (O2Oc) | | | 15E, 17K, |
| | C20diacid)17RAQDFVR | | | | | | 24R, 27L, |
| | WLLA(Aib)G-amide | | | | | | 28A, 29(Aib), |
| | | | | | | | 30G |
| Peptide | H(Aib)QGTFTSDVSK | 528 | C18diacid | γE-γE- | 17 | Amide | 2(Aib), 10V, |
| 517 | (αMePhe)LEAK(O2Oc- | | | (O2Oc)- | | | 13(αMePhe), |
| | O2Oc-γE-γE- | | | (O2Oc) | | | 15E, 16A, |
| | C18diacid)17AAREFIAW | | | | | | 17K, 18A, |
| | LLET-amide | | | | | | 20R, 21E, |
| | | | | | | | 23I, 24A, |
| | | | | | | | 27L, 28E |
| Peptide | H(Aib)QGTFTSDVSK | 529 | C18diacid | γE-γE- | 17 | Acid | 2(Aib), 10V, |
| 518 | (αMePhe)LDTK(O2Oc- | | | (O2Oc)- | | | 13(αMePhe), |
| | O2Oc-γE-γE- | | | (O2Oc) | | | 16T, 17K, |
| | C18diacid)17RARDFVQ | | | | | | 20R, 27L, |
| | WLLE(Aib)G-acid | | | | | | 28E, 29(Aib), |
| | | | | | | | 30G |
| Peptide | H(Aib)QGTFTSDVSK | 530 | C18diacid | γE-γE- | 17 | Acid | 2(Aib), 10V, |
| 519 | (αMePhe)LDTK(γE-γE- | | | (O2Oc)- | | | 13(αMePhe), |
| | O2Oc-O2Oc-γE-γE- | | | (O2Oc)- | | | 16T, 17K, |
| | C18diacid)17RARDFVQ | | | γE-γE | | | 20R, 27L, |
| | WLLE(Aib)G-acid | | | | | | 28E, 29(Aib), |
| | | | | | | | 30G |
| Peptide | H(Aib)QGTFTSDVSK | 531 | C20diacid | γE-γE- | 17 | Acid | 2(Aib), 10V, |
| 520 | (αMePhe)LDTK(γE-γE- | | | (O2Oc)- | | | 13(αMePhe), |
| | O2Oc-O2Oc-γE-γE- | | | (O2Oc)- | | | 16T, 17K, |
| | C20diacid)17RARDFVQ | | | γE-γE | | | 20R, 27L, |
| | WLLE(Aib)G-acid | | | | | | 28E, 29(Aib), |
| | | | | | | | 30G |

In some aspects, the peptide comprises the sequence: H-X2-X3-G-X5-X6-T-S-D-X10-S-X12-X13-L-X15-X16-X17-X18-X19-X20-X21-X22-X23-X24-X25-X26-X27-X28-X29-X30-X31-Z, wherein X2 is S, Aminoisobutyric acid (Aib), αMethyl-Serine (αMeS), D-serine (dSer), 1-aminocyclopropane-1-carboxylic acid (Acpr), or S, 1-aminocyclobutane-1-carboxylic acid (Acbu), X3 is Q, H, αMethyl-Glutamine (αMeGln), N-Methyl-Glutamine (N-MeGln), D-glutamine (dGln) or β-dimethylglutamine (β-dimethylGln), X5 is T or S, X6 is F or αMethyl-Phenylalanine (αMePhe), X10 is Y or V, X12 is K, E, or R, X13 is Y, αMePhe, or Aib, X15 is D or E, X16 is S, T, E, or Aib, X17 is R, Q, or E, X18 is R, A, Aib, or S, X19 is A or V, X20 is Q or K, wherein the K can comprise an acyl moiety and/or can be lipidated, X21 is D or L, X22 is F or αMethyl-Phenylalanine (αMePhe), X23 is V or I, X24 is Q, E, A, or R, X25 is W, Aib, or S, X26 is L or I, X27 is M, A, L, E, I, or V, X28 is N, E, (PEG)4, Aib, S, or A, X29 is T, not present, E, or G, X30 is not present, E, T, or G, X31 is not present or G, and Z is amide or acid (SEQ ID NO: 413), wherein the peptide does not comprise SEQ ID NO: 1.

In some aspects, the residue at position 20 is acylated. In some aspects, the residue at position 20 is lipidated. In some aspects, the lipid is selected from the group consisting of a palmitoyl group, stearoyl group, lauryl group, myristoyl group, margaroyl group, octadecanedioic acid (C18diacid), and icosanedioic acid (C20diacid). In some aspects, the lipid is selected from the group consisting of a stearoyl group, octadecanedioic acid (C18diacid), and icosanedioic acid (C20diacid).

In some aspects, the lipid attached to the residue at position 20 is attached via a linker. In some aspects, the linker is selected from the group consisting of (O2Oc), (O2Oc)-(O2Oc), (O2Oc)-γE-(O2Oc), (PEG)2-(PEG)2-γE-γE, (PEG)2-γE-(PEG)2-γE, γE, γE-(O2Oc), γE-(O2Oc)-(O2Oc), γE-(O2Oc)-γE-(O2Oc), γE-(PEG)2-(PEG)2, γE-(PEG)2-γE-(PEG)2, γE-(PEG)4, γE-γE, γE-γE-(O2Oc), γE-γE-(O2Oc)-(O2Oc), γE-γE-(PEG)12, γE-γE-(PEG)2-(PEG)2, γE-γE-(PEG)2-γE-γE, γE-γE-(PEG)4, and γE-γE-(PEG)8. In some aspects, the linker is selected from the group consisting of γE, γE-γE, γE-γE-(O2Oc), γE-(O2Oc)-(O2Oc), γE-γE-(O2Oc)-(O2Oc), γE-(O2Oc), γE-γE-(PEG)2-(PEG)2, γE-(O2Oc)-γE-(O2Oc), YE-(PEG)4, γE-γE-(PEG)4, (O2Oc)-γE-(O2Oc), (O2Oc)-(O2Oc), and (O2Oc).

In some aspects, the peptide comprises the sequence: H-Aminoisobutyric acid (Aib)-Q-G-T-F-T-S-D-X10-S-X12-αMethyl-Phenylalanine (αMePhe)-L-D-X16-X17-X18-A-K-D-F—V-X24-W-X26-X27-X28-X29-X30-Z, wherein X10 is V or Y, X12 is K or E, X16 is S or Aib, X17 is R or E, X18 is R or A, X24 is A, R, or Q, X26 is L or I, X27 is E, L, A, or I, X28 is A, E, Aib, S, or N, X29 is G, Aib, T, or E, X30 is G, E, T, or not present, and Z is amide or acid (SEQ ID NO: 414).

In some aspects, the lysine at position 20 is acylated. In some aspects, the lysine at position 20 is lipidated. In some aspects, the lipid is selected from the group consisting of a palmitoyl group, stearoyl group, lauryl group, myristoyl group, margaroyl group, octadecanedioic acid (C18diacid), and icosanedioic acid (C20diacid). In some aspects, the lipid is octadecanedioic acid (C18diacid).

In some aspects, the lipid is linked to the lysine at position 20 via a linker. In some aspects, the linker is selected from the group consisting of (O2Oc), (O2Oc)-(O2Oc), (O2Oc)-YE-(O2Oc), (PEG)2-(PEG)2-γE-γE, (PEG)2-γE-(PEG)2-γE, γE, γE-(O2Oc), γE-(O2Oc)-(O2Oc), γE-(O2Oc)-γE-(O2Oc), γE-(PEG)2-(PEG)2, γE-(PEG)2-γE-(PEG)2, γE-(PEG)4, γE-γE, γE-γE-(O2Oc), γE-γE-(O2Oc)-(O2Oc), γE-γE-(PEG)12, γE-γE-(PEG)2-(PEG)2, γE-γE-(PEG)2-γE-γE, γE-γE-(PEG)4, and γE-γE-(PEG)8. In some aspects, the linker is selected from the group consisting of γE, γE-(O2Oc)-(O2Oc), and γE-γE (O2Oc)-(O2Oc)-γE-γE.

In some aspects, the peptide comprises any one of SEQ ID NOs: 207-347 and 532-537. In some aspects, the peptide comprises SEQ ID NO: 228. In some aspects, the peptide comprises SEQ ID NO: 233.

In some aspects, the peptide has the structure of any one of the structures depicted in FIGS. 4A-4B. In some aspects, the peptide has the structure of FIG. 4A. In some aspects, the peptide has the structure of FIG. 4B.

In some aspects, the peptide is any one of the peptides in Table 2.

TABLE 2
Peptides Modified at Position 20

| | | | | Linker | | | Sequence |
|---|---|---|---|---|---|---|---|
| | | SEQ | Albumin | (described | Acyl- | | modification |
| | | ID | binding | N→C | ation | C- | with respect |
| Peptide | Sequence | NO | moiety | term) | site | term. | to glucagon |
| Peptide | H(Aib)HGS(αMePhe)TS | 207 | C18diacid | γE- | 20 | Amide | 2Aib, 3H, 5S, |
| 203 | DVSK(αMePhe)LDSQAA | | | (O2Oc)- | | | 6αMePhe, |
| | K(O2Oc-O2Oc-γE- | | | (O2Oc) | | | 10V, |
| | C18diacid)20DFVQWIA | | | | | | 13αMePhe, |
| | NT-amide | | | | | | 17Q, 18A, |
| | | | | | | | 20K, 26I, 27A |
| Peptide | H(Aib)QGT(αMePhe)TS | 208 | C18diacid | γE- | 20 | Amide | 2Aib, 5S, |
| 204 | DVSK(αMePhe)LDSRAA | | | (O2Oc)- | | | 6αMePhe, |
| | K(O2Oc-02Oc-γE- | | | (O2Oc) | | | 10V, |
| | C18diacid)20DFVQWIA | | | | | | 13αMePhe, |
| | NT-amide | | | | | | 18A, 20K, |
| | | | | | | | 26I, 27A |
| Peptide | H(Aib)HGS(αMePhe)TS | 209 | C18diacid | γE- | 20 | Amide | 2Aib, 3H, 5S, |
| 205 | DVSK(αMePhe)LDSRAA | | | (O2Oc)- | | | 6αMePhe, |
| | K(O2Oc-O2Oc-γE- | | | (O2Oc) | | | 10V, |
| | C18diacid)20DFVQWIA | | | | | | 13αMePhe, |
| | NT-amide | | | | | | 18A, 20K, |
| | | | | | | | 26I, 27A |
| Peptide | H(Aib)HGS(αMePhe)TS | 210 | C18diacid | γE- | 20 | Amide | 2Aib, 3H, 5S, |
| 206 | DVSK(ααMePhe)LDSR | | | (O2Oc)- | | | 6αMePhe, |
| | (Aib)AK(O2Oc-O2Oc-γE- | | | (O2Oc) | | | 10V, |
| | C18diacid)20DFVQWIA | | | | | | 13αMePhe, |
| | NT-amide | | | | | | 18Aib, 20K, |
| | | | | | | | 26I, 27A |
| Peptide | H(Aib)HGS(αMePhe)TS | 211 | C18diacid | γE- | 20 | Amide | 2Aib, 3H, 5S, |
| 207 | DVSK(αMePhe)LDSQAA | | | (O2Oc)- | | | 6αMePhe, |
| | K(O2Oc-O2Oc-γE- | | | (O2Oc) | | | 10V, |
| | C18diacid)20DFVQ(Aib) | | | | | | 13αMePhe, |
| | IANT-amide | | | | | | 17Q, 18A, |
| | | | | | | | 20K, 25Aib, |
| | | | | | | | 26I, 27A |
| Peptide | H(Aib)QGTFTSDVSK | 212 | C18diacid | γE | 20 | Amide | 2Aib, 10V, |
| 208 | (αMePhe)LDSRAAK(γE- | | | | | | 13αMePhe, |
| | C18diacid)20DFVQWIA | | | | | | 18A, 20K, |
| | NT-amide | | | | | | 26I, 27A |
| Peptide | H(Aib)HGS(αMePhe)TS | 213 | C18diacid | γE- | 20 | Amide | 2Aib, 3H, 5S, |
| 209 | DVSK(αMePhe)LDSQAA | | | (O2Oc)- | | | 6αMePhe, |
| | K(O2Oc-O2Oc-γE- | | | (O2Oc) | | | 10V, |
| | C18diacid)20DFVEWIA | | | | | | 13αMePhe, |
| | NT-amide | | | | | | 17Q, 18A, |
| | | | | | | | 20K, 24E, |
| | | | | | | | 26I, 27A |
| Peptide | H(Aib)HGS(αMePhe)TS | 214 | C18diacid | γE-γE- | 20 | Amide | 2Aib, 3H, 5S, |
| 210 | DVSK(αMePhe)LDSQAA | | | (O2Oc)- | | | 6αMePhe, |
| | K(O2Oc-O2Oc-γE-γE- | | | (O2Oc) | | | 10V, |
| | C18diacid)20DFVEWIA | | | | | | 13αMePhe, |
| | NT-amide | | | | | | 17Q, 18A, |
| | | | | | | | 20K, 24E, |
| | | | | | | | 26I, 27A |
| Peptide | H(Aib)HGS(αMePhe)TS | 215 | C20diacid | γE-γE- | 20 | Amide | 2Aib, 3H, 5S, |
| 211 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid)20DFVQWIA | | | | | | 13αMePhe, |
| | NT-amide | | | | | | 18A, 20K, |
| | | | | | | | 26I, 27A |
| Peptide | H(Aib)HGS(αMePhe)TS | 216 | C20diacid | γE- | 20 | Amide | 2Aib, 3H, 5S, |
| 212 | DVSK(αMePhe)LDSRAA | | | (O2Oc)- | | | 6αMePhe, |
| | K(O2Oc-O2Oc-γE- | | | (O2Oc) | | | 10V, |
| | C20diacid)20DFVEWIA | | | | | | 13αMePhe, |
| | NT-amide | | | | | | 18A, 20K, |
| | | | | | | | 24E, 26I, 27A |
| Peptide | H(Aib)HGS(αMePhe)TS | 217 | C20diacid | γE-γE- | 20 | Amide | 2Aib, 3H, 5S, |
| 213 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid)20DFVEWIA | | | | | | 13αMePhe, |
| | NT-amide | | | | | | 18A, 20K, |
| | | | | | | | 24E, 26I, 27A |
| Peptide | H(Aib)HGS(αMePhe)TS | 218 | C20diacid | γE- | 20 | Amide | 2Aib, 3H, 5S, |
| 214 | DVSK(αMePhe)LDSRAA | | | (O2Oc)- | | | 6αMePhe, |
| | K(O2Oc-O2Oc-γE- | | | (O2Oc) | | | 10V, |
| | C20diacid)20DFVQWIA | | | | | | 13αMePhe, |
| | NT-amide | | | | | | 18A, 20K, |
| | | | | | | | 26I, 27A |
| Peptide | H(Aib)HGS(αMePhe)TS | 219 | C20diacid | γE- | 20 | Amide | 2Aib, 3H, 5S, |
| 215 | DVSK(αMePhe)LDSRAA | | | (O2Oc)- | | | 6αMePhe, |
| | K(O2Oc-O2Oc-γE- | | | (O2Oc) | | | 10V, |
| | C20diacid)20D | | | | | | 13αMePhe, |
| | (αMePhe)VQWIANT- | | | | | | 18A, 20K, |
| | amide | | | | | | 22αMePhe, |
| | | | | | | | 26I, 27A |
| Peptide | H(Aib)HGS(αMePhe)TS | 220 | C20diacid | γE- | 20 | Amide | 2Aib, 3H, 5S, |
| 216 | DVSK(αMePhe)LDSRAA | | | (O2Oc)- | | | 6αMePhe, |
| | K(O2Oc-O2Oc-γE- | | | (O2Oc) | | | 10V, |
| | C20diacid)20D | | | | | | 13αMePhe, |
| | (αMePhe)VEWIANT- | | | | | | 18A, 20K, |
| | amide | | | | | | 22αMePhe, |
| | | | | | | | 24E, 26I, 27A |
| Peptide | H(Aib)HGS(αMePhe)TS | 221 | C20diacid | γE-γE- | 20 | Amide | 2Aib, 3H, 5S, |
| 217 | DVSK(αMePhe)LDSR | | | (O2Oc)- | | | 6αMePhe, |
| | (Aib)AK(O2Oc-O2Oc- | | | (O2Oc) | | | 10V, |
| | γE-γE- | | | | | | 13αMePhe, |
| | C20diacid)20DFVQWIA | | | | | | 18Aib, 20K, |
| | NT-amide | | | | | | 26I, 27A |
| Peptide | H(Aib)HGS(αMePhe)TS | 222 | C20diacid | γE-γE- | 20 | Amide | 2Aib, 3H, 5S, |
| 218 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid)20DFVQWIA | | | | | | 13αMePhe, |
| | NTG-amide | | | | | | 18A, 20K, |
| | | | | | | | 26I, 27A, |
| | | | | | | | 30G |
| Peptide | H(Aib)HGS(αMePhe)TS | 223 | C20diacid | γE-γE- | 20 | Amide | 2Aib, 3H, 5S, |
| 219 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid)20DFVQWIA | | | | | | 13αMePhe, |
| | NTGG-amide | | | | | | 18A, 20K, |
| | | | | | | | 26I, 27A, |
| | | | | | | | 30G, 31G |
| Peptide | H(Aib)HGS(αMePhe)TS | 224 | C20diacid | γE-γE- | 20 | Amide | 2Aib, 3H, 5S, |
| 220 | DVSK(αMePhe)LDSR | | | (O2Oc)- | | | 6αMePhe, |
| | (Aib)AK(O2Oc-O2Oc- | | | (O2Oc) | | | 10V |
| | γE-γE- | | | | | | 13αMePhe, |
| | C20diacid)20DFVQ(Aib) | | | | | | 18Aib, 20K, |
| | IANT-amide | | | | | | 25Aib, 26I, |
| | | | | | | | 27A |
| Peptide | H(Aib)HGS(αMePhe)TS | 225 | C20diacid | γE-γE- | 20 | Amide | 2Aib, 3H, 5S, |
| 221 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid)20DFVQ(Aib) | | | | | | 13αMePhe, |
| | IANT-amide | | | | | | 18A, 20K, |
| | | | | | | | 25Aib, 26I, |
| | | | | | | | 27A |
| Peptide | H(Aib)HGS(αMePhe)TS | 226 | C18diacid | γE- | 20 | Amide | 2Aib, 3H, 5S, |
| 222 | DVSK(αMePhe)LDSQAA | | | (O2Oc)- | | | 6αMePhe, |
| | K(O2Oc-O2Oc-γE- | | | (O2Oc) | | | 10V, |
| | C18diacid)20DFVQSIAN | | | | | | 13αMePhe, |
| | T-amide | | | | | | 17Q, 18A, |
| | | | | | | | 20K, 25S, |
| | | | | | | | 26I, 27A |
| Peptide | H(Aib)HGS(αMePhe)TS | 227 | C18diacid | γE- | 20 | Amide | 2Aib, 3H, 5S, |
| 223 | DVSK(αMePhe)LDSR | | | (O2Oc)- | | | 6αMePhe, |
| | (Aib)AK(O2Oc-O2Oc- | | | (O2Oc) | | | 10V, |
| | γE-C18diacid)20DFVQS | | | | | | 13αMePhe, |
| | IANT-amide | | | | | | 18Aib, 20K, |
| | | | | | | | 25S, 26I, 27A |
| Peptide | H(Aib)HGS(αMePhe)TS | 228 | C18diacid | γE- | 20 | Amide | 2Aib, 3H, 5S, |
| 224 | DVSK(αMePhe)LDSRAA | | | (O2Oc)- | | | 6αMePhe, |
| | K(O2Oc-O2Oc-γE- | | | (O2Oc) | | | 10V |
| | C18diacid) 20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 229 | C18diacid | γE- | 20 | Amide | 2Aib, 3H, 5S, |
| 225 | DVSK(αMePhe)LDSRAA | | | (O2Oc)- | | | 6αMePhe, |
| | K(O2Oc-O2Oc-γE- | | | (O2Oc) | | | 10V |
| | C18diacid)20D | | | | | | 13αMePhe, |
| | (αMePhe)VQ(Aib) | | | | | | 18A, 20K, |
| | IANTGG-amide | | | | | | 22αMePhe, |
| | | | | | | | 25Aib, 26I, |
| | | | | | | | 27A, 30G, |
| | | | | | | | 31G |
| Peptide | H(Aib)HGS(αMePhe)TS | 230 | C18diacid | γE- | 20 | Amide | 2Aib, 3H, 5S, |
| 226 | DVSK(αMePhe)LDSRAA | | | (O2Oc)- | | | 6αMePhe, |
| | K(O2Oc-O2Oc-γE- | | | (O2Oc) | | | 10V |
| | C18diacid)20D | | | | | | 13αMePhe, |
| | (αMePhe)VE(Aib)IANT- | | | | | | 18A, 20K, |
| | amide | | | | | | 22αMePhe, |
| | | | | | | | 24E, 25Aib, |
| | | | | | | | 26I, 27A |
| Peptide | H(Aib)HGS(αMePhe)TS | 231 | C18diacid | γE- | 20 | Amide | 2Aib, 3H, 5S, |
| 227 | DVSK(αMePhe)LDSRAA | | | (O2Oc)- | | | 6αMePhe, |
| | K(O2Oc-O2Oc-γE- | | | (O2Oc) | | | 10V, |
| | C18diacid)20D | | | | | | 13αMePhe, |
| | (αMePhe)IA(Aib)IANT- | | | | | | 18A, 20K, |
| | amide | | | | | | 22αMePhe, |
| | | | | | | | 23I, 24A, |
| | | | | | | | 25Aib, 26I, |
| | | | | | | | 27A |
| Peptide | H(Aib)HGS(αMePhe)TS | 232 | C20diacid | γE-γE- | 20 | Amide | 2Aib, 3H, 5S, |
| 228 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid) 20D | | | | | | 13αMePhe, |
| | (αMePhe)VQWIANT- | | | | | | 18A, 20K, |
| | amide | | | | | | 22αMePhe, |
| | | | | | | | 26I, 27A |
| Peptide | H(Aib)HGS(αMePhe)TS | 233 | C20diacid | γE-γE- | 20 | Amide | 2Aib, 3H, 5S, |
| 229 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid) 20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 234 | C18diacid | γE- | 20 | Amide | 2Aib, 3H, 5S, |
| 230 | DVSK(αMePhe)LDSRAA | | | (O2Oc)- | | | 6αMePhe, |
| | K(O2Oc-O2Oc-γE- | | | (O2Oc) | | | 10V, |
| | C18diacid)20D | | | | | | 13αMePhe, |
| | (αMePhe)IAWIANT- | | | | | | 18A, 20K, |
| | amide | | | | | | 22αMePhe, |
| | | | | | | | 26I, 27A |
| Peptide | H(Aib)HGS(αMePhe)TS | 235 | C18diacid | γE-γE- | 20 | Amide | 2Aib, 3H, 5S, |
| 231 | DVSK(αMePhe)LDSRAA | | | (O2Oc)- | | | 6αMePhe, |
| | K(O2Oc-O2Oc-γE-γE- | | | (O2Oc) | | | 10V, |
| | C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 236 | C20diacid | γE-γE- | 20 | Amide | 2Aib, 3H, 5S, |
| 232 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid)20D | | | | | | 13αMePhe, |
| | (αMePhe)VQ(Aib) | | | | | | 18A, 20K, |
| | IANTGG-amide | | | | | | 22αMePhe, |
| | | | | | | | 25Aib, 26I, |
| | | | | | | | 27A, 30G, |
| | | | | | | | 31G |
| Peptide | H(Aib)HGS(αMePhe)TS | 237 | C20diacid | γE-γE- | 20 | Amide | 2Aib, 3H, 5S, |
| 233 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid)20D | | | | | | 13αMePhe, |
| | (αMePhe)VE(Aib)IANT- | | | | | | 18A, 20K, |
| | amide | | | | | | 22αMePhe, |
| | | | | | | | 24E, 25Aib, |
| | | | | | | | 26I, 27A |
| Peptide | H(Aib)HGS(αMePhe)TS | 238 | C20diacid | γE-γE- | 20 | Amide | 2Aib, 3H, 5S, |
| 234 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid)20DFVQ(Aib) | | | | | | 13αMePhe, |
| | IANTG-amide | | | | | | 18A, 20K, |
| | | | | | | | 25Aib, 26I, |
| | | | | | | | 27A, 30G |
| Peptide | H(Aib)HGS(αMePhe)TS | 239 | C20diacid | γE-γE- | 20 | Amide | 2Aib, 3H, 5S, |
| 235 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid)20DFVQ(Aib) | | | | | | 13αMePhe, |
| | IANTGG-amide | | | | | | 18A, 20K, |
| | | | | | | | 22αMePhe, |
| | | | | | | | 25Aib, 26I, |
| | | | | | | | 27A, 30G, |
| | | | | | | | 31G |
| Peptide | H(Aib)HGS(αMePhe)TS | 240 | C18diacid | γE-γE- | 20 | Amide | 2Aib, 3H, 5S, |
| 236 | DVSK(αMePhe)LDSRAA | | | (O2Oc)- | | | 6αMePhe, |
| | K(O2Oc-O2Oc-γE-γE- | | | (O2Oc) | | | 10V, |
| | C18diacid)20DFVQ(Aib) | | | | | | 13αMePhe, |
| | IANTG-amide | | | | | | 18A, 20K, |
| | | | | | | | 25Aib, 26I, |
| | | | | | | | 27A, 30G |
| Peptide | H(Aib)HGS(αMePhe)TS | 241 | C18diacid | γE-γE- | 20 | Amide | 2Aib, 3H, 5S, |
| 237 | DVSK(αMePhe)LDSRAA | | | (O2Oc)- | | | 6αMePhe, |
| | K(O2Oc-O2Oc-γE-γE- | | | (O2Oc) | | | 10V, |
| | C18diacid)20DFVQ(Aib) | | | | | | 13αMePhe, |
| | IANTGG-amide | | | | | | 18A, 20K, |
| | | | | | | | 22αMePhe, |
| | | | | | | | 25Aib, 26I, |
| | | | | | | | 27A, 30G, |
| | | | | | | | 31G |
| Peptide | H(Aib)HGS(αMePhe)TS | 242 | C20diacid | γE-γE- | 20 | Amide | 2Aib, 3H, 5S, |
| 238 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid)20D | | | | | | 13αMePhe, |
| | (αMePhe)VQWIANTG- | | | | | | 18A, 20K, |
| | amide | | | | | | 22αMePhe, |
| | | | | | | | 26I, 27A, |
| | | | | | | | 30G |
| Peptide | H(Aib)HGS(αMePhe)TS | 243 | C20diacid | γE-γE- | 20 | Amide | 2Aib, 3H, 5S, |
| 239 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid)20D | | | | | | 13αMePhe, |
| | (αMePhe)VQWIANTGG- | | | | | | 18A, 20K, |
| | amide | | | | | | 22αMePhe, |
| | | | | | | | 26I, 27A, |
| | | | | | | | 30G, 31G |
| Peptide | H(Aib)HGS(αMePhe)TS | 244 | C18diacid | γE-γE- | 20 | Amide | 2Aib, 3H, 5S, |
| 240 | DVSK(αMePhe)LDSRAA | | | (O2Oc)- | | | 6αMePhe, |
| | K(O2Oc-O2Oc-γE-γE- | | | (O2Oc) | | | 10V, |
| | C18diacid)20D | | | | | | 13αMePhe, |
| | (αMePhe)VQWIANTG- | | | | | | 18A, 20K, |
| | amide | | | | | | 22αMePhe, |
| | | | | | | | 26I, 27A, |
| | | | | | | | 30G |
| Peptide | H(Aib) HGS(αMePhe)TS | 245 | C18diacid | γE-γE- | 20 | Amide | 2Aib, 3H, 5S, |
| 241 | DVSK(αMePhe)LDSRAA | | | (O2Oc)- | | | 6αMePhe, |
| | K(O2Oc-O2Oc-γE-γE- | | | (O2Oc) | | | 10V, |
| | C18diacid)20D | | | | | | 13αMePhe, |
| | (αMePhe)VQWIANTGG- | | | | | | 18A, 20K, |
| | amide | | | | | | 22αMePhe, |
| | | | | | | | 26I, 27A, |
| | | | | | | | 30G, 31G |
| Peptide | H(Aib)HGS(αMePhe)TS | 246 | C20diacid | γE-γE- | 20 | Amide | 2Aib, 3H, 5S, |
| 242 | DVSK(αMePhe)LDSR | | | (O2Oc)- | | | 6αMePhe, |
| | (Aib)AK(O2Oc-O2Oc-γE- | | | (O2Oc) | | | 10V, |
| | γE- | | | | | | 13αMePhe, |
| | C20diacid)20DFVQSIAN | | | | | | 18Aib, 20K, |
| | T-amide | | | | | | 25S, 26I, 27A |
| Peptide | H(Aib)HGS(αMePhe)TS | 247 | C18diacid | γE- | 20 | Amide | 2Aib, 3H, 5S, |
| 243 | DVSK(αMePhe)LDSRAA | | | (O2Oc)- | | | 6αMePhe, |
| | K(O2Oc-O2Oc-γE- | | | (O2Oc) | | | 10V, |
| | C18diacid)20D | | | | | | 13αMePhe, |
| | (αMePhe)VQSIANT- | | | | | | 18A, 20K, |
| | amide | | | | | | 22αMePhe, |
| | | | | | | | 25S, 26I, 27A |
| Peptide | H(Aib)HGS(αMePhe)TS | 248 | C20diacid | γE-γE- | 20 | Amide | 2Aib, 3H, 5S, |
| 244 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid)20D | | | | | | 13αMePhe, |
| | (αMePhe)VQSIANT- | | | | | | 18A, 20K, |
| | amide | | | | | | 22αMePhe, |
| | | | | | | | 25S, 26I, 27A |
| Peptide | H(Aib)HGS(αMePhe)TS | 249 | C18diacid | γE- | 20 | Amide | 2Aib, 3H, 5S, |
| 245 | DVSK(αMePhe)LDSRAA | | | (O2Oc)- | | | 6αMePhe, |
| | K(O2Oc-O2Oc-γE- | | | (O2Oc) | | | 10V, |
| | C18diacid)20D | | | | | | 13αMePhe, |
| | (αMePhe)VESIANT- | | | | | | 18A, 20K, |
| | amide | | | | | | 22αMePhe, |
| | | | | | | | 24E, 25S, |
| | | | | | | | 26I, 27A |
| Peptide | H(Aib)HGS(αMePhe)TS | 250 | C20diacid | γE-γE- | 20 | Amide | 2Aib, 3H, 5S, |
| 246 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid)20D | | | | | | 13αMePhe, |
| | (αMePhe)VESIANT- | | | | | | 18A, 20K, |
| | amide | | | | | | 22αMePhe, |
| | | | | | | | 24E, 25S, |
| | | | | | | | 26I, 27A |
| Peptide | H(Aib)HGS(αMePhe)TS | 251 | C18diacid | γE-γE- | 20 | Amide | 2Aib, 3H, 5S, |
| 247 | DVSK(αMePhe)LDSRAA | | | (O2Oc)- | | | 6αMePhe, |
| | K(O2Oc-O2Oc-γE-γE- | | | (O2Oc) | | | 10V, |
| | C18diacid)20D | | | | | | 13αMePhe, |
| | (αMePhe)VQWIANT- | | | | | | 18A, 20K, |
| | amide | | | | | | 22αMePhe, |
| | | | | | | | 26I, 27A |
| Peptide | (NHis)SHGS(αMePhe)T | 252 | C20diacid | γE-γE- | 20 | Amide | 2Aib, 3H, 5S, |
| 248 | SDVSK(αMePhe)LDSRA | | | (O2Oc)- | | | 6αMePhe, |
| | AK(O2Oc-O2Oc-γE-γE- | | | (O2Oc) | | | 10V, |
| | C20diacid)20D | | | | | | 13αMePhe, |
| | (αMePhe)VQWIANT- | | | | | | 18A, 20K, |
| | amide | | | | | | 22αMePhe, |
| | | | | | | | 26I, 27A |
| Peptide | H(Aib)HGS(αMePhe)TS | 253 | C20diacid | γE-γE- | 20 | Amide | 2Aib, 3H, 5S, |
| 249 | DVSK(αMePhe)LDSR | | | (O2Oc)- | | | 6αMePhe, |
| | (Aib)AK(O2Oc-O2Oc- | | | (O2Oc) | | | 10V, |
| | γE-γE- | | | | | | 13αMePhe, |
| | C20diacid)20D | | | | | | 18Aib, 20K, |
| | (αMePhe)VESIANT- | | | | | | 22αMePhe, |
| | amide | | | | | | 24E, 25S, |
| | | | | | | | 26I, 27A |
| Peptide | H(Aib)HGS(αMePhe)TS | 254 | C18diacid | γE-γE- | 20 | Amide | 2Aib, 3H, 5S, |
| 250 | DVSK(αMePhe)LDSR | | | (O2Oc)- | | | 6αMePhe, |
| | (Aib)AK(O2Oc-O2Oc- | | | (O2Oc) | | | 10V, |
| | γE-γE- | | | | | | 13αMePhe, |
| | C18diacid)20D | | | | | | 18Aib, 20K, |
| | (αMePhe)VESIANT-amide | | | | | | 22αMePhe, |
| | | | | | | | 24E, 25S, |
| | | | | | | | 26I, 27A |
| Peptide | H(Aib)HGS(αMePhe)TS | 255 | C18diacid | γE- | 20 | Amide | 2Aib, 3H, 5S, |
| 251 | DVSK(αMePhe)LDSR | | | (O2Oc)- | | | 6αMePhe, |
| | (Aib)AK(O2Oc-O2Oc- | | | (O2Oc) | | | 10V, |
| | γE-C18diacid)20DFVES | | | | | | 13αMePhe, |
| | IANT-amide | | | | | | 18Aib, 20K, |
| | | | | | | | 24E, 25S, |
| | | | | | | | 26I, 27A |
| Peptide | H(Aib)HGS(αMePhe)TS | 256 | C20diacid | γE- | 20 | Amide | 2Aib, 3H, 5S, |
| 252 | DVSK(αMePhe)LDSR | | | (O2Oc)- | | | 6αMePhe, |
| | (Aib)AK(O2Oc-O2Oc- | | | (O2Oc) | | | 10V, |
| | γE-C20diacid)20DFVES | | | | | | 13αMePhe, |
| | IANT-amide | | | | | | 18Aib, 20K, |
| | | | | | | | 24E, 25S, |
| | | | | | | | 26I, 27A |
| Peptide | H(Aib)HGS(αMePhe)TS | 257 | C18diacid | γE- | 20 | Amide | 2Aib, 3H, 5S, |
| 253 | DVSK(αMePhe)LDSR | | | (O2Oc) | | | 6αMePhe, |
| | (Aib)AK(O2Oc-γE- | | | | | | 10V, |
| | C18diacid)20DFVESIAN | | | | | | 13αMePhe, |
| | T-amide | | | | | | 18Aib, 20K, |
| | | | | | | | 24E, 25S, |
| | | | | | | | 26I, 27A |
| Peptide | H(Aib)HGS(αMePhe)TS | 258 | C18diacid | γE | 20 | Amide | 2Aib, 3H, 5S, |
| 254 | DVSK(αMePhe)LDSR | | | | | | 6αMePhe, |
| | (Aib)AK(γE- | | | | | | 10V, |
| | C18diacid)20DFVESIAN | | | | | | 13αMePhe, |
| | T-amide | | | | | | 18Aib, 20K, |
| | | | | | | | 24E, 25S, |
| | | | | | | | 26I, 27A |
| Peptide | H(Aib)HGS(αMePhe)TS | 259 | C18diacid | γE- | 20 | Amide | 2Aib, 3H, 5S, |
| 255 | DVSK(αMePhe)LDSQAA | | | (O2Oc)- | | | 6αMePhe, |
| | K(O2Oc-O2Oc-γE- | | | (O2Oc) | | | 10V, |
| | C18diacid)20D | | | | | | 13αMePhe, |
| | (αMePhe)VQ(Aib)IANT- | | | | | | 17Q, 18A, |
| | amide | | | | | | 20K, |
| | | | | | | | 22αMePhe, |
| | | | | | | | 25Aib, 26I, |
| | | | | | | | 27A |
| Peptide | H(Aib)HGS(αMePhe)TS | 260 | C20diacid | γE-γE- | 20 | Amide | 2Aib, 3H, 5S, |
| 256 | DVSK(αMePhe)LDSQAA | | | (O2Oc)- | | | 6αMePhe, |
| | K(O2Oc-O2Oc-γE-γE- | | | (O2Oc) | | | 10V, |
| | C20diacid)20D | | | | | | 13αMePhe, |
| | (αMePhe)VQ(Aib)IANT- | | | | | | 17Q, 18A, |
| | amide | | | | | | 20K, |
| | | | | | | | 22αMePhe, |
| | | | | | | | 25Aib, 26I, |
| | | | | | | | 27A |
| Peptide | H(Aib)HGS(αMePhe)TS | 26 | Stearoyl | γE-γE- | 20 | Amide | 2Aib, 3H, 5S, |
| 257 | DVSK(αMePhe)LDSRAA | | | (O2Oc)- | | | 6αMePhe, |
| | K(O2Oc-O2Oc-γE-γE- | | | (O2Oc) | | | 10V, |
| | Stearoyl)20D(αMePhe) | | | | | | 13αMePhe, |
| | VQ(Aib)IANT-amide | | | | | | 18A, 20K, |
| | | | | | | | 22αMePhe, |
| | | | | | | | 25Aib, 26I, |
| | | | | | | | 27A |
| Peptide | H(Aib)HGS(αMePhe)TS | 262 | C18diacid | γE-γE- | 20 | Amide | 2Aib, 3H, 5S, |
| 258 | DVSK(αMePhe)LDSRAA | | | (PEG)2- | | | 6αMePhe, |
| | K((PEG)2-(PEG)2-γE- | | | (PEG)2 | | | 10V, |
| | γE-C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 263 | C18diacid | (O2Oc)- | 20 | Amide | 2Aib, 3H, 5S, |
| 259 | DVSK(αMePhe)LDSRAA | | | γE- | | | 6αMePhe, |
| | K(O2Oc-γE-O2Oc- | | | (O2Oc) | | | 10V, |
| | C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 264 | C18diacid | γE- | 20 | Amide | 2Aib, 3H, 5S, |
| 260 | DVSK(αMePhe)LDSRAA | | | (O2Oc)- | | | 6αMePhe, |
| | K(O2Oc-γE-O2Oc-γE- | | | γE- | | | 10V, |
| | C18diacid)20D | | | (O2Oc) | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 265 | C18diacid | γE- | 20 | Amide | 2Aib, 3H, 5S, |
| 261 | DVSK(αMePhe)LDSRAA | | | (PEG)4 | | | 6αMePhe, |
| | K((PEG)4-γE- | | | | | | 10V, |
| | C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib) HGS(αMePhe)TS | 266 | C18diacid | γE- | 20 | Amide | 2Aib, 3H, 5S, |
| 262 | DVSK(αMePhe)LDSRAA | | | (O2Oc) | | | 6αMePhe, |
| | K(O2Oc-γE- | | | | | | 10V, |
| | C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 267 | C18diacid | γE-γE- | 20 | Amide | 2Aib, 3H, 5S, |
| 263 | DVSK(αMePhe)LDSRAA | | | (PEG)4 | | | 6αMePhe, |
| | K((PEG)4-γE-γE- | | | | | | 10V, |
| | C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 268 | C20diacid | γE-γE- | 20 | Amide | 2Aib, 3H, 5S, |
| 264 | DVSK(αMePhe)LDSRAA | | | (PEG)2- | | | 6αMePhe, |
| | K((PEG)2-(PEG)2-γE- | | | (PEG)2 | | | 10V, |
| | γE-C20diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 269 | C20diacid | (O2Oc)- | 20 | Amide | 2Aib, 3H, 5S, |
| 265 | DVSK(αMePhe)LDSRAA | | | γE- | | | 6αMePhe, |
| | K(O2Oc-γE-O2Oc- | | | (O2Oc) | | | 10V, |
| | C20diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 270 | C20diacid | γE- | 20 | Amide | 2Aib, 3H, 5S, |
| 266 | DVSK(αMePhe)LDSRAA | | | (O2Oc)- | | | 6αMePhe, |
| | K(O2Oc-γE-O2Oc-γE- | | | γE- | | | LOV, |
| | C20diacid)20D | | | (O2Oc) | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 271 | C20diacid | γE- | 20 | Amide | 2Aib, 3H, 5S, |
| 267 | DVSK(αMePhe)LDSRAA | | | (PEG)4 | | | 6αMePhe, |
| | K((PEG)4-γE- | | | | | | 10V, |
| | C20diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib) HGS(αMePhe)TS | 272 | C20diacid | γE- | 20 | Amide | 2Aib, 3H, 5S, |
| 268 | DVSK(αMePhe)LDSRAA | | | (O2Oc) | | | 6αMePhe, |
| | K(O2Oc-γE- | | | | | | 10V, |
| | C20diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 273 | C20diacid | γE-γE- | 20 | Amide | 2Aib, 3H, 5S, |
| 269 | DVSK(αMePhe)LDSRAA | | | (PEG)4 | | | 6αMePhe, |
| | K((PEG)4-γE-γE- | | | | | | 10V, |
| | C20diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 274 | C18diacid | γE-γE | 20 | Amide | 2Aib, 3H, 5S, |
| 270 | DVSK(αMePhe)LDSRAA | | | | | | 6αMePhe, |
| | K(γE-γE- | | | | | | 10V, |
| | C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 275 | C18diacid | γE | 20 | Amide | 2Aib, 3H, 5S, |
| 271 | DVSK(αMePhe)LDSRAA | | | | | | 6αMePhe, |
| | K(γE- | | | | | | 10V, |
| | C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 276 | C18diacid | (O2Oc)- | 20 | Amide | 2Aib, 3H, 5S, |
| 272 | DVSK(αMePhe)LDSRAA | | | (O2Oc) | | | 6αMePhe, |
| | K(O2Oc-O2Oc- | | | | | | 10V |
| | C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 277 | C18diacid | (O2Oc) | 20 | Amide | 2Aib, 3H, 5S, |
| 273 | DVSK(αMePhe)LDSRAA | | | | | | 6αMePhe, |
| | K(O2Oc- | | | | | | 10V, |
| | C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 278 | C18diacid | γE-γE- | 20 | Amide | 2Aib, 3H, 5S, |
| 274 | DVSK(αMePhe)LDSRAA | | | (O2Oc) | | | 6αMePhe, |
| | K(O2Oc-γE-γE- | | | | | | 10V |
| | C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 279 | C20diacid | γE-γE | 20 | Amide | 2Aib, 3H, 5S, |
| 275 | DVSK(αMePhe)LDSRAA | | | | | | 6αMePhe, |
| | K(γE-γE- | | | | | | 10V, |
| | C20diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 280 | C20diacid | γE | 20 | Amide | 2Aib, 3H, 5S, |
| 276 | DVSK(αMePhe)LDSRAA | | | | | | 6αMePhe, |
| | K(γE- | | | | | | 10V |
| | C20diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 281 | C20diacid | (O2Oc)- | 20 | Amide | 2Aib, 3H, 5S, |
| 277 | DVSK(αMePhe)LDSRAA | | | (O2Oc) | | | 6αMePhe, |
| | K(O2Oc-O2Oc- | | | | | | 10V, |
| | C20diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 282 | C20diacid | (O2Oc) | 20 | Amide | 2Aib, 3H, 5S, |
| 278 | DVSK(αMePhe)LDSRAA | | | | | | 6αMePhe, |
| | K(O2Oc- | | | | | | 10V, |
| | C20diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 283 | C20diacid | γE-γE- | 20 | Amide | 2Aib, 3H, 5S, |
| 279 | DVSK(αMePhe)LDSRAA | | | (O2Oc) | | | 6αMePhe, |
| | K(O2Oc-γE-γE- | | | | | | 10V, |
| | C20diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 284 | C20diacid | γE- | 20 | Amide | 2Aib, 3H, 5S, |
| 280 | DVSK(αMePhe)LDSRAA | | | (O2Oc)- | | | 6αMePhe, |
| | K(O2Oc-O2Oc-γE- | | | (O2Oc) | | | 10V, |
| | C20diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | HSHGS(αMePhe)TSDVS | 285 | C20diacid | γE-γE- | 20 | Amide | 3H, 5S, |
| 281 | K(αMePhe)LDSRAAK | | | (O2Oc)- | | | 6αMePhe, |
| | (O2Oc-O2Oc-γE-γE- | | | (O2Oc) | | | 10V, |
| | C20diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGSFTSDVSK | 286 | C20diacid | γE-γE- | 20 | Amide | 2Aib, 3H, 5S, |
| 282 | (αMePhe)LDSRAAK | | | (O2Oc)- | | | 10V, |
| | (O2Oc-O2Oc-γE-γE- | | | (O2Oc) | | | 13αMePhe, |
| | C20diacid)20D | | | | | | 18A, 20K, |
| | (αMePhe)VQ(Aib)IANT- | | | | | | 22αMePhe, |
| | amide | | | | | | 25Aib, 26I, |
| | | | | | | | 27A |
| Peptide | H(Aib)HGS(αMePhe)TS | 287 | C20diacid | γE-γE- | 20 | Amide | 2Aib, 3H, 5S, |
| 283 | DVSKYLDSRAAK(O2Oc- | | | (O2Oc)- | | | 6αMePhe, |
| | O2Oc-γE-γE- | | | (O2Oc) | | | 10V, 18A, |
| | C20diacid)20D | | | | | | 20K, |
| | (αMePhe)VQ(Aib)IANT- | | | | | | 22αMePhe, |
| | amide | | | | | | 25Aib, 26I, |
| | | | | | | | 27A |
| Peptide | HSHGS(αMePhe)TSDVS | 288 | C18diacid | γE- | 20 | Amide | 3H, 5S, |
| 284 | K(αMePhe)LDSRAAK | | | (O2Oc)- | | | 6αMePhe, |
| | (O2Oc-O2Oc-γE- | | | (O2Oc) | | | 10V, |
| | C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGSFTSDVSK | 289 | C18diacid | γE- | 20 | Amide | 2Aib, 3H, 5S, |
| 285 | (αMePhe)LDSRAAK | | | (O2Oc)- | | | 10V, |
| | (O2Oc-O2Oc-γE- | | | (O2Oc) | | | 13αMePhe, |
| | C18diacid)20D | | | | | | 18A, 20K, |
| | (αMePhe)VQ(Aib)IANT- | | | | | | 22αMePhe, |
| | amide | | | | | | 25Aib, 26I, |
| | | | | | | | 27A |
| Peptide | H(Aib)HGS(αMePhe)TS | 290 | C18diacid | γE- | 20 | Amide | 2Aib, 3H, 5S, |
| 286 | DVSKYLDSRAAK(O2Oc- | | | (O2Oc)- | | | 6αMePhe, |
| | O2Oc-γE- | | | (O2Oc) | | | 10V, 18A, |
| | C18diacid)20D | | | | | | 20K, |
| | (αMePhe)VQ(Aib)IANT- | | | | | | 22αMePhe, |
| | amide | | | | | | 25Aib, 26I, |
| | | | | | | | 27A |
| Peptide | H(Aib) HGS(αMePhe)TS | 291 | C18diacid | γE- | 20 | Amide | 2Aib, 3H, 5S, |
| 287 | DVSK(αMePhe)LDSRAA | | | (O2Oc)- | | | 6αMePhe, |
| | K(O2Oc-O2Oc-γE- | | | (O2Oc) | | | 10V, |
| | C18diacid)20DFVQ(Aib) | | | | | | 13αMePhe, |
| | IANT-amide | | | | | | 18A, 20K, |
| | | | | | | | 25Aib, 26I, |
| | | | | | | | 27A |
| Peptide | H(Aib)HGS(αMePhe)TS | 292 | C18diacid | γE- | 20 | Amide | 2Aib, 3H, 5S, |
| 288 | DVSK(αMePhe)LDSRAA | | | (O2Oc)- | | | 6αMePhe, |
| | K(O2Oc-O2Oc-γE- | | | (O2Oc) | | | 10V, |
| | C18diacid)20D | | | | | | 13αMePhe, |
| | (αMePhe)VQWIANT- | | | | | | 18A, 20K, |
| | amide | | | | | | 22αMePhe, |
| | | | | | | | 26I, 27A |
| Peptide | H(Aib)HGS(αMePhe)TS | 293 | C20diacid | γE-γE- | 20 | Amide | 2Aib, 3H, 5S, |
| 289 | DVSKYLDSRAAK(O2Oc- | | | (O2Oc)- | | | 6αMePhe, |
| | O2Oc-γE-γE- | | | (O2Oc) | | | 10V, 18A, |
| | C20diacid)20DFVQWIA | | | | | | 20K, 26I, 27A |
| | NT-amide | | | | | | |
| Peptide | H(Aib)HGSFTSDVSK | 294 | C20diacid | γE-γE- | 20 | Amide | 2Aib, 3H, 5S, |
| 290 | (αMePhe)LDSRAAK | | | (O2Oc)- | | | 10V, |
| | (O2Oc-O2Oc-γE-γE- | | | (O2Oc) | | | 13αMePhe, |
| | C20diacid)20DFVQWIA | | | | | | 18A, 20K, |
| | NT-amide | | | | | | 26I, 27A |
| Peptide | H(Aib)HGSFTSDVSKYLD | 295 | C20diacid | γE-γE- | 20 | Amide | 2Aib, 3H, 5S, |
| 291 | SRAAK(O2Oc-O2Oc-γE- | | | (O2Oc)- | | | 10V, 18A, |
| | γE- | | | (O2Oc) | | | 20K, |
| | C20diacid)20D | | | | | | 22αMePhe, |
| | (αMePhe)VQWIANT- | | | | | | 26I, 27A |
| | amide | | | | | | |
| Peptide | H(Aib)HGS(αMePhe)TS | 296 | C18diacid | γE- | 20 | Amide | 2Aib, 3H, 5S, |
| 292 | DVSKYLDSRAAK(O2Oc- | | | (O2Oc)- | | | 6αMePhe, |
| | O2Oc-γE- | | | (O2Oc) | | | 10V, 18A, |
| | C18diacid)20DFVQWIA | | | | | | 20K, 26I, 27A |
| | NT-amide | | | | | | |
| Peptide | H(Aib)HGSFTSDVSK | 297 | C18diacid | γE- | 20 | Amide | 2Aib, 3H, 5S, |
| 293 | (αMePhe)LDSRAAK | | | (O2Oc)- | | | 10V, |
| | (O2Oc-O2Oc-γE- | | | (O2Oc) | | | 13αMePhe, |
| | C18diacid)20DFVQWIA | | | | | | 18A, 20K, |
| | NT-amide | | | | | | 26I, 27A |
| Peptide | H(Aib)HGSFTSDVSKYLD | 298 | C18diacid | γE- | 20 | Amide | 2Aib, 3H, 5S, |
| 294 | SRAAK(O2Oc-O2Oc-γE- | | | (O2Oc)- | | | 10V, 18A, |
| | C18diacid)20D | | | (O2Oc) | | | 20K, |
| | (αMePhe)VQWIANT- | | | | | | 22αMePhe, |
| | amide | | | | | | 26I, 27A |
| Peptide | H(Aib)HGS(αMePhe)TS | 299 | C20diacid | γE-γE- | 20 | Amide | 2Aib, 3H, 5S, |
| 295 | DVSK(Aib)LDSRAAK | | | (O2Oc)- | | | 6αMePhe, |
| | (O2Oc-O2Oc-γE-γE- | | | (O2Oc) | | | 10V, 13Aib, |
| | C20diacid)20D | | | | | | 18A, 20K, |
| | (αMePhe)VQ(Aib)IANT- | | | | | | 22αMePhe, |
| | amide | | | | | | 25Aib, 26I, |
| | | | | | | | 27A |
| Peptide | H(Aib)HGS(αMePhe)TS | 300 | C18diacid | γE- | 20 | Amide | 2Aib, 3H, 5S, |
| 296 | DVSK(Aib)LDSRAAK | | | (O2Oc)- | | | 6αMePhe, |
| | (O2Oc-O2Oc-γE- | | | (O2Oc) | | | 10V, 13Aib, |
| | C18diacid)20D | | | | | | 18A, 20K, |
| | (αMePhe)VQ(Aib)IANT- | | | | | | 22αMePhe, |
| | amide | | | | | | 25Aib, 26I, |
| | | | | | | | 27A |
| Peptide | H(Aib)HGS(αMePhe)TS | 301 | C18diacid | γE- | 20 | Amide | 2Aib, 3H, 5S, |
| 297 | DVSR(αMePhe)LDSRAA | | | (O2Oc)- | | | 6αMePhe, |
| | K(O2Oc-O2Oc-γE- | | | (O2Oc) | | | 10V, 12R, |
| | C18diacid)20D | | | | | | 13αMePhe, |
| | (αMePhe)VQ(Aib) | | | | | | 18A, 20K, |
| | IANT-amide | | | | | | 22αMePhe, |
| | | | | | | | 25Aib, 26I, |
| | | | | | | | 27A |
| Peptide | H(Aib)HGS(αMePhe)TS | 302 | C20diacid | γE-γE- | 20 | Amide | 2Aib, 3H, 5S, |
| 298 | DVSR(αMePhe)LDSRAA | | | (O2Oc)- | | | 6αMePhe, |
| | K(O2Oc-O2Oc-γE-γE- | | | (O2Oc) | | | 10V, 12R, |
| | C20diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)HGS(αMePhe)TS | 303 | C20diacid | γE-γE- | 20 | Amide | 2Aib, 3H, 5S, |
| 299 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid)20D | | | | | | 13αMePhe, |
| | (αMePhe)VQ(Aib)IAET- | | | | | | 18A, 20K, |
| | amide | | | | | | 22αMePhe, |
| | | | | | | | 25Aib, 26I, |
| | | | | | | | 27A, 28E |
| Peptide | H(Aib)HGS(αMePhe)TS | 304 | C18diacid | γE- | 20 | Amide | 2Aib, 3H, 5S, |
| 300 | DVSK(αMePhe)LDSRAA | | | (O2Oc)- | | | 6αMePhe, |
| | K(O2Oc-O2Oc-γE- | | | (O2Oc) | | | 10V, |
| | C18diacid)20D | | | | | | 13αMePhe, |
| | (αMePhe)VQ(Aib)IAET- | | | | | | 18A, 20K, |
| | amide | | | | | | 22αMePhe, |
| | | | | | | | 25Aib, 26I, |
| | | | | | | | 27A, 28E |
| Peptide | H(Aib)HGS(αMePhe)TS | 305 | C18diacid | γE- | 20 | Amide | 2Aib, 3H, 5S, |
| 301 | DVSK(αMePhe)LDSRAA | | | (O2Oc)- | | | 6αMePhe, |
| | K(O2Oc-O2Oc-γE- | | | (O2Oc) | | | 10V, |
| | C18diacid)20D | | | | | | 13αMePhe, |
| | (αMePhe)IA(Aib)IAET- | | | | | | 18A, 20K, |
| | amide | | | | | | 22αMePhe, |
| | | | | | | | 231, 24A, |
| | | | | | | | 25Aib, 26I, |
| | | | | | | | 27A, 28E |
| Peptide | H(Aib)HGS(αMePhe)TS | 306 | C20diacid | γE-γE- | 20 | Amide | 2Aib, 3H, 5S, |
| 302 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid)20D | | | | | | 13αMePhe, |
| | (αMePhe)IA(Aib)IAET- | | | | | | 18A, 20K, |
| | amide | | | | | | 22αMePhe, |
| | | | | | | | 23I, 24A, |
| | | | | | | | 25Aib, 26I, |
| | | | | | | | 27A, 28E |
| Peptide | H(Aib)HGS(αMePhe)TS | 307 | C18diacid | γE- | 20 | Acid | 2Aib, 3H, 5S, |
| 303 | DVSK(αMePhe)LDSRAA | | | (O2Oc)- | | | 6αMePhe, |
| | K(O2Oc-O2Oc-γE- | | | (O2Oc) | | | 10V, |
| | C18diacid)20D | | | | | | 13αMePhe, |
| | (αMePhe)VQ(Aib)IANT- | | | | | | 18A, 20K, |
| | acid | | | | | | 22αMePhe, |
| | | | | | | | 25Aib, 26I, |
| | | | | | | | 27A |
| Peptide | H(Aib)HGS(αMePhe)TS | 308 | C20diacid | γE-γE- | 20 | Acid | 2Aib, 3H, 5S, |
| 304 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid)20D | | | | | | 13αMePhe, |
| | (αMePhe)VQ(Aib)IANT- | | | | | | 18A, 20K, |
| | acid | | | | | | 22αMePhe, |
| | | | | | | | 25Aib, 26I, |
| | | | | | | | 27A |
| Peptide | H(Aib)HGTFTSDVSK | 309 | C20diacid | γE-γE- | 20 | Amide | 2Aib, 3H, |
| 305 | (αMePhe)LDSRAAK | | | (O2Oc)- | | | 10V, |
| | (O2Oc-O2Oc-γE-γE- | | | (O2Oc) | | | 13αMePhe, |
| | C20diacid)20DFVQWIA | | | | | | 18A, 20K, |
| | NT-amide | | | | | | 26I, 27A |
| Peptide | H(Aib)QGTFTSDVSK | 310 | C18diacid | γE- | 20 | Amide | 2Aib, 10V, |
| 306 | (αMePhe)LDSRAAK | | | (O2Oc)- | | | 13αMePhe, |
| | (O2Oc-O2Oc-γE- | | | (O2Oc) | | | 18A, 20K, |
| | C18diacid)20DFVEWIA | | | | | | 24E, 26I, 27A |
| | NT-amide | | | | | | |
| Peptide | H(Aib)QGTFTSDVSK | 311 | C18diacid | γE- | 20 | Amide | 2Aib, 10V, |
| 307 | (αMePhe)LDSRAAK | | | (O2Oc)- | | | 13αMePhe, |
| | (O2Oc-O2Oc-γE- | | | (O2Oc) | | | 18A, 20K, |
| | C18diacid)20DFVRWIA | | | | | | 24R, 26I, |
| | NT-amide | | | | | | 27A |
| Peptide | H(Aib)QGTFTSDVSK | 312 | C18diacid | γE- | 20 | Amide | 2Aib, 10V, |
| 308 | (αMePhe)LDTRAAK | | | (O2Oc)- | | | 13αMePhe, |
| | (O2Oc-O2Oc-γE- | | | (O2Oc) | | | 16T, 18A, |
| | C18diacid)20DFVQWIA | | | | | | 20K, 26I, 27A |
| | NT-amide | | | | | | |
| Peptide | H(Aib)QGTFTSDVSK | 313 | C18diacid | γE- | 20 | Amide | 2Aib, 10V, |
| 309 | (αMePhe)LDERAAK | | | (O2Oc)- | | | 13αMePhe, |
| | (O2Oc-O2Oc-γE- | | | (O2Oc) | | | 16E, 18A, |
| | C18diacid)20DFVQWIA | | | | | | 20K, 26I, 27A |
| | NT-amide | | | | | | |
| Peptide | H(Aib)QGTFTSDVSK | 314 | C18diacid | γE- | 20 | Amide | 2Aib, 10V, |
| 310 | (αMePhe)LDSRAAK | | | (O2Oc)- | | | 13αMePhe, |
| | (O2Oc-O2Oc-γE- | | | (O2Oc) | | | 18A, 20K, |
| | C18diacid)20DFVQWLE | | | | | | 27E, 28A, |
| | AGG-amide | | | | | | 29G, 30G |
| Peptide | H(Aib)QGTFTSDVSK | 315 | C18diacid | γE- | 20 | Amide | 2Aib, 10V, |
| 311 | (αMePhe)LDSRAAK | | | (O2Oc)- | | | 13αMePhe, |
| | (O2Oc-O2Oc-γE- | | | (O2Oc) | | | 18A, 20K, |
| | C18diacid)20DFVQWLV | | | | | | 27V, 28E |
| | ET-amide | | | | | | |
| Peptide | H(Aib)QGTFTSDYSK | 316 | C18diacid | γE- | 20 | Amide | 2Aib, 10V, |
| 312 | (αMePhe)LDSRRAK | | | (O2Oc)- | | | 13αMePhe, |
| | (O2Oc-O2Oc-γE- | | | (O2Oc) | | | 18A, 20K, |
| | C18diacid)20DFVQWLV | | | | | | 27V, |
| | ((PEG)4)-amide | | | | | | 28(PEG)4, |
| | | | | | | | des29 |
| Peptide | H(Aib)QGTFTSDVSK | 317 | C18diacid | γE- | 20 | Acid | 2Aib, 10V, |
| 313 | (αMePhe)LDSR(Aib)AK | | | (O2Oc)- | | | 13αMePhe, |
| | (O2Oc-O2Oc-γE- | | | (O2Oc) | | | 18Aib, 20K, |
| | C18diacid)20DFVQWLL | | | | | | 27L |
| | NT-acid | | | | | | |
| Peptide | H(Aib)QGTFTSDVSK | 318 | C18diacid | γE- | 20 | Amide | 2Aib, 10V, |
| 314 | (αMePhe)LDSR(Aib)AK | | | (O2Oc)- | | | 13αMePhe, |
| | (O2Oc-O2Oc-γE- | | | (O2Oc) | | | 18Aib, 20K, |
| | C18diacid)20DFVQ(Aib) | | | | | | 25Aib, 27V, |
| | LVAT-amide | | | | | | 28A |
| Peptide | H(Aib)QGTFTSDVSK | 319 | C18diacid | γE- | 20 | Amide | 2Aib, 10V, |
| 315 | (αMePhe)LDSRAAK | | | (O2Oc)- | | | 13αMePhe, |
| | (O2Oc-O2Oc-γE- | | | (O2Oc) | | | 18A, 20K, |
| | C18diacid)20DFVQ(Aib) | | | | | | 25Aib, 27V, |
| | LVAT-amide | | | | | | 28A |
| Peptide | HSQGTFTSDYSK | 320 | C18diacid | γE- | 20 | Amide | 13αMeF, |
| 316 | (αMePhe)LEEEAVK | | | (O2Oc)- | | | 15E, 16E, |
| | (O2Oc-O2Oc-γE- | | | (O2Oc) | | | 17E, 18A, |
| | stearOOH)20LFIRWLM | | | | | | 19V, 20K, |
| | NT-amide | | | | | | 21L, 23I, 24R |
| Peptide | H(Aib)QGTFTSDVSK | 321 | C18diacid | γE- | 20 | Amide | 2Aib, 10V, |
| 317 | (αMePhe)LD(Aib)RAAK | | | (O2Oc)- | | | 13αMePhe, |
| | (O2Oc-O2Oc-γE- | | | (O2Oc) | | | 16Aib, 18A, |
| | C18diacid)20DFVQWIA | | | | | | 20K, 25Aib, |
| | NT-amide | | | | | | 26I, 27A |
| Peptide | H(αMeSer)QGTFTSDVS | 322 | C18diacid | γE- | 20 | Amide | 2αMeSer, |
| 318 | K(αMePhe)LDSRAAK | | | (O2Oc)- | | | 10V, |
| | (O2Oc-O2Oc-γE- | | | (O2Oc) | | | 13αMePhe, |
| | C18diacid)20DFVQWIA | | | | | | 18A, 20K, |
| | NT-amide | | | | | | 26I, 27A |
| Peptide | HS(αMeGln)GTFTSDVS | 323 | C18diacid | γE- | 20 | Amide | 3αMeGln, |
| 319 | K(αMePhe)LDSRAAK | | | (O2Oc)- | | | 10V, |
| | (O2Oc-O2Oc-γE- | | | (O2Oc) | | | 13αMePhe, |
| | C18diacid)20DFVQWIA | | | | | | 18A, 20K, |
| | NT-amide | | | | | | 26I, 27A |
| Peptide | H(DSer)QGTFTSDVSK | 324 | C18diacid | γE- | 20 | Amide | 2dS, 10V, |
| 320 | (αMePhe)LDSRAAK | | | (O2Oc)- | | | 13αMePhe, |
| | (O2Oc-O2Oc-γE- | | | (O2Oc) | | | 18A, 20K, |
| | C18diacid)20DFVQWIA | | | | | | 26I, 27A |
| | NT-amide | | | | | | |
| Peptide | H(Aib)QGS(αMePhe)TS | 325 | C18diacid | γE- | 20 | Amide | 2Aib, 5S, |
| 321 | DVSK(αMePhe)LDSRAA | | | (O2Oc)- | | | 6αMePhe, |
| | K(O2Oc-O2Oc-γE- | | | (O2Oc) | | | 10V, |
| | C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)QGS(αMePhe)TS | 326 | C20diacid | γE- | 20 | Amide | 2Aib, 5S, |
| 322 | DVSK(αMePhe)LDSRAA | | | (O2Oc)- | | | 6αMePhe, |
| | K(O2Oc-O2Oc-γE-γE- | | | (O2Oc) | | | 10V, |
| | C20diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | HS(αMeGln)GS(αMePh | 327 | C18diacid | γE- | 20 | Amide | 3αMeGln, |
| 323 | e)TSDVSK(αMePhe)LDS | | | (O2Oc)- | | | 5S, |
| | RAAK(O2Oc-O2Oc-γE- | | | (O2Oc) | | | 6αMePhe, |
| | C18diacid)20D | | | | | | 10V, |
| | (αMePhe)VQ(Aib)IANT- | | | | | | 13αMePhe, |
| | amide | | | | | | 18A, 20K, |
| | | | | | | | 22αMePhe, |
| | | | | | | | 25Aib, 26I, |
| | | | | | | | 27A |
| Peptide | HS(β- | 328 | C18diacid | γE- | 20 | Amide | 3β- |
| 324 | dimethylGln)GS | | | (O2Oc)- | | | dimethylGln, |
| | (αMePhe)TSDVSK | | | (O2Oc) | | | 5S, |
| | (αMePhe)LDSRAAK | | | | | | 6αMePhe, |
| | (O2Oc-O2Oc-γE- | | | | | | 10V, |
| | C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | HS(N- | 329 | C18diacid | γE- | 20 | Amide | 3N-MeGln, |
| 325 | MeGln)GS(αMePhe)TS | | | (O2Oc)- | | | 5S, |
| | DVSK(αMePhe)LDSRAA | | | (O2Oc) | | | 6αMePhe, |
| | K(O2Oc-O2Oc-γE- | | | | | | 10V, |
| | C18diacid)20D | | | | | | 13MePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | HS(dGln)GS(αMePhe)T | 330 | C18diacid | γE- | 20 | Amide | 3dGln, 5S, |
| 326 | SDVSK(αMePhe)LDSRA | | | (O2Oc)- | | | 6αMePhe, |
| | AK(O2Oc-O2Oc-γE- | | | (O2Oc) | | | 10V, |
| | C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Acpr)QGS(αMePhe)T | 331 | C18diacid | γE- | 20 | Amide | 2Acpr, 5S, |
| 327 | SDVSK(αMePhe)LDSRA | | | (O2Oc)- | | | 6αMePhe, |
| | AK(O2Oc-O2Oc-γE- | | | (O2Oc) | | | 10V, |
| | C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Acbu)QGS(αMePhe) | 332 | C18diacid | γE- | 20 | Amide | 2Acbu, 5S, |
| 328 | TSDVSK(αMePhe)LDSR | | | (O2Oc)- | | | 6αMePhe, |
| | AAK(O2Oc-O2Oc-γE- | | | (O2Oc) | | | 10V, |
| | C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(αMeSer)QGS(αMePh | 333 | C18diacid | γE- | 20 | Amide | 2αMeSer, |
| 329 | e)TSDVSK(αMePhe)LDS | | | (O2Oc)- | | | 5S, |
| | RAAK(O2Oc-O2Oc-γE- | | | (O2Oc) | | | 6αMePhe, |
| | C18diacid)20D | | | | | | 10V, |
| | (αMePhe)VQ(Aib)IANT- | | | | | | 13αMePhe, |
| | amide | | | | | | 18A, 20K, |
| | | | | | | | 22αMePhe, |
| | | | | | | | 25Aib, 26I, |
| | | | | | | | 27A |
| Peptide | H(dSer)QGS(αMePhe)T | 334 | C18diacid | γE- | 20 | Amide | 2dSer, 5S, |
| 330 | SDVSK(αMePhe)LDSRA | | | (O2Oc)- | | | 6αMePhe, |
| | AK(O2Oc-O2Oc-γE- | | | (O2Oc) | | | 10V, |
| | C18diacid)20D | | | | | | 13αMePhe, |
| | (αMePhe)VQ(Aib)IANT- | | | | | | 18A, 20K, |
| | amide | | | | | | 22αMePhe, |
| | | | | | | | 25Aib, 26I, |
| | | | | | | | 27A |
| Peptide | H(Aib)QGTFTSDVSK | 335 | C18diacid | γE- | 20 | Acid | 2Aib, 10V, |
| 331 | (αMePhe)LDSRRAK | | | (O2Oc)- | | | 13αMePhe, |
| | (O2Oc-O2Oc-γE- | | | (O2Oc) | | | 20K, 24R, |
| | C18diacid)20DFVRWLL | | | | | | 27L, 28E, |
| | E(Aib)G-acid | | | | | | 29Aib, 30G |
| Peptide | H(Aib)QGTFTSDVSK | 336 | C18diacid | γE- | 20 | Acid | 2Aib, 10V, |
| 332 | (αMePhe)LDSRRAK | | | (O2Oc)- | | | 13αMePhe, |
| | (O2Oc-O2Oc-γE- | | | (O2Oc) | | | 20K, 27L, |
| | C18diacid)20DFVQWLL | | | | | | 28E, 29Aib, |
| | E(Aib)G-acid | | | | | | 30G |
| Peptide | H(Aib)QGTFTSDVSK | 337 | C18diacid | γE- | 20 | Acid | 2Aib, 10V, |
| 333 | (αMePhe)LDSRRAK | | | (O2Oc)- | | | 13αMePhe, |
| | (O2Oc-O2Oc-γE- | | | (O2Oc) | | | 20K, 27L, |
| | C18diacid)20DFVQWLL | | | | | | 28Aib, 30E |
| | (Aib)TE-acid | | | | | | |
| Peptide | H(Aib)QGTFTSDYSK | 338 | C18diacid | γE | 20 | Acid | 2Aib, |
| 334 | (αMePhe)LDSRRAK(γE- | | | | | | 13αMePhe, |
| | C18diacid)20DFVQWLA | | | | | | 20K, 27A, |
| | (Aib)E-acid | | | | | | 28Aib, 30E |
| Peptide | H(Aib)QGTFTSDYSK | 339 | C18diacid | γE | 20 | Acid | 2Aib, |
| 335 | (αMePhe)LDSRRAK(γE- | | | | | | 13αMePhe, |
| | C18diacid)20DFVQWLL | | | | | | 20K, 27L, |
| | E(Aib)G-acid | | | | | | 28E, 29Aib, |
| | | | | | | | 30G |
| Peptide | H(Aib)QGTFTSDYSK | 340 | C18diacid | γE | 20 | Acid | 2Aib, |
| 336 | (αMePhe)LDSRRAK(γE- | | | | | | 13αMePhe, |
| | C18diacid)20DFVQWLIS | | | | | | 20K, 27I, |
| | E-acid | | | | | | 28S, 29E |
| Peptide | H(Aib)QGTFTSDYSK | 341 | C18diacid | γE | 20 | Acid | 2Aib, |
| 337 | (αMePhe)LDSRRAK(γE- | | | | | | 13αMePhe, |
| | C18diacid)20DFVQWLL | | | | | | 20K, 27L, |
| | (Aib)T-acid | | | | | | 28Aib |
| Peptide | H(Aib)QGTFTSDYSK | 342 | C18diacid | γE | 20 | Acid | 2Aib, |
| 338 | (αMePhe)LD(Aib)RRAK | | | | | | 13αMePhe, |
| | (γE-C18diacid)20DFVQ | | | | | | 16Aib, 20K, |
| | WLL(Aib)T-acid | | | | | | 27L, 28Aib |
| Peptide | H(Aib)QGTFTSDYSK | 343 | C18diacid | γE | 20 | Acid | 2Aib, |
| 339 | (αMePhe)LDSRRAK(γE- | | | | | | 13αMePhe, |
| | C18diacid)20DFVQWLL | | | | | | 20K, 27L, |
| | (Aib)E-acid | | | | | | 28Aib, 29E |
| Peptide | H(Aib)QGTFTSDYSK | 344 | C18diacid | γE | 20 | Acid | 2Aib, |
| 340 | (αMePhe)LDSRRAK(γE- | | | | | | 13αMePhe, |
| | C18diacid)20DFVQWLL | | | | | | 20K, 27L, |
| | (Aib)TE-acid | | | | | | 28Aib, 30E |
| Peptide | H(Aib)QGTFTSDYSK | 345 | C18diacid | γE | 20 | Acid | 2Aib, |
| 341 | (αMePhe)LDSRRAK(γE- | | | | | | 13αMePhe, |
| | C18diacid)20DFVQWLL | | | | | | 20K, 27L, |
| | E(Aib)T-acid | | | | | | 28E, 29Aib, |
| | | | | | | | 30T |
| Peptide | H(Aib)QGTFTSDVSK | 346 | C18diacid | γE | 20 | Acid | 2Aib, 10V, |
| 342 | (αMePhe)LDSRAAK(γE- | | | | | | 13αMePhe, |
| | C18diacid)20DFVQWIA | | | | | | 18A, 20K, |
| | NT-acid | | | | | | 26I, 27A |
| Peptide | H(Aib)QGTFTSDVSK | 347 | C18diacid | γE- | 20 | Amide | 2Aib, 10V, |
| 343 | (αMePhe)LDSRAAK(γE- | | | γE(O2Oc)- | | | 13αMePhe, |
| | γE-O2Oc-O2Oc-γE-γE- | | | (O2Oc)- | | | 18A, 20K, |
| | C18diacid)20DFVQWIA | | | γE-γE | | | 26I, 27A |
| | NT-amide | | | | | | |
| Peptide | H(Aib)QGTFTSDVSK | 532 | C18diacid | γE- | 20 | Acid | 2Aib, 10V, |
| 521 | (αMePhe)LDSERAK | | | (O2Oc)- | | | 13αMePhe, |
| | (O2Oc-O2Oc-γE- | | | (O2Oc) | | | 17E, 20K, |
| | C18diacid)20DFVQWLE | | | | | | 27E, 28A, |
| | AGG-acid | | | | | | 29G, 30G |
| Peptide | H(Aib)QGTFTSDVSK | 533 | C18diacid | γE- | 20 | Acid | 2Aib, 10V, |
| 522 | (αMePhe)LDSERAK | | | (O2Oc)- | | | 13αMePhe, |
| | (O2Oc-O2Oc-γE- | | | (O2Oc) | | | 17E, 20K, |
| | C18diacid)20DFVAWLE | | | | | | 24A, 27E, |
| | AGG-acid | | | | | | 28A, 29G, |
| | | | | | | | 30G |
| Peptide | H(Aib)QGTFTSDVSK | 534 | C20diacid | γE- | 20 | Acid | 2Aib, 10V, |
| 523 | (αMePhe)LDSERAK | | | (O2Oc)- | | | 13αMePhe, |
| | (O2Oc-O2Oc-γE- | | | (O2Oc) | | | 17E, 20K, |
| | C20diacid)20DFVAWLE | | | | | | 24A, 27E, |
| | AGG-acid | | | | | | 28A, 29G, |
| | | | | | | | 30G |
| Peptide | H(Aib)QGTFTSDVSE | 535 | C18diacid | γE- | 20 | Acid | 2Aib, 10V, |
| 524 | (αMePhe)LDSERAK | | | (O2Oc)- | | | 12E, |
| | (O2Oc-O2Oc-γE- | | | (O2Oc) | | | 13αMePhe, |
| | C18diacid)20DFVRWLE | | | | | | 17E, 20K, |
| | AGG-acid | | | | | | 24R, 27E, |
| | | | | | | | 28A, 29G, |
| | | | | | | | 30G |
| Peptide | H(Aib)QGTFTSDVSK | 536 | C18diacid | γE- | 20 | Acid | 2Aib, 10V, |
| 525 | (αMePhe)LDSERAK | | | (O2Oc)- | | | 13αMePhe, |
| | (O2Oc-O2Oc-γE- | | | (O2Oc) | | | 17E, 20K, |
| | C18diacid)20DFVQWLE | | | | | | 27E, 28A, |
| | A(Aib)G-acid | | | | | | 29Aib, 30G |
| Peptide | H(Aib)QGTFTSDVSK | 537 | C18diacid | γE- | 20 | Acid | 2Aib, 10V, |
| 526 | (αMePhe)LDSERAK | | | (O2Oc)- | | | 13αMePhe, |
| | (O2Oc-O2Oc-γE- | | | (O2Oc) | | | 17E, 20K, |
| | C18diacid)20DFVAWLE | | | | | | 24A, 27E, |
| | A(Aib)G-acid | | | | | | 28A, 29Aib, |
| | | | | | | | 30G |

In some aspects, the peptide comprises the sequence: X1-X2-X3-G-X5-X6-T-S-D-X10-S-K-X13-L-X15-X16-X17-X18-X19-X20-X21-X22-X23-X24-X25-X26-X27-X28-X29-X30-X31-Z, wherein X1 is H or ((1H-imidazol-4-yl)methyl)glycine (NHis), X2 is S or Aminoisobutyric acid (Aib), X3 is Q, H, or I, X5 is T or S, X6 is F or αMethyl-Phenylalanine (αMePhe), X10 is Y or V, X13 is Y, αMePhe, Aib, or Diphenylalanine (Dip), X15 is D or E, X16 is S, E, or L, X17 is R, Q, or E, X18 is R, A, or Aib, X19 is A or V, X20 is Q or R, X21 is D or L, X22 is F or αMePhe, X23 is V or I, X24 is Q or K, wherein the K can comprise an acyl moiety and/or can be lipidated, X25 is W, Aib, or S, X26 is L or I, X27 is M, V, L, or A, X28 is N, E, or Aib, X29 is T, Aib, G, or not present, X30 is not present, Aib, or G, X31 is not present or G, and Z is amide or acid (SEQ ID NO: 415), wherein the peptide does not comprise SEQ ID NO: 1.

In some aspects, the residue at position 24 is acylated. In some aspects, the residue at position 24 is lipidated. In some aspects, the lipid is selected from the group consisting of a palmitoyl group, stearoyl group, lauryl group, myristoyl group, margaroyl group, octadecanedioic acid (C18diacid), and icosanedioic acid (C20diacid). In some aspects, the lipid is selected from the group consisting of an octadecanedioic acid (C18diacid), and icosanedioic acid (C20diacid).

In some aspects, the lipid attached to the residue at position 24 is attached via a linker. In some aspects, the linker is selected from the group consisting of (O2Oc), (O2Oc)-(O2Oc), (O2Oc)-γE-(O2Oc), (PEG)2-(PEG)2-γE-γE, (PEG)2-γE-(PEG)2-γE, γE, γE-(O2Oc), γE-(O2Oc)-(O2Oc), γE-(O2Oc)-γE-(O2Oc), γE-(PEG)2-(PEG)2, γE-(PEG)2-γE-(PEG)2, γE-(PEG)4, γE-γE, γE-γE-(O2Oc), γE-γE-(O2Oc)-(O2Oc), γE-γE-(PEG)12, γE-γE-(PEG)2-(PEG)2, γE-γE-(PEG)2-γE-γE, γE-γE-(PEG)4, and γE-γE-(PEG)8. In some aspects, linker is selected from the group consisting of γE, γE-(O2Oc)-(O2Oc), γE-γE-(PEG)2-(PEG)2, γE-γE-(PEG)2-γE-γE, and γE-γE-(O2Oc)-(O2Oc).

In some aspects, the peptide comprises the sequence: H-Aminoisobutyric acid (Aib)-Q-G-T-F-T-S-D-V-S-K-αMethyl-Phenylalanine (αMePhe)-L-X15-X16-R-R-A-Q-D-F-V-K-W-L-X27-X28-X29-X30-Z, wherein X15 is D or E, X16 is S or L, X27 is V or L, X28 is E or Aib, X29 is T, Aib or G, X30 is G or Aib or not present, Z is amide or acid (SEQ ID NO: 416).

In some aspects, the lysine at position 24 is acylated. In some aspects, the lipid is selected from the group consisting of a palmitoyl group, stearoyl group, lauryl group, myristoyl group, margaroyl group, octadecanedioic acid (C18diacid), and icosanedioic acid (C20diacid). In some aspects, the lysine at position 24 is lipidated In some aspects, the lipid is octadecanedioic acid (C18diacid).

In some aspects, the lipid is linked to the lysine at position 24 via a linker. In some aspects, the linker is selected from the group consisting of (O2Oc), (O2Oc)-(O2Oc), (O2Oc)-γE-(O2Oc), (PEG)2-(PEG)2-γE-γE, (PEG)2-γE-(PEG)2-γE, γE, γE-(O2Oc), γE-(O2Oc)-(O2Oc), γE-(O2Oc)-γE-(O2Oc), γE-(PEG)2-(PEG)2, γE-(PEG)2-γE-(PEG)2, γE-(PEG)4, γE-γE, γE-γE-(O2Oc), γE-γE-(O2Oc)-(O2Oc), γE-γE-(PEG)12, γE-γE-(PEG)2-(PEG)2, γE-γE-(PEG)2-γE-γE, γE-γE-(PEG)4, and γE-γE-(PEG)8. In some aspects, the linker is γE-(O2Oc)-(O2Oc).

In certain aspects, GLP-1/glucagon agonist peptides as disclosed have desirable potencies at the glucagon and GLP-1 receptors.

In some aspects, the peptide comprises any one of SEQ ID NOs: 348-395.

In some aspects, the peptide is any one any of the peptides in Table 3.

TABLE 3
Peptides Modified at Position 24

| | | | | Linker | | | Sequence |
|---|---|---|---|---|---|---|---|
| | | | Albumin | (described | Acyl- | | modification |
| | | | binding | N→C | ation | C- | with respect |
| Peptide | Sequence | | moiety | term) | site | term. | to glucagon |
| Peptide | H(Aib)QGSFTSDVSK(Dip) | 348 | C18diacid | γE | 24 | Amide | 2Aib, 5S, 10V, |
| 343 | LDSRAAQDFVK(γE- | | | | | | 13Dip, 18A, |
| | C18diacid)24WIANT- | | | | | | 24K, 26I, 27A |
| | amide | | | | | | |
| Peptide | HSQGS(αMePhe)TSDVS | 349 | C18diacid | γE- | 24 | Amide | 5S, |
| 344 | K(Aib)LDSRAAQD | | | (O2Oc)- | | | 6αMePhe, |
| | (αMePhe)VK(O2OC-O2OC- | | | (O2Oc) | | | 10V, 13Aib, |
| | γE- | | | | | | 18A, |
| | C18diacid)24(Aib)IAN- | | | | | | 22αMePhe, |
| | amide | | | | | | 24K, 25Aib, |
| | | | | | | | 26I, 27A, |
| | | | | | | | des29T |
| Peptide | H(Aib)HGTFTSDVSK | 350 | C18diacid | γE- | 24 | Amide | 2Aib, 3H, |
| 345 | (αMePhe)LDSQAAQDFVK | | | (O2Oc)- | | | 10V, |
| | (O2Oc-O2Oc-γE- | | | (O2Oc) | | | 13αMePhe, |
| | C18diacid)24WIANT- | | | | | | 17Q, 18A, |
| | amide | | | | | | 24K, 26I, 27A |
| Peptide | H(Aib)HGTFTSDVSK | 351 | C18diacid | γE | 24 | Amide | 2Aib, 3H, |
| 346 | (αMePhe)LDSQAAQDFVK | | | | | | 10V, |
| | (γE- | | | | | | 13αMePhe, |
| | C18diacid)24WIANT- | | | | | | 17Q, 18A, |
| | amide | | | | | | 24K, 26I, 27A |
| Peptide | H(Aib)HGTFTSDVSK | 352 | C20diacid | γE | 24 | Amide | 2Aib, 3H, |
| 347 | (αMePhe)LDSQAAQDFVK | | | | | | 10V, |
| | (yE- | | | | | | 13αMePhe, |
| | C20diacid)24WIANT- | | | | | | 17Q, 18A, |
| | amide | | | | | | 24K, 26I, 27A |
| Peptide | H(Aib)QGS(αMePhe)TS | 353 | C18diacid | γE- | 24 | Amide | 2Aib, 5S, |
| 348 | DVSK(αMePhe)LDSQAA | | | (O2Oc)- | | | 6αMePhe, |
| | QDFVK(O2Oc-O2Oc-γE- | | | (O2Oc) | | | 10V, |
| | C18diacid)24WIANT- | | | | | | 13αMePhe, |
| | amide | | | | | | 17Q, 18A, |
| | | | | | | | 24K, 26I, 27A |
| Peptide | H(Aib)HGS(αMePhe)TS | 354 | C18diacid | γE- | 24 | Amide | 2Aib, 3H, 5S, |
| 349 | DVSK(αMePhe)LDSQAA | | | (O2Oc)- | | | 6αMePhe, |
| | QDFVK(O2Oc-O2Oc-γE- | | | (O2Oc) | | | 10V, |
| | C18diacid)24WIANT- | | | | | | 13αMePhe, |
| | amide | | | | | | 17Q, 18A, |
| | | | | | | | 24K, 26I, 27A |
| Peptide | H(Aib)HGS(αMePhe)TS | 355 | C18diacid | γE- | 24 | Amide | 2Aib, 3H, 5S, |
| 350 | DVSK(αMePhe)LDSQAA | | | (O2Oc)- | | | 6αMePhe, |
| | QDFVK(O2Oc-O2Oc-γE- | | | (O2Oc) | | | 10V, |
| | C18diacid)24WIANTGG- | | | | | | 13αMePhe, |
| | amide | | | | | | 17Q, 18A, |
| | | | | | | | 24K, 26I, 27A, |
| | | | | | | | 30G, 31G |
| Peptide | H(Aib)HGS(αMePhe)TS | 356 | C18diacid | γE | 24 | Amide | 2Aib, 3H, 5S, |
| 351 | DVSK(αMePhe)LDSQAA | | | | | | 6αMePhe, |
| | QDFVK(γE- | | | | | | 10V, |
| | C18diacid)24WIANT- | | | | | | 13αMePhe, |
| | amide | | | | | | 17Q, 18A, |
| | | | | | | | 24K, 26I, 27A |
| Peptide | H(Aib)HGS(αMePhe)TS | 357 | C18diacid | γE-γE- | 24 | Amide | 2Aib, 3H, 5S, |
| 352 | DVSK(αMePhe)LDSQAA | | | (PEG)2- | | | 6αMePhe, |
| | QDFVK((PEG)2-(PEG)2- | | | (PEG)2 | | | 10V, |
| | γE-γE- | | | | | | 13αMePhe, |
| | C18diacid)24WIANT- | | | | | | 17Q, 18A, |
| | amide | | | | | | 24K, 26I, 27A |
| Peptide | H(Aib)HGS(αMePhe)TS | 358 | C18diacid | γE-γE- | 24 | Amide | 2Aib, 3H, 5S, |
| 353 | DVSK(αMePhe)LDSQAA | | | (PEG)2- | | | 6αMePhe, |
| | QDFVK(γE-γE-(PEG)2- | | | γE-γE | | | 10V, |
| | γE-γE- | | | | | | 13αMePhe, |
| | C18diacid)24WIANT- | | | | | | 17Q, 18A, |
| | amide | | | | | | 24K, 26I, 27A |
| Peptide | H(Aib)HGS(αMePhe)TS | 359 | C20diacid | γE | 24 | Amide | 2Aib, 3H, 5S, |
| 354 | DVSK(αMePhe)LDSQAA | | | | | | 6αMePhe, |
| | QDFVK(γE- | | | | | | 10V, |
| | C20diacid)24WIANT- | | | | | | 13αMePhe, |
| | amide | | | | | | 17Q, 18A, |
| | | | | | | | 24K, 26I, 27A |
| Peptide | H(Aib)HGS(αMePhe)TS | 360 | C20diacid | γE-γE- | 24 | Amide | 2Aib, 3H, 5S, |
| 355 | DVSK(αMePhe)LDSQAA | | | (PEG)2- | | | 6α MePhe, |
| | QDFVK((PEG)2-(PEG)2- | | | (PEG)2 | | | 10V, |
| | γE-γE- | | | | | | 13αMePhe, |
| | C20diacid)24WIANT- | | | | | | 17Q, 18A, |
| | amide | | | | | | 24K, 26I, 27A |
| Peptide | H(Aib)HGS(αMePhe)TS | 361 | C20diacid | γE- | 24 | Amide | 2Aib, 3H, 5S, |
| 356 | DVSK(αMePhe)LDSQAA | | | (O2Oc)- | | | 6αMePhe, |
| | QDFVK(O2OC-O2OC- | | | (O2Oc) | | | 10V, |
| | γE- | | | | | | 13αMePhe, |
| | C20diacid)24WIANT- | | | | | | 17Q, 18A, |
| | amide | | | | | | 24K, 26I, 27A |
| Peptide | H(Aib)HGS(αMePhe)TS | 362 | C20diacid | γE-γE- | 24 | Amide | 2Aib, 3H, 5S, |
| 357 | DVSK(αMePhe)LDSQAA | | | (PEG)2- | | | 6αMePhe, |
| | QDFVK(γE-γE-(PEG)2- | | | γE-γE | | | 10V, |
| | γE-γE- | | | | | | 13αMePhe, |
| | C20diacid)24WIANT- | | | | | | 17Q, 18A, |
| | amide | | | | | | 24K, 26I, 27A |
| Peptide | H(Aib)IGS(αMePhe)TSD | 363 | C18diacid | γE- | 24 | Amide | 2Aib, 3I, 5S, |
| 358 | VSK(αMePhe)LDSQAA | | | (O2Oc)- | | | 6αMePhe, |
| | QDFVK(O2Oc-O2Oc-γE- | | | (O2Oc) | | | 10V, |
| | C18diacid)24WIANT- | | | | | | 13αMePhe, |
| | amide | | | | | | 17Q, 18A, |
| | | | | | | | 24K, 26I, 27A |
| Peptide | H(Aib)IGS(αMePhe)TSD | 364 | C18diacid | γE- | 24 | Amide | 2Aib, 3I, 5S, |
| 359 | VSK(αMePhe)LDSRAAQ | | | (O2Oc)- | | | 6αMePhe, |
| | DFVK(O2Oc-O2Oc-γE- | | | (O2Oc) | | | 10V, |
| | C18diacid)24WIANT- | | | | | | 13αMePhe, |
| | amide | | | | | | 18A, 24K, 26I, |
| | | | | | | | 27A |
| Peptide | H(Aib)IGS(αMePhe)TSD | 365 | C20diacid | γE- | 24 | Amide | 2Aib, 3I, 5S, |
| 360 | VSK(αMePhe)LDSRAAQ | | | (O2Oc)- | | | 6αMePhe, |
| | DFVK(O2OC-O2OC-γE- | | | (O2Oc) | | | 10V, |
| | C20diacid)24WIANT- | | | | | | 13αMePhe, |
| | amide | | | | | | 18A, 24K, 26I, |
| | | | | | | | 27A |
| Peptide | H(Aib)HGS(αMePhe)TS | 366 | C18diacid | γE- | 24 | Amide | 2Aib, 3H, 5S, |
| 361 | DVSK(αMePhe)LDSRAA | | | (O2Oc)- | | | 6αMePhe, |
| | QDFVK(O2Oc-O2Oc-γE- | | | (O2Oc) | | | 10V, |
| | C18diacid)24WIANT- | | | | | | 13αMePhe, |
| | amide | | | | | | 18A, 24K, 26I, |
| | | | | | | | 27A |
| Peptide | H(Aib)HGS(αMePhe)TS | 367 | C18diacid | γE-γE- | 24 | Amide | 2Aib, 3H, 5S, |
| 362 | DVSK(αMePhe)LDSRAA | | | (O2Oc)- | | | 6αMePhe, |
| | QDFVK(O2IC-O2OC- | | | (O2Oc) | | | 10V |
| | γE-γE- | | | | | | 13αMePhe, |
| | C18diacid)24WIANT- | | | | | | 18A, 24K, 26I, |
| | amide | | | | | | 27A |
| Peptide | H(Aib)HGS(αMePhe)TS | 368 | C18diacid | γE-γE- | 24 | Amide | 2Aib, 3H, 5S, |
| 363 | DVSK(αMePhe)LDSRAA | | | (O20c)- | | | 6αMePhe, |
| | QDFVK(O2OC-O2OC- | | | (O2Oc) | | | 10V, |
| | γE-γE- | | | | | | 13αMePhe, |
| | C18diacid)24WIANTG- | | | | | | 18A, 24K, 26I, |
| | amide | | | | | | 27A, 30G |
| Peptide | H(Aib)HGS(αMePhe)TS | 369 | C18diacid | γE-γE- | 24 | Amide | 2Aib, 3H, 5S, |
| 364 | DVSK(αMePhe)LDSRAA | | | (O2Oc)- | | | 6αMePhe, |
| | QDFVK(O2OC-O2OC- | | | (O2Oc) | | | 10V, |
| | γE-γE- | | | | | | 13αMePhe, |
| | C18diacid)24WIANTGG- | | | | | | 18A, 24K, 26I, |
| | amide | | | | | | 27A, 31G |
| Peptide | H(Aib)HGS(αMePhe)TS | 370 | C18diacid | γE- | 24 | Amide | 2Aib, 3H, 5S, |
| 365 | DVSK(αMePhe)LDSR | | | (O2Oc)- | | | 6αMePhe, |
| | (Aib)AQDFVK(O2Oc- | | | (O2Oc) | | | 10V, |
| | O2Oc-γE- | | | | | | 13αMePhe, |
| | C18diacid)24WIANT- | | | | | | 18Aib, 24K, |
| | amide | | | | | | 26I, 27A |
| Peptide | H(Aib)HGS(αMePhe)TS | 371 | C18diacid | γE- | 24 | Amide | 2Aib, 3H, 5S, |
| 366 | DVSK(αMePhe)LDSRAA | | | (O2Oc)- | | | 6αMePhe, |
| | QDFVK(O2Oc-O2Oc-γE- | | | (O2Oc) | | | 10V, |
| | C18diacid) 24(Aib) | | | | | | 13αMePhe, |
| | IANT-amide | | | | | | 18A, 24K, |
| | | | | | | | 25Aib, 26I, |
| | | | | | | | 27A |
| Peptide | H(Aib)HGS(αMePhe)TS | 372 | C20diacid | γE- | 24 | Amide | 2Aib, 3H, 5S, |
| 367 | DVSK(αMePhe)LDSRAA | | | (O2Oc)- | | | 6αMePhe, |
| | QDFVK(O2OC-O2OC- | | | (O2Oc) | | | 10V, |
| | γE- | | | | | | 13αMePhe, |
| | C20diacid) 24WIANT- | | | | | | 18A, 24K, 26I, |
| | amide | | | | | | 27A |
| Peptide | H(Aib)HGS(αMePhe)TS | 373 | C20diacid | γE- | 24 | Amide | 2Aib, 3H, 5S, |
| 368 | DVSK(αMePhe)LDSR | | | (O2Oc)- | | | 6αMePhe, |
| | (Aib)AQDFVK(O2OC- | | | (O2Oc) | | | 10V, |
| | O2OC-γE- | | | | | | 13αMePhe, |
| | C20diacid)24WIANT- | | | | | | 18Aib, 24K, |
| | amide | | | | | | 26I, 27A |
| Peptide | H(Aib)HGS(αMePhe)TS | 374 | C20diacid | γE- | 24 | Amide | 2Aib, 3H, 5S, |
| 369 | DVSK(αMePhe)LDSRAA | | | (O2Oc)- | | | 6αMePhe, |
| | QDFVK(O2OC-O2OC- | | | (O2Oc) | | | 10V, |
| | γE- | | | | | | 13αMePhe, |
| | C20diacid)24(Aib)IANT- | | | | | | 18A, 24K, |
| | amide | | | | | | 25Aib, 26I, |
| | | | | | | | 27A |
| Peptide | H(Aib)HGS(αMePhe)TS | 375 | C18diacid | γE- | 24 | Amide | 2Aib, 3H, 5S, |
| 370 | DVSK(αMePhe)LDSQAA | | | (O2Oc)- | | | 6αMePhe, |
| | QD(αMePhe)VK(O2Oc- | | | (O2Oc) | | | 10V |
| | O2Oc-yE- | | | | | | 13αMePhe, |
| | C18diacid)24WIANT- | | | | | | 17Q, 18A, |
| | amide | | | | | | 22αMePhe, |
| | | | | | | | 24K, 26I, 27A |
| Peptide | H(Aib)HGS(αMePhe)TS | 376 | C18diacid | γE-γE- | 24 | Amide | 2Aib, 3H, 5S, |
| 371 | DVSK(αMePhe)LDSRAA | | | (O2Oc)- | | | 6αMePhe, |
| | QD(αMePhe)VK(O2OC₂- | | | (O2Oc) | | | 10V, |
| | γE-γE- | | | | | | 13αMePhe, |
| | C18diacid)24WIANT- | | | | | | 18A, |
| | amide | | | | | | 22αMePhe, |
| | | | | | | | 24K, 26I, 27A |
| Peptide | H(Aib)HGS(αMePhe)TS | 377 | C18diacid | γE-γE- | 24 | Amide | 2Aib, 3H, 5S, |
| 372 | DVSK(αMePhe)LDSRAA | | | (O2Oc)- | | | 6αMePhe, |
| | QD(αMePhe)VK(O2OC₂- | | | (O2Oc) | | | 10V, |
| | γE-γE- | | | | | | 13αMePhe, |
| | C18diacid)24(Aib)IANT- | | | | | | 18A, |
| | amide | | | | | | 22αMePhe, |
| | | | | | | | 24K, 25Aib, |
| | | | | | | | 26I, 27A |
| Peptide | H(Aib)HGS(αMePhe)TS | 378 | C18diacid | γE-γE- | 24 | Amide | 2Aib, 3H, 5S, |
| 373 | DVSK(αMePhe)LDSRAA | | | (O2Oc)- | | | 6αMePhe, |
| | QD(αMePhe)VK(O2OC₂- | | | (O2Oc) | | | 10V, |
| | γE-γE- | | | | | | 13αMePhe, |
| | C18diacid)24(Aib)IANT | | | | | | 18A, |
| | GG-amide | | | | | | 22αMePhe, |
| | | | | | | | 24K, 25Aib, |
| | | | | | | | 26I, 27A, |
| | | | | | | | 30G, 31G |
| Peptide | H(Aib)HGS(αMePhe)TS | 379 | C20diacid | γE-γE- | 24 | Amide | 2Aib, 3H, 5S, |
| 374 | DVSK(αMePhe)LDSRAA | | | (O2Oc)- | | | 6αMePhe, |
| | QD(αMePhe)VK(O2OC₂- | | | (O2Oc) | | | 10V, |
| | γE-γE- | | | | | | 13αMePhe, |
| | C20diacid)24WIANT- | | | | | | 18A, |
| | amide | | | | | | 22αMePhe, |
| | | | | | | | 24K, 26I, 27A |
| Peptide | H(Aib)HGS(αMePhe)TS | 380 | C20diacid | γE-γE- | 24 | Amide | 2Aib, 3H, 5S, |
| 375 | DVSK(αMePhe)LDSRAA | | | (O2Oc)- | | | 6αMePhe, |
| | QD(αMePhe)VK(O2OC₂- | | | (O2Oc) | | | 10V, |
| | γE-γE- | | | | | | 13αMePhe, |
| | C20diacid)24(Aib)IANT- | | | | | | 18A, |
| | amide | | | | | | 22αMePhe, |
| | | | | | | | 24K, 25Aib, |
| | | | | | | | 26I, 27A |
| Peptide | H(Aib)HGS(αMePhe)TS | 381 | C20diacid | γE-γE- | 24 | Amide | 2Aib, 3H, 5S, |
| 376 | DVSK(αMePhe)LDSRAA | | | (20c)- | | | 6αMePhe, |
| | QD(αMePhe)VK(O2OC₂- | | | (O2Oc) | | | 10V, |
| | γE-γE- | | | | | | 13αMePhe, |
| | C20diacid)24(Aib)IANT | | | | | | 18A, |
| | GG-amide | | | | | | 22αMePhe, |
| | | | | | | | 24K, 25Aib, |
| | | | | | | | 26I, 27A, |
| | | | | | | | 30G, 31G |
| Peptide | H(Aib)HGS(αMePhe)TS | 382 | C20diacid | γE-γE- | 24 | Amide | 2Aib, 3H, 5S, |
| 377 | DVSK(αMePhe)LDSRAA | | | (O2Oc)- | | | 6αMePhe, |
| | QDFVK(O2OC₂-γE-γE- | | | (O2Oc) | | | 10V, |
| | C20diacid)24WIANT- | | | | | | 13αMePhe, |
| | amide | | | | | | 18A, 24K, 26I, |
| | | | | | | | 27A |
| Peptide | H(Aib)HGS(αMePhe)TS | 383 | C18diacid | γE-γE- | 24 | Amide | 2Aib, 3H, 5S, |
| 378 | DVSK(αMePhe)LDSRAA | | | (O2Oc)- | | | 6αMePhe, |
| | QD(αMePhe)VK(O2OC₂- | | | (O2Oc) | | | 10V, |
| | γE-γE- | | | | | | 13αMePhe, |
| | C18diacid)24SIANT- | | | | | | 18A, |
| | amide | | | | | | 22αMePhe, |
| | | | | | | | 24K, 25S, 26I, |
| | | | | | | | 27A |
| Peptide | H(Aib)HGS(αMePhe)TS | 384 | C18diacid | γE-yE- | 24 | Amide | 2Aib, 3H, 5S, |
| 379 | DVSK(αMePhe)LDSRAA | | | (O2Oc)- | | | 6αMePhe, |
| | QD(αMePhe)VK(O2OC₂- | | | (O2Oc) | | | 10V, |
| | γE-C18diacid)24SIANT- | | | | | | 13αMePhe, |
| | amide | | | | | | 18A, |
| | | | | | | | 22αMePhe, |
| | | | | | | | 24K, 25S, 26I, |
| | | | | | | | 27A |
| Peptide | (NHis)SHGS(αMePhe)T | 385 | C20diacid | γE-yE- | 24 | Amide | 1NHis, 3H, |
| 380 | SDVSK(αMePhe)LDSRA | | | (O20c)- | | | 5S, |
| | AQDFVK(O2OC₂-γE-γE- | | | (O2Oc) | | | 6αMePhe, |
| | C20diacid)24WIANT- | | | | | | 10V, |
| | amide | | | | | | 13αMePhe, |
| | | | | | | | 18A, 24K, 26I, |
| | | | | | | | 27A |
| Peptide | H(Aib)HGS(αMePhe)TS | 386 | C18diacid | γE-γE- | 24 | Amide | 2Aib, 3H, 5S, |
| 381 | DVSK(αMePhe)LDSRAA | | | (O2Oc)- | | | 6αMePhe, |
| | QDFVK(O2OC-O2OC- | | | (O2Oc) | | | 10V, |
| | γE-yE- | | | | | | 13αMePhe, |
| | C18diacid)24SIANTGG- | | | | | | 18A, 24K, |
| | amide | | | | | | 25S, 26I, 27A, |
| | | | | | | | 30G, 31G |
| Peptide | H(Aib)HGS(αMePhe)TS | 387 | C18diacid | γE-γE- | 24 | Amide | 2Aib, 3H, 5S, |
| 382 | DVSK(αMePhe)LDSR | | | (O2Oc)- | | | 6αMePhe, |
| | (Aib)AQDFVK(O2OC- | | | (O2Oc) | | | 10V, |
| | O2OC-γE-γE- | | | | | | 13αMePhe, |
| | C18diacid)24SIANTGG- | | | | | | 18Aib, 24K, |
| | amide | | | | | | 25S, 26I, 27A, |
| | | | | | | | 30G, 31G |
| Peptide | H(Aib)HGS(αMePhe)TS | 388 | C18diacid | γE- | 24 | Amide | 2Aib, 3H, 5S, |
| 383 | DVSK(αMePhe)LDSQAA | | | (O2Oc)- | | | 6αMePhe, |
| | QD(αMePhe)VK(O2Oc- | | | (O2Oc) | | | 10V |
| | O2Oc-γE- | | | | | | 13αMePhe, |
| | C18diacid)24(Aib)IANT- | | | | | | 17Q, 18A, |
| | amide | | | | | | 22αMePhe, |
| | | | | | | | 24K, 25Aib, |
| | | | | | | | 26I, 27A |
| Peptide | H(Aib)HGS(αMePhe)TS | 389 | C20diacid | γE-γE- | 24 | Amide | 2Aib, 3H, 5S, |
| 384 | DVSK(αMePhe)LDSQAA | | | (O2Oc)- | | | 6αMePhe, |
| | QD(αMePhe)VK(O2OC- | | | (O2Oc) | | | 10V, |
| | O2OC-γE-γE- | | | | | | 13αMePhe, |
| | C20diacid)24(Aib)IANT- | | | | | | 17Q, 18A, |
| | amide | | | | | | 22αMePhe, |
| | | | | | | | 24K, 25Aib, |
| | | | | | | | 26I, 27A |
| Peptide | HSQGTFTSDYSK(αMePhe) | 390 | C18diacid | γE- | 24 | Amide | 13MePhe, |
| 385 | LEEEAVRLFIK(O2OC- | | | (O2Oc)- | | | 15E, 16E, |
| | O20C-γE- | | | (O2Oc) | | | 17E, 18A, |
| | stearOOH)24WLMNT- | | | | | | 19V, 20R, |
| | amide | | | | | | 21L, 23I, 24K, |
| Peptide | H(Aib)QGTFTSDVSK | 391 | C18diacid | γE- | 24 | Acid | 2Aib, 10V, |
| 386 | (αMePhe)LDSRRAQDFVK | | | (O2Oc)- | | | 13αMePhe, |
| | (O2OC-O2OC-γE- | | | (O2Oc) | | | 24K, 25Aib, |
| | C18diacid)24WLVE(Aib) | | | | | | 27V, 28E, |
| | G-acid | | | | | | 29Aib, 30G |
| Peptide | H(Aib)QGTFTSDVSK | 392 | C18diacid | γE- | 24 | Acid | 2Aib, 10V, |
| 387 | (αMePhe)LESRRAQDFVK | | | (O2Oc)- | | | 13αMePhe, |
| | (O20C-O2OC-yE- | | | (O2Oc) | | | 15E, 24K, |
| | C18diacid)24WLVE(Aib) | | | | | | 25Aib, 27V, |
| | G-acid | | | | | | 28E, 29Aib, |
| | | | | | | | 30G |
| Peptide | H(Aib)QGTFTSDVSK | 393 | C18diacid | γE- | 24 | Acid | 2Aib, 10V, |
| 388 | (αMePhe)LDLRRAQDFVK | | | (O2Oc)- | | | 13αMePhe, |
| | (O2OC-O2OC-γE- | | | (O2Oc) | | | 16L, 24K, |
| | C18diacid)24WLVE(Aib) | | | | | | 25Aib, 27V, |
| | G-acid | | | | | | 28E, 29Aib, |
| | | | | | | | 30G |
| Peptide | H(Aib)QGTFTSDVSK | 394 | C18diacid | γE- | 24 | Acid | 2Aib, 10V, |
| 389 | (αMePhe)LDSRRAQDFVK | | | (O2Oc)- | | | 13αMePhe, |
| | (O2OC-O2OC-γE- | | | (O2Oc) | | | 24K, 25Aib, |
| | C18diacid)24WLL(Aib) | | | | | | 27L, 28Aib |
| | T-acid | | | | | | |
| Peptide | H(Aib)QGTFTSDVSK | 395 | C18diacid | γE- | 24 | Acid | 2Aib, 10V, |
| 390 | (αMePhe)LDSRRAQDFVK | | | (O2Oc)- | | | 13αMePhe, |
| | (O2OC-O2OC-γE- | | | (O2Oc) | | | 24K, 25Aib, |
| | C18diacid)24WLVEG | | | | | | 27V, 28E, |
| | (Aib)-acid | | | | | | 29G, 30Aib |

In some aspects, the peptide comprises the sequence: H-X2-X3-G-X5-X6-T-S-D-X10-S-K-X13-L-D-S-X17-X18-A-Q-D-X22-V-X24-X25-X26-X27-N-T-X(30)-X(31)-Z, wherein X2 is S or Aminoisobutyric acid (Aib), X3 is Q or H, X5 is T or S, X6 is F or α-methylphenylalanine (αMePhe), X10 is Y, V, or K, wherein the K can comprise an acyl moiety and/or can be lipidated, X13 is Y, αMePhe, I, or Diphenylalanine (Dip), or K wherein the K can comprise an acyl moiety and/or can be lipidated, X17 is R, Q, or β-dimethylarganine (β-diMeArg), X18 is R or A, X22 is F or αMePhe, X24 is Q or E, X25 is W, Aib, or H, X26 is L or I, X27 is M or A, X (30) is not present, X (31) is not present, and Z is amide or acid (SEQ ID NO: 417), wherein the peptide does not comprise SEQ ID NO: 1.

In some aspects, the residue at position 10 or 13 is acylated. In some aspects, the residue at position 10 or 13 is lipidated. In some aspects, the lipid is selected from the group consisting of a palmitoyl group, stearoyl group, lauryl group, myristoyl group, margaroyl group, octadecanedioic acid (C18diacid), and icosanedioic acid (C20diacid). In some aspects, the lipid is selected from the group consisting of an octadecanedioic acid (C18diacid), and icosanedioic acid (C20diacid), stearoyl group, and palmitoyl group.

In some aspects, the lipid attached to the residue at position 10 or 13 is attached via a linker. In some aspects, the linker is selected from the group consisting of (O2Oc), (O2Oc)-(O2Oc), (O2Oc)-γE-(O2Oc), (PEG)2-(PEG)2-γE-γE, (PEG)2-γE-(PEG)2-γE, γE, γE-(O2Oc), γE-(O2Oc)-(O2Oc), γE-(O2Oc)-γE-(O2Oc), γE-(PEG)2-(PEG)2, γE-(PEG)2-γE-(PEG)2, γE-(PEG)4, γE-γE, γE-γE-(O2Oc), γE-γE-(O2Oc)-(O2Oc), γE-γE-(PEG)12, γE-γE-(PEG)2-(PEG)2, γE-γE-(PEG)2-γE-γE, γE-γE-(PEG)4, and γE-γE-(PEG)8. In some aspects, the linker is selected from the group consisting of γE-γE-(O2Oc)-(O2Oc), γE-(O2Oc)-(O2Oc), γE-γE-(PEG)2-(PEG)2, and γE-(PEG)2-(PEG)2.

In some aspects, the peptide comprises any one of SEQ ID NOs: 396-411.

In some aspects, the peptide is any one of the peptides in Table 4 or Table 5.

TABLE 4
Peptides Modified at Position 10

| | | | | Linker | | | Sequence |
|---|---|---|---|---|---|---|---|
| | | | Albumin | (described | Acyl- | | modification |
| | | | binding | N→C | ation | C- | with respect |
| Peptide | Sequence | | moiety | term) | site | term. | to glucagon |
| Peptide | H(Aib)HGS(αMePhe)TS | 396 | C20diacid | γE-γE- | 10 | Amide | 2Aib, 3H, 5S, |
| 390 | DK(O20C-O2OC-γE-γE- | | | (O2Oc)- | | | 6αMePhe, |
| | C20diacid)10SK | | | (O2Oc) | | | 10K, |
| | (αMePhe)LDSRAAQD | | | | | | 13αMePhe, |
| | (αMePhe)VQ(Aib)IANT- | | | | | | 18A, |
| | amide | | | | | | 22αMePhe, |
| | | | | | | | 25Aib, 26I, |
| | | | | | | | 27A |
| Peptide | H(Aib)HGS(αMePhe)TS | 397 | C18diacid | γE- | 10 | Amide | 2Aib, 3H, 5S, |
| 391 | DK(O2OC-O2OC-γE- | | | (O2Oc)- | | | 6αMePhe, |
| | C18diacid)10SK | | | (O2Oc) | | | 10K, |
| | (αMePhe)LDSRAAQD | | | | | | 13αMePhe, |
| | (αMePhe)VQ(Aib)IANT- | | | | | | 18A, |
| | amide | | | | | | 22αMePhe, |
| | | | | | | | 25Aib, 26I, |
| | | | | | | | 27A |
| Peptide | HSQGS(αMePhe)TSDK | 398 | Stearoyl | γE-γE- | 10 | Amide | 5S, |
| 392 | ((PEG)2-(PEG)2-γE-yE- | | | (PEG)2- | | | 6αMePhe, |
| | Stearoyl)10SK(Dip) | | | (PEG)2 | | | 10K, 13Dip, |
| | LDS(β- | | | | | | 17β- |
| | dimethylArg)AAQD | | | | | | dimethylR, |
| | (αMePhe)VE(Aib)LANT- | | | | | | 18A, |
| | amide | | | | | | 22αMePhe, |
| | | | | | | | 24E, 25Aib, |
| | | | | | | | 27A |
| Peptide | HSQGS(αMePhe)TSDK | 399 | Palmitoyl | γE-γE- | 10 | Amide | 5S, |
| 393 | ((PEG)2-(PEG)2-γE-γE- | | | (PEG)2- | | | 6αMePhe, |
| | Palmitoyl)10SK(Dip) | | | (PEG)2 | | | 10K, 13Dip, |
| | LDS(β- | | | | | | 17β- |
| | dimethylArg)AAQD | | | | | | dimethylR, |
| | (αMePhe)VE(Aib)LANT- | | | | | | 18A, |
| | amide | | | | | | 22αMePhe, |
| | | | | | | | 24E, 25Aib, |
| | | | | | | | 27A |
| Peptide | H(Aib)QGSFTSDK((PEG) | 400 | Palmitoyl | γE- | 10 | Amide | 2Aib, 5S, |
| 394 | 2-(PEG)2-γE- | | | (PEG)2- | | | 10K, 13I, |
| | Palmitoyl)10SKILDSRAA | | | (PEG)2 | | | 18A, 24E, |
| | QDFVEWIANT-amide | | | | | | 261, 27A |
| Peptide | H(Aib)QGS(αMePhe)TS | 401 | Palmitoyl | γE- | 10 | Amide | 2Aib, 5S, |
| 395 | DK((PEG)2-(PEG)2-γE- | | | (PEG)2- | | | 6αMePhe, |
| | Palmitoyl)10SKILDSRAA | | | (PEG)2 | | | 10K, 13I, |
| | QD(αMePhe)VEWIANT- | | | | | | 18A, |
| | amide | | | | | | 22αMePhe, |
| | | | | | | | 24E, 26I, 27A |
| Peptide | HSQGS(αMePhe)TSDK | 402 | Stearoyl | γE-γE- | 10 | Amide | 5S, |
| 396 | ((PEG)2-(PEG)2-γE-γE- | | | (PEG)2- | | | 6αMePhe, |
| | Stearoyl)10SK(Dip)LDS | | | (PEG)2 | | | 10K, 13Dip, |
| | QAAQD(αMePhe)VE | | | | | | 17Q, 18A, |
| | (Aib)LANT-amide | | | | | | 22αMePhe, |
| | | | | | | | 24E, 25Aib, |
| | | | | | | | 27A |
| Peptide | HSQGS(αMePhe)TSDK | 403 | Stearoyl | γE-γE- | 10 | Amide | 5S, |
| 397 | ((PEG)2-(PEG)2-γE-γE- | | | (PEG)2- | | | 6αMePhe, |
| | Stearoyl) 10SK(Dip) | | | (PEG)2 | | | 10K, 13Dip, |
| | LDS(β- | | | | | | 17β- |
| | dimethylArg)AAQD | | | | | | dimethylR, |
| | (αMePhe)VEHLANT-amide | | | | | | 18A, |
| | | | | | | | 22αMePhe, |
| | | | | | | | 24E, 25H, |
| | | | | | | | 27A |

TABLE 5
Peptides Modified at Position 13

| | | | | Linker | | | Sequence |
|---|---|---|---|---|---|---|---|
| | | SEQ | Albumin | (described | Acyl- | | modification |
| | | ID | binding | N→C | ation | C- | with respect |
| Peptide | Sequence | NO | moiety | term.) | site | term. | to glucagon |
| Peptide | HSQGS(αMePhe)TSDVS | 404 | Stearoyl | γE-γE- | 13 | Amide | 5S, |
| 398 | KK((PEG)2-(PEG)2-γE- | | | (PEG)2- | | | 6αMePhe, |
| | γE-Stearoyl)13LDS | | | (PEG)2 | | | 10V, 13K, |
| | (β-dimethylArg)AAQD | | | | | | 17β- |
| | (αMePhe)VE(Aib)LANT- | | | | | | dimethylR, |
| | amide | | | | | | 18A, |
| | | | | | | | 22αMePhe, |
| | | | | | | | 24E, 25Aib, |
| | | | | | | | 27A |
| Peptide | HSQGS(αMePhe)TSDVS | 405 | Palmitoyl | γE-γE- | 13 | Amide | 5S, |
| 399 | KK((PEG)2-(PEG)2-γE- | | | (PEG)2- | | | 6αMePhe, |
| | γE-Palmitoyl)13LDS(β- | | | (PEG)2 | | | 10V, 13K, |
| | dimethylArg)AAQD | | | | | | 17β- |
| | (αMePhe)VE(Aib)LANT- | | | | | | dimethylR, |
| | amide | | | | | | 18A, |
| | | | | | | | 22αMePhe, |
| | | | | | | | 24E, 25Aib, |
| | | | | | | | 27A |
| Peptide | H(Aib)QGS(αMePhe)TS | 406 | Palmitoyl | γE- | 13 | Amide | 2Aib, 5S, |
| 340 | DVSKK((PEG)2-(PEG)2- | | | (PEG)2- | | | 6αMePhe, |
| | γE- | | | (PEG)2 | | | 10V, 13K, |
| | Palmitoyl)13LDSRAAQ | | | | | | 18A, |
| | D(αMePhe)VEWIANT- | | | | | | 22αMePhe, |
| | amide | | | | | | 24E, 26I, 27A |
| Peptide | H(Aib)QGSFTSDVSKK | 407 | Palmitoyl | γE- | 13 | Amide | 2Aib, 5S, |
| 341 | ((PEG)2-(PEG)2-γE- | | | (PEG)2- | | | 10V, 13K, |
| | Palmitoyl)13LDSRAAQ | | | (PEG)2 | | | 18A, 24E, |
| | DFVEWIANT-amide | | | | | | 26I, 27A |
| Peptide | HSQGS(αMePhe)TSDVS | 408 | Stearoyl | γE-γE- | 13 | Amide | 5S, |
| 342 | KK((PEG)2-(PEG)2-γE- | | | (PEG)2- | | | 6αMePhe, |
| | γE- | | | (PEG)2 | | | 10V, 13K, |
| | Stearoyl)13LDSQAAQD | | | | | | 17Q, 18A, |
| | (αMePhe)VE(Aib)LANT- | | | | | | 22αMePhe, |
| | amide | | | | | | 24E, 25Aib, |
| | | | | | | | 27A |
| Peptide | HSQGS(αMePhe)TSDVS | 409 | Stearoyl | γE-γE- | 13 | Amide | 5S, |
| 343 | KK((PEG)2-(PEG)2-γE- | | | (PEG)2- | | | 6αMePhe, |
| | γE-Stearoyl)13LDS(β- | | | (PEG)2 | | | 10V, 13K, |
| | dimethylArg)AAQD | | | | | | 17β- |
| | (αMePhe)VEHLANT-amide | | | | | | dimethylR, |
| | | | | | | | 18A, |
| | | | | | | | 22αMePhe, |
| | | | | | | | 24E, 25H, |
| | | | | | | | 27A |
| Peptide | H(Aib)HGS(αMePhe)TS | 410 | C20diacid | γE-γE- | 13 | Amide | 2Aib, 3H, 5S, |
| 344 | DVSKK(O2OC-O2OC-yE- | | | (O2Oc)- | | | 6αMePhe, |
| | γE- | | | (O2Oc) | | | 10V, 13K, |
| | C20diacid)13LDSRAAQ | | | | | | 18A, |
| | D(αMePhe)VQ(Aib)IAN | | | | | | 22αMePhe, |
| | T-amide | | | | | | 25Aib, 26I, |
| | | | | | | | 27A |
| Peptide | H(Aib)HGS(αMePhe)TS | 411 | C18diacid | γE- | 13 | Amide | 2Aib, 3H, 5S, |
| 345 | DVSKK(O2OC-O2OC-γE- | | | (O2Oc)- | | | 6αMePhe, |
| | C18diacid)13LDSRAAQ | | | (O2Oc) | | | 10V, 13K, |
| | D(αMePhe)VQ(Aib)IAN | | | | | | 18A, |
| | T-amide | | | | | | 22αMePhe, |
| | | | | | | | 25Aib, 26I, |
| | | | | | | | 27A |

In some aspects, the peptide comprises the sequence: H-X2-X3-G-X5-X6-T-S-D-X10-S-X12-αMethyl-Phenylalanine (αMePhe)-L-X15-X16-X17-X18-A-X20-X21-X22-X23-X24-X25-X26-X27-X28-X29-X30-X31-Z, wherein X2 is Aminoisobutyric acid (Aib), S, or A, X3 is Q, H, or E, X5 is T or S, X6 is F or αMePhe, X10 is V, K or Y, X11 is S, X12 is K, E, or S, X15 is D or E, X16 is T, S, or G, X17 is K, R, E, or Q, X18 is R or A, X20 is R, K, or Q, X21 is D or E, X22 is αMePhe or F, X23 is V or I, X24 is Q or A, X25 is Aib or W, X26 is L or I, X27 is L, A, E, V, or M, X28 is E, N, A, R, or K, X29 is Aib, T, or G, X30 is G, R, or not present, X31 is G or not present, and Z is amide or acid (SEQ ID NO: 540).

In some aspects, X2 is Aib. In some aspects, X3 is Q. In some aspects, X3 is H. In some aspects, X5 is T. In some aspects, X5 is S. In some aspects, X6 is F. In some aspects, X6 is αMePhe. In some aspects, X10 is V. In some aspects, X12 is K. In some aspects, X15 is D. In some aspects, X16 is T. In some aspects, X16 is S. In some aspects, X17 is K. In some aspects, X17 is R. In some aspects, X18 is R. In some aspects, X18 is A. In some aspects, X20 is R. In some aspects, X20 is K. In some aspects, X21 is D. In some aspects, X22 is F. In some aspects, X22 is αMePhe. In some aspects, X23 is V. In some aspects, X24 is Q. In some aspects, X25 is W. In some aspects, X25 is Aib. In some aspects, X26 is L. In some aspects, X26 is I. In some aspects, X27 is L. In some aspects, X27 is A. In some aspects, X28 is E. In some aspects, X28 is N. In some aspects, X29 is Aib. In some aspects, X29 is T. In some aspects, X30 is G. In some aspects, X30 is not present. In some aspects, X31 is not present. In some aspects, Z is amide. In some aspects, Z is acid.

In some aspects, X2 is Aib, X12 is K, and X24 is Q. In some aspects, X16 is T, X17 is K, X27 is L, X28, is E, and X29 is Aib.

In some aspects, X3 is Q, X5 is T, X6 is F, X10 is V, X12 is K, X15 is D, X16 is T, X17 is K, X18 is R, X20 is R, X21 is D, X22 is F, X23 is V, X24 is Q, X25 is W, X26 is L, X27 is L, X28 is E, X29 is Aib, X30 is G, and X31 is not present.

In some aspects, X3 is H, X5 is S, X6 is αMePhe, X10 is V, X12 is K, X15 is D, X16 is S, X17 is R, X18 is A, X20 is K, X21 is D, X22 is αMePhe, X23 is V, X24 is Q, X25 is Aib, X26 is I, X27 is A, X28 is N, X29 is T, X30 is not present, and X31 is not present.

In some aspects, one or more lysine residues are acylated. In some aspects, the lysine at position 17 is acylated. In some aspects, the lysine at position 20 is acylated.

In some aspects, one or more lysine resides are lipidated. In some aspects, the lysine at position 17 is lipidated. In some aspects, the lysine at position 20 is lipidated.

In some aspects, the lipid is selected from the group consisting of octadecanedioic acid (C18diacid) and icosanedioic acid (C20diacid). In some aspects, the lipid is octadecanedioic acid (C18diacid). In some aspects, the lipid is icosanedioic acid (C20diacid).

In some aspects, the lipid is linked to the residue at position 17 or 20 via a linker. In some aspects, the linker is γE-(O2Oc)-(O2Oc) or γE-γE-(O2Oc)-(O2Oc). In some aspects, the linker is γE-(O2Oc)-(O2Oc). In some aspects, the linker is γE-γE-(O2Oc)-(O2Oc). In some aspects, the linker is linked to the epsilon amino group of the residue at position 17 or 20.

Certain aspects of the disclosure are directed to a peptide comprising the sequence of

(SEQ ID NO: 541)
H-Aib-Q-G-T-F-T-S-D-V-S-K-αMePhe-L-D-T-K-R-A-R-
D-F-V-Q-W-LL-E-Aib-G-acid.

In some aspects, the lysine at position 17 is acylated and lipidated, the lipid is linked to the acylated lysine via a (E-(O2Oc)-(O2Oc)-γE-C18diacid).

In some aspects, the lysine at position 17 is acylated and lipidated, the lipid is linked to the acylated lysine at position 17 via a (E-(O2Oc)-(O2Oc)-γE-C20diacid).

Certain aspects of the disclosure are directed to a peptide comprising the sequence H-Aib-H-G-S-αMePhe-T-S-D-V-S-K-αMePhe-L-D-S-R-A-A-K (E-(O2Oc)-(O2Oc)-γE-C18diacid) 20-D-αMePhe-V-Q-Aib-I-A-N-T-amide (SEQ ID NO: 228).

Certain aspects of the disclosure are directed to a peptide comprising the sequence H-Aib-H-G-S-αMePhe-T-S-D-V-S-K-αMePhe-L-D-S-R-A-A-K (E-(O2Oc)-(O2Oc)-γE-γE-C20diacid) 20-D-αMePhe-V-Q-Aib-I-A-N-T-amide (SEQ ID NO: 233).

In some aspects, the peptide binds to the GLP-1 receptor (GLP-1R), binds to the glucagon receptor (GCGR), or binds to both a GLP-1 receptor and a glucagon receptor. In some aspects, the GLP-1R is a human GLP-1R. In some aspects, the GCGR is a human GCGR. In some aspects, the peptide is an agonist of GLP-1 activity, an agonist of glucagon activity, or an agonist of both GLP-1 and glucagon activity.

In some aspects, the peptide has increased proteolytic-resistance relative to the natural ligand of the GLP-1R and/or GCGR.

In some aspects, the peptide is isolated.

In some aspects, the peptide has at least 50%, at least 60%, at least 70%, at least 75%, at least 80%, at least 85%, at least 90%, at least 95, or 100% of intact peptide remaining after incubation with a protease at 37° C. for 5 min, 10 min, 15 min, 30 min, 2 hr, 4 hr or 24 hr. In some aspects, the protease is selected from the group consisting of neprilysin, pepsin, pancreatin, simulated gastric fluid with pepsin, and simulated intestinal fluid with pancreatin.

In some aspects, the peptide has a half-life in cynomolgus monkeys after intravenous administration of at least 45 hours, at least 50 hours, at least 60 hours, at least 70 hours, at least 80 hours, at least 90 hours, at least 100 hours, at least 110 hours, at least 120 hours, or about 130 hours.

In some aspects, the peptide has an s.c. bioavaiablity in cynomolgus monkeys of at least 75%, at least 80%, at least 90%, or about 95%.

### III. Methods of Making GLP-1/Glucagon Agonist Peptides

GLP-1/glucagon agonist peptides for uses provided herein can be made by any suitable method. For example, in some aspects provided herein, the GLP-1/glucagon agonist peptides for uses provided herein are chemically synthesized by methods well known to those of ordinary skill in the art, e.g., by solid phase synthesis as described by Merrifield (1963, J. Am. Chem. Soc. 85:2149-2154). Solid phase peptide synthesis can be accomplished, e.g., by using automated synthesizers, using standard reagents, e.g., as explained in Example 1 of WO 2014/091316, which is herein incorporated by reference in its entirety.

Alternatively, GLP-1/glucagon agonist peptides for uses provided herein can be produced recombinantly using a convenient vector/host cell combination as would be well known to the person of ordinary skill in the art. A variety of methods are available for recombinantly producing GLP-1/glucagon agonist peptides. Generally, a polynucleotide sequence encoding the GLP-1/glucagon agonist peptide is inserted into an appropriate expression vehicle, e.g., a vector which contains the necessary elements for the transcription and translation of the inserted coding sequence. The nucleic acid encoding the GLP-1/glucagon agonist peptide is inserted into the vector in proper reading frame. The expression vector is then transfected into a suitable host cell which will express the GLP-1/glucagon agonist peptide. Suitable host cells include without limitation bacteria, yeast, or mammalian cells. A variety of commercially-available host-expression vector systems can be utilized to express the GLP-1/glucagon agonist peptides described herein.

### Pharmaceutical Compositions

Further provided are compositions, e.g., pharmaceutical compositions, that contain an effective amount of a GLP-1/glucagon agonist peptide as provided herein, formulated for the treatment of metabolic diseases, e.g., obesity, type 2 diabetes, and/or NASH.

Compositions of the disclosure can be formulated according to known methods. Suitable preparation methods are described, for example, in Remington's Pharmaceutical Sciences, 19th Edition, A. R. Gennaro, ed., Mack Publishing Co., Easton, PA (1995), which is incorporated herein by reference in its entirety. Composition can be in a variety of forms, including, but not limited to an aqueous solution, an emulsion, a gel, a suspension, lyophilized form, or any other form known in the art. In addition, the composition can contain pharmaceutically acceptable additives including, for example, diluents, binders, stabilizers, and preservatives. Once formulated, compositions of the invention can be administered directly to the subject.

In some aspects, the pharmaceutical composition is a solid composition. In some aspects, the pharmaceutical composition is a liquid composition.

### IV. Methods of Using GLP-1/Glucacon Agonist Peptides

As provided herein, GLP-1/glucagon agonist peptides can be used to improve glycemic control, reduce weight, type 2 diabetes mellitus (T2DM), and/or treat or prevent non-alcohlic steatohepatitis (NASH).

In some aspects, administration of the GLP-1/glucagon agonist peptides decreases body weight of the subject, increases insulin secretion in the subject, delays gastric emptying in the subject, decreases food intake in the subject, increases mitochondria function in the subject, inhibits de novo lipogenesis in the subject, decreases HbA1c in the subject, enhances fatty oxidation in the subject, decreases hepatic mitochondrial oxidative stress in the subject, decreases steatosis in the subject, decreases fibrosis in the subject, decreases glycogen synthesis in the subject, increases gluconeogenesis in the subject, halts disease progression in the subject, reverses fibrosis in the subject, and/or reduces risk of death due to cirrhosis, hepatocellular carcinoma, and/or cardiorenal disease in the subject.

As provided herein a method of improving glycemic control or reduce weight in a human subject with T2DM and/or NASH can comprise administering to the subject a GLP-1/glucagon agonist peptide.

This disclosure also provides a GLP-1/glucagon agonist peptide for use in the manufacture of a medicament for improving glycemic control or reduce weight in a human subject with T2DM and/or NASH.

In some aspects, the peptide is administered about once a week.

In some aspects, the GLP-1/glucagon agonist peptide is administered to treat or prevent a disease or condition cause or characterized by excess body weight. In some aspects, the disease or condition is obesity. In some aspects, the disease or condition is type 2 diabetes.

In some aspects, the GLP-1/glucagon agonist peptide is administered to treat or non-alcohlic steatohepatitis (NASH). In some aspects, the GLP-1/glucagon agonist peptide is administered by injection. In some aspects, the GLP-1/glucagon agonist peptide is administered orally. In some aspects, administration of the GLP-1/glucagon agonist peptide decreases body weight of the subject, increases insulin secretion in the subject, delays gastric emptying in the subject, decreases food intake in the subject, increases mitochondria function in the subject, inhibits de novo lipogenesis in the subject, decreases HbA1c in the subject, enhances fatty oxidation in the subject, decreases hepatic mitochondrial oxidative stress in the subject, decreases steatosis in the subject, decreases fibrosis in the subject, decreases glycogen synthesis in the subject, increases gluconeogenesis in the subject, halts disease progression in the subject, reverses fibrosis in the subject, and/or reduces risk of death due to cirrhosis, hepatocellular carcinoma, and/or cardiorenal disease in the subject. In some aspects, the subject is a human. In some aspects, the peptide is administered about once a week.

### EXAMPLES

### Example 1: Lipidated-GLP-1/Glucagon Dual Agonist Peptide Analogue Preparation

Lipidated-GLP-1R/GCGR dual agonist peptides were synthesized as C-terminal corboxamides or carboxylic acids using rink amide MBHA resin (100-200 mesh) or Wang resin (100-200 mesh). All peptides were prepared by automated synthesis using a PTI Prelude solid phase peptide synthesizer using the 9-fluorenylmethoxycarbonyl (Fmoc)/tert-butyl (′Bu) protocol. Manufacturer-supplied protocols were applied for coupling of amino acids in N,N-dimethylformamide (DMF) and deprotection of Fmoc protecting group using piperidine in DMF (20% v/v). Asparagine (Asn), glutamine (Gln) and histidine (His) were incorporated as their sidechain triphenylmethyl, trityl (Trt) derivatives. Lysine (Lys) was incorporated as the sidechain tert-butyloxycarbonyl (Boc) derivative. Serine (Ser), threonine (Thr) and tyrosine (Tyr) were incorporated as sidechain ^{t}Bu ethers, and aspartate (Asp) and glutamate (Glu) as their sidechain O'Bu esters. Arginine (Arg) was incorporated as the sidechain 2,2,4,6,7-pentamethyldihydrobenzofuran-5-sulfonyl (Pbf) derivative. Other amino acids were incorporated with an appropriate sidechain protection.

Lys(Mmt) was incorporated when a subsequent chemical modification of the lysine side chain was required. Upon completion of the peptide chain elongation, Mmt side chain protection was removed by treatment of the resin with selective deprotection cocktail (1% trifluoroacetic acid (TFA), 5% TIPS in dichloromethane (DCM)) at 100 mL/mmol for 1 min, and repeated at least 10 times until Mmt group deprotection was completed. The reaction was quenched with 10% N,N-diisopropylethylamine (DIPEA)/NMP. Subsequent coupling of a albumin binding moiety, such as a lipid and linker, was performed manually using 1-[Bis(dimethylamino)methylene]-1H-1,2,3-triazolo[4,5-b]pyridinium 3-oxid hexafluorophosphate (HATU) as a coupling reagent in the presence of DIPEA.

Peptides were cleaved from the solid support by treatment with a mixture of TFA: triisopropylsilane (TIS):water (92.5.5:2.5v/v) for 4 h with agitation at room temperature. Thereafter, the cleavage mixtures were filtered, concentrated in vacuo, precipitated and washed with diethyl ether and solids were isolated by centrifugation. The crude peptides were dried under a flow of nitrogen and dissolved in 20% acetonitrile (MeCN)/water (v/v) and filtered. The crude peptides were purified using a preparative reversed-phase high-performance liquid chromatography (RP-HPLC) on a Varian SD-1 Prep Star binary pump system, monitoring by ultraviolet (UV) absorption at 210 nm using an Xbridge C18-A stationary phase (19.0×250 mm, 5 micron) column eluting a linear solvent gradient of 25-70% MeCN (0.1% TFA v/v) in water (0.1% TFA v/v) over 25 min. The purified fractions were pooled, frozen and lyophilised.

Liquid chromatography/mass spectrometry (LC/MS) characterization of purified peptides were performed on a Waters MassLynx 3100 platform using a XBridge C18 stationary phase (4.6×100 mm, 3 micron) eluting a linear binary gradient of 10-90% MeCN (0.1% TFA v/v) in water (0.1% TFA v/v) over 10 minutes at 1.5 mL/min at ambient temperature. Analytes were detected by both UV absorption at 210 nm and ionization using a Waters 3100 mass detector (electrospray ionisation (ESI)+ mode). Analytical RP-HPLC characterization was performed on an Agilent 1260 Infinity system using an Agilent Polaris C8-A stationary phase (4.6×100 mm, 3 micron) eluting a linear binary gradient of 10-90% MeCN (0.1% TFA v/v) in water (0.1% TFA v/v) at 1.5 mL/min over 15 minutes at 40° C.

### Example 2: GLP-1 Receptor and Glucagon Receptor In Vitro Functional Assay

The functional activities of lipidated-GLP-1R/GCGR peptides, such as cAMP production, were tested in CHO cell line with stable recombinant expression of human GLP-1 receptor (hGLP-1R) or human glucagon receptor (hGCGR).

Cryopreserved cell stocks were thawed rapidly in a water-bath, suspended in assay buffer (0.1% BSA (Sigma #A3059) in HBSS (Sigma #H8264) with 25 mM HEPES, pH 7.4 and containing 0.5 mM IBMX (Sigma #17018)) and centrifuged at 240×g for 5 minutes. Cells were re-suspended in assay buffer at a batch-dependent optimized concentration (typically hGLP-1R cells at 1×10⁵ cells/mL, hGCGR cells at 2×10⁵ cells/mL).

The test peptide stock was prepared in DMSO and serially diluted in assay buffer to prepare 11-point concentration response curves, in duplicate, in 384-well low volume microtiter assay plates (Corning #4514) using a non-contact liquid dispenser (ECHO™, LabCyte). Cells were added to the assay plate using a multidrop dispenser and incubated at room temperature for 30 minutes before measuring the cAMP level using a cAMP dynamic 2 homogeneous time resolved Fluorescence (HTRF) kit (Cisbio Bioassays #62AM4PEJ) following the two-step protocol as per manufacturer's recommendations. In brief, anti-cAMP cryptate (donor fluorophore) and cAMP-d2 (acceptor fluorophore) were made up separately by diluting each 1 in 20 in conjugate and lysis buffer provided in the kit. Anti-CAMP cryptate was added to all wells of the assay plate, followed by cAMP-d2 added to all wells except non-specific binding (NSB) wells (to which conjugate and lysis buffer was added). Plates were incubated at room temperature for one hour and then read on an Envision (Perkin Elmer) using an excitation wavelength of 320 nm and emission wavelengths of 620 nm & 665 nm.

Data was transformed to % Delta F as described in the manufacturer's guidelines and analyzed by 4-parameter logistic fit to determine EC₅₀ values. The selectivity ratio of a peptide to hGLP-1R vs hGCGR is defined as: % Relative Potency Ratio=% GLP-1R activity relative to GLP-1/% GlucR activity relative to glucagon. Data is shown as the geometric mean EC₅₀ (pM) from >n=2 independent experiments.

The relative potency ratios of peptides is listed in Tables 6-10.

TABLE 6
Potency of Peptides with Modification
on Amino Acid at Position 17

| | hGLP-1R | hGluc-R | % Relative |
|---|---|---|---|
| Peptide | EC₅₀ (pM) | EC₅₀ (pM) | Potency Ratio |
| GLP-1(7-36) amide | 2.196 | — | — |
| Glucagon | — | 1.110 | — |
| 1 | 3197.264 | >27100 | — |
| 2 | 1281.484 | 4192.851 | 6.5 |
| 3 | 69.013 | 149.198 | 4.3 |
| 4 | 240.004 | 1167.129 | 9.6 |
| 5 | 282.756 | 7137.899 | 49.9 |
| 6 | 567.937 | 516.038 | 1.8 |
| 7 | 84.551 | 30.666 | 0.7 |
| 8 | 87.845 | 53.635 | 1.2 |
| 9 | 241.876 | 278.726 | 2.3 |
| 10 | 657.981 | 376.720 | 1.1 |
| 11 | 156.625 | 117.572 | 1.5 |
| 12 | 14.531 | 47.666 | 6.5 |
| 13 | 15.895 | 44.043 | 5.5 |
| 14 | 93.788 | 462.195 | 9.7 |
| 15 | 33.122 | 61.419 | 3.7 |
| 16 | 32.959 | 104.788 | 6.3 |
| 17 | 44.976 | 134.979 | 5.9 |
| 18 | 20.493 | 121.569 | 11.7 |
| 19 | 38.345 | 133.477 | 6.9 |
| 20 | 152.448 | 135.633 | 1.8 |
| 21 | 377.683 | 68.754 | 0.4 |
| 22 | 1226.703 | 2724.463 | 4.4 |
| 23 | 266.349 | 321.178 | 2.4 |
| 24 | 82.803 | 101.130 | 2.4 |
| 25 | 725.678 | 1311.347 | 3.6 |
| 26 | 80.600 | 8904.381 | 218.5 |
| 27 | 18.189 | 1438.437 | 156.4 |
| 28 | 129.321 | 259.647 | 4.0 |
| 29 | 106.638 | 629.359 | 11.7 |
| 30 | 52.683 | 234.982 | 8.8 |
| 31 | 41.292 | 56.793 | 2.7 |
| 40 | 29.909 | 123.325 | 8.2 |
| 41 | 662.690 | 5737.086 | 17.1 |
| 42 | 665.048 | 8189.848 | 24.4 |
| 136 | 45.196 | 509.273 | 22.3 |
| 137 | 143.435 | 267.783 | 3.7 |
| 138 | 104.274 | 183.767 | 3.5 |
| 139 | 630.031 | 983.762 | 3.1 |
| 140 | 22.725 | 46.408 | 4.0 |
| 141 | 263.906 | 392.259 | 2.9 |
| 142 | 59.361 | 107.506 | 3.6 |
| 143 | 19.134 | 32.638 | 3.4 |
| 144 | 45.230 | 702.417 | 30.7 |
| 145 | 22.618 | 4587.203 | 401.2 |
| 146 | 24.628 | 1634.605 | 131.3 |
| 147 | 15.328 | 43.496 | 5.6 |
| 148 | 40.233 | 567.838 | 27.9 |
| 149 | 8.707 | 2653.203 | 602.8 |
| 150 | 166.473 | 355.432 | 4.2 |
| 151 | 111.202 | 178.430 | 3.2 |
| 152 | 11.912 | 20.350 | 3.4 |
| 153 | 41.161 | 99.002 | 4.8 |
| 158 | 303.611 | 332.670 | 2.2 |
| 159 | 145.208 | 144.564 | 2.0 |
| 160 | 55.178 | 416.851 | 14.9 |
| 162 | 59.567 | 1027.507 | 34.1 |
| 161 | 50.942 | 1030.580 | 40.0 |
| 166 | 48.005 | 269.638 | 11.1 |
| 167 | 76.814 | 310.656 | 8.0 |
| 43 | 1.894 | 5.520 | 5.8 |
| 44 | 2.117 | 18.235 | 17.0 |
| 45 | 2.028 | 14.943 | 14.6 |
| 46 | 3.395 | 5.914 | 3.4 |
| 47 | 1.715 | 2.381 | 2.7 |
| 48 | 19.862 | 43.244 | 4.3 |
| 49 | 1.738 | 1.728 | 2.0 |
| 50 | 4.903 | 8.738 | 3.5 |
| 51 | 18.101 | 41.930 | 4.6 |
| 52 | 22.733 | 6.646 | 0.6 |
| 53 | 3.025 | 18.850 | 12.3 |
| 54 | 2.810 | 2.588 | 1.8 |
| 55 | 18.800 | 19.085 | 2.0 |
| 56 | 4.860 | 10.909 | 4.4 |
| 57 | 9.093 | 16.912 | 3.7 |
| 58 | 3.900 | 11.329 | 5.7 |
| 59 | 6.181 | 28.350 | 9.1 |
| 60 | 5.161 | 21.118 | 8.1 |
| 61 | 4.125 | 9.554 | 4.6 |
| 62 | 10.098 | 15.204 | 3.0 |
| 63 | 3.139 | 2.488 | 1.6 |
| 64 | 2.574 | 3.065 | 2.4 |
| 65 | 1230.000 | 2060.580 | 3.3 |
| 66 | 1.524 | 6.426 | 8.3 |
| 67 | 2.432 | 9.488 | 7.7 |
| 68 | 1.095 | 4.670 | 8.4 |
| 69 | 1.145 | 6.442 | 11.1 |
| 70 | 1.550 | 30.865 | 39.4 |
| 71 | 2.174 | 53.722 | 48.9 |
| 72 | 3.418 | 73.660 | 42.6 |
| 73 | 0.275 | 21.956 | 157.9 |
| 74 | 1.831 | 10.893 | 11.8 |
| 75 | 18.897 | 2803.488 | 293.5 |
| 76 | 1.776 | 339.882 | 378.6 |
| 77 | 8.682 | 243.406 | 55.5 |
| 78 | 1.135 | 8.972 | 15.6 |
| 79 | 1.733 | 10.679 | 12.2 |
| 80 | 1.033 | 9.179 | 17.6 |
| 81 | 1.188 | 13.543 | 22.6 |
| 82 | 0.839 | 7.774 | 18.3 |
| 83 | 1.043 | 15.604 | 29.6 |
| 84 | 2.798 | 44.887 | 31.7 |
| 85 | 1.038 | 47.687 | 90.9 |
| 86 | 0.772 | 6.459 | 16.6 |
| 87 | 1.289 | 8.465 | 13.0 |
| 88 | 1.040 | 6.467 | 12.3 |
| 89 | 3.769 | 15.282 | 8.0 |
| 90 | 1.866 | 6.606 | 7.0 |
| 91 | 1.570 | 7.797 | 9.8 |
| 92 | 0.895 | 10.899 | 24.1 |
| 93 | 0.472 | 8.920 | 37.4 |
| 94 | 0.515 | 5.397 | 20.7 |
| 95 | 0.608 | 12.464 | 40.6 |
| 96 | 0.851 | 30.053 | 69.9 |
| 97 | 1.055 | 12.962 | 24.3 |
| 98 | 2.442 | 54.125 | 43.8 |
| 99 | 2.212 | 33.196 | 29.7 |
| 100 | 0.258 | 40.473 | 310.3 |
| 101 | 0.713 | 119.000 | 330.1 |
| 102 | 0.231 | 26.508 | 227.0 |
| 103 | 0.532 | 59.381 | 220.8 |
| 104 | 246.670 | 45.636 | 0.4 |
| 105 | 64.817 | 121.121 | 3.7 |
| 106 | 6.819 | 36.794 | 10.7 |
| 107 | 65.867 | 99.074 | 3.0 |
| 108 | 3197.264 | >27100.000 | — |
| 109 | 697.596 | >26800.000 | — |
| 110 | 0.627 | 48.465 | 152.9 |
| 111 | 1.918 | 2.090 | 2.2 |
| 112 | 3.364 | 2.689 | 1.6 |
| 113 | 1.613 | 4.603 | 5.6 |
| 114 | 3.647 | 2.648 | 1.4 |
| 117 | 15.615 | 2.990 | 0.4 |
| 116 | 24.329 | 10.308 | 0.8 |
| 117 | 1.398 | 1.848 | 2.6 |
| 118 | 1.068 | 1.201 | 2.2 |
| 119 | 1.392 | 1.700 | 2.4 |
| 120 | 1.810 | 2.616 | 2.9 |
| 121 | 1.502 | 1.200 | 1.6 |
| 122 | 1.417 | 0.857 | 1.2 |
| 123 | 0.496 | 12.883 | 51.4 |
| 124 | 1.274 | 9.790 | 15.2 |
| 125 | 0.575 | 6.676 | 23.0 |
| 128 | 0.594 | 10.997 | 36.6 |
| 127 | 0.998 | 12.649 | 25.1 |
| 128 | 0.788 | 4.695 | 11.8 |
| 129 | 0.462 | 4.100 | 17.6 |
| 130 | 1.087 | 32.889 | 59.9 |
| 131 | 0.511 | 17.811 | 68.9 |
| 132 | 0.949 | 34.315 | 71.5 |
| 133 | 1.060 | 29.964 | 55.9 |
| 134 | 0.592 | 15.008 | 50.1 |
| 135 | 0.407 | 14.984 | 72.8 |
| 407 | 208.719 | 462.060 | 4.4 |
| 408 | 370.323 | 828.459 | 4.4 |
| 409 | 22.673 | 203.000 | 17.7 |
| 410 | 78.013 | 455.043 | 11.5 |
| 411 | 98.122 | 377.956 | 7.6 |
| 412 | 185.707 | 397.402 | 4.2 |
| 413 | 406.086 | 1023.554 | 5.0 |
| 414 | 32.273 | 26.904 | 1.6 |
| 415 | 14.693 | 301.237 | 40.6 |
| 416 | 67.741 | 581.428 | 17.0 |
| 417 | 33.876 | 16.440 | 1.0 |
| 418 | 88.091 | 221.794 | 5.0 |
| 419 | 96.888 | 264.191 | 5.4 |
| 420 | 23.104 | 36.906 | 3.2 |
| 421 | 87.299 | 179.876 | 4.1 |
| 422 | 27.559 | 123.460 | 8.9 |
| 423 | 26.123 | 2219.743 | 168.1 |
| 424 | 15.540 | 129.497 | 16.5 |
| 425 | 11.490 | 826.662 | 142.3 |
| 426 | 7.303 | 61.169 | 16.6 |
| 427 | 12.433 | 47.225 | 7.5 |
| 428 | 33.081 | 10.501 | 0.6 |
| 429 | 15.013 | 144.446 | 19.0 |
| 430 | 24.421 | 160.262 | 13.0 |
| 431 | 44.587 | 193.903 | 8.6 |
| 432 | 1377.330 | 1164.734 | 1.7 |
| 433 | 4464.399 | 10463.230 | 4.6 |
| 434 | 152.763 | 293.928 | 3.8 |
| 435 | 22.659 | 240.076 | 21.0 |
| 436 | 7.427 | 68.750 | 18.3 |
| 437 | 26.892 | 96.876 | 7.1 |
| 438 | 17.923 | 68.908 | 7.6 |
| 439 | 30.650 | 57.730 | 3.7 |
| 440 | 153.518 | 81.008 | 1.0 |
| 441 | 62.730 | 72.810 | 2.3 |
| 442 | 19.052 | 35.819 | 3.7 |
| 443 | 20.559 | 22.434 | 2.2 |
| 444 | 247.415 | 1758.388 | 14.1 |
| 445 | 33.016 | 683.673 | 41.0 |
| 446 | 32.889 | 365.880 | 22.0 |
| 447 | 169.598 | 666.503 | 7.8 |
| 448 | 53.134 | 28.953 | 1.1 |
| 449 | 207.17 | 241.70 | 2.3 |
| 450 | 57.318 | 57.789 | 2.0 |
| 451 | 894.881 | 503.273 | 1.1 |
| 452 | 495.050 | 150.934 | 0.6 |
| 453 | 169.523 | 161.692 | 1.9 |
| 454 | 173.343 | 273.630 | 3.1 |
| 455 | 127.140 | 114.432 | 1.8 |
| 456 | 132.866 | 80.328 | 1.2 |
| 457 | 109.278 | 202.834 | 3.7 |
| 458 | 114.344 | 384.540 | 6.7 |
| 459 | 32.674 | 36.947 | 2.2 |
| 460 | 116.724 | 66.478 | 1.1 |
| 461 | 167.051 | Not tested | — |
| 462 | 39.497 | 89.240 | 4.5 |
| 463 | 273.563 | Not tested | — |
| 464 | Not tested | Not tested | — |
| 465 | 485.020 | 436.382 | 1.8 |
| 466 | 35.571 | 131.195 | 7.3 |
| 467 | Not tested | Not tested | — |
| 468 | 67.532 | 292.374 | 8.6 |
| 469 | 254.776 | 538.155 | 4.2 |
| 470 | 55.908 | 30.269 | 1.1 |
| 471 | 101.756 | 63.584 | 1.2 |
| 472 | 7.627 | 11.060 | 2.9 |
| 473 | 11.463 | 14.135 | 2.4 |
| 474 | 8.043 | 56.901 | 14.0 |
| 475 | 6.748 | 61.516 | 18.0 |
| 476 | 57.309 | 103.924 | 3.6 |
| 477 | 54.091 | 117.426 | 4.3 |
| 478 | 9.178 | 189.932 | 40.9 |
| 479 | 34.154 | 206.779 | 12.0 |
| 480 | 12.573 | 136.201 | 21.4 |
| 481 | 52.209 | 349.837 | 13.3 |
| 482 | 190.311 | 691.097 | 7.2 |
| 483 | 177.661 | 168.551 | 1.9 |
| 484 | 338.595 | 168.884 | 1.0 |
| 485 | 219.193 | 90.425 | 0.8 |
| 486 | 420.381 | 166.466 | 0.8 |
| 487 | 65.455 | 164.468 | 5.0 |
| 488 | 201.998 | 300.737 | 2.9 |
| 489 | 22.787 | 167.608 | 14.5 |
| 490 | 69.046 | 396.488 | 11.4 |
| 491 | 73.584 | 231.822 | 6.2 |
| 492 | 212.412 | 374.486 | 3.5 |
| 493 | 23.450 | 165.822 | 14.0 |
| 494 | 98.727 | 212.207 | 4.3 |
| 495 | 23.165 | 379.702 | 32.4 |
| 496 | 64.887 | 482.010 | 14.7 |
| 497 | 37.397 | 118.244 | 6.3 |
| 498 | 156.406 | 169.974 | 2.1 |
| 499 | 40.647 | 78.787 | 3.8 |
| 500 | 101.880 | 101.482 | 2.0 |
| 501 | 26.516 | 75.630 | 5.6 |
| 502 | 82.090 | 174.964 | 4.2 |
| 503 | 81.209 | 84.902 | 2.1 |
| 504 | 123.927 | 551.274 | 8.8 |
| 505 | 42.121 | 190.780 | 9.0 |
| 506 | 162.104 | 148.973 | 1.8 |
| 507 | 52.850 | 86.602 | 3.2 |
| 508 | 67.930 | 2802.216 | 81.6 |
| 509 | 594.123 | 3020.408 | 10.1 |
| 510 | 176.244 | 117.832 | 1.3 |
| 511 | 117.715 | 139.920 | 2.4 |
| 512 | 159.226 | 2308.999 | 28.7 |
| 513 | 93.821 | 1340.613 | 28.3 |
| 514 | 76.660 | 1373.731 | 35.4 |
| 515 | 296.991 | 2659.628 | 17.7 |
| 516 | 137.100 | 784.568 | 11.3 |
| 517 | 145.751 | 737.919 | 10.0 |
| 136 | 45.196 | 509.273 | 22.3 |
| 137 | 143.435 | 267.783 | 3.7 |
| 138 | 104.274 | 183.767 | 3.5 |
| 139 | 630.031 | 983.762 | 3.1 |
| 140 | 22.725 | 46.408 | 4.0 |
| 141 | 263.906 | 392.259 | 2.9 |
| 142 | 59.361 | 107.506 | 3.6 |
| 143 | 19.134 | 32.638 | 3.4 |
| 144 | 45.230 | 702.417 | 30.7 |
| 145 | 22.618 | 4587.203 | 401.2 |
| 146 | 24.628 | 1634.605 | 131.3 |
| 147 | 15.328 | 43.496 | 5.6 |
| 148 | 40.233 | 567.838 | 27.9 |
| 149 | 8.707 | 2653.203 | 602.8 |
| 150 | 166.473 | 355.432 | 4.2 |
| 151 | 111.202 | 178.430 | 3.2 |
| 152 | 11.912 | 20.350 | 3.4 |
| 153 | 41.161 | 99.002 | 4.8 |
| 158 | 303.611 | 332.670 | 2.2 |
| 159 | 145.208 | 144.564 | 2.0 |
| 160 | 55.178 | 416.851 | 14.9 |
| 162 | 59.567 | 1027.507 | 34.1 |
| 165 | 50.942 | 1030.580 | 40.0 |
| 166 | 48.005 | 269.638 | 11.1 |
| 167 | 76.814 | 310.656 | 8.0 |
| 518 | 31.000 | 77.320 | 4.9 |
| 519 | 172.230 | 385.880 | 4.4 |
| 520 | 542.600 | 1616.230 | 5.9 |
| 32 | 12.953 | 240.474 | 36.7 |
| 33 | 213.123 | 548.248 | 5.1 |
| 34 | 82.377 | 160.622 | 3.9 |
| 35 | 83.697 | 334.904 | 7.9 |
| 36 | 43.060 | 277.733 | 12.8 |
| 37 | 68.620 | 2748.412 | 79.2 |
| 38 | 75.589 | 441.481 | 11.6 |
| 39 | 76.068 | 193.860 | 5.0 |
| 161 | 17.234 | 188.040 | 21.6 |
| 163 | 90.154 | 143.715 | 3.2 |
| 168 | 447.505 | 886.110 | 3.9 |
| 169 | 210.397 | 1404.311 | 13.2 |
| 170 | 147.744 | 835.556 | 11.2 |
| 171 | 106.863 | 376.304 | 7.0 |
| 172 | 213.354 | 1273.732 | 11.8 |
| 173 | 134.317 | 874.131 | 12.9 |
| 174 | 127.027 | 420.420 | 6.5 |
| 175 | 68.376 | 2018.944 | 58.4 |
| 176 | 59.942 | 480.338 | 15.9 |
| 177 | 23.777 | 306.464 | 25.5 |
| 178 | 51.770 | 104.397 | 4.0 |
| 179 | 130.928 | 203.431 | 3.1 |
| 180 | 24.377 | 71.735 | 5.8 |
| 181 | 56.000 | 33.440 | 1.2 |
| 182 | 29.060 | 22.580 | 1.5 |
| 183 | 23.730 | 81.430 | 6.8 |
| 184 | 22.040 | 29.620 | 2.7 |
| 185 | 44.113 | 229.651 | 10.3 |
| 186 | 44.923 | 100.835 | 4.4 |
| 188 | 20.847 | 34.713 | 3.3 |
| 189 | 11.315 | 44.301 | 7.7 |
| 192 | 107.568 | 227.117 | 4.2 |
| 193 | 282.849 | 476.105 | 3.3 |
| 194 | 424.799 | 592.863 | 2.8 |
| 195 | 86.016 | 94.577 | 2.2 |
| 196 | 26.953 | 240.329 | 17.6 |
| 197 | 64.040 | 111.890 | 3.5 |
| 198 | 211.810 | 302.200 | 2.8 |
| 199 | 31.604 | 180.232 | 11.3 |
| 200 | 22.687 | 97.711 | 8.5 |
| 201 | 22.724 | 182.560 | 15.9 |
| 202 | 17.824 | 242.436 | 26.9 |

TABLE 7
Potency of Peptides with Modification
on Amino Acid at Position 20

| | hGLP-1R | hGluc-R | % Relative |
|---|---|---|---|
| Peptide | EC₅₀ (pM) | EC₅₀ (pM) | Potency Ratio |
| 203 | 77.466 | 34.112 | 0.9 |
| 204 | 43.306 | 595.006 | 27.2 |
| 205 | 29.901 | 8.136 | 0.5 |
| 206 | 70.949 | 18.170 | 0.5 |
| 207 | 177.618 | 96.175 | 1.1 |
| 208 | 64.412 | 658.298 | 20.2 |
| 209 | 126.544 | 42.591 | 0.7 |
| 210 | 156.403 | 93.732 | 1.2 |
| 211 | 166.600 | 60.904 | 0.7 |
| 212 | 531.595 | 97.476 | 0.4 |
| 213 | 534.307 | 140.535 | 0.5 |
| 214 | 2339.231 | 809.862 | 0.7 |
| 215 | 7623.287 | 2609.521 | 0.7 |
| 216 | 18055.470 | 5672.548 | 0.6 |
| 217 | 263.939 | 126.681 | 0.9 |
| 218 | 138.770 | 422.882 | 6.0 |
| 219 | 94.472 | 297.862 | 6.2 |
| 220 | 240.450 | 405.230 | 3.3 |
| 221 | 122.449 | 122.801 | 2.0 |
| 222 | 151.493 | 243.967 | 3.2 |
| 223 | 48.761 | 103.522 | 4.2 |
| 224 | 32.095 | 28.693 | 1.8 |
| 225 | 34.873 | 144.655 | 8.2 |
| 226 | 82.675 | 27.435 | 0.7 |
| 227 | 70.467 | 27.208 | 0.8 |
| 228 | 200.219 | 176.842 | 1.7 |
| 229 | 68.511 | 198.144 | 5.7 |
| 230 | 170.050 | 37.108 | 0.4 |
| 231 | 49.851 | 58.542 | 2.3 |
| 232 | 110.306 | 1023.865 | 18.4 |
| 233 | 440.364 | 407.411 | 1.8 |
| 234 | 68.020 | 1285.410 | 37.4 |
| 235 | 94.790 | 1262.350 | 26.3 |
| 236 | 22.063 | 214.558 | 19.2 |
| 237 | 26.745 | 298.747 | 22.1 |
| 238 | 154.658 | 990.172 | 12.7 |
| 239 | 105.489 | 704.613 | 13.2 |
| 240 | 57.099 | 332.464 | 11.5 |
| 241 | 35.843 | 175.226 | 9.7 |
| 242 | 248.687 | 1234.326 | 9.8 |
| 243 | 59.292 | 50.633 | 1.7 |
| 244 | 143.475 | 420.232 | 5.8 |
| 245 | 110.173 | 62.682 | 1.1 |
| 246 | 320.437 | 556.602 | 3.4 |
| 247 | 84.851 | 51.143 | 1.2 |
| 248 | 1089.266 | 13553.228 | 24.6 |
| 249 | 558.256 | 545.142 | 1.9 |
| 250 | 601.166 | 1566.333 | 5.2 |
| 251 | 79.751 | 248.071 | 6.2 |
| 252 | 119.246 | 1309.962 | 21.7 |
| 253 | 75.451 | 53.089 | 1.4 |
| 254 | 101.863 | 125.276 | 2.4 |
| 255 | 147.550 | 145.739 | 2.0 |
| 256 | 345.977 | 1306.254 | 7.5 |
| 257 | 7.348 | 1.659 | 0.4 |
| 258 | 34.046 | 93.123 | 5.4 |
| 259 | 19.118 | 34.833 | 3.6 |
| 260 | 33.080 | 76.881 | 4.6 |
| 261 | 16.152 | 29.390 | 3.6 |
| 262 | 13.017 | 11.700 | 1.8 |
| 263 | 25.386 | 64.646 | 5.0 |
| 264 | 69.537 | 297.466 | 8.5 |
| 265 | 71.223 | 130.903 | 3.6 |
| 266 | 66.478 | 293.480 | 8.7 |
| 267 | 65.581 | 127.683 | 3.9 |
| 268 | 39.521 | 29.251 | 1.5 |
| 269 | 51.951 | 271.143 | 10.3 |
| 270 | 24.445 | 31.318 | 2.5 |
| 271 | 54.604 | 27.999 | 1.0 |
| 272 | 15.650 | 14.309 | 1.8 |
| 273 | 22.118 | 23.336 | 2.1 |
| 274 | 27.112 | 31.277 | 2.3 |
| 275 | 110.638 | 175.236 | 3.1 |
| 276 | 143.383 | 91.395 | 1.3 |
| 277 | 49.494 | 57.270 | 2.3 |
| 278 | 78.855 | 83.493 | 2.1 |
| 279 | 86.544 | 150.224 | 3.4 |
| 280 | 78.346 | 154.623 | 3.9 |
| 281 | 155.104 | 1042.071 | 13.3 |
| 282 | 1230.958 | 1135.696 | 1.8 |
| 283 | 129.487 | 552.440 | 8.4 |
| 284 | 43.422 | 118.686 | 5.4 |
| 285 | 300.590 | 87.044 | 0.6 |
| 286 | 31.616 | 52.951 | 3.3 |
| 287 | 27.519 | 26.538 | 1.9 |
| 288 | 42.719 | 26.875 | 1.2 |
| 289 | 118.366 | 148.058 | 2.5 |
| 290 | 346.546 | 260.062 | 1.5 |
| 291 | 1509.011 | 847.167 | 1.1 |
| 292 | 28.705 | 20.397 | 1.4 |
| 293 | 91.968 | 33.473 | 0.7 |
| 294 | 510.108 | 102.417 | 0.4 |
| 295 | 312.053 | 17071.588 | 108.2 |
| 296 | 64.149 | 3253.412 | 100.3 |
| 297 | 30.440 | 103.940 | 6.8 |
| 298 | 75.450 | 572.856 | 15.0 |
| 299 | 75.820 | 193.220 | 5.0 |
| 300 | 19.742 | 18.360 | 1.8 |
| 301 | 55.339 | 31.826 | 1.1 |
| 302 | 151.070 | 270.815 | 3.5 |
| 303 | 99.136 | 99.581 | 2.0 |
| 304 | 238.821 | 687.098 | 5.7 |
| 305 | 211.193 | 154.945 | 1.5 |
| 306 | 47.214 | 775.451 | 32.5 |
| 307 | 22.180 | 1126.909 | 100.5 |
| 308 | 27.499 | 1716.004 | 123.4 |
| 309 | 70.607 | 17377.186 | 486.8 |
| 310 | 56.382 | 1836.826 | 64.4 |
| 311 | 35.100 | 468.731 | 26.4 |
| 312 | 152.183 | 2775.255 | 36.1 |
| 313 | 138.271 | 1227.004 | 17.6 |
| 314 | 30.764 | 6832.600 | 439.3 |
| 315 | 18.118 | 5853.796 | 639.1 |
| 318 | 55.250 | 12001.370 | 429.7 |
| 321 | 22.635 | 4402.316 | 384.7 |
| 322 | 68.302 | >24663.650 | — |
| 323 | 11406.357 | >25553.360 | — |
| 324 | 1329.728 | 1927.309 | 2.9 |
| 325 | 928.583 | >25546.770 | — |
| 326 | 6741.438 | >25645.230 | — |
| 327 | 30.097 | >25671.500 | — |
| 328 | 15.357 | 1698.302 | 218.8 |
| 330 | 487.667 | >25645.230 | — |
| 331 | 62.577 | 66.606 | 2.1 |
| 332 | 77.474 | 58.853 | 1.5 |
| 333 | 134.727 | 212.627 | 3.1 |
| 334 | 249.757 | 461.777 | 3.7 |
| 335 | 1060.440 | 311.556 | 0.6 |
| 336 | 631.959 | 304.533 | 1.0 |
| 337 | 400.475 | 89.638 | 0.4 |
| 338 | 307.997 | 25.677 | 0.2 |
| 339 | 311.774 | 70.289 | 0.4 |
| 340 | 312.126 | 94.738 | 0.6 |
| 341 | 362.926 | 72.771 | 0.4 |
| 342 | 220.158 | 4832.731 | 43.4 |
| 343 | 198.013 | 19629.289 | 196.1 |
| 521 | 677.458 | 10099.850 | 29.5 |
| 522 | 508.426 | 6363.603 | 24.8 |
| 523 | 615.272 | >24743.230 | — |
| 524 | 365.830 | >24393.160 | — |
| 525 | 177.315 | 2454.700 | 27.4 |
| 526 | 194.459 | 689.991 | 7.0 |

TABLE 8
Potency of Peptides with Modification
on Amino Acid at Position 24

| | hGLP-1R | hGluc-R | % Relative |
|---|---|---|---|
| Peptide | EC₅₀ (pM) | EC₅₀ (pM) | Potency Ratio |
| 343 | 58.553 | 125.666 | 4.2 |
| 344 | 134.933 | >26900.000 | — |
| 345 | 2860.629 | 167.753 | 0.1 |
| 346 | 7318.019 | 202.791 | 0.1 |
| 347 | 4971.036 | 596.962 | 0.2 |
| 348 | 70.074 | 4107.919 | 116.0 |
| 349 | 121.061 | 83.684 | 1.4 |
| 350 | 88.617 | 383.622 | 8.6 |
| 351 | 605.282 | 216.444 | 0.7 |
| 352 | 192.562 | 139.241 | 1.4 |
| 353 | 311.480 | 162.628 | 1.0 |
| 354 | 1106.278 | 901.878 | 1.6 |
| 355 | 424.009 | 478.456 | 2.2 |
| 356 | 304.243 | 315.728 | 2.1 |
| 357 | 916.728 | 550.189 | 1.2 |
| 358 | 74.855 | 16049.922 | 424.1 |
| 359 | 46.101 | 5048.643 | 216.6 |
| 360 | 107.773 | 5640.213 | 103.5 |
| 361 | 35.698 | 23.565 | 1.3 |
| 362 | 54.824 | 42.799 | 1.5 |
| 363 | 18.268 | 47.517 | 5.1 |
| 364 | 23.354 | 30.435 | 2.6 |
| 365 | 51.340 | 8.774 | 0.3 |
| 366 | 31.038 | 27.411 | 1.7 |
| 367 | 139.499 | 72.449 | 1.0 |
| 368 | 186.325 | 290.003 | 3.1 |
| 369 | 103.653 | 201.633 | 3.8 |
| 370 | 323.469 | 182.253 | 1.1 |
| 371 | 190.869 | 87.178 | 0.9 |
| 372 | 71.789 | 35.094 | 1.0 |
| 373 | 66.677 | 242.458 | 7.2 |
| 374 | 434.390 | 260.160 | 1.2 |
| 375 | 225.465 | 154.858 | 1.4 |
| 376 | 155.910 | 853.499 | 10.8 |
| 377 | 149.787 | 189.515 | 2.5 |
| 378 | 158.745 | 157.793 | 2.0 |
| 379 | 71.766 | 85.739 | 2.4 |
| 380 | 871.183 | 14947.910 | 33.9 |
| 381 | 174.069 | 1308.052 | 14.9 |
| 382 | 56.012 | 329.333 | 11.6 |
| 383 | 197.271 | 231.689 | 2.3 |
| 384 | 796.819 | 1244.347 | 3.1 |
| 386 | 31.186 | 140.185 | 8.9 |
| 387 | 82.603 | 1573.075 | 37.7 |
| 388 | 52.732 | 71.929 | 2.7 |
| 389 | 224.224 | 135.698 | 1.2 |

TABLE 9
Potency of Peptides with Modification
on Amino Acid at Position 10

| | hGLP-1R | hGluc-R | % Relative |
|---|---|---|---|
| Peptide | EC₅₀ (pM) | EC₅₀ (pM) | Potency Ratio |
| 391 | 892.536 | 22172.801 | 49.1 |
| 392 | 312.355 | 6922.306 | 43.8 |
| 393 | 0.458 | 2.158 | 9.3 |
| 394 | 0.652 | 2.408 | 7.3 |
| 395 | 0.774 | 9.554 | 24.4 |
| 396 | 0.583 | 11.835 | 40.2 |
| 397 | 5.360 | 10.003 | 3.7 |
| 398 | 3.280 | 2.449 | 1.5 |

TABLE 10
Potency of Peptides with Modification
on Amino Acid at Position 13

| | hGLP-1R | hGluc-R | % Relative |
|---|---|---|---|
| Peptide | EC₅₀ (pM) | EC₅₀ (pM) | Potency Ratio |
| 399 | 0.672 | 5.567 | 16.4 |
| 400 | 1.268 | 10.714 | 16.7 |
| 401 | 0.585 | 9.542 | 32.3 |
| 402 | 1.044 | 18.081 | 34.3 |
| 404 | 5.652 | 21.821 | 7.6 |
| 405 | 1041.139 | 8585.500 | 16.3 |
| 406 | 457.645 | 7308.311 | 31.6 |

### Example 3: Mouse Acute Food Intake Study

Male C57Bl/6 mice obtained from Jackson Laboratories or Charles River at 8-9 weeks of age and were housed one per cage in BioDaq (Research Diets) cages. Mice were placed on Alpha Dri bedding, standard chow diet (Envigo, 2018) and given water pouches. Mice were allowed to acclimate for 1-2 weeks. Body weight was measured to ensure appropriate acclimation. After acclimation, mice were sham dosed a minimum of 2 times prior to initiation of food intake study. On study day, mice were placed in clean cage bottoms, weighed, and fasted for 6-8 hours. Mice were sorted into groups based on average 24-hour food intake data and body weight. 1-2 hours prior to lights out, mice were dosed subcutaneously with one vehicle or test peptide dissolved in an appropriate vehicle at 5 mL/kg. Mice were left undisturbed and allowed access to food and water for 48 hours. Automated food intake was monitored in the BioDaq system was monitored during this time. Discrete food intake data was exported into MS Excel from which cumulative food intake data was generated and analyzed. Food intake at 24 hours compared to vehicle food intake (percent) is provided in Table 11.

TABLE 11
Food Intake of Mice Administered Lipidated Peptides

| | | | 24 hr % Vehicle | Standard Error of |
|---|---|---|---|---|
| Peptide | Dose | | Food Intake | the Mean (SEM) |
| 43 | 10 | nmol/kg | 53.2 | 4.9 |
| 46 | 10 | nmol/kg | 72.1 | 4.3 |
| 47 | 10 | nmol/kg | 51.4 | 4.9 |
| 49 | 10 | nmol/kg | 69.2 | 9.0 |
| 50 | 10 | nmol/kg | 78.5 | 4.1 |
| 69 | 10 | nmol/kg | 74.3 | 4.6 |
| 68 | 10 | nmol/kg | 74.2 | 4.2 |
| 96 | 10 | nmol/kg | 51.9 | 5.5 |
| 62 | 10 | nmol/kg | 52.7 | 3.1 |
| 63 | 10 | nmol/kg | 37.9 | 7.0 |
| 71 | 10 | nmol/kg | 80.6 | 11.5 |
| 45 | 10 | nmol/kg | 76.2 | 4.7 |
| 399 | 10 | nmol/kg | 72.3 | 9.6 |
| 102 | 10 | nmol/kg | 81.9 | 14.6 |
| 73 | 10 | nmol/kg | 63.4 | 4.2 |
| 7 | 10 | nmol/kg | 55.0 | 4.0 |
| 349 | 10 | nmol/kg | 79.9 | 4.2 |
| 203 | 10 | nmol/kg | 81.4 | 7.3 |
| 361 | 10 | nmol/kg | 78.8 | 5.3 |
| 364 | 10 | nmol/kg | 63.7 | 5.5 |
| 366 | 10 | nmol/kg | 83.5 | 9.4 |
| 223 | 10 | nmol/kg | 63.6 | 6.3 |
| 229 | 10 | nmol/kg | 54.8 | 5.6 |
| 224 | 10 | nmol/kg | 66.5 | 7.8 |
| 375 | 10 | nmol/kg | 66.0 | 7.7 |
| 247 | 10 | nmol/kg | 66.9 | 5.5 |
| 257 | 3 | nmol/kg | 94.7 | 6.6 |
| 257 | 10 | nmol/kg | 96.5 | 3.7 |
| 224 | 1 | nmol/kg | 91.3 | 10.9 |
| 224 | 3 | nmol/kg | 76.6 | 3.3 |
| 224 | 10 | nmol/kg | 72.5 | 8.3 |
| 229 | 1 | nmol/kg | 86.0 | 4.7 |
| 229 | 3 | nmol/kg | 72.5 | 3.6 |
| 229 | 10 | nmol/kg | 50.5 | 3.7 |
| 289 | 3 | nmol/kg | 76.1 | 6.9 |
| 289 | 10 | nmol/kg | 49.6 | 6.7 |
| 299 | 30 | nmol/kg | 43.7 | 7.3 |
| 301 | 10 | nmol/kg | 54.9 | 7.6 |
| 300 | 10 | nmol/kg | 45.2 | 6.5 |
| 302 | 30 | nmol/kg | 46.4 | 9.5 |
| 140 | 10 | nmol/kg | 65.4 | 5.5 |
| 195 | 10 | nmol/kg | 58.2 | 8.7 |
| 188 | 10 | nmol/kg | 60.7 | 6.3 |
| 188 | 1 | nmol/kg | 99.9 | 6.3 |
| 188 | 3 | nmol/kg | 80.6 | 5.6 |
| 188 | 10 | nmol/kg | 49.8 | 5.1 |
| 195 | 1 | nmol/kg | 102.1 | 16.9 |
| 195 | 3 | nmol/kg | 84.0 | 13.3 |
| 195 | 10 | nmol/kg | 49.9 | 4.4 |

### Example 4: Evaluating Proteolytic-Resistance of Lipidated Peptides to Fasted State

Stimulated Gastric Fluid (FasSSGF) Containing Porcine Pancreatic Pepsin Lyophilized porcine pancreatic pepsin (Sigma: P7012) was reconstituted to 0.5 mg/mL (˜2500 units/mL) in freshly prepared FasSSGF (Biorelevant media) to give the enzyme stock solution. Peptide stock solution was prepared to a concentration of 250 μM (˜1.0 mg/mL) in FasSSGF. 200 μL (10 μg, ˜250 units) of pepsin stock solution was added to 200 μL of peptide solution (1.0 mg/mL, ˜100 μg of peptide, ˜25 nmoles) and the mixture was co-incubated in a temperature-regulated incubator at 37° C. for the duration of the experiment. 30 μL aliquot of the peptide-enzyme mixture was periodically withdrawn (t=0, 5, 10, 15, and 30 min) and quenched immediately by addition of 80 μL of 0.1M ammonium bicarbonate solution in water/acetonitrile (4:1, pH 8) to arrest proteolytic activity. 30 μL aliquot was analyzed by analytical RP-HPLC. Analytical RP-HPLC method: Agilent Polaris C8-A column (4.6×100 mm, 3 micron) eluted with a linear binary gradient of 10-90% MeCN (0.1% TFA v/v) in water (0.1% TFA v/v) over either 10 or 15 mins at 1.5 mL min-1 at 40° C. with detection by UV absorption at 210 nm. Manual integration (AUC) allowed estimation of remaining intact peptide over the time course of the experiment. Peptide stability data is provided in Table 12.

TABLE 12
Stability of Lipidated Peptides Incubated with FasSSGF

% intact peptide over time (min)

| Peptide | 0 | 5 | 10 | 15 | 30 |
|---|---|---|---|---|---|
| Semaglutide | 100 | 15 | 13 | 12 | 14 |
| 7 | 100 | 81 | 60 | 35 | 18 |
| 229 | 100 | 100 | 97 | 91 | 79 |
| 15 | 100 | ND | ND | 91 | 77 |
| 78 | 100 | ND | ND | 100 | 100 |
| 290 | 100 | ND | ND | 0 | 0 |
| 282 | 100 | ND | ND | 83 | 76 |
| 283 | 100 | ND | ND | 68 | 48 |
| 287 | 100 | ND | ND | 0 | 0 |
| 367 | 100 | ND | ND | 0 | 0 |

### Example 5: Evaluating Proteolytic Resistance of Peptides to Neprilysin

10.0 μg (˜10 units) recombinant Neprilysin (R&D Systems: 1182-ZNC-010) was reconstituted to 100 μL (100 μg/mL, ˜100 units/mL) in assay buffer (50 mM Tris, 50 mM NaCl, 50 mM NaHCO₃, adjusted to pH 8.3) to give the enzyme stock solution. Peptide stock solutions were prepared to a concentration of ˜250 μM (˜1.0 mg/mL of 4 kDa peptide) in assay buffer. 100 μL (10 μg, ˜10 units) of neprilysin stock solution was added to 100 μL of peptide stock solution (1.0 mg/mL, ˜100 μg of peptide) and the mixture was co-incubated in a temperature regulated incubator at 37° C. for the duration of the experiment. 25 μL aliquots (˜ 12.5 μg initial peptide) of the peptide-enzyme mixture were periodically withdrawn (t=0, 30 mins, 1 h, 2 h, 4 h and 24 h) and quenched immediately by addition to an equal volume (75 μL) of 10% TFA (v/v) in 1:1 water/acetonitrile to arrest proteolytic activity. The quenched aliquot centrifuged at 7800 rpm, and 30 μL of the supernatant was analyzed by analytical RP-HPLC as follows: Analytical RP-HPLC method: Agilent Polaris C8-A column (4.6×100 mm, 3 micron) eluted with a linear binary gradient of 10-90% MeCN (0.1% TFA v/v) in water (0.1% TFA v/v) over either 10 or 15 mins at 1.5 mL min-1 at 40° C. with detection by UV absorption at 210 nm. Manual integration (AUC) allowed estimation of remaining intact peptide over the time course of the experiment. Peptide stability data is provided in Table 13.

TABLE 13
Stability of Lipidated Peptides Incubated with Neprilysin

% intact peptide over time

| Peptide | 0 | 30 min | 2 hr | 4 hr | 24 hr |
|---|---|---|---|---|---|
| Semaglutide | 100 | 100 | 100 | 83 | 48 |
| 7 | 100 | 100 | 100 | 97 | 84 |
| 229 | 100 | 100 | 100 | 100 | 100 |
| 15 | 100 | ND | ND | 100 | 83 |
| 78 | 100 | ND | ND | 100 | 100 |
| 290 | 100 | ND | ND | 89 | 82 |
| 282 | 100 | ND | ND | 100 | 97 |
| 283 | 100 | ND | ND | 81 | 63 |
| 287 | 100 | ND | ND | 77 | 59 |

### Example 6: Evaluating Proteolytic-Resistance of Mono-Lipidated Peptides to Fasted-State Simulated Intestinal Fluid (FasSSIF/Pancreatin)

A fresh suspension of FasSSIF/P (Fasted-State Simulated Intestinal Fluid+USP Pancreatin®) was prepared according to that described by Galia, Nicolaides, Hörter, Löbenberg, Reppas and Dressman: Pharm. Res. 15 (1998) 698-705, and USP XL requirements. The preparation of FasSSIF/P is provided in Table 14. The resulting preparation was used immediately without storage.

TABLE 14
Preparation of FasSSIF/Pancreatin

| | Conc. | Mol. | Mass | |
|---|---|---|---|---|
| Composition | mM | Weight | L − 1 | Supplier data |
| Sodium Taurocholate | 3 | 537.68 | 1.61 g | Sigma: 86339 |
| hydrate | | | | |
| L-α-phosphatidylcholine | 0.5 | av. 768 | 0.38 g | Sigma: 44924 |
| (soybean) | | | | |
| Dibasic sodium phosphate | 30 | 141.96 | 4.26 g | Sigma: S7907 |
| Sodium hydroxide | 10 | 40.00 | 0.40 g | Sigma: S8045 |
| Sodium Chloride | 100 | 58.44 | 5.84 g | Sigma: S7653 |
| Pancreatin (8 × USP) | | — | 1.25 g | Sigma: P7545 |

Peptide for evaluation (1.0 mg) was dissolved in pre-warmed FasSSIF without Pancreatin® (200 μL). To this fresh FasSSIF/Pancreatin® (100 μL) was added to initiate potential proteolysis. Following momentary vortexing of the reaction tube the mixture was incubated at 37° C. in a thermostatic water bath for the duration of the experiment. 25 μL aliquot of the co-incubated peptide-enzyme mixture was periodically withdrawn (t=0, 5, 10 15 and 30 min) and quenched immediately by addition to a solution of 10% TFA in 1:1 water/acetonitrile (75 μL) to arrest proteolytic activity. Quenched samples were centrifuged (7800 RPM, 3 mins) to pellet solids and 10 μL aliquots of the supernatant solution were analyzed using analytical RP-HPLC as follows: Analytical RP-HPLC method: Agilent Polaris C8-A column (4.6×100 mm, 3 micron) eluted with a linear binary gradient of 10-90% MeCN (0.1% TFA v/v) in water (0.1% TFA v/v) over either 10 or 15 mins at 1.5 mL min⁻¹ at 40° C. with detection by UV absorption at 210 nm. Manual integration (AUC) allowed estimation of remaining intact peptide over the time course of the experiment. Peptide stability data is provided in Table 15.

TABLE 15
Stability of Lipidated Peptides Incubated with FasSSIF.

% intact peptide over time (min)

| Peptide | 0 | 5 | 10 | 15 | 30 |
|---|---|---|---|---|---|
| Semaglutide | 100 | 22 | 0 | 0 | 0 |
| 7 | 100 | 96 | 98 | 96 | 94 |
| 229 | 100 | 60 | 46 | 33 | 26 |
| 78 | 100 | 83 | 76 | 69 | 58 |
| 47 | 100 | 90 | 88 | 83 | 75 |
| 49 | 100 | 97 | 96 | 95 | 90 |
| 117 | 100 | 54 | 35 | 22 | 8 |
| 43 | 100 | 83 | 33 | 29 | 19 |
| 48 | 100 | ND | ND | ND | 94 |
| 50 | 100 | ND | ND | ND | 98 |
| 51 | 100 | ND | ND | ND | 91 |
| 52 | 100 | ND | ND | ND | 90 |
| 53 | 100 | ND | ND | ND | 92 |

### Example 7: Glucose Levels in GLP-1R Knockout Mice Treated with GLP-1/Glucagon Peptides

GLP-1R knockout (KO) and wildtype (WT) mice at 8-14 weeks of age were singly housed on standard chow diet (Envigo, 2918) and automatic water. They acclimated for a minimum of ˜1-2 weeks, and mice were sorted into groups (n=4-8/group) based on body weight. On day of study, mice were fasted briefly for 2 hrs prior to study start. In some studies, mice were pre-treated with octreotide (BaChem, 10 mg/kg) at 30 minutes prior to peptide injection. Mice were dosed by subcutaneous injection, with peptide administration at doses listed in the Tables 16 and 17. Glucose was measured via glucometer at various timepoints including-30 min (prior to octreotide when used in studies), 0 min (before peptide dose), 30, 60, 120, 180, 240, and 360 minutes. Since these studies are acute in nature, mice were re-used for multiple studies (no more than 3) and always allowed at least a minimum wash-out period of 1 week. Glucose changes represented as percent glucose change (at 60 or 120 min) from time 0 min are provided in Tables 16 and 17.

TABLE 16
Glucose Change in GLP-1R KO and WT Mice
Treated with GLP-1/Glucagon Peptides

| | | | | | GLP-1R | | |
|---|---|---|---|---|---|---|---|
| | | | WT % | | KO % | KO % | |
| | | | glucose | | glucose | glucose | |
| | | | change | WT | change | change | Somatostatin |
| Peptide | Dose | | 120 min | SEM | 120 min | SEM | pretreatment? |
| Vehicle | 5 | mL/kg | 0.9 | 6.3 | −0.7 | 7.6 | No |
| 78 | 3 | nmol/kg | −42.0 | 2.1 | 51.3 | 15.4 | No |
| Vehicle | 5 | mL/kg | −19.2 | 6.7 | −9.1 | 9.5 | No |
| 69 | 3 | nmol/kg | −27.2 | 7.7 | 17.6 | 4.8 | No |
| 43 | 3 | nmol/kg | −56.0 | 4.7 | 63.9 | 39.2 | No |
| Vehicle | 5 | mL/kg | 43.3 | 12.6 | 72.4 | 24.9 | yes 30 min prior to cpd |
| 49 | 10 | nmol/kg | −1.2 | 7.2 | 193.3 | 16.2 | yes 30 min prior to cpd |
| 49 | 30 | nmol/kg | 16.8 | 25.9 | 154.3 | 17.0 | yes 30 min prior to cpd |
| Vehicle | 5 | mL/kg | 28.6 | 6.2 | 23.4 | 8.7 | yes 30 min prior to cpd |
| 97 | 10 | nmol/kg | −41.9 | 3.0 | 91.1 | 25.5 | yes 30 min prior to cpd |
| 97 | 30 | nmol/kg | −14.7 | 7.8 | 150.8 | 17.3 | yes 30 min prior to cpd |
| Vehicle | 5 | mL/kg | 25.8 | 12.9 | 14.1 | 6.9 | yes 30 min prior to cpd |
| 63 | 10 | nmol/kg | −26.4 | 5.2 | 68.2 | 10.2 | yes 30 min prior to cpd |
| 63 | 30 | nmol/kg | −23.8 | 12.1 | 113.0 | 40.1 | yes 30 min prior to cpd |
| Vehicle | 5 | mL/kg | 30.0 | 17.4 | 29.2 | 14 | yes 30 min prior to cpd |
| 126 | 10 | nmol/kg | −26.6 | 6.1 | 76.1 | 16.6 | yes 30 min prior to cpd |
| 126 | 30 | nmol/kg | −46.2 | 3.9 | 92.4 | 30.2 | yes 30 min prior to cpd |
| Vehicle | 5 | mL/kg | 2.2 | 5.2 | 20.5 | 7 | yes 30 min prior to cpd |
| 399 | 10 | nmol/kg | −35.4 | 3.6 | 151.0 | 52.7 | yes 30 min prior to cpd |
| 399 | 30 | nmol/kg | −45.0 | 2.9 | 121.3 | 15.6 | yes 30 min prior to cpd |
| Vehicle | 5 | mL/kg | 11.2 | 6.5 | 12.0 | 8.7 | yes 30 min prior to cpd |
| 203 | 10 | nmol/kg | −29.5 | 4.9 | 54.6 | 17.3 | yes 30 min prior to cpd |
| 203 | 30 | nmol/kg | −36.5 | 5.1 | 106.9 | 19.5 | yes 30 min prior to cpd |
| Vehicle | 5 | mL/kg | 14.4 | 7.0 | 49.9 | 17.7 | yes 30 min prior to cpd |
| 361 | 10 | nmol/kg | −23.0 | 10.6 | 91.5 | 14.5 | yes 30 min prior to cpd |
| 361 | 30 | nmol/kg | −38.1 | 4.7 | 93.8 | 21.1 | yes 30 min prior to cpd |
| Vehicle | 5 | mL/kg | 10.7 | 8.9 | 31.4 | 16.6 | yes 30 min prior to cpd |
| 7 | 1.5 | nmol/kg | 5.4 | 8.3 | 80.8 | 14.3 | yes 30 min prior to cpd |
| 7 | 5 | nmol/kg | −21.2 | 9.8 | 152.9 | 43.0 | yes 30 min prior to cpd |
| Vehicle | 5 | mL/kg | 51.6 | 16.5 | 15.5 | 13.8 | yes 30 min prior to cpd |
| 349 | 10 | nmol/kg | 4.9 | 14.3 | 27.8 | 13.5 | yes 30 min prior to cpd |
| 349 | 30 | nmol/kg | −17.3 | 7.3 | 100.2 | 21.8 | yes 30 min prior to cpd |
| Vehicle | 5 | mL/kg | 19.9 | 9.0 | 39.1 | 10.6 | yes 30 min prior to cpd |
| 224 | 3 | nmol/kg | −46.3 | 4.8 | 100.3 | 34 | yes 30 min prior to cpd |
| 224 | 10 | nmol/kg | −40.0 | 5.0 | 132.3 | 32.6 | yes 30 min prior to cpd |
| Vehicle | 5 | mL/kg | 2.5 | 4.4 | 10.2 | 4.9 | No |
| 229 | 10 | nmol/kg | −36.7 | 5.2 | 14.2 | 9.0 | No |
| 229 | 30 | nmol/kg | −40.6 | 2.3 | 44.5 | 5.3 | No |
| Vehicle | 5 | mL/kg | 7.3 | 9.2 | 19.2 | 4.1 | No |
| 289 | 10 | nmol/kg | −8.5 | 7.7 | 8.9 | 4.6 | No |
| 289 | 30 | nmol/kg | −16.1 | 5.9 | 7.7 | 5.6 | No |

TABLE 17
Glucose Change in GLP-1R KO and WT Mice
Treated with GLP-1/Glucagon Peptides

| | | | | | GLP-1R | | |
|---|---|---|---|---|---|---|---|
| | | | WT % | | KO % | KO % | Somato- |
| | | | glucose | | glucose | glucose | statin |
| | | | change | WT | change - | change | pretreat- |
| Peptide | Dose | | 60 min | SEM | 60 min | SEM | ment? |
| Vehicle | 5 | mL/kg | −4.1 | 4.4 | 16.2 | 7.4 | No |
| 301 | 10 | nmol/kg | −43 | 3.2 | 29.1 | 16.1 | No |
| 301 | 30 | nmol/kg | −35.7 | 4.3 | 17.9 | 7.7 | No |
| Vehicle | 5 | mL/kg | 3.7 | 3.7 | 6.3 | 2.1 | No |
| 140 | 10 | nmol/kg | −32.1 | 2.8 | 46.2 | 6.8 | No |
| 140 | 30 | nmol/kg | −29.0 | 3.0 | 59.8 | 11.4 | No |
| Vehicle | 5 | mL/kg | 9.9 | 6.9 | 2.8 | 3.4 | No |
| 188 | 10 | nmol/kg | −23.2 | 3.9 | 64 | 15.6 | No |
| 188 | 30 | nmol/kg | −31.3 | 2.5 | 44.6 | 11.0 | No |
| Vehicle | 5 | mL/kg | 10.5 | 5.4 | 7.8 | 4.0 | No |
| 195 | 10 | nmol/kg | 1.4 | 3.4 | 35.3 | 5.4 | No |
| 195 | 30 | nmol/kg | −17.0 | 4.9 | 48.0 | 15.1 | No |

### Example 8: Pharmacokinetic Profiling in Pre-Clinical Species

Selected test peptides (n=8) were profiled for pharmacokinetic (PK) properties in the mouse after single intravenous (i.v.) or subcutaneous (s.c.) dose. A subset (n=4) meeting the acceptance criteria was subsequently profiled in the dog (single i.v. dose) and non-human primate (single i.v. and s.c. dose). After administration, blood was sampled up to 48 hrs in the mouse, 120 hrs in the dog and 168 hrs in the non-human primate, at time points selected to accurately determine the full pharmacokinetic profile in each species. Low-binding plasticware containers were used during preparation and sample handling to avoid non-specific binding.

Mouse: PK was studied in lean male C57BL/6 (Peptide 140, Peptide 188, Peptide 195, Peptide 420, Peptide 477 and Peptide 472) or male C57BL/6 DIO pre-conditioned mice on high-fat diet (Peptide 224, Peptide 229). After dosing, PK samples were collected from the dorsal metatarsal vein. Blood of each sample was transferred into plastic micro centrifuge tubes containing EDTA-K2, inverted several times for proper mixing of contents and then placed on wet ice. The blood samples were centrifuged at 4° C. to obtain plasma and stored at −75° C. prior to analysis.

Dog: PK was studied in non-naïve male Beagle dogs. After dosing, PK samples were collected from the jugular vein. Blood of each sample was transferred into plastic micro centrifuge tubes containing EDTA-K2, inverted several times for proper mixing of contents and then placed on wet ice. Plasma was separated by centrifugation and stored frozen in matrix tubes at a temperature set to maintain-20° C. until analysis.

Non-human primate: PK was studied in non-naïve male cynomolgus monkeys. After dosing, PK samples were collected from the femoral vein. Blood of each sample was transferred into plastic micro centrifuge tubes containing EDTA-K2, inverted several times for proper mixing of contents and then placed on wet ice. The blood samples were centrifuged under refrigerated (2° C. to 8° C.) conditions within 30 minutes following sample collection to obtain plasma and stored at −60° C. to −90° C.° C. prior to analysis.

Formulation: Test articles were formulated in 20 mM Sodium Phosphate, 220 mM Sorbitol pH 7.5 (Peptide 224, Peptide 229, Peptide 140, Peptide 188, Peptide 195) or 20 mM sodium acetate 220 mM mannitol pH 4.5 (Peptide 420, Peptide 477, Peptide 472).

Samples analysis: Samples were analyzed by LC-MS/MS. Calibration standards were injected at the beginning and end of each batch and the determined concentration for each prepared standard was used to construct a calibration curve. Plasma and dose aliquot sample concentrations were determined from the plasma calibration curve.

Data analysis: Plasma concentration data was analyzed by non-compartmental analysis (NCA) using Phoenix Winnonlin v. 8.3.3.33. Linear trapezoidal rule was used for increasing values and log trapezoidal rule was used for decreasing values, applying uniform weighting for lambda_z calculations. Subcutaneous bioavailability was calculated as the ratio AUC(s.c.)_{0-inf}/AUC(i.v.)_{0-inf}. Reported values (Table 18) represent the mean of the individual estimates.

Results: Test peptide half-life after intravenous administration and absolute bioavailability after subcutaneous administration calculated by NCA are reported in Table 18. Generally, test peptides displayed pro-longed circulation half-lives in all studied pre-clinical species, and additionally a significant extension in higher order species as compared to rodent. In the mouse, estimated half-lives spanned 2.9-19 hrs, in the dog 86-71 hrs and in non-human primates 49-130 hrs. The bioavailability after subcutaneous administration was generally ≥50%, with comparable levels in the mice and non-human primates. Test peptides evaluated in non-human primates all displayed high bioavailability ≥75%.

TABLE 18
Pre-clinical PK. Half-life after intravenous administration and
absolute subcutaneous bioavailability of profiled test peptides
in mouse, dog and non-human primate at specified doses.

| | | | | Half-life i.v. | S.c. |
|---|---|---|---|---|---|
| | | | Dose | administration | bioavailability |
| Species | Strain | Alias | nmol/kg | h | % |
| Mouse | C57bl/6 | Peptide | 10 | 6.4 | 102 |
| | | 140 | | | |
| | | Peptide | 10 | 7.9 | 98 |
| | | 188 | | | |
| | | Ppetide | 10 | 11 | 66 |
| | | 195 | | | |
| | | Peptide | 10 | 5.7 | 49 |
| | | 420 | | | |
| | | Peptide | 10 | 2.9 | 83 |
| | | 472 | | | |
| | | Peptide | 10 | 8.9 | 50 |
| | | 477 | | | |
| | C57bl/6 | Peptide | 10 | 9.5 | 109 |
| | (DIO) | 224 | | | |
| | | Peptide | 10 | 19 | 86 |
| | | 229 | | | |
| Dog | Beagle | Peptide | 10 | 75 | n.d. |
| | | 224 | | | |
| | | Peptide | 10 | 86 | n.d. |
| | | 229 | | | |
| | | Peptide | 10 | 71 | n.d. |
| | | 188 | | | |
| | | Peptide | 10 | 78 | n.d. |
| | | 195 | | | |
| NHP | Cyno- | Peptide | 10 | 100 | 95 |
| | molgus | 224 | | | |
| | | Peptide | 20 | 130 | 75 |
| | | 229 | | | |
| | | Peptide | 10 | 67 | 79 |
| | | 188 | | | |
| | | Peptide | 10 | 49 | 77 |
| | | 195 | | | |

n.d.: not determined.

1-79. (canceled)

80. A peptide comprising the sequence: H-X2-X3-G-X5-X6-T-S-D-X10-S-X12-αMethyl-Phenylalanine (αMePhe)-L-X15-X16-X17-X18-A-X20-X21-X22-X23-X24-X25-X26-X27-X28-X29-X30-X31-Z, wherein
X2 is Aminoisobutyric acid (Aib), S, or A,
X3 is Q, H, or E,
X5 is T or S,
X6 is F or αMePhe,
X10 is V, K or Y,
X12 is K, E, or S,
X15 is D or E,
X16 is T, S, or G,
X17 is K, R, E, or Q,
X18 is R or A,
X20 is R, K, or Q,
X21 is D or E,
X22 is αMePhe or F,
X23 is V or I,
X24 is Q or A,
X25 is Aib or W,
X26 is L or I,
X27 is L, A, E, V, or M,
X28 is E, N, A, R, or K,
X29 is Aib, T, or G,
X30 is G, R, or not present,
X31 is G or not present, and
Z is amide or acid (SEQ ID NO: 540).

81. The peptide of claim 80, wherein X2 is Aib, X12 is K, and X24 is Q.

82. The peptide of claim 80, wherein X16 is T, X17 is K, X27 is L, X28 is E, and X29 is Aib.

83. The peptide of claim 80, wherein X3 is Q, X5 is T, X6 is F, X10 is V, X12 is K, X15 is D, X16 is T, X17 is K, X18 is R, X20 is R, X21 is D, X22 is F, X23 is V, X24 is Q, X25 is W, X26 is L, X27 is L, X28 is E, X29 is Aib, X30 is G, X31 is not present, and Z is acid.

84. The peptide of claim 80, wherein X3 is H, X5 is S, X6 is αMePhe, X10 is V, X12 is K, X15 is D, X16 is S, X17 is R, X18 is A, X20 is K, X21 is D, X22 is αMePhe, X23 is V, X24 is Q, X25 is Aib, X26 is I, X27 is A, X28 is N, X29 is T, X30 is not present, X31 is not present, and Z is amide.

85. The peptide of claim 80, wherein one or more lysine residues are acylated.

86. The peptide of claim 85, wherein the lysine at position 17 is acylated.

87. The peptide of claim 85, wherein the lysine at position 20 is acylated.

88. The peptide of claim 80, wherein one or more lysine resides are lipidated.

89. The peptide of claim 88, wherein the lysine at position 17 is lipidated.

90. The peptide of claim 88, wherein the lysine at position 20 is lipidated.

91. The peptide of claim 88, wherein the lipid is selected from the group consisting of octadecanedioic acid (C18diacid) and icosanedioic acid (C20diacid).

92. The peptide of claim 91, wherein the lipid is octadecanedioic acid (C18diacid).

93. The peptide of claim 91, wherein the lipid is icosanedioic acid (C20diacid).

94. The peptide of claim 88, wherein the lipid is linked to the epsilon amino group of lysine at position 17 or 20 via a linker.

95. The peptide of claim 94, wherein the linker is ((O2Oc)-(O2Oc)-γE) or ((O2Oc)-(O2Oc)-γE-γE) in the C- to N-terminal orientation.

96. The peptide of claim 94, wherein the linker is ((O2Oc)-(O2Oc)-γE) in the C- to N-terminal orientation.

97. The peptide of claim 95, wherein the linker is ((O2Oc)-(O2Oc)-γE-γE) in the C- to N-terminal orientation.

98. The peptide of claim 94, wherein the linker is linked to the epsilon amino group of the residue at position 17 or 20.

99. The peptide of claim 80, wherein the peptide binds to a GLP-1 receptor (GLP-1R), binds to a glucagon receptor (GCGR), or binds to both a GLP-1 receptor and a glucagon receptor.

100. The peptide of claim 99, wherein the GLP-1R is a human GLP-1R.

101. The peptide of claim 99, wherein the GCGR is a human GCGR.

102. The peptide of claim 80, wherein the peptide is an agonist of GLP-1 activity, an agonist of glucagon activity, or an agonist of both GLP-1 and glucagon activity.

103. The peptide of claim 80, wherein the peptide has increased proteolytic-resistance relative to a natural ligand of the GLP-1R and/or GCGR.

104. The peptide of claim 80, wherein the peptide is isolated.

105. The peptide of claim 80, wherein the peptide has at least 50%, at least 60%, at least 70%, at least 75%, at least 80%, at least 85%, at least 90%, at least 95, or 100% of intact peptide remaining after incubation with a protease at 37° C. for 5 min, 10 min, 15 min, 30 min, 2 hr, 4 hr or 24 hr.

106. The peptide of claim 105, wherein the protease is selected from the group consisting of neprilysin, pepsin, pancreatin, simulated gastric fluid with pepsin, and simulated intestinal fluid with pancreatin.

107. The peptide of claim 80, wherein the peptide has a half-life in cynomolgus monkeys after intravenous administration of at least 45 hours, at least 50 hours, at least 60 hours, at least 70 hours, at least 80 hours, at least 90 hours, at least 100 hours, at least 110 hours, at least 120 hours, or about 130 hours.

108. The peptide of claim 80, wherein the peptide has an s.c. bioavailability in cynomolgus monkeys of at least 75%, at least 80%, at least 90%, or about 95%.

109. A pharmaceutical composition comprising the peptide of claim 80.

110. The composition of claim 109, wherein the composition is a solid composition.

111. The composition of claim 109, wherein the composition is a liquid composition.


SEQUENCE LISTING (541 sequences)

SEQ ID NO: 1 - protein, 29 aa, synthetic construct
HSQGTFTSDYSKYLDSRRAQDFVQWLMNT

SEQ ID NO: 2 - protein, 30 aa, synthetic construct
HAEGTFTSDVSSYLEGQAAKEFIAWLVKGR

SEQ ID NO: 3 - protein, 31 aa, synthetic construct
HAEGTFTSDVSSYLEGQAAKEFIAWLVKGRG

SEQ ID NO: 4-5 - skipped (2 entries)

SEQ ID NO: 6 - protein, 28 aa, synthetic construct
  MOD_RES 6: note = alpha-methyl-L-phenylalanine
  MOD_RES 13: note = 2-Aminoisobutyric acid
  MOD_RES 17: note = Lys, wherein the side chain of Lys is connected to O2Oc-O2Oc-gamma-Glu-C18diacid
  MOD_RES 22: note = alpha-methyl-L-phenylalanine
  MOD_RES 25: note = 2-Aminoisobutyric acid
HSQGSXTSDVSKXLDSXAAQDXVQXIAN

SEQ ID NO: 7 - protein, 29 aa, synthetic construct
  MOD_RES 6: note = alpha-methyl-L-phenylalanine
  MOD_RES 13: note = 2-Aminoisobutyric acid
  MOD_RES 17: note = Lys, wherein the side chain of Lys is connected to O2Oc-O2Oc-gamma-Glu-C20diacid
  MOD_RES 22: note = alpha-methyl-L-phenylalanine
HSQGSXTSDVSKXLDSXAAQDXVQWIANT

SEQ ID NO: 8 - protein, 29 aa, synthetic construct
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to gamma-Glu-C18diacid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-C18diacid
HSQGSXTSDVSKXLDSXAAQDFVQWIANT

SEQ ID NO: 9 - protein, 29 aa, synthetic construct
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-C18diacid
HSQGSXTSDVSKXLDSXAAQDFVQWIANT

SEQ ID NO: 10 - protein, 28 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu- gamma-Glu-C18diacid
  MOD_RES 22: note = alpha-methyl-L-phenylalanine
  MOD_RES 25: note = 2-Aminoisobutyric acid
HXQGSXTSDVSKXLDSXAAQDXVQXIAN

SEQ ID NO: 11 - protein, 28 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 22: note = alpha-methyl-L-phenylalanine
HXHGSXTSDVSKXLDSXAAQDXVEWIAN

SEQ ID NO: 12 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to gamma-Glu-C18diacid
HXHGSXTSDVSKXLDSXAAQDXVEWIANT

SEQ ID NO: 13 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-C18diacid
HXHGSXTSDVSKXLDSXAAQDFVQWIANT

SEQ ID NO: 14 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-C18diacid
HXGSMXTSDVSKXLDSXAARDFVQWIANT

SEQ ID NO: 15 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-C18diacid
HXHGSXTSDVSKXLDSXAARDFVEWIANT

SEQ ID NO: 16 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 25: note = 2-Aminoisobutyric acid
HXHGSXTSDVSKXLDSXAAQDFVQXIANT

SEQ ID NO: 17 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 25: note = 2-Aminoisobutyric acid
HXHGSXTSDVSKXLDSXAARDFVQXIANT

SEQ ID NO: 18 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 22: note = alpha-methyl-L-phenylalanine
  MOD_RES 25: note = 2-Aminoisobutyric acid
HXHGSXTSDVSKXLDSXAARDXVQXIANT

SEQ ID NO: 19 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-gamma-Glu-gamma-Glu-C20diacid
  MOD_RES 25: note = 2-Aminoisobutyric acid
  MOD_RES 22: note = alpha-methyl-L-phenylalanine
HXHGSXTSDVSKXLDSXAARDXVQXIANT

SEQ ID NO: 20 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 22: note = alpha-methyl-L-phenylalanine
  MOD_RES 25: note = 2-Aminoisobutyric acid
HXHGSXTSDVSKXLDSXAARDXVEXIANT

SEQ ID NO: 21 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 22: note = alpha-methyl-L-phenylalanine
  MOD_RES 25: note = 2-Aminoisobutyric acid
HXHGSXTSDVSKXLDSXAARDXIAXIANT

SEQ ID NO: 22 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 22: note = alpha-methyl-L-phenylalanine
HXHGSXTSDVSKXLDSXAARDXVESIANT

SEQ ID NO: 23 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 22: note = alpha-methyl-L-phenylalanine
HXHGSXTSDVSKXLDSXAARDXVQSIANT

SEQ ID NO: 24 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-gamma-Glu-gamma-Glu-C18diacid
  MOD_RES 22: note = alpha-methyl-L-phenylalanine
  MOD_RES 25: note = 2-Aminoisobutyric acid
HXHGSXTSDVSKXLDSXAARDXVQXIANT

SEQ ID NO: 25 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-gamma-Glu-gamma-Glu-C18diacid
  MOD_RES 22: note = alpha-methyl-L-phenylalanine
  MOD_RES 25: note = 2-Aminoisobutyric acid
HXHGSXTSDVSKXLDSXAARDXVEXIANT

SEQ ID NO: 26 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to gamma-Glu-C18diacid
  MOD_RES 22: note = alpha-methyl-L-phenylalanine
HXHGSXTSDVSKXLDSXAAQDXVESIANT

SEQ ID NO: 27 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C20diacid
  MOD_RES 22: note = alpha-methyl-L-phenylalanine
HXHGSXTSDVSKXLDSXAAQDXVESIANT

SEQ ID NO: 28 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 22: note = alpha-methyl-L-phenylalanine
HXHGSXTSDVSKXLDSXAAQDXVESIANT

SEQ ID NO: 29 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 22: note = alpha-methyl-L-phenylalanine
  MOD_RES 25: note = 2-Aminoisobutyric acid
HXHGSXTSDVSKXLDSXAAQDXVQXIANT

SEQ ID NO: 30 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C20diacid
  MOD_RES 22: note = alpha-methyl-L-phenylalanine
  MOD_RES 25: note = 2-Aminoisobutyric acid
HXHGSXTSDVSKXLDSXAAQDXVQXIANT

SEQ ID NO: 31 - protein, 28 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 22: note = alpha-methyl-L-phenylalanine
HXQGSXTSDVSKXLDSXAAQDXVQWIAN

SEQ ID NO: 32 - protein, 28 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 22: note = alpha-methyl-L-phenylalanine
HXQGSXTSDVSKXLDSXAAQDXVEWIAN

SEQ ID NO: 33 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRAQDFVRWLLXT

SEQ ID NO: 34 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 20: note = 2-Aminoisobutyric acid
  MOD_RES 28: note = 2-Aminoisobutyric acid
HXQGTFTSDVSKXLDSXRAXDFVQWLLXT

SEQ ID NO: 35 - protein, 31 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRAQDFVRWLLEXTG

SEQ ID NO: 36 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLDSXRAQDFVRWLLEXG

SEQ ID NO: 37 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to gamma-Glu-C18diacid
  MOD_RES 20: note = 2-Aminoisobutyric acid
HXQGTFTSDVSKXLDSXRAXDFVQWIANT

SEQ ID NO: 38 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRAQDFVQWLEXT

SEQ ID NO: 39 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRAQDFVQWLLXE

SEQ ID NO: 40 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDTXRAQDFVQWLLXT

SEQ ID NO: 41 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRARDFVQWLLXT

SEQ ID NO: 42 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRAEDFVQWLLXT

SEQ ID NO: 43 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRAQDFVEWLLXT

SEQ ID NO: 44 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRAQDFVRWLLXTE

SEQ ID NO: 45 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRAQDFVQWLLXT

SEQ ID NO: 46 - protein, 28 aa, synthetic construct
  MOD_RES 2: note = 2-Aminoisobutyric acid
  MOD_RES 13: note = alpha-methyl-L-phenylalanine
  MOD_RES 28: note = polyethylene glycol
  MOD_RES 17: note = Lys, wherein the side chain of Lys is connected to O2Oc-O2Oc-gamma-Glu-C18diacid
HXQGTFTSDYSKXLDSXRAQDFVQWLVX

SEQ ID NO: 47 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 25: note = 2-Aminoisobutyric acid
HXQGTFTSDVSKXLDSXRAQDFVQXLVAT

SEQ ID NO: 48 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to gamma-Glu-gamma-Glu-O2Oc-O2Oc-gamma-Glu-gamma-Glu-C18diaci d
  MOD_RES 29: note = 2-Aminoisobutyric acid
HXQGTFTSDVSKXLDSXRARDFVRWLLEXG

SEQ ID NO: 49 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to gamma-Glu-gamma-Glu-O2Oc-O2Oc-gamma-Glu-gamma-Glu-C18diaci d
  MOD_RES 30: note = 2-Aminoisobutyric acid
HXQGTFTSDVSKXLDSXRAQDFVRWLLEGX

SEQ ID NO: 50 - protein, 30 aa, synthetic construct; identical to SEQ ID NO: 36
  MOD_RES 2: note = 2-Aminoisobutyric acid
  MOD_RES 13: note = alpha-methyl-L-phenylalanine
  MOD_RES 17: note = Lys, wherein the side chain of Lys is connected to gamma-Glu-gamma-Glu-O2Oc-O2Oc-gamma-Glu-gamma-Glu-C18diaci d
  MOD_RES 29: note = 2-Aminoisobutyric acid

SEQ ID NO: 51 - protein, 30 aa, synthetic construct; identical to SEQ ID NO: 36
  MOD_RES 2: note = 2-Aminoisobutyric acid
  MOD_RES 13: note = alpha-methyl-L-phenylalanine
  MOD_RES 17: note = Lys, wherein the side chain of Lys is connected to gamma-Glu-gamma-Glu-O2Oc-O2Oc-gamma-Glu-gamma-Glu=C18diaci d
  MOD_RES 29: note = 2-Aminoisobutyric acid

SEQ ID NO: 52 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLDTXRAQDFVRWLLEXG

SEQ ID NO: 53 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLDSXRAQDFVAWLLEXG

SEQ ID NO: 54 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLDSXRARDFVAWLLEXG

SEQ ID NO: 55 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLDSXRARDFVQWLLEXG

SEQ ID NO: 56 - protein, 30 aa, synthetic construct; identical to SEQ ID NO: 48
  MOD_RES 2: note = 2-Aminoisobutyric acid
  MOD_RES 13: note = alpha-methyl-L-phenylalanine
  MOD_RES 17: note = Lys, wherein the side chain of Lys is connected to gamma-Glu-gamma-Glu-O2Oc-O2Oc-gamma-Glu-gamma-Glu-C18diaci d
  MOD_RES 29: note = 2-Aminoisobutyric acid

SEQ ID NO: 57 - protein, 31 aa, synthetic construct
  MOD_RES 2: note = 2-Aminoisobutyric acid
  MOD_RES 17: note = Lys, wherein the side chain of Lys is connected to O2Oc-O2Oc-gamma-Glu-C18diacid
  MOD_RES 13: note = alpha-methyl-L-phenylalanine
HXQGTFTSDVSKXLDSXRAQDFVRWLASRGI

SEQ ID NO: 58 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDSXRAQDFVRWLASR

SEQ ID NO: 59 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLDSXRAQDFVRWLEAXG

SEQ ID NO: 60 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLDSXRAQDFVRWIAEXG

SEQ ID NO: 61 - protein, 30 aa, synthetic construct
  MOD_RES 2: note = 2-Aminoisobutyric acid
  MOD_RES 13: note = alpha-methyl-L-phenylalanine
  MOD_RES 17: note = O2Oc-O2Oc-gamma-Glu-C18diacid
  MOD_RES 29: note = 2-Aminoisobutyric acid
HXQGTFTSDVSKXLDSXRAQDFVRWVVEXG

SEQ ID NO: 62 - protein, 30 aa, synthetic construct
  MOD_RES 2: note = 2-Aminoisobutyric acid
  MOD_RES 14: note = alpha-methyl-L-phenylalanine
  MOD_RES 17: note = O2Oc-O2Oc-gamma-Glu-C18diacid
  MOD_RES 28: note = 2-Aminoisobutyric acid
HXQGTFTSDVSKXLDSXRAQDFVQWLLXTE

SEQ ID NO: 63 - protein, 30 aa, synthetic construct; identical to SEQ ID NO: 44
  MOD_RES 2: note = 2-Aminoisobutyric acid
  MOD_RES 13: note = alpha-methyl-L-phenylalanine
  MOD_RES 17: note = O2Oc-O2Oc-gamma-Glu-C18diacid
  MOD_RES 28: note = 2-Aminoisobutyric acid

SEQ ID NO: 64 - protein, 29 aa, synthetic construct
  MOD_RES 2: note = 2-Aminoisobutyric acid
  MOD_RES 13: note = alpha-methyl-L-phenylalanine
  MOD_RES 17: note = O2Oc-O2Oc-gamma-Glu-C18diacid
  MOD_RES 28: note = 2-Aminoisobutyric acid
HXQGTFTSDVSKXLDSXRAQDFVRWLLXE

SEQ ID NO: 65 - protein, 29 aa, synthetic construct
  MOD_RES 2: note = 2-Aminoisobutyric acid
  MOD_RES 13: note = alpha-methyl-L-phenylalanine
  MOD_RES 17: note = O2Oc-O2Oc-gamma-Glu-C20diacid
  MOD_RES 28: note = 2-Aminoisobutyric acid
HXQGTFTSDVSKXLDSXRAQDFVRWLLXE

SEQ ID NO: 66 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to gamma-Glu-C18diacid
  MOD_RES 28: note = 2-Aminoisobutyric acid
HXQGTFTSDVSKXLDSXRAQDFVQWLLXT

SEQ ID NO: 67 - protein, 30 aa, synthetic construct; identical to SEQ ID NO: 36
  MOD_RES 2: note = 2-Aminoisobutyric acid
  MOD_RES 13: note = alpha-methyl-L-phenylalanine
  MOD_RES 17: note = Lys, wherein the side chain of Lys is connected to gamma-Glu-C18diacid
  MOD_RES 29: note = 2-Aminoisobutyric acid

SEQ ID NO: 68 - protein, 30 aa, synthetic construct; identical to SEQ ID NO: 49
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 30: note = 2-Aminoisobutyric acid

SEQ ID NO: 69 - protein, 30 aa, synthetic construct; identical to SEQ ID NO: 49
  MOD_RES 2: note = 2-Aminoisobutyric acid
  MOD_RES 13: note = alpha-methyl-L-phenylalanine
  MOD_RES 17: note = Lys, wherein the side chain of Lys is connected to gamma-Glu-C18diacid
  MOD_RES 30: note = 2-Aminoisobutyric acid

SEQ ID NO: 70 - protein, 30 aa, synthetic construct; identical to SEQ ID NO: 36
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
  MOD_RES 29: note = 2-Aminoisobutyric acid

SEQ ID NO: 71 - protein, 30 aa, synthetic construct; identical to SEQ ID NO: 52
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
  MOD_RES 29: note = 2-Aminoisobutyric acid

SEQ ID NO: 72 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLESXRAQDFVRWLLEXG

SEQ ID NO: 73 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLEAXRARDFVAWLLEXG

SEQ ID NO: 74 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRAQDFVRWIAXTE

SEQ ID NO: 75 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLDSXRAQDFVRWLLEXA

SEQ ID NO: 76 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 30: note = 2-Aminoisobutyric acid
HXQGTFTSDVSKXLDSXRAQDFVRWLLEAX

SEQ ID NO: 77 - protein, 30 aa, synthetic construct
  MOD_RES 2: note = 2-Aminoisobutyric acid
  MOD_RES 23: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRARDFVRWLLXTE

SEQ ID NO: 78 - protein, 30 aa, synthetic construct; identical to SEQ ID NO: 77
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid

SEQ ID NO: 79 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRARDFVAWLLXTE

SEQ ID NO: 80 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRALDFVRWLLXTE

SEQ ID NO: 81 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLELXRAQDFVRWLLXTE

SEQ ID NO: 82 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDEXRAQDFVRWLLXTE

SEQ ID NO: 83 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRAQDFVRWLEXTE

SEQ ID NO: 84 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLESXRAQDFVRWLLXTE

SEQ ID NO: 85 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLESXRAQDFVRWLLXE

SEQ ID NO: 86 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDTXRAQDFVRWLLXTE

SEQ ID NO: 87 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRAQDFVRWLAXTE

SEQ ID NO: 88 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRAQDFVRWLLXTA

SEQ ID NO: 89 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRAQDFVRWLLXTK

SEQ ID NO: 90 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDKXRAQDFVRWLLXTE

SEQ ID NO: 91 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDSXRAQDFVRWLLEAG

SEQ ID NO: 92 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLDSXRAQDFVRWLLAXG

SEQ ID NO: 93 - protein, 30 aa, synthetic construct; identical to SEQ ID NO: 36
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-C18diacid
  MOD_RES 29: note = 2-Aminoisobutyric acid

SEQ ID NO: 94 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLDKXRAQDFVRWLLEXG

SEQ ID NO: 95 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLDSXRAQDFVRWLLEXK

SEQ ID NO: 96 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-C18diacid
  MOD_RES 29: note = 2-Aminoisobutyric acid
HXQGTFTSDVSKXLDKXRAQDFVRWLLAXK

SEQ ID NO: 97 - protein, 30 aa, synthetic construct; identical to SEQ ID NO: 52
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid

SEQ ID NO: 98 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDTXRAQDFVRWLLEAG

SEQ ID NO: 99 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLDTXRARDFVRWLVEXG

SEQ ID NO: 100 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLDTXRARDFVQWLLEXG

SEQ ID NO: 101 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLDTXRARDFVRWLLEXG

SEQ ID NO: 102 - protein, 30 aa, synthetic construct
  MOD_RES 2: note = 2-Aminoisobutyric acid
  MOD_RES 13: note = alpha-methyl-L-phenylalanine
  MOD_RES 17: note = O2Oc-O2Oc-gamma-Glu-C20diacid
  MOD_RES 29: note = 2-Aminoisobutyric acid
HXQGTFTSDVSKXLDTXRAQDFVRWLVEXG

SEQ ID NO: 103 - protein, 30 aa, synthetic construct; identical to SEQ ID NO: 102
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid

SEQ ID NO: 104 - protein, 29 aa, synthetic construct
  MOD_RES 2: note = 2-Aminoisobutyric acid
  MOD_RES 13: note = alpha-methyl-L-phenylalanine
  MOD_RES 17: note = O2Oc-O2Oc-gamma-Glu-C18diacid
  MOD_RES 29: note = 2-Aminoisobutyric acid
HXQGTFTSDVSKXLDTXRAQDFVRWLLXE

SEQ ID NO: 105 - protein, 29 aa, synthetic construct
  MOD_RES 2: note = 2-Aminoisobutyric acid
  MOD_RES 13: note = alpha-methyl-L-phenylalanine
  MOD_RES 17: note = O2Oc-O2Oc-gamma-Glu-C20diacid
  MOD_RES 28: note = 2-Aminoisobutyric acid
HXQGTFTSDVSKXLDTXRAQDFVRWLLXE

SEQ ID NO: 106 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C20diacid
  MOD_RES 28: note = 2-Aminoisobutyric acid
HXQGTFTSDVSKXLDTXRAQDFVRWLLXE

SEQ ID NO: 107 - protein, 30 aa, synthetic construct; identical to SEQ ID NO: 100
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
  MOD_RES 29: note = 2-Aminoisobutyric acid

SEQ ID NO: 108 - protein, 29 aa, synthetic construct
  METAL 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDSXRAQDFVRWLIXE

SEQ ID NO: 109 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDTXRARDFVRWLLXE

SEQ ID NO: 110 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 28: note = 2-Aminoisobutyric acid
HXQGTFTSDVSKXLDTXRARDFIAWLLXE

SEQ ID NO: 111 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLDTXRARDFIAWLLEXG

SEQ ID NO: 112 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLDTXRARDFVAWLLEXG

SEQ ID NO: 113 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 29: note = 2-Aminoisobutyric acid
HXQGTFTSDVSKXLDTXRARDFVAWLEAXG

SEQ ID NO: 114 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
  MOD_RES 29: note = 2-Aminoisobutyric acid
HXQGTFTSDVSKXLDTXRARDFVQWLEAXG

SEQ ID NO: 115 - protein, 30 aa, synthetic construct
  MOD_RES 2: note = 2-Aminoisobutyric acid
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 4-gamma-Glu-gamma-Glu-Palmitoyl
  MOD_RES 22: note = alpha-methyl-L-phenylalanine
  MOD_RES 25: note = 2-Aminoisobutyric acid
HXQGSXTSDVSKXLDSXRAQDXVEXLEAGG

SEQ ID NO: 116 - protein, 30 aa, synthetic construct
  MOD_RES 2: note = 2-Aminoisobutyric acid
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 2-polyethylene glycol 2-gamma-Glu-gamma-Glu-Stearoyl
  MOD_RES 22: note = alpha-methyl-L-phenylalanine
  MOD_RES 25: note = 2-Aminoisobutyric acid
HXQGSXTSDVSKXLDSXAAQDXVEXLEAGG

SEQ ID NO: 117 - protein, 29 aa, synthetic construct
  MOD_RES 2: note = 2-Aminoisobutyric acid
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 2-polyethylene glycol 2-gamma-Glu-gamma-Glu-Stearoyl
  MOD_RES 22: note = alpha-methyl-L-phenylalanine
  MOD_RES 25: note = 2-Aminoisobutyric acid
HXQGSXTSDVSKXLDSXAAQDXVEXLANT

SEQ ID NO: 118 - protein, 30 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 2-polyethylene glycol 2-gamma-Glu-gamma-Glu-Stearoyl
  MOD_RES 22: note = alpha-methyl-L-phenylalanine
  MOD_RES 25: note = 2-Aminoisobutyric acid
HSQGSXTSDVSKXLDSXAAQDXVEXLEAGG

SEQ ID NO: 119 - protein, 29 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 2-polyethylene glycol 2-gamma-Glu-gamma-Glu-Stearoyl
  MOD_RES 22: note = alpha-methyl-L-phenylalanine
  MOD_RES 25: note = 2-Aminoisobutyric acid
HSQGSXTSDVSKXLDSXAAQDXVEXLANT

SEQ ID NO: 120 - protein, 29 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 2-polyethylene glycol 2-gamma-Glu-gamma-Glu-Stearoyl
  MOD_RES 22: note = alpha-methyl-L-phenylalanine
  MOD_RES 25: note = 2-Aminoisobutyric acid
HSQGSXTSDVSKXLDSXAAQDXVEXLISG

SEQ ID NO: 121 - protein, 29 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 2-polyethylene glycol 2-gamma-Glu-gamma-Glu-Stearoyl
  MOD_RES 22: note = alpha-methyl-L-phenylalanine
  MOD_RES 25: note = 2-Aminoisobutyric acid
HSQGSXTSDVSKXLDSXAAQDXVEXIANT

SEQ ID NO: 122 - protein, 29 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 2-polyethylene glycol 2-gamma-Glu-gamma-Glu-Stearoyl
  MOD_RES 22: note = alpha-methyl-L-phenylalanine
  MOD_RES 25: note = 2-Aminoisobutyric acid
HSQGSXTSDVSKXLDSXAAQDXVEXIINT

SEQ ID NO: 123 - protein, 29 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 2-polyethylene glycol 2-gamma-Glu-gamma-Glu-Stearoyl
  MOD_RES 22: note = alpha-methyl-L-phenylalanine
  MOD_RES 25: note = 2-Aminoisobutyric acid
HSQGSXTSDVSKXLDSXAAQDXVEXLLNT

SEQ ID NO: 124 - protein, 29 aa, synthetic construct
  MOD_RES 26: note = cyclohexylpropanoic acid
HSQGSXTSDVSKXLDSXAAQDXVEXXANT

SEQ ID NO: 125 - protein, 30 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 2-polyethylene glycol 2-gamma-Glu-gamma-Glu-Stearoyl
  MOD_RES 22: note = alpha-methyl-L-phenylalanine
  MOD_RES 25: note = 2-Aminoisobutyric acid
HSQGSXTSDVSKXLDSXAAQDXVEXVVEGG

SEQ ID NO: 126 - protein, 28 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 2-polyethylene glycol 2-gamma-Glu-gamma-Glu-Stearoyl
  MOD_RES 22: note = alpha-methyl-L-phenylalanine
  MOD_RES 25: note = diphenylpropanoic acid
HSQGSXTSDVSKXLDSXAAQDXVQXLEA

SEQ ID NO: 127 - protein, 29 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 4-gamma-Glu-gamma-Glu-Stearoyl
  MOD_RES 22: note = alpha-methyl-L-phenylalanine
  MOD_RES 25: note = 2-Aminoisobutyric acid
HSQGSXTSDVSKXLDSXAAQDXVEXIINT

SEQ ID NO: 128 - protein, 29 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 4-gamma-Glu-gamma-Glu-Stearoyl
  MOD_RES 22: note = alpha-methyl-L-phenylalanine
  MOD_RES 25: note = 2-Aminoisobutyric acid
HSQGSXTSDVSKXLDSXAAQDXVEXIINT

SEQ ID NO: 129 - protein, 29 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to-polyethylene glycol 8-gamma-Glu-gamma-Glu-Stearoyl
  MOD_RES 22: note = alpha-methyl-L-phenylalanine
  MOD_RES 25: note = 2-Aminoisobutyric acid
HSQGSXTSDVSKXLDSXAAQDXVEXIINT

SEQ ID NO: 130 - protein, 29 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 12-gamma-Glu-gamma-Glu-Stearoyl
  MOD_RES 22: note = alpha-methyl-L-phenylalanine
  MOD_RES 25: note = 2-Aminoisobutyric acid
HSQGSXTSDVSKXLDSXAAQDXVEXIINT

SEQ ID NO: 131 - protein, 29 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to gamma-Glu-gamma-Glu-polyethylene glycol 2-polyethylene glycol 2-Stearoyl
  MOD_RES 25: note = 2-Aminoisobutyric acid
  MOD_RES 22: note = alpha-methyl-L-phenylalanine
HSQGSXTSDVSKXLDSXAAQDXVEXIINT

SEQ ID NO: 132 - protein, 29 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to gamma-Glu- polyethylene glycol 2-gamma-Glu-polyethylene glycol 2-Stearoyl
  MOD_RES 22: note = alpha-methyl-L-phenylalanine
  MOD_RES 25: note = 2-Aminoisobutyric acid
HSQGSXTSDVSKXLDSXAAQDXVEXIINT

SEQ ID NO: 133 - protein, 29 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 2-gamma-Glu-polyethylene glycol 2-gamma-Glu-Stearoyl
  MOD_RES 22: note = alpha-methyl-L-phenylalanine
  MOD_RES 25: note = 2-Aminoisobutyric acid
HSQGSXTSDVSKXLDSXAAQDXVEXIINT

SEQ ID NO: 134 - protein, 29 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 2-polyethylene glycol 2-gamma-Glu-gamma-Glu-Lauryl
  MOD_RES 22: note = alpha-methyl-L-phenylalanine
  MOD_RES 25: note = 2-Aminoisobutyric acid
HSQGSXTSDVSKXLDSXAAQDXVEXIINT

SEQ ID NO: 135 - protein, 29 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 2-polyethylene glycol 2-gamma-Glu-gamma-Glu-Myristyl
  MOD_RES 22: note = alpha-methyl-L-phenylalanine
  MOD_RES 25: note = 2-Aminoisobutyric acid
HSQGSXTSDVSKXLDSXAAQDXVEXIINT

SEQ ID NO: 136 - protein, 29 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 2-polyethylene glycol 2-gamma-Glu-gamma-Glu-Stearoyl
  MOD_RES 22: note = alpha-methyl-L-phenylalanine
  MOD_RES 25: note = 2-Aminoisobutyric acid
HSQGSXTSDVSKXLDSXAAQDXVEXIINT

SEQ ID NO: 137 - protein, 29 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 2-polyethylene glycol 2-gamma-Glu-gamma-Glu-C18diacid
  MOD_RES 22: note = alpha-methyl-L-phenylalanine
  MOD_RES 25: note = 2-Aminoisobutyric acid
HSQGSXTSDVSKXLDSXAAQDXVEXIINT

SEQ ID NO: 138 - protein, 28 aa, synthetic construct
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 2-polyethylene glycol 2-gamma-Glu-gamma-Glu-Stearyl
  MOD_RES 22: note = alpha-methyl-L-phenylalanine
  MOD_RES 25: note = 2-Aminoisobutyric acid
HXQGSXTSDVSKXLDSXAAQDXVQXIAN

SEQ ID NO: 139 - protein, 28 aa, synthetic construct
  MOD_RES 2: note = 2-Aminoisobutyric acid
  MOD_RES 6: note = alpha-methyl-L-phenylalanine
  MOD_RES 13: note = diphenylpropanoic acid
  MOD_RES 17: note = Lys, wherein the side chain of Lys is connected to polyethylene glycol 2-polyethylene glycol 2-gamma-Glu-Palmitoyl
  MOD_RES 22: note = alpha-methyl-L-phenylalanine
  MOD_RES 25: note = 2-Aminoisobutyric acid
HXQGSXTSDVSKXLDSXAAQDXVQXIAN

SEQ ID NO: 140 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to polyethylene glycol 4-gamma-Glu-Palmitoy
  MOD_RES 22: note = alpha-methyl-L-phenylalanine
  MOD_RES 27: note = aminohexanoic acid
HXQGTFTSDVSKXLDVXRAQDXVEXLXET

SEQ ID NO: 141 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to polyethylene glycol 2-polyethylene glycol 2-gamma-Glu-gamma-Glu-Stearyl
  MOD_RES 22: note = alpha-methyl-L-phenylalanine
  MOD_RES 25: note = 2-Aminoisobutyric acid
  MOD_RES 27: note = aminohexanoic acid
HXQGTFTSDVSKXLDSXRAQDXVQXLXET

SEQ ID NO: 142 - protein, 30 aa, synthetic construct; identical to SEQ ID NO: 115
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 4-gamma-Glu-gamma-Glu-Stearoyl
  MOD_RES 22: note = alpha-methyl-L-phenylalanine
  MOD_RES 25: note = 2-Aminoisobutyric acid

SEQ ID NO: 143 - protein, 30 aa, synthetic construct; identical to SEQ ID NO: 116
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = polyethylene glycol 4-gamma-Glu-gamma-Glu-Stearoyl
  MOD_RES 22: note = alpha-methyl-L-phenylalanine
  MOD_RES 25: note = 2-Aminoisobutyric acid

SEQ ID NO: 144 - protein, 30 aa, synthetic construct; identical to SEQ ID NO: 116
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 4-gamma-Glu-gamma-Glu- Palmitoyl
  MOD_RES 22: note = alpha-methyl-L-phenylalanine
  MOD_RES 25: note = 2-Aminoisobutyric acid

SEQ ID NO: 145 - protein, 28 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Palmitoyl
  MOD_RES 22: note = alpha-methyl-L-phenylalanine
  MOD_RES 25: note = 2-Aminoisobutyric acid
HXQGSXTSDVSKXLDSXAAQDXVQXIAN

SEQ ID NO: 146 - protein, 30 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Stearoyl
  MOD_RES 18: note = dimethylGln
  MOD_RES 22: note = alpha-methyl-L-phenylalanine
  MOD_RES 25: note = 2-Aminoisobutyric acid
HXQGSXTSDVSKXLDSXAQDXXVEXLEAGG

SEQ ID NO: 147 - protein, 28 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Palmitoy
HXQGSXTSDVSKXLDSXAAQDIVQIIAN

SEQ ID NO: 148 - protein, 30 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu-Stearoyl
  MOD_RES 22: note = alpha-methyl-L-phenylalanine
  MOD_RES 25: note = 2-Aminoisobutyric acid
HXQGSXTSDVSKXLDSXAAQDXVQXVVEGG

SEQ ID NO: 149 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu-Stearoyl
  MOD_RES 22: note = alpha-methyl-L-phenylalanine
  MOD_RES 25: note = 2-Aminoisobutyric acid
HXQGSXTSDVSKXLDSXAAQDXVQXLISG

SEQ ID NO: 150 - protein, 30 aa, synthetic construct
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu-Stearoyl
  MOD_RES 22: note = alpha-methyl-L-phenylalanine
  MOD_RES 25: note = 2-Aminoisobutyric acid
HSQGSXTSDVSKXLDSXAAQDXVQXLEAGG

SEQ ID NO: 151 - protein, 30 aa, synthetic construct; identical to SEQ ID NO: 150
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu-Palmitoyl
  MOD_RES 22: note = alpha-methyl-L-phenylalanine
  MOD_RES 25: note = 2-Aminoisobutyric acid

SEQ ID NO: 152 - protein, 30 aa, synthetic construct; identical to SEQ ID NO: 150
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Margaroyl
  MOD_RES 22: note = alpha-methyl-L-phenylalanine
  MOD_RES 25: note = 2-Aminoisobutyric acid

SEQ ID NO: 153 - protein, 30 aa, synthetic construct; identical to SEQ ID NO: 118
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Stearoyl
  MOD_RES 22: note = alpha-methyl-L-phenylalanine
  MOD_RES 25: note = 2-Aminoisobutyric acid

SEQ ID NO: 154 - protein, 29 aa, synthetic construct
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Stearoyl
  MOD_RES 22: note = alpha-methyl-L-phenylalanine
  MOD_RES 25: note = 2-Aminoisobutyric acid
HSQGSXTSDVSKXLDSXAAQDXVQXIANT

SEQ ID NO: 155 - protein, 29 aa, synthetic construct
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Stearoyl
  MOD_RES 22: note = alpha-methyl-L-phenylalanine
  MOD_RES 25: note = 2-Aminoisobutyric acid
HSQGSXTSDVSKXLDSXAAQDXVQXIINT

SEQ ID NO: 156 - protein, 29 aa, synthetic construct
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Stearoyl
  MOD_RES 22: note = alpha-methyl-L-phenylalanine
  MOD_RES 25: note = 2-Aminoisobutyric acid
HSQGSXTSDVSKXLDSXAAQDXVQXLLNT

SEQ ID NO: 157 - protein, 30 aa, synthetic construct
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Stearoyl
  MOD_RES 22: note = alpha-methyl-L-phenylalanine
  MOD_RES 25: note = 2-Aminoisobutyric acid
HSQGSXTSDVSKXLDSXAAQDXVQXVVEGG

SEQ ID NO: 158 - protein, 28 aa, synthetic construct
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Stearoyl
  MOD_RES 22: note = alpha-methyl-L-phenylalanine
  MOD_RES 25: note = 2-Aminoisobutyric acid
HSQGSXTSDVSKXLDSXAAQDXVQXIAN

SEQ ID NO: 159 - protein, 28 aa, synthetic construct
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Palmitoyl
  MOD_RES 22: note = alpha-methyl-L-phenylalanine
  MOD_RES 25: note = 2-Aminoisobutyric acid
HSQGSXTSDVSKXLDSXAAQDXVQXIAN

SEQ ID NO: 160 - protein, 28 aa, synthetic construct
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Margaroyl
  MOD_RES 22: note = alpha-methyl-L-phenylalanine
  MOD_RES 25: note = 2-Aminoisobutyric acid
HSQGSXTSDVSKXLDSXAAQDXVQXIAN

SEQ ID NO: 161 - protein, 28 aa, synthetic construct
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Palmitoyl
  MOD_RES 22: note = alpha-methyl-L-phenylalanine
HSQGSXTSDVSKXLDSXAAQDXVQIIAN

SEQ ID NO: 162 - protein, 28 aa, synthetic construct
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Palmitoyl
  MOD_RES 22: note = alpha-methyl-L-phenylalanine
  MOD_RES 25: note = 2-Aminoisobutyric acid
HSQGSXTSDVSKXLDSXAAQDXVEXIAN

SEQ ID NO: 163 - protein, 28 aa, synthetic construct
  MOD_RES 6: note = alpha-methyl-L-phenylalanine
  MOD_RES 13: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Palmitoyl
  MOD_RES 22: note = alpha-methyl-L-phenylalanine
  MOD_RES 25: note = 2-Aminoisobutyric acid
HSQGSXTSDVSKXLDSXAAQDXVQXIAG

SEQ ID NO: 164 - protein, 28 aa, synthetic construct
  MOD_RES 6: note = alpha-methyl-L-phenylalanine
  MOD_RES 13: note = 2-Aminoisobutyric acid
  MOD_RES 17: note = Lys, wherein the side chain of Lys is connected to polyethylene glycol 2- polyethylene glycol 2-gamma-Glu-gamma-Glu- Stearoyl
  MOD_RES 22: note = alpha-methyl-L-phenylalanine
  MOD_RES 25: note = 2-Aminoisobutyric acid
HSQGSXTSDVSKXLDSXAAQDXVQXLEA

SEQ ID NO: 165 - protein, 28 aa, synthetic construct
  MOD_RES 6: note = alpha-methyl-L-phenylalanine
  MOD_RES 13: note = 2-Aminoisobutyric acid
  MOD_RES 17: note = Lys, wherein the side chain of Lys is connected to polyethylene glycol 2- polyethylene glycol 2-gamma-Glu-gamma-Glu- Stearoyl
  MOD_RES 22: note = alpha-methyl-L-phenylalanine
  MOD_RES 25: note = 2-Aminoisobutyric acid
HSQGSXTSDVSKXLDSXAAQDXVQXLAN

SEQ ID NO: 166 - protein, 28 aa, synthetic construct
  MOD_RES 6: note = alpha-methyl-L-phenylalanine
  MOD_RES 13: note = 2-Aminoisobutyric acid
  MOD_RES 17: note = Lys, wherein the side chain of Lys is connected to polyethylene glycol 2- polyethylene glycol 2-gamma-Glu-gamma-Glu- Stearoyl
  MOD_RES 22: note = alpha-methyl-L-phenylalanine
  MOD_RES 25: note = 2-Aminoisobutyric acid
HSQGSXTSDVSKXLDSXAAQDXVQXLSE

SEQ ID NO: 167 - protein, 29 aa, synthetic construct
  MOD_RES 6: note = alpha-methyl-L-phenylalanine
  MOD_RES 13: note = 2-Aminoisobutyric acid
  MOD_RES 17: note = Lys, wherein the side chain of Lys is connected to polyethylene glycol 2- polyethylene glycol 2-gamma-Glu-gamma-Glu- Stearoyl
  MOD_RES 22: note = alpha-methyl-L-phenylalanine
  MOD_RES 25: note = 2-Aminoisobutyric acid
HSQGSXTSDVSKXLDSXAAQDXVQXLANT

SEQ ID NO: 168 - protein, 29 aa, synthetic construct
  MOD_RES 6: note = alpha-methyl-L-phenylalanine
  MOD_RES 13: note = 2-Aminoisobutyric acid
  MOD_RES 17: note = Lys, wherein the side chain of Lys is connected to polyethylene glycol 2- polyethylene glycol 2-gamma-Glu-gamma-Glu- Stearoyl
  MOD_RES 22: note = alpha-methyl-L-phenylalanine
  MOD_RES 25: note = 2-Aminoisobutyric acid
HSQGSXTSDVSKXLDSXAAQDXVQXLANT

SEQ ID NO: 169 - protein, 28 aa, synthetic construct
  MOD_RES 6: note = alpha-methyl-L-phenylalanine
  MOD_RES 13: note = 2-Aminoisobutyric acid
  MOD_RES 17: note = Lys, wherein the side chain of Lys is connected to polyethylene glycol 2- polyethylene glycol 2-gamma-Glu-gamma-Glu- Stearoyl
  MOD_RES 22: note = alpha-methyl-L-phenylalanine
  MOD_RES 25: note = 2-Aminoisobutyric acid
HSQGSXTSDVSKXLDSXAAQDXVQXIAN

SEQ ID NO: 170 - protein, 28 aa, synthetic construct
  MOD_RES 6: note = alpha-methyl-L-phenylalanine
  MOD_RES 13: note = 2-Aminoisobutyric acid
  MOD_RES 17: note = Lys, wherein the side chain of Lys is connected to polyethylene glycol 2- polyethylene glycol 2-gamma-Glu-gamma-Glu- Stearoyl
  MOD_RES 22: note = alpha-methyl-L-phenylalanine
  MOD_RES 25: note = 2-Aminoisobutyric acid
HSQGSXTSDVSKXLDSXAAQDXVQXIIE

SEQ ID NO: 171 - protein, 28 aa, synthetic construct
  MOD_RES 6: note = alpha-methyl-L-phenylalanine
  MOD_RES 13: note = 2-Aminoisobutyric acid
  MOD_RES 17: note = Lys, wherein the side chain of Lys is connected to polyethylene glycol 2- polyethylene glycol 2-gamma-Glu-gamma-Glu- Palmitoyl
  MOD_RES 22: note = alpha-methyl-L-phenylalanine
  MOD_RES 25: note = 2-Aminoisobutyric acid
HSQGSXTSDVSKXLDSXAAQDXVQXIAN

SEQ ID NO: 172 - protein, 29 aa, synthetic construct
  MOD_RES 2: note = 2-Aminoisobutyric acid
  MOD_RES 6: note = alpha-methyl-L-phenylalanine
  MOD_RES 13: note = 2-Aminoisobutyric acid
  MOD_RES 17: note = Lys, wherein the side chain of Lys is connected to polyethylene glycol 2- polyethylene glycol 2-gamma-Glu-gamma-Glu- Stearyl
  MOD_RES 22: note = alpha-methyl-L-phenylalanine
  MOD_RES 25: note = 2-Aminoisobutyric acid
HXQGSXTSDVSKXLDSXLAAQDXVQXIAN

SEQ ID NO: 173 - protein, 28 aa, synthetic construct
  MOD_RES 2: note = 2-Aminoisobutyric acid
  MOD_RES 6: note = alpha-methyl-L-phenylalanine
  MOD_RES 13: note = 2-Aminoisobutyric acid
  MOD_RES 17: note = Lys, wherein the side chain of Lys is connected to polyethylene glycol 2- polyethylene glycol 2-gamma-Glu-gamma-Glu- Stearyl
  MOD_RES 22: note = alpha-methyl-L-phenylalanine
  MOD_RES 25: note = 2-Aminoisobutyric acid
HXQGSXTSDVSKXLDSXAAQDXVQXIAN

SEQ ID NO: 174 - protein, 28 aa, synthetic construct
  MOD_RES 2: note = 2-Aminoisobutyric acid
  MOD_RES 6: note = alpha-methyl-L-phenylalanine
  MOD_RES 13: note = 2-Aminoisobutyric acid
  MOD_RES 17: note = Lys, wherein the side chain of Lys is connected to polyethylene glycol 2- polyethylene glycol 2-gamma-Glu-gamma-Glu- Stearyl
  MOD_RES 22: note = alpha-methyl-L-phenylalanine
  MOD_RES 25: note = 2-Aminoisobutyric acid
HXQGSXTSDVSKXLDSXRAQDXVQXIAN

SEQ ID NO: 175 - protein, 28 aa, synthetic construct
  MOD_RES 2: note = 2-Aminoisobutyric acid
  MOD_RES 6: note = alpha-methyl-L-phenylalanine
  MOD_RES 13: note = 2-Aminoisobutyric acid
  MOD_RES 17: note = Lys, wherein the side chain of Lys is connected to polyethylene glycol 2- polyethylene glycol 2-gamma-Glu-gamma-Glu- Stearyl
  MOD_RES 22: note = alpha-methyl-L-phenylalanine
HXQGSXTSDVSKXLDSXAAQDXVQWIAN

SEQ ID NO: 176 - protein, 28 aa, synthetic construct
  MOD_RES 6: note = alpha-methyl-L-phenylalanine
  MOD_RES 13: note = 2-Aminoisobutyric acid
  MOD_RES 17: note = Lys, wherein the side chain of Lys is connected to polyethylene glycol 2- polyethylene glycol 2-gamma-Glu-gamma-Glu- Stearyl
  MOD_RES 22: note = alpha-methyl-L-phenylalanine
  MOD_RES 25: note = 2-Aminoisobutyric acid
HTQGSXTSDVSKXLDSXAAQDXVQXIAN

SEQ ID NO: 177 - protein, 29 aa, synthetic construct
  MOD_RES 6: note = alpha-methyl-L-phenylalanine
  MOD_RES 13: note = 2-Aminoisobutyric acid
  MOD_RES 17: note = Lys, wherein the side chain of Lys is connected to polyethylene glycol 2- polyethylene glycol 2-gamma-Glu-gamma-Glu- Palmitoyl
  MOD_RES 22: note = alpha-methyl-L-phenylalanine
  MOD_RES 25: note = 2-Aminoisobutyric acid
HSQGSXTSDVSKXLDSXQAQDXVQAXIAN

SEQ ID NO: 178 - protein, 28 aa, synthetic construct
  MOD_RES 6: note = alpha-methyl-L-phenylalanine
  MOD_RES 13: note = 2-Aminoisobutyric acid
  MOD_RES 17: note = Lys, wherein the side chain of Lys is connected to polyethylene glycol 2- polyethylene glycol 2-gamma-Glu-gamma-Glu- Palmitoyl
  MOD_RES 22: note = alpha-methyl-L-phenylalanine
HSQGSXTSDVSKXLDSXAAQDXVQHIAN

SEQ ID NO: 179 - protein, 28 aa, synthetic construct
  MOD_RES 6: note = alpha-methyl-L-phenylalanine
  MOD_RES 13: note = 2-Aminoisobutyric acid
  MOD_RES 17: note = Lys, wherein the side chain of Lys is connected to polyethylene glycol 2- polyethylene glycol 2-gamma-Glu-gamma-Glu- Palmitoyl
  MOD_RES 22: note = alpha-methyl-L-phenylalanine
HSQGSXTSDVSKXLDSXQAQDXVQHIAN

SEQ ID NO: 180 - protein, 28 aa, synthetic construct
  MOD_RES 6: note = alpha-methyl-L-phenylalanine
  MOD_RES 13: note = 2-Aminoisobutyric acid
  MOD_RES 17: note = Lys, wherein the side chain of Lys is connected to O2Oc-O2Oc- gamma-Glu -C18diacid
  MOD_RES 22: note = alpha-methyl-L-phenylalanine
  MOD_RES 25: note = 2-Aminoisobutyric acid
HSQGSXTSDVSKXLDSXAAQDXVQXIAN

SEQ ID NO: 181 - protein, 28 aa, synthetic construct
  MOD_RES 6: note = alpha-methyl-L-phenylalanine
  MOD_RES 12: note = Acetyllysine
  MOD_RES 13: note = 2-Aminoisobutyric acid
  MOD_RES 17: note = Lys, wherein the side chain of Lys is connected to O2Oc-O2Oc- gamma-Glu -C18diacid
  MOD_RES 22: note = alpha-methyl-L-phenylalanine
  MOD_RES 25: note = 2-Aminoisobutyric acid
HSQGSXTSDVSXXLDSXAAQDXVQXIAN

SEQ ID NO: 182 - protein, 28 aa, synthetic construct
  MOD_RES 6: note = alpha-methyl-L-phenylalanine
  MOD_RES 12: note = Acetyllysine
  MOD_RES 13: note = 2-Aminoisobutyric acid
  MOD_RES 17: note = Lys, wherein the side chain of Lys is connected to polyethylene glycol 2- polyethylene glycol 2-gamma-Glu-gamma-Glu- Stearyl
  MOD_RES 22: note = alpha-methyl-L-phenylalanine
  MOD_RES 25: note = 2-Aminoisobutyric acid
HSQGSXTSDVSXXLDSXAAQDXVQXIAN

SEQ ID NO: 183 - protein, 28 aa, synthetic construct
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Palmitoyl
  MOD_RES 22: note = alpha-methyl-L-phenylalanine
  MOD_RES 25: note = 2-Aminoisobutyric acid
HSQGSXTSDVSKILDSXAAQDXVQXIAN

SEQ ID NO: 184 - protein, 28 aa, synthetic construct
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Palmitoyl
  MOD_RES 22: note = alpha-methyl-L-phenylalanine
HSQGSXTSDVSKILDSXAAQDXVQIIAN

SEQ ID NO: 185 - protein, 28 aa, synthetic construct
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Palmitoyl
  MOD_RES 22: note = alpha-methyl-L-phenylalanine
HSQGSXTSDVSKILDSXAAQDXVQIIAG

SEQ ID NO: 186 - protein, 28 aa, synthetic construct
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Palmitoyl
  MOD_RES 22: note = alpha-methyl-L-phenylalanine
HSQGSXTSDVSKILDSXAAQDXVEIIAN

SEQ ID NO: 187 - protein, 28 aa, synthetic construct
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Palmitoyl
  MOD_RES 22: note = alpha-methyl-L-phenylalanine
  MOD_RES 25: note = alpha-methyl-L-phenylalanine
HSQGSXTSDVSKILDSXAAQDXVQXIAN

SEQ ID NO: 188 - protein, 29 aa, synthetic construct
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Palmitoyl
  MOD_RES 22: note = alpha-methyl-L-phenylalanine
HSQGSXTSDVSKILDSXAAQDXVEWIINT

SEQ ID NO: 189 - protein, 29 aa, synthetic construct
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Stearoyl
  MOD_RES 22: note = alpha-methyl-L-phenylalanine
  MOD_RES 25: note = 2-Aminoisobutyric acid
HSQGSXTSDVSKILDSXAAQDXVQXIINT

SEQ ID NO: 190 - protein, 29 aa, synthetic construct
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Palmitoyl
  MOD_RES 22: note = alpha-methyl-L-phenylalanine
  MOD_RES 25: note = 2-Aminoisobutyric acid
HSQGSXTSDVSKILDSXAAQDXVQXIINT

SEQ ID NO: 191 - protein, 29 aa, synthetic construct
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu-gamma-Glu- Myristoyl
  MOD_RES 22: note = alpha-methyl-L-phenylalanine
  MOD_RES 25: note = 2-Aminoisobutyric acid
HSQGSXTSDVSKILDSXAAQDXVQXIINT

SEQ ID NO: 192 - protein, 29 aa, synthetic construct
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu- Stearoyl
  MOD_RES 22: note = alpha-methyl-L-phenylalanine
  MOD_RES 25: note = 2-Aminoisobutyric acid
HSQGSXTSDVSKILDSXAAQDXVQXIINT

SEQ ID NO: 193 - protein, 29 aa, synthetic construct
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu- Palmitoyl
  MOD_RES 22: note = alpha-methyl-L-phenylalanine
  MOD_RES 25: note = 2-Aminoisobutyric acid
HSQGSXTSDVSKILDSXAAQDXVQXIINT

SEQ ID NO: 194 - protein, 29 aa, synthetic construct
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu- Myristoyl
  MOD_RES 22: note = alpha-methyl-L-phenylalanine
  MOD_RES 25: note = 2-Aminoisobutyric acid
HSQGSXTSDVSKILDSXAAQDXVQXIINT

SEQ ID NO: 195 - protein, 28 aa, synthetic construct
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu- gamma-Glu- Palmitoyl
  MOD_RES 22: note = alpha-methyl-L-phenylalanine
HXQGSXTSDVSKILDSXAAQDXVQIIAN

SEQ ID NO: 196 - protein, 29 aa, synthetic construct
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu- gamma-Glu- Stearoyl
  MOD_RES 22: note = alpha-methyl-L-phenylalanine
  MOD_RES 25: note = 2-Aminoisobutyric acid
HXQGSXTSDVSKILDSXAAQDXVQXIINT

SEQ ID NO: 197 - protein, 29 aa, synthetic construct
  MOD_RES 2: note = 2-Aminoisobutyric acid
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu- gamma-Glu- Palmitoyl
  MOD_RES 22: note = alpha-methyl-L-phenylalanine
  MOD_RES 25: note = 2-Aminoisobutyric acid
HXQGSXTSDVSKILDSXAAQDXVQXIINT

SEQ ID NO: 198 - protein, 29 aa, synthetic construct
  MOD_RES 2: note = 2-Aminoisobutyric acid
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu- gamma-Glu- Myristoyl
  MOD_RES 22: note = alpha-methyl-L-phenylalanine
  MOD_RES 25: note = 2-Aminoisobutyric acid
HXQGSXTSDVSKILDSXAAQDXVQXIINT

SEQ ID NO: 199 - protein, 29 aa, synthetic construct
  MOD_RES 2: note = 2-Aminoisobutyric acid
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu- gamma-Glu- Stearoyl
  MOD_RES 22: note = alpha-methyl-L-phenylalanine
  MOD_RES 25: note = 2-Aminoisobutyric acid
HXQGSXTSDVSKILDSXAAQDXVQXIINT

SEQ ID NO: 200 - protein, 29 aa, synthetic construct
  MOD_RES 2: note = 2-Aminoisobutyric acid
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu- Palmitoyl
  MOD_RES 22: note = alpha-methyl-L-phenylalanine
  MOD_RES 25: note = 2-Aminoisobutyric acid
HXQGSXTSDVSKILDSXAAQDXVQXIINT

SEQ ID NO: 201 - protein, 29 aa, synthetic construct
  MOD_RES 2: note = 2-Aminoisobutyric acid
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu- Myristoyl
  MOD_RES 22: note = alpha-methyl-L-phenylalanine
  MOD_RES 25: note = 2-Aminoisobutyric acid
HXQGSXTSDVSKILDSXAAQDXVQXIINT

SEQ ID NO: 202 - protein, 29 aa, synthetic construct
  MOD_RES 2: note = alpha-methyl-L-serine
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu- gamma-Glu- Stearoyl
  MOD_RES 22: note = alpha-methyl-L-phenylalanine
  MOD_RES 25: note = 2-Aminoisobutyric acid
HXQGSXTSDVSKILDSXAAQDXVQXIINT

SEQ ID NO: 203 - protein, 29 aa, synthetic construct
  MOD_RES 2: note = alpha-methyl-L-serine
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu- gamma-Glu- Palmitoyl
  MOD_RES 22: note = alpha-methyl-L-phenylalanine
  MOD_RES 25: note = 2-Aminoisobutyric acid
HXQGSXTSDVSKILDSXAAQDXVQXIINT

SEQ ID NO: 204 - protein, 29 aa, synthetic construct
  MOD_RES 2: note = alpha-methyl-L-serine
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu- gamma-Glu- Myristoyl
  MOD_RES 22: note = alpha-methyl-L-phenylalanine
  MOD_RES 25: note = 2-Aminoisobutyric acid
HXQGSXTSDVSKILDSXAAQDXVQXIINT

SEQ ID NO: 205 - protein, 29 aa, synthetic construct
  MOD_RES 2: note = alpha-methyl-L-serine
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu- Stearoyl
  MOD_RES 22: note = alpha-methyl-L-phenylalanine
  MOD_RES 25: note = 2-Aminoisobutyric acid
HXQGSXTSDVSKILDSXAAQDXVQXIINT

SEQ ID NO: 206 - protein, 29 aa, synthetic construct
  MOD_RES 2: note = alpha-methyl-L-serine
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu- Palmitoyl
  MOD_RES 22: note = alpha-methyl-L-phenylalanine
  MOD_RES 25: note = 2-Aminoisobutyric acid
HXQGSXTSDVSKILDSXAAQDXVQXIINT

SEQ ID NO: 207 - protein, 29 aa, synthetic construct
  MOD_RES 2: note = alpha-methyl-L-serine
  MOD_RES 6: note = alpha-methyl-L-phenylalanine
  MOD_RES 17: note = Lys, wherein the side chain of Lys is connected to polyethylene glycol 2- polyethylene glycol 2-gamma-Glu- Myristoyl
  MOD_RES 22: note = alpha-methyl-L-phenylalanine
  MOD_RES 25: note = 2-Aminoisobutyric acid
HXQGSXTSDVSKILDSXAAQDXVQXIINT

SEQ ID NO: 208 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu-C18diacid
HXHGSXTSDVSKXLDSQAAXDFVQWIANT

SEQ ID NO: 209 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu-C18diacid
HXQGTXTSDVSKXLDSRAAXDFVQWIANT

SEQ ID NO: 210 - protein, 29 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to O2Oc-O2Oc-gamma-Glu-C18diacid
HXHGSXTSDVSKXLDSRAAXDFVQWIANT

SEQ ID NO: 211 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 18: note = 2-Aminoisobutyric acid
  MOD_RES 20: note = Lys, wherein the side chain of Lys is connected to O2Oc-O2Oc-gamma-Glu-C18diacid
HXHGSXTSDVSKXLDSRXAXDFVQWIANT

SEQ ID NO: 212 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu-C18diacid
  MOD_RES 25: note = 2-Aminoisobutyric acid
HXHGSXTSDVSKXLDSQAAXDFVQXIANT

SEQ ID NO: 213 - protein, 29 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to gamma-Glu-C18diacid
HXQGTFTSDVSKXLDSRAAXDFVQWIANT

SEQ ID NO: 214 - protein, 29 aa, synthetic construct
  MOD_RES 2: note = 2-Aminoisobutyric acid
  MOD_RES 6: note = alpha-methyl-L-phenylalanine
  MOD_RES 20: note = Lys, wherein the side chain of Lys is connected to O2Oc-O2Oc-gamma-Glu-C18diacid
  MOD_RES 13: note = alpha-methyl-L-phenylalanine
HXHGSXTSDVSKXLDSQAAXDFVEWIANT

SEQ ID NO: 215 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu-C18diacid
HXHGSXTSDVSKXLDSQAAXDFVEWIANT

SEQ ID NO: 216 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu- gamma-Glu -C20diacid
HXHGSXTSDVSKXLDSRAAXDFVQWIANT

SEQ ID NO: 217 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu- C20diacid
HXHGSXTSDVSKXLDSRAAXDFVEWIANT

SEQ ID NO: 218 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu-gamma-Glu C20diacid
HXHGSXTSDVSKXLDSRAAXDFVEWIANT

SEQ ID NO: 219 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc- gamma-Glu-C20diacid
HXHGSXTSDVSKXLDSRAAXDFVQWIANT

SEQ ID NO: 220 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc- gamma-Glu C20diacid
  MOD_RES 22: note = alpha-methyl-L-phenylalanine
HXHGSXTSDVSKXLDSRAAXDXVQWIANT

SEQ ID NO: 221 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc- gamma-Glu C20diacid
  MOD_RES 22: note = alpha-methyl-L-phenylalanine
HXHGSXTSDVSKXLDSRAAXDXVEWIANT

SEQ ID NO: 222 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 18: note = 2-Aminoisobutyric acid
  MOD_RES 20: note = O2Oc-O2Oc- gamma-Glu- gamma-Glu-C20diacid
HXHGSXTSDVSKXLDSRXAXDFVQWIANT

SEQ ID NO: 223 - protein, 30 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = O2Oc-O2Oc- gamma-Glu- gamma-Glu-C20diacid
HXHGSXTSDVSKXLDSRAAXDFVQWIANTG

SEQ ID NO: 224 - protein, 31 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = O2Oc-O2Oc- gamma-Glu- gamma-Glu-C20diacid
HXHGSXTSDVSKXLDSRAAXDFVQWIANTGG

SEQ ID NO: 225 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 18: note = 2-Aminoisobutyric acid
  MOD_RES 20: note = Lys, wherein the side chain of Lys is connected to O2Oc-O2Oc- gamma-Glu- gamma-Glu-C20diacid
  MOD_RES 25: note = 2-Aminoisobutyric acid
HXHGSXTSDVSKXLDSRXAXDFVQXIANT

SEQ ID NO: 226 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc- gamma-Glu- gamma-Glu-C20diacid
  MOD_RES 25: note = 2-Aminoisobutyric acid
HXHGSXTSDVSKXLDSRAAXDFVQXIANT

SEQ ID NO: 227 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = O2Oc-O2Oc-gamma-Glu-C20diacid
HXHGSXTSDVSKXLDSQAAXDFVQSIANT

SEQ ID NO: 228 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = O2Oc-O2Oc-gamma-Glu-C18diacid
  MOD_RES 25: note = 2-Aminoisobutyric acid
  MOD_RES 22: note = alpha-methyl-L-phenylalanine
HXHGSXTSDVSKXLDSRAAXDXIAXIANT

SEQ ID NO: 229 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu-C18diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 230 - protein, 31 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu-C18diacid
  MOD_RES 22: note = alpha-methyl-L-phenylalanine
  MOD_RES 25: note = 2-Aminoisobutyric acid
HXHGSXTSDVSKXLDSRAAXDXVQXIANTGG

SEQ ID NO: 231 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu-C18diacid
  MOD_RES 22: note = alpha-methyl-L-phenylalanine
  MOD_RES 25: note = 2-Aminoisobutyric acid
HXHGSXTSDVSKXLDSRAAXDXVEXIANT

SEQ ID NO: 232 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu-C18diacid
  MOD_RES 22: note = alpha-methyl-L-phenylalanine
  MOD_RES 25: note = 2-Aminoisobutyric acid
HXHGSXTSDVSKXLDSRAAXDXIAXIANT

SEQ ID NO: 233 - protein, 31 aa, synthetic construct; identical to SEQ ID NO: 230
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu-gamma-Glu-C20diacid
  MOD_RES 22: note = alpha-methyl-L-phenylalanine
  MOD_RES 25: note = 2-Aminoisobutyric acid

SEQ ID NO: 234 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu-gamma-Glu-C20diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 235 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc- gamma-Glu-C18diacid
  MOD_RES 22: note = alpha-methyl-L-phenylalanine
HXHGSXTSDVSKXLDSRAAXDXIAWIANT

SEQ ID NO: 236 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc- gamma-Glu- gamma-Glu-C18diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 237 - protein, 31 aa, synthetic construct; identical to SEQ ID NO: 230
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc- gamma-Glu- gamma-Glu-C20diacid
  MOD_RES 22: note = alpha-methyl-L-phenylalanine
  MOD_RES 25: note = 2-Aminoisobutyric acid

SEQ ID NO: 238 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc- gamma-Glu- gamma-Glu-C20diacid
  MOD_RES 22: note = alpha-methyl-L-phenylalanine
  MOD_RES 25: note = 2-Aminoisobutyric acid
HXHGSXTSDVSKXLDSRAAXDXVEXIANT

SEQ ID NO: 239 - protein, 30 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc- gamma-Glu- gamma-Glu-C20diacid
  MOD_RES 25: note = 2-Aminoisobutyric acid
HXHGSXTSDVSKXLDSRAAXDFVQXIANTG

SEQ ID NO: 240 - protein, 31 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc- gamma-Glu- gamma-Glu-C20diacid
  MOD_RES 25: note = 2-Aminoisobutyric acid
HXHGSXTSDVSKXLDSRAAXDFVQXIANTGG

SEQ ID NO: 241 - protein, 30 aa, synthetic construct; identical to SEQ ID NO: 239
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = O2Oc-O2Oc- gamma-Glu- gamma-Glu-C18diacid
  MOD_RES 25: note = 2-Aminoisobutyric acid

SEQ ID NO: 242 - protein, 31 aa, synthetic construct; identical to SEQ ID NO: 240
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc- gamma-Glu- gamma-Glu-C18diacid
  MOD_RES 25: note = 2-Aminoisobutyric acid

SEQ ID NO: 243 - protein, 30 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc- gamma-Glu- gamma-Glu-C20diacid
  MOD_RES 22: note = alpha-methyl-L-phenylalanine
HXHGSXTSDVSKXLDSRAAXDXVQWIANTG

SEQ ID NO: 244 - protein, 31 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc- gamma-Glu- gamma-Glu-C20diacid
  MOD_RES 22: note = alpha-methyl-L-phenylalanine
HXHGSXTSDVSKXLDSRAAXDXVQWIANTGG

SEQ ID NO: 245 - protein, 30 aa, synthetic construct; identical to SEQ ID NO: 243
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc- gamma-Glu- gamma-Glu-C18diacid
  MOD_RES 22: note = alpha-methyl-L-phenylalanine

SEQ ID NO: 246 - protein, 31 aa, synthetic construct; identical to SEQ ID NO: 244
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc- gamma-Glu- gamma-Glu-C18diacid
  MOD_RES 22: note = alpha-methyl-L-phenylalanine

SEQ ID NO: 247 - protein, 28 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 18: note = 2-Aminoisobutyric acid
  MOD_RES 20: note = Lys, wherein the side chain of Lys is connected to O2Oc-O2Oc- gamma-Glu- gamma-Glu-C20diacid
HXHGSXTSDVSKXLDSRXAXDFVQSIAN

SEQ ID NO: 248 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = O2Oc-O2Oc- gamma-Glu- gamma-Glu-C18diacid
  MOD_RES 22: note = alpha-methyl-L-phenylalanine
HXHGSXTSDVSKXLDSRAAXDXVQSIANT

SEQ ID NO: 249 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc- gamma-Glu- gamma-Glu-C20diacid
  MOD_RES 22: note = alpha-methyl-L-phenylalanine
HXHGSXTSDVSKXLDSRAAXDXVQSIANT

SEQ ID NO: 250 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc- gamma-Glu-C18diacid
  MOD_RES 22: note = alpha-methyl-L-phenylalanine
HXHGSXTSDVSKXLDSRAAXDXVESIANT

SEQ ID NO: 251 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu-gamma-Glu-C20diacid
  MOD_RES 22: note = alpha-methyl-L-phenylalanine
HXHGSXTSDVSKXLDSRAAXDXVESIANT

SEQ ID NO: 252 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu-gamma-Glu-C18diacid
  MOD_RES 22: note = alpha-methyl-L-phenylalanine
HXHGSXTSDVSKXLDSRAAXDXVQWIANT

SEQ ID NO: 253 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu-gamma-Glu-C20diacid
  MOD_RES 22: note = alpha-methyl-L-phenylalanine
NXHGSXTSDVSKXLDSRAAXDXVQWIANT

SEQ ID NO: 254 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 18: note = 2-Aminoisobutyric acid
  MOD_RES 20: note = Lys, wherein the side chain of Lys is connected to O2Oc-O2Oc-gamma-Glu-gamma-Glu-C20diacid
  MOD_RES 22: note = alpha-methyl-L-phenylalanine
HXHGSXTSDVSKXLDSRXAXDXVESIANT

SEQ ID NO: 255 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 18: note = 2-Aminoisobutyric acid
  MOD_RES 20: note = Lys, wherein the side chain of Lys is connected to O2Oc-O2Oc-gamma-Glu-gamma-Glu-C18diacid
  MOD_RES 22: note = alpha-methyl-L-phenylalanine
HXHGSXTSDVSKXLDSRXAXDXVESIANT

SEQ ID NO: 256 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 18: note = 2-Aminoisobutyric acid
  MOD_RES 20: note = Lys, wherein the side chain of Lys is connected to O2Oc-O2Oc-gamma-Glu-gamma-Glu-C18diacid
HXHGSXTSDVSKXLDSRXAXDFVESIANT

SEQ ID NO: 257 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 18: note = 2-Aminoisobutyric acid
  MOD_RES 20: note = Lys, wherein the side chain of Lys is connected to O2Oc-O2Oc-gamma-Glu-C20diacid
HXHGSXTSDVSKXLDSRXAXDFVESIANT

SEQ ID NO: 258 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 18: note = 2-Aminoisobutyric acid
  MOD_RES 20: note = Lys, wherein the side chain of Lys is connected to O2Oc-gamma-Glu-C18diacid
HXHGSXTSDVSKXLDSRXAXDFVESIANT

SEQ ID NO: 259 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 18: note = 2-Aminoisobutyric acid
  MOD_RES 20: note = Lys, wherein the side chain of Lys is connected to gamma-Glu-C18diacid
HXHGSXTSDVSKXLDSRXAXDFVESIANT

SEQ ID NO: 260 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu-C18diacid
  MOD_RES 22: note = alpha-methyl-L-phenylalanine
  MOD_RES 25: note = 2-Aminoisobutyric acid
HXHGSXTSDVSKXLDSQAAXDXVQXIANT

SEQ ID NO: 261 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu- gamma-Glu -C20diacid
  MOD_RES 22: note = alpha-methyl-L-phenylalanine
  MOD_RES 25: note = 2-Aminoisobutyric acid
HXHGSXTSDVSKXLDSQAAXDXVQXIANT

SEQ ID NO: 262 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu- gamma-Glu-Stearoyl
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 263 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to polyethylene glycol 2- polyethylene glycol 2-gamma-Glu- gamma-Glu- C18diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 264 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc- gamma-Glu-O2Oc- C18diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 265 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc- gamma-Glu-O2Oc- gamma-Glu-C18diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 266 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to polyethylene glycol 4- gamma-Glu- C18diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 267 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc- gamma-Glu-C18diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 268 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to polyethylene glycol 4- gamma-Glu-gamma-Glu-C18diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 269 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to polyethylene glycol 2- polyethylene glycol 2- gamma-Glu-gamma-Glu-C20diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 270 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc- gamma-Glu- O2Oc-C20diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 271 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc- gamma-Glu- O2Oc-C20diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 272 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to polyethylene glycol 4- gamma-Glu- C20diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 273 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc- gamma-Glu-C20diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 274 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to polyethylene glycol 4- gamma-Glu- gamma-Glu -C20diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 275 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to gamma-Glu- gamma-Glu -C18diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 276 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  METAL 20: note = Lys, wherein the side chain of Lys is connected to gamma-Glu-C18diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 277 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc- O2Oc-C18diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 278 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-C18diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 279 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc- gamma-Glu- gamma-Glu -C18diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 280 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to gamma-Glu- gamma-Glu -C20diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 281 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to gamma-Glu- C20diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 282 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = O2Oc- O2Oc-C20diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 283 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-C20diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 284 - protein, 29 aa, synthetic construct
  MOD_RES 1: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc- gamma-Glu- gamma-Glu -C20diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 285 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-02Oc-gamma-Glu-C20diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 286 - protein, 29 aa, synthetic construct
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu- gamma-Glu -C20diacid
  MOD_RES 22: note = alpha-methyl-L-phenylalanine
  MOD_RES 25: note = 2-Aminoisobutyric acid
HSHGSXTSDVSKXLDSRAAXDXVQXIANT

SEQ ID NO: 287 - protein, 29 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to O2Oc-O2Oc-gamma-Glu- gamma-Glu -C20diacid
  MOD_RES 22: note = alpha-methyl-L-phenylalanine
  MOD_RES 25: note = 2-Aminoisobutyric acid
HXHGSFTSDVSKXLDSRAAXDXVQXIANT

SEQ ID NO: 288 - protein, 29 aa, synthetic construct
  MOD_RES 2: note = 2-Aminoisobutyric acid
  MOD_RES 6: note = alpha-methyl-L-phenylalanine
  MOD_RES 20: note = Lys, wherein the side chain of Lys is connected to O2Oc-O2O- gamma-Glu- gamma-Glu -C20diacid
  MOD_RES 22: note = alpha-methyl-L-phenylalanine
  MOD_RES 25: note = 2-Aminoisobutyric acid
HXHGSXTSDVSKYLDSRAAXDXVQXIANT

SEQ ID NO: 289 - protein, 29 aa, synthetic construct
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc-O2Oc-gamma-Glu-C18diacid
  MOD_RES 22: note = alpha-methyl-L-phenylalanine
  MOD_RES 25: note = 2-Aminoisobutyric acid
HSHGSXTSDVSKXLDSRAAXDXVQXIANT

SEQ ID NO: 290 - protein, 29 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to O2Oc-O2Oc gamma-Glu-C18diacid
  MOD_RES 22: note = alpha-methyl-L-phenylalanine
  MOD_RES 25: note = 2-Aminoisobutyric acid
HXHGSFTSDVSKXLDSRAAXDXVQXIANT

SEQ ID NO: 291 - protein, 29 aa, synthetic construct
  MOD_RES 2: note = 2-Aminoisobutyric acid
  MOD_RES 6: note = alpha-methyl-L-phenylalanine
  MOD_RES 20: note = Lys, wherein the side chain of Lys is connected to O2Oc-O2Oc- gamma-Glu -C18diacid
  MOD_RES 22: note = alpha-methyl-L-phenylalanine
  MOD_RES 25: note = 2-Aminoisobutyric acid
HXHGSXTSDVSKYLDSRAAXDXVQXIANT

SEQ ID NO: 292 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc- O2Oc-gamma-Glu-C18diacid
  MOD_RES 25: note = 2-Aminoisobutyric acid
HXHGSXTSDVSKXLDSRAAXDFVQXIANT

SEQ ID NO: 293 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc- O2Oc - gamma-Glu -C18diacid
  MOD_RES 22: note = alpha-methyl-L-phenylalanine
HXHGSXTSDVSKXLDSRAAXDXVQWIANT

SEQ ID NO: 294 - protein, 29 aa, synthetic construct
  MOD_RES 2: note = 2-Aminoisobutyric acid
  MOD_RES 6: note = alpha-methyl-L-phenylalanine
  MOD_RES 20: note = Lys, wherein the side chain of Lys is connected to O2Oc- O2Oc - gamma-Glu -gamma-Glu-C20diacid
HXHGSXTSDVSKYLDSRAAXDFVQWIANT

SEQ ID NO: 295 - protein, 29 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to O2Oc- O2Oc - gamma-Glu - gamma-Glu C20diacid
HXHGSFTSDVSKXLDSRAAXDFVQWIANT

SEQ ID NO: 296 - protein, 29 aa, synthetic construct
  MOD_RES 2: note = 2-Aminoisobutyric acid
  MOD_RES 20: note = Lys, wherein the side chain of Lys is connected to O2Oc- O2Oc - gamma-Glu - gamma-Glu C20diacid
  MOD_RES 22: note = alpha-methyl-L-phenylalanine
HXHGSFTSDVSKYLDSRAAXDXVQWIANT

SEQ ID NO: 297 - protein, 29 aa, synthetic construct
  MOD_RES 2: note = 2-Aminoisobutyric acid
  MOD_RES 20: note = Lys, wherein the side chain of Lys is connected to O2Oc- O2Oc-gamma-Glu-C18diacid
HXHGSXTSDVSKYLDSRAAXDFVQWIANT

SEQ ID NO: 298 - protein, 29 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to O2Oc- O2Oc - gamma-Glu - C18diacid
HXHGSFTSDVSKXLDSRAAXDFVQWIANT

SEQ ID NO: 299 - protein, 29 aa, synthetic construct
  MOD_RES 2: note = 2-Aminoisobutyric acid
  MOD_RES 20: note = Lys, wherein the side chain of Lys is connected to O2Oc- O2Oc - gamma-Glu-C18diacid
  MOD_RES 22: note = alpha-methyl-L-phenylalanine
HXHGSFTSDVSKYLDSRAAXDXVQWIANT

SEQ ID NO: 300 - protein, 29 aa, synthetic construct
  MOD_RES 2: note = 2-Aminoisobutyric acid
  MOD_RES 6: note = alpha-methyl-L-phenylalanine
  MOD_RES 13: note = 2-Aminoisobutyric acid
  MOD_RES 20: note = Lys, wherein the side chain of Lys is connected to O2Oc- O2Oc - gamma-Glu- gamma-Glu-C20diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 301 - protein, 29 aa, synthetic construct
  MOD_RES 2: note = 2-Aminoisobutyric acid
  MOD_RES 6: note = alpha-methyl-L-phenylalanine
  MOD_RES 13: note = 2-Aminoisobutyric acid
  MOD_RES 20: note = Lys, wherein the side chain of Lys is connected to O2Oc- O2Oc -gamma-Glu-C18diacid
  MOD_RES 22: note = alpha-methyl-L-phenylalanine
  MOD_RES 25: note = 2-Aminoisobutyric acid
HXHGSXTSDVSKXLDSRAAXDXVQXIANT

SEQ ID NO: 302 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc- O2Oc - gamma-Glu-C18diacid
  MOD_RES 22: note = alpha-methyl-L-phenylalanine
  MOD_RES 25: note = 2-Aminoisobutyric acid
HXHGSXTSDVSRXLDSRAAXDXVQXIANT

SEQ ID NO: 303 - protein, 29 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 20: note = Lys, wherein the side chain of Lys is connected to O2Oc- O2Oc - gamma-Glu- gamma-Glu -C20diacid
HXHGSXTSDVSRXLDSRAAXDXVQXIANT

SEQ ID NO: 304 - protein, 29 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 20: note = Lys, wherein the side chain of Lys is connected to O2Oc- O2Oc - gamma-Glu- gamma-Glu -C20diacid
HXHGSXTSDVSKXLDSRAAXDXVQXIAET

SEQ ID NO: 305 - protein, 29 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 20: note = Lys, wherein the side chain of Lys is connected to O2Oc- O2Oc - gamma-Glu -C18diacid
HXHGSXTSDVSKXLDSRAAXDXVQXIAET

SEQ ID NO: 306 - protein, 29 aa, synthetic construct
  MOD_RES 2: note = 2-Aminoisobutyric acid
  MOD_RES 25: note = 2-Aminoisobutyric acid
  METAL 6: note = alpha-methyl-L-phenylalanine
  MOD_RES 13: note = alpha-methyl-L-phenylalanine
  MOD_RES 22: note = alpha-methyl-L-phenylalanine
  MOD_RES 20: note = O2Oc- O2Oc - gamma-Glu -C18diacid
HXHGSXTSDVSKXLDSRAAXDXIAXIAET

SEQ ID NO: 307 - protein, 29 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 20: note = Lys, wherein the side chain of Lys is connected to O2Oc- O2Oc - gamma-Glu - gamma-Glu-C20diacid
HXHGSXTSDVSKXLDSRAAXDXIAXIAET

SEQ ID NO: 308 - protein, 29 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 20: note = Lys, wherein the side chain of Lys is connected to O2Oc- O2Oc - gamma-Glu-C18diacid
HXHGSXTSDVSKXLDSRAAXDXVQXIANT

SEQ ID NO: 309 - protein, 29 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 20: note = Lys, wherein the side chain of Lys is connected to O2Oc- O2Oc - gamma-Glu- gamma-Glu-C20diacid
HXHGSXTSDVSKXLDSRAAXDXVQXIANT

SEQ ID NO: 310 - protein, 29 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to O2Oc- O2Oc - gamma-Glu- gamma-Glu-C20diacid
HXHGTFTSDVSKXLDSRAAXDFVQWIANT

SEQ ID NO: 311 - protein, 29 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to O2Oc- O2Oc-gamma-Glu-C18diacid
HXQGTFTSDVSKXLDSRAAXDFVEWIANT

SEQ ID NO: 312 - protein, 29 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to O2Oc- O2Oc-gamma-Glu-C18diacid
HXQGTFTSDVSKXLDSRAAXDFVRWIANT

SEQ ID NO: 313 - protein, 29 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to O2Oc- O2Oc-gamma-Glu-C18diacid
HXQGTFTSDVSKXLDTRAAXDFVQWIANT

SEQ ID NO: 314 - protein, 29 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to O2Oc- O2Oc-gamma-Glu-C18diacid
HXQGTFTSDVSKXLDERAAXDFVQWIANT

SEQ ID NO: 315 - protein, 30 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to O2Oc- O2Oc-gamma-Glu-C18diacid
HXQGTFTSDVSKXLDSRAAXDFVQWLEAGG

SEQ ID NO: 316 - protein, 29 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to O2Oc- O2Oc-gamma-Glu-C18diacid
HXQGTFTSDVSKXLDSRAAXDFVQWLVET

SEQ ID NO: 317 - protein, 28 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to O2Oc- O2Oc-gamma-Glu-C18diacid
  MOD_RES 28: note = polyethylene glycol
HXQGTFTSDYSKXLDSRRAXDFVQWLVX

SEQ ID NO: 318 - protein, 29 aa, synthetic construct
  MOD_RES 2: note = 2-Aminoisobutyric acid
  MOD_RES 18: note = 2-Aminoisobutyric acid
  MOD_RES 13: note = alpha-methyl-L-phenylalanine
  MOD_RES 20: note = Lys, wherein the side chain of Lys is connected to O2Oc- O2Oc-gamma-Glu-C18diacid
HXQGTFTSDVSKXLDSRXAXDFVQWLLNT

SEQ ID NO: 319 - protein, 29 aa, synthetic construct
  MOD_RES 2: note = 2-Aminoisobutyric acid
  MOD_RES 25: note = 2-Aminoisobutyric acid
  MOD_RES 18: note = 2-Aminoisobutyric acid
  MOD_RES 13: note = alpha-methyl-L-phenylalanine
  MOD_RES 20: note = Lys, wherein the side chain of Lys is connected to O2Oc- O2Oc-gamma-Glu-C18diacid
HXQGTFTSDVSKXLDSRXAXDFVQXLVAT

SEQ ID NO: 320 - protein, 29 aa, synthetic construct
  MOD_RES 2: note = 2-Aminoisobutyric acid
  MOD_RES 25: note = 2-Aminoisobutyric acid
  MOD_RES 13: note = alpha-methyl-L-phenylalanine
  MOD_RES 20: note = Lys, wherein the side chain of Lys is connected to O2Oc- O2Oc-gamma-Glu-C18diacid
HXQGTFTSDVSKXLDSRAAXDFVQXLVAT

SEQ ID NO: 321 - protein, 29 aa, synthetic construct
  MOD_RES 20: note = Lys, wherein the side chain of Lys is connected to O2Oc- O2Oc-gamma-Glu-stearoyl
  MOD_RES 13: note = alpha-methyl-L-phenylalanine
HSQGTFTSDYSKXLEEEAVXLFIRWLMNT

SEQ ID NO: 322 - protein, 29 aa, synthetic construct
  MOD_RES 2: note = 2-Aminoisobutyric acid
  MOD_RES 16: note = 2-Aminoisobutyric acid
  MOD_RES 13: note = alpha-methyl-L-phenylalanine
  MOD_RES 20: note = Lys, wherein the side chain of Lys is connected to O2Oc- O2Oc-gamma-Glu-C18diacid
HXQGTFTSDVSKXLDXRAAXDFVQWIANT

SEQ ID NO: 323 - protein, 29 aa, synthetic construct
  MOD_RES 2: note = alpha-methyl-L-serine
  MOD_RES 13: note = alpha-methyl-L-phenylalanine
  MOD_RES 20: note = Lys, wherein the side chain of Lys is connected to O2Oc- O2Oc-gamma-Glu-C18diacid
HXQGTFTSDVSKXLDSRAAXDFVQWIANT

SEQ ID NO: 324 - protein, 29 aa, synthetic construct
  MOD_RES 3: note = alpha-Methyl-glutamine
  MOD_RES 13: note = alpha-methyl-L-phenylalanine
  MOD_RES 20: note = Lys, wherein the side chain of Lys is connected to O2Oc- O2Oc-gamma-Glu-C18diacid
HSXGTFTSDVSKXLDSRAAXDFVQWIANT

SEQ ID NO: 325 - protein, 29 aa, synthetic construct
  MOD_RES 2: note = D-serine
  MOD_RES 13: note = alpha-methyl-L-phenylalanine
  MOD_RES 20: note = 2-Aminoisobutyric acid Lys, wherein the side chain of Lys is connected to O2Oc- O2Oc-gamma-Glu-C18diacid
HXQGTFTSDVSKXLDSRAAXDFVQWIANT

SEQ ID NO: 326 - protein, 29 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 13: note = alpha-methyl-L-phenylalanine
  MOD_RES 20: note = Lys, wherein the side chain of Lys is connected to O2Oc- O2Oc-gamma-Glu-C18diacid
  MOD_RES 22: note = alpha-methyl-L-phenylalanine
HXQGSXTSDVSKXLDSRAAXDXVQXIANT

SEQ ID NO: 327 - protein, 29 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 20: note = Lys, wherein the side chain of Lys is connected to O2Oc- O2Oc-gamma-Glu- gamma-Glu -C20diacid
HXQGSXTSDVSKXLDSRAAXDXVQXIANT

SEQ ID NO: 328 - protein, 29 aa, synthetic construct
  MOD_RES 3: note = alpha-Methyl-glutamine
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 20: note = Lys, wherein the side chain of Lys is connected to O2Oc- O2OcGlu- gamma-Glu -C18diacid
  MOD_RES 25: note = 2-Aminoisobutyric acid
HSXGSXTSDVSKXLDSRAAXDXVQXIANT

SEQ ID NO: 329 - protein, 29 aa, synthetic construct
  MOD_RES 3: note = Beta-dimethylglutamine
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 20: note = Lys, wherein the side chain of Lys is connected to O2Oc- O2Oc-gamma-Glu-C18diacid
  MOD_RES 25: note = 2-Aminoisobutyric acid
HSXGSXTSDVSKXLDSRAAXDXVQXIANT

SEQ ID NO: 330 - protein, 29 aa, synthetic construct
  MOD_RES 3: note = N-Methyl-Glutamine
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 20: note = Lys, wherein the side chain of Lys is connected to O2Oc- O2Oc- gamma-Glu -C18diacid
  MOD_RES 25: note = 2-Aminoisobutyric acid
HSXGSXTSDVSKXLDSRAAXDXVQXIANT

SEQ ID NO: 331 - protein, 29 aa, synthetic construct
  MOD_RES 3: note = dimethylglutamine
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 20: note = Lys, wherein the side chain of Lys is connected to O2Oc- O2Oc-gamma-Glu-C18diacid
  MOD_RES 25: note = 2-Aminoisobutyric acid
HSXGSXTSDVSKXLDSRAAXDXVQXIANT

SEQ ID NO: 332 - protein, 29 aa, synthetic construct
  MOD_RES 2: note = aminocyclopropane-1-carboxylic acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 20: note = Lys, wherein the side chain of Lys is connected to O2Oc- O2Oc-Glu- gamma-C18diacid
  MOD_RES 25: note = 2-Aminoisobutyric acid
HSXGSXTSDVSKXLDSRAAXDXVQXIANT

SEQ ID NO: 333 - protein, 29 aa, synthetic construct
  MOD_RES 2: note = 1-aminocyclobutane-1-carboxylic acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 20: note = Lys, wherein the side chain of Lys is connected to O2Oc- O2Oc-gamma-Glu -C18diacid
  MOD_RES 25: note = 2-Aminoisobutyric acid
HXQGSXTSDVSKXLDSRAAXDXVQXIANT

SEQ ID NO: 334 - protein, 29 aa, synthetic construct
  MOD_RES 2: note = alpha-methyl-L-serine
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 20: note = Lys, wherein the side chain of Lys is connected to O2Oc- O2Oc-gamma-Glu -C18diacid
  MOD_RES 25: note = 2-Aminoisobutyric acid
HXQGSXTSDVSKXLDSRAAXDXVQXIANT

SEQ ID NO: 335 - protein, 29 aa, synthetic construct
  MOD_RES 2: note = D-serine
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 20: note = Lys, wherein the side chain of Lys is connected to O2Oc-O2Oc-gamma-Glu-C18diacid
  MOD_RES 25: note = 2-Aminoisobutyric acid
HXQGSXTSDVSKXLDSRAAXDXVQXIANT

SEQ ID NO: 336 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 20: note = Lys, wherein the side chain of Lys is connected to O2Oc- O2Oc- gamma-Glu -C18diacid
HXQGTFTSDVSKXLDSRRAXDFVRWLLEXG

SEQ ID NO: 337 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 20: note = Lys, wherein the side chain of Lys is connected to O2Oc- O2Oc- gamma-Glu -C18diacid
HXQGTFTSDVSKXLDSRRAXDFVQWLLEXG

SEQ ID NO: 338 - protein, 30 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 20: note = Lys, wherein the side chain of Lys is connected to O2Oc- O2Oc- gamma-Glu -C18diacid
HXQGTFTSDVSKXLDSRRAXDFVQWLLXTE

SEQ ID NO: 339 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 20: note = Lys, wherein the side chain of Lys is connected to gamma-Glu -C18diacid
HXQGTFTSDYSKXLDSRRAXDFVQWLAXE

SEQ ID NO: 340 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 20: note = Lys, wherein the side chain of Lys is connected to gamma-Glu -C18diacid
HXQGTFTSDYSKXLDSRRAXDFVQWLLEXG

SEQ ID NO: 341 - protein, 29 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to gamma-Glu -C18diacid
HXQGTFTSDYSKXLDSRRAXDFVQWLISE

SEQ ID NO: 342 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 20: note = Lys, wherein the side chain of Lys is connected to gamma-Glu -C18diacid
HXQGTFTSDYSKXLDSRRAXDFVQWLLXT

SEQ ID NO: 343 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 20: note = Lys, wherein the side chain of Lys is connected to gamma-Glu -C18diacid
  MOD_RES 16: note = alpha-methyl-L-phenylalanine
HXQGTFTSDYSKXLDXRRAXDFVQWLLXT

SEQ ID NO: 344 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 20: note = Lys, wherein the side chain of Lys is connected to gamma-Glu -C18diacid
HXQGTFTSDYSKXLDSRRAXDFVQWLLXE

SEQ ID NO: 345 - protein, 30 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 20: note = Lys, wherein the side chain of Lys is connected to gamma-Glu -C18diacid
HXQGTFTSDYSKXLDSRRAXDFVQWLLXTE

SEQ ID NO: 346 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 20: note = Lys, wherein the side chain of Lys is connected to gamma-Glu -C18diacid
HXQGTFTSDYSKXLDSRRAXDFVQWLLEXT

SEQ ID NO: 347 - protein, 29 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to gamma-Glu -C18diacid
HXQGTFTSDVSKXLDSRAAXDFVQWIANT

SEQ ID NO: 348 - protein, 29 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to gamma-Glu-gamma-Glu-O2Oc- O2Oc- gamma-Glu -gamma-Glu-C18diacid
HXQGTFTSDVSKXLDSRAAXDFVQWIANT

SEQ ID NO: 349 - protein, 29 aa, synthetic construct
  MOD_RES 2: note = 2-Aminoisobutyric acid
  MOD_RES 13: note = Diphenylalanine
  MOD_RES 24: note = Lys, wherein the side chain of Lys is connected to gamma-Glu -C18diacid
HXQGSFTSDVSKXLDSRAAQDFVXWIANT

SEQ ID NO: 350 - protein, 28 aa, synthetic construct
  MOD_RES 6: note = alpha-methyl-L-phenylalanine
  MOD_RES 22: note = alpha-methyl-L-phenylalanine
  MOD_RES 13: note = 2-Aminoisobutyric acid
  MOD_RES 25: note = 2-Aminoisobutyric acid
  MOD_RES 24: note = Lys, wherein the side chain of Lys is connected to O2Oc- O2Oc- gamma-Glu -C18diacid
HSQGSXTSDVSKXLDSRAAQDXVXXIAN

SEQ ID NO: 351 - protein, 29 aa, synthetic construct
  MOD_RES 2: note = 2-Aminoisobutyric acid
  MOD_RES 13: note = alpha-methyl-L-phenylalanine
  MOD_RES 24: note = Lys, wherein the side chain of Lys is connected to O2Oc- O2Oc- gamma-Glu -C18diacid
HXHGTFTSDVSKXLDSQAAQDFVXWIANT

SEQ ID NO: 352 - protein, 29 aa, synthetic construct
  MOD_RES 2: note = 2-Aminoisobutyric acid
  MOD_RES 13: note = alpha-methyl-L-phenylalanine
  MOD_RES 24: note = Lys, wherein the side chain of Lys is connected to gamma-Glu -C18diacid
HXHGTFTSDVSKXLDSQAAQDFVXWIANT

SEQ ID NO: 353 - protein, 29 aa, synthetic construct
  MOD_RES 2: note = 2-Aminoisobutyric acid
  MOD_RES 13: note = alpha-methyl-L-phenylalanine
  MOD_RES 24: note = Lys, wherein the side chain of Lys is connected to gamma-Glu -C20diacid
HXHGTFTSDVSKXLDSQAAQDFVXWIANT

SEQ ID NO: 354 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to O2Oc- O2Oc- gamma-Glu -C18diacid
HXQGSXTSDVSKXLDSQAAQDFVXWIANT

SEQ ID NO: 355 - protein, 29 aa, synthetic construct
  MOD_RES 2: note = 2-Aminoisobutyric acid
  MOD_RES 6: note = alpha-methyl-L-phenylalanine
  MOD_RES 24: note = Lys, wherein the side chain of Lys is connected to O2Oc- O2Oc- gamma-Glu -C18diacid
HXHGSXTSDVSKXLDSQAAQDFVXWIANT

SEQ ID NO: 356 - protein, 31 aa, synthetic construct
  MOD_RES 2: note = 2-Aminoisobutyric acid
  MOD_RES 13: note = alpha-methyl-L-phenylalanine
  MOD_RES 24: note = Lys, wherein the side chain of Lys is connected to O2Oc- O2Oc- gamma-Glu -C18diacid
HXHGSXTSDVSKXLDSQAAQDFVXWIANTGG

SEQ ID NO: 357 - protein, 24 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to gamma-Glu -C18diacid
HXHGSXTSDVSKXLDSQAAQDFVX

SEQ ID NO: 358 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to polyethylene glycol 2-polyethylene glycol 2-gamma-Glu-gamma-Glu-C20diacid
HXHGSXTSDVSKXLDSQAAQDFVXWIANT

SEQ ID NO: 359 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to gamma-Glu-gamma-Glu-polyethylene glycol 2-gamma-Glu-gamma-Glu-C28diacid
HXHGSXTSDVSKXLDSQAAQDFVXWIANT

SEQ ID NO: 360 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to gamma-Glu -C20diacid
HXHGSXTSDVSKXLDSQAAQDFVXWIANT

SEQ ID NO: 361 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to polyethylene glycol 2-polyethylene glycol 2-gamma-Glu-gamma-Glu-C20diacid
HXHGSXTSDVSKXLDSQAAQDFVXWIANT

SEQ ID NO: 362 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to O2Oc- O2Oc- gamma-Glu -C20diacid
HXHGSXTSDVSKXLDSQAAQDFVXWIANT

SEQ ID NO: 363 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to -gamma-Glu-gamma-Glu- polyethylene glycol 2-gamma-Glu-gamma-Glu-C20diacid
HXHGSXTSDVSKXLDSQAAQDFVXWIANT

SEQ ID NO: 364 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to O2Oc- O2Oc- gamma-Glu -C18diacid
HXIGSXTSDVSKXLDSQAAQDFVXWIANT

SEQ ID NO: 365 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to O2Oc- O2Oc- gamma-Glu -C18diacid
HXIGSXTSDVSKXLDSRAAQDFVXWIANT

SEQ ID NO: 366 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to O2Oc- O2Oc- gamma-Glu -C20diacid
HXIGSXTSDVSKXLDSRAAQDFVXWIANT

SEQ ID NO: 367 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to O2Oc- O2Oc- gamma-Glu -C18diacid
HXHGSXTSDVSKXLDSRAAQDFVXWIANT

SEQ ID NO: 368 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to O2Oc- O2Oc- gamma-Glu-gamma-Glu-C18diacid
HXHGSXTSDVSKXLDSRAAQDFVXWIANT

SEQ ID NO: 369 - protein, 30 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to O2Oc- O2Oc- gamma-Glu -gamma-Glu-C18diacid
HXHGSXTSDVSKXLDSRAAQDFVXWIANTG

SEQ ID NO: 370 - protein, 31 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to O2Oc- O2Oc- gamma-Glu -gamma-Glu-C18diacid
HXHGSXTSDVSKXLDSRAAQDFVXWIANTGG

SEQ ID NO: 371 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 18: note = 2-Aminoisobutyric acid
  MOD_RES 20: note = Lys, wherein the side chain of Lys is connected to O2Oc- O2Oc- gamma-Glu -C18diacid
HXHGSXTSDVSKXLDSRXAQDFVXWIANT

SEQ ID NO: 372 - protein, 29 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  METAL 13: note = alpha-methyl-L-phenylalanine
  MOD_RES 24: note = Lys, wherein the side chain of Lys is connected to O2Oc- O2Oc- gamma-Glu -C18diacid
HXHGSXTSDVSKXLDSRAAQDFVXXIANT

SEQ ID NO: 373 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to O2Oc- O2Oc- gamma-Glu -C20diacid
HXHGSXTSDVSKXLDSRAAQDFVXWIANT

SEQ ID NO: 374 - protein, 29 aa, synthetic construct
  MOD_RES 2: note = 2-Aminoisobutyric acid
  MOD_RES 18: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to O2Oc- O2Oc- gamma-Glu -C20diacid
HXHGSXTSDVSKXLDSRXAQDFVXWIANT

SEQ ID NO: 375 - protein, 29 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to O2Oc- O2Oc- gamma-Glu -C20diacid
HXHGSXTSDVSKXLDSRAAQDFVXXIANT

SEQ ID NO: 376 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to O2Oc- O2Oc- gamma-Glu -C18diacid
HXHGSXTSDVSKXLDSQAAQDXVXWIANT

SEQ ID NO: 377 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 24: note = Lys, wherein the side chain of Lys is connected to O2Oc-O2Oc-gamma-Glu- gamma-Glu -C18diacid
HXHGSXTSDVSKXLDSRAAQDXVXWIANT

SEQ ID NO: 378 - protein, 29 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 24: note = Lys, wherein the side chain of Lys is connected to O2Oc- O2Oc- gamma-Glu-gamma-GluC18diacid
HXHGSXTSDVSKXLDSRAAQDXVXXIANT

SEQ ID NO: 379 - protein, 31 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 24: note = Lys, wherein the side chain of Lys is connected to O2Oc- O2Oc- gamma-Glu -gamma-Glu-C18diacid
HXHGSXTSDVSKXLDSRAAQDXVXXIANTGG

SEQ ID NO: 380 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 24: note = Lys, wherein the side chain of Lys is connected to O2Oc- O2Oc-gamma-Glu- gamma-Glu -C20diacid
HXHGSXTSDVSKXLDSRAAQDXVXWIANT

SEQ ID NO: 381 - protein, 29 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 24: note = Lys, wherein the side chain of Lys is connected to O2Oc- O2Oc- gamma-Glu -gamma-Glu-C20diacid
HXHGSXTSDVSKXLDSRAAQDXVXXIANT

SEQ ID NO: 382 - protein, 31 aa, synthetic construct; identical to SEQ ID NO: 379
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 24: note = Lys, wherein the side chain of Lys is connected to O2Oc- O2Oc-gamma-Glu- gamma-Glu -C20diacid

SEQ ID NO: 383 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to O2Oc- O2Oc- gamma-Glu -gamma-Glu-C24diacid
HXHGSXTSDVSKXLDSRAAQDFVXWIANT

SEQ ID NO: 384 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 24: note = Lys, wherein the side chain of Lys is connected to O2Oc- O2Oc- gamma-Glu -gamma-Glu-C18diacid
HXHGSXTSDVSKXLDSRAAQDXVXSIANT

SEQ ID NO: 385 - protein, 29 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 24: note = Lys, wherein the side chain of Lys is connected to O2Oc- O2Oc- gamma-Glu -C18diacid
HXHGSXTSDVSKXLDSRAAQDXVXSIANT

SEQ ID NO: 386 - protein, 29 aa, synthetic construct
  MOD_RES 1: note = 1H-imidazol-4-yl)methyl)glycine
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to O2Oc- O2Oc- gamma-Glu -gamma-Glu-C20diacid
XSHGSXTSDVSKXLDSRAAQDFVXWIANT

SEQ ID NO: 387 - protein, 31 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to O2Oc- O2Oc- gamma-Glu -gamma-Glu-C18diacid
HXHGSXTSDVSKXLDSRAAQDFVXSIANTGG

SEQ ID NO: 388 - protein, 31 aa, synthetic construct
  MOD_RES 2: note = 2-Aminoisobutyric acid
  MOD_RES 6: note = alpha-methyl-L-phenylalanine
  MOD_RES 13: note = alpha-methyl-L-phenylalanine
  MOD_RES 24: note = Lys, wherein the side chain of Lys is connected to O2Oc- O2Oc- gamma-Glu -gamma-Glu-C18diacid
HXHGSXTSDVSKXLDSRXAQDFVXSIANTGG

SEQ ID NO: 389 - protein, 29 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 24: note = Lys, wherein the side chain of Lys is connected to O2Oc- O2Oc- gamma-Glu -C18diacid
HXHGSXTSDVSKXLDSQAAQDXVXXIANT

SEQ ID NO: 390 - protein, 29 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 24: note = Lys, wherein the side chain of Lys is connected to O2Oc- O2Oc- gamma-Glu -gamma-Glu-C20diacid
HXHGSXTSDVSKXLDSQAAQDXVXXIANT

SEQ ID NO: 391 - protein, 29 aa, synthetic construct
  MOD_RES 13: note = alpha-methyl-L-phenylalanine
  MOD_RES 24: note = Lys, wherein the side chain of Lys is connected to O2Oc- O2Oc- gamma-Glu -stearoyl
HSQGTFTSDYSKXLEEEAVRLFIXWLMNT

SEQ ID NO: 392 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 24: note = Lys, wherein the side chain of Lys is connected to O2Oc- O2Oc- gamma-Glu -C18diacid
HXQGTFTSDVSKXLDSRRAQDFVXWLVEXG

SEQ ID NO: 393 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 24: note = Lys, wherein the side chain of Lys is connected to O2Oc- O2Oc- gamma-Glu -C18diacid
HXQGTFTSDVSKXLESRRAQDFVXWLVEXG

SEQ ID NO: 394 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 24: note = Lys, wherein the side chain of Lys is connected to O2Oc- O2Oc- gamma-Glu -C18diacid
HXQGTFTSDVSKXLDLRRAQDFVXWLVEXG

SEQ ID NO: 395 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 24: note = Lys, wherein the side chain of Lys is connected to O2Oc- O2Oc- gamma-Glu -C18diacid
HXQGTFTSDVSKXLDSRRAQDFVXWLLXT

SEQ ID NO: 396 - protein, 30 aa, synthetic construct
  MOD_RES 2: note = 2-Aminoisobutyric acid
  MOD_RES 30: note = 2-Aminoisobutyric acid
  MOD_RES 13: note = alpha-methyl-L-phenylalanine
  MOD_RES 24: note = Lys, wherein the side chain of Lys is connected to O2Oc- O2Oc- gamma-Glu -C18diacid
HXQGTFTSDVSKXLDSRRAQDFVXWLVEGX

SEQ ID NO: 397 - protein, 29 aa, synthetic construct
  MOD_RES 2: note = 2-Aminoisobutyric acid
  MOD_RES 25: note = 2-Aminoisobutyric acid
  MOD_RES 10: note = Lys, wherein the side chain of Lys is connected to O2Oc- O2Oc- gamma-Glu -gamma-Glu-C20diacid
  MOD_RES 6: note = alpha-methyl-L-phenylalanine
  MOD_RES 13: note = alpha-methyl-L-phenylalanine
  MOD_RES 22: note = alpha-methyl-L-phenylalanine
HXHGSXTSDXSKXLDSRAAQDXVQXIANT

SEQ ID NO: 398 - protein, 29 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 10: note = Lys, wherein the side chain of Lys is connected to O2Oc- O2Oc- gamma-Glu -C18diacid
HXHGSXTSDXSKXLDSRAAQDXVQXIANT

SEQ ID NO: 399 - protein, 29 aa, synthetic construct
  MOD_RES 6: note = alpha-methyl-L-phenylalanine
  MOD_RES 22: note = alpha-methyl-L-phenylalanine
  MOD_RES 10: note = Lys, wherein the side chain of Lys is connected to polyethylene glycol 2-polyethylene glycol 2-gamma-Glu-gamma-Glu-Stearoyl
  MOD_RES 13: note = diphenylpropanoic acid
  MOD_RES 17: note = Beta-dimethylglutamine
HSQGSXTSDXSKXLDSXAAQDXVEXLANT

SEQ ID NO: 400 - protein, 29 aa, synthetic construct
  MOD_RES 6: note = alpha-methyl-L-phenylalanine
  MOD_RES 22: note = alpha-methyl-L-phenylalanine
  MOD_RES 10: note = Lys, wherein the side chain of Lys is connected to polyethylene glycol 2-polyethylene glycol 2-gamma-Glu-gamma-Glu-Palmitoyl
  MOD_RES 13: note = diphenylpropanoic acid
  MOD_RES 17: note = Beta-dimethylglutamine
HSQGSXTSDXSKXLDSXAAQDXVEXLANT

SEQ ID NO: 401 - protein, 29 aa, synthetic construct
  MOD_RES 2: note = 2-Aminoisobutyric acid
  MOD_RES 10: note = Lys, wherein the side chain of Lys is connected to polyethylene glycol 2-polyethylene glycol 2-gamma-Glu-Palmitoyl
HXQGSFTSDXSKILDSRAAQDFVEWIANT

SEQ ID NO: 402 - protein, 29 aa, synthetic construct
  MOD_RES 2: note = 2-Aminoisobutyric acid
  MOD_RES 10: note = Lys, wherein the side chain of Lys is connected to polyethylene glycol 2-polyethylene glycol 2-gamma-Glu-Palmitoyl
  MOD_RES 22: note = alpha-methyl-L-phenylalanine
  MOD_RES 6: note = alpha-methyl-L-phenylalanine
HXQGSXTSDXSKILDSRAAQDXVEWIANT

SEQ ID NO: 403 - protein, 29 aa, synthetic construct
  MOD_RES 6: note = alpha-methyl-L-phenylalanine
  MOD_RES 22: note = alpha-methyl-L-phenylalanine
  MOD_RES 10: note = Lys, wherein the side chain of Lys is connected to polyethylene glycol 2-polyethylene glycol 2-gamma-Glu-gamma-Glu-Stearoyl
  MOD_RES 13: note = diphenylpropanoic acid
HSQGSXTSDXSKXLDSQAAQDXVEXLANT

SEQ ID NO: 404 - protein, 29 aa, synthetic construct
  MOD_RES 6: note = alpha-methyl-L-phenylalanine
  MOD_RES 22: note = alpha-methyl-L-phenylalanine
  MOD_RES 10: note = Lys, wherein the side chain of Lys is connected to polyethylene glycol 2-polyethylene glycol 2-gamma-Glu-gamma-Glu-Stearoyl
  MOD_RES 13: note = diphenylpropanoic acid
  MOD_RES 17: note = Beta-dimethylglutamine
HSQGSXTSDXSKXLDSXAAQDXVEHLANT

SEQ ID NO: 405 - protein, 29 aa, synthetic construct
  MOD_RES 6: note = alpha-methyl-L-phenylalanine
  MOD_RES 22: note = alpha-methyl-L-phenylalanine
  MOD_RES 13: note = Lys, wherein the side chain of Lys is connected to polyethylene glycol 2-polyethylene glycol 2-gamma-Glu-gamma-Glu-Stearoyl)
  MOD_RES 17: note = Beta-dimethylglutamine
HSQGSXTSDVSKXLDSXAAQDXVEXLANT

SEQ ID NO: 406 - protein, 29 aa, synthetic construct
  MOD_RES 6: note = alpha-methyl-L-phenylalanine
  MOD_RES 22: note = alpha-methyl-L-phenylalanine
  MOD_RES 13: note = Lys, wherein the side chain of Lys is connected to polyethylene glycol 2-polyethylene glycol 2-gamma-Glu-gamma-Glu-Palmitoyl
  MOD_RES 17: note = Beta-dimethylglutamine
HSQGSXTSDVSKXLDSXAAQDXVEXLANT

SEQ ID NO: 407 - protein, 29 aa, synthetic construct
  MOD_RES 2: note = 2-Aminoisobutyric acid
  MOD_RES 6: note = alpha-methyl-L-phenylalanine
  MOD_RES 22: note = alpha-methyl-L-phenylalanine
  MOD_RES 13: note = Lys, wherein the side chain of Lys is connected to polyethylene glycol 2-polyethylene glycol 2-gamma-Glu-Palmitoyl
HXQGSXTSDVSKXLDSRAAQDXVEWIANT

SEQ ID NO: 408 - protein, 29 aa, synthetic construct
  MOD_RES 2: note = 2-Aminoisobutyric acid
  MOD_RES 13: note = Lys, wherein the side chain of Lys is connected to polyethylene glycol 2-polyethylene glycol 2-gamma-Glu-Palmitoyl
HXQGSFTSDVSKXLDSRAAQDFVEWIANT

SEQ ID NO: 409 - protein, 29 aa, synthetic construct
  MOD_RES 6: note = alpha-methyl-L-phenylalanine
  MOD_RES 22: note = alpha-methyl-L-phenylalanine
  MOD_RES 13: note = Lys, wherein the side chain of Lys is connected to polyethylene glycol 2-polyethylene glycol 2-gamma-Glu-gamma-Glu-Stearoyl
  MOD_RES 25: note = 2-Aminoisobutyric acid
HSQGSXTSDVSKXLDSQAAQDXVEXLANT

SEQ ID NO: 410 - protein, 29 aa, synthetic construct
  MOD_RES 6: note = alpha-methyl-L-phenylalanine
  MOD_RES 22: note = alpha-methyl-L-phenylalanine
  MOD_RES 13: note = Lys, wherein the side chain of Lys is connected to polyethylene glycol 2-polyethylene glycol 2-gamma-Glu-gamma-Glu-Stearoyl
  MOD_RES 17: note = Beta-dimethylglutamine
HSQGSXTSDVSKXLDSXAAQDXVEHLANT

SEQ ID NO: 411 - protein, 29 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 22: note = alpha-methyl-L-phenylalanine
  MOD_RES 13: note = Lys, wherein the side chain of Lys is connected to O2Oc- O2Oc- gamma-Glu -gamma-Glu-C20diacid
HXHGSXTSDVSKXLDSRAAQDXVQXIANT

SEQ ID NO: 412 - protein, 29 aa, synthetic construct
  MOD_RES 2: note = 2-Aminoisobutyric acid
  MOD_RES 25: note = 2-Aminoisobutyric acid
  MOD_RES 6: note = alpha-methyl-L-phenylalanine
  MOD_RES 22: note = alpha-methyl-L-phenylalanine
  MOD_RES 13: note = Lys, wherein the side chain of Lys is connected to O2Oc- O2Oc- gamma-Glu -C18diacid
HXHGSXTSDVSKXLDSRAAQDXVQXIANT

SEQ ID NO: 413-417 - skipped (5 entries)

SEQ ID NO: 418 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLDTXRAQDFVRWLEAXG

SEQ ID NO: 419 - protein, 30 aa, synthetic construct; identical to SEQ ID NO: 418
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C20diacid

SEQ ID NO: 420 - protein, 30 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDTXRARDFVAWLLXTE

SEQ ID NO: 421 - protein, 30 aa, synthetic construct; identical to SEQ ID NO: 420
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 422 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDTXRAQDFVAWLEAGG

SEQ ID NO: 423 - protein, 30 aa, synthetic construct; identical to SEQ ID NO: 422
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 424 - protein, 30 aa, synthetic construct; identical to SEQ ID NO: 422
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C20diacid

SEQ ID NO: 425 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDKXRARDFVRWLLXE

SEQ ID NO: 426 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLESXRAQDFVRWLEAXG

SEQ ID NO: 427 - protein, 30 aa, synthetic construct; identical to SEQ ID NO: 426
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 428 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C18diacid
HXQGTFTSDVSKXLDTXRAQDFVAWLEAXG

SEQ ID NO: 429 - protein, 30 aa, synthetic construct; identical to SEQ ID NO: 428
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C20diacid

SEQ ID NO: 430 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDTXRAQDFVRWLEXE

SEQ ID NO: 431 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDKXRAQDFVRWLLXE

SEQ ID NO: 432 - protein, 30 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDRXRAQDFVRWLLXTE

SEQ ID NO: 433 - protein, 30 aa, synthetic construct; identical to SEQ ID NO: 432
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid

SEQ ID NO: 434 - protein, 30 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDKXRAQDFVRWLKXTE

SEQ ID NO: 435 - protein, 30 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDKXRAQDFVRWLLXTK

SEQ ID NO: 436 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = 2-Aminoisobutyric acid Lys, wherein the side chain of Lys is connected to O2Oc-O2Oc-gamma-Glu-C18diacid
HXQGTFTSDVSKXLDKXRAQDFVRWLKAXG

SEQ ID NO: 437 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDKXRAQDFVRWLLRXK

SEQ ID NO: 438 - protein, 30 aa, synthetic construct; identical to SEQ ID NO: 100
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid

SEQ ID NO: 439 - protein, 30 aa, synthetic construct; identical to SEQ ID NO: 100
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C18diacid

SEQ ID NO: 440 - protein, 30 aa, synthetic construct; identical to SEQ ID NO: 59
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid

SEQ ID NO: 441 - protein, 30 aa, synthetic construct; identical to SEQ ID NO: 59
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C18diacid

SEQ ID NO: 442 - protein, 30 aa, synthetic construct; identical to SEQ ID NO: 428
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C18diacid

SEQ ID NO: 443 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 25: note = biphenyl-alanine
HXQGTFTSDVSKXLDSXRAQDFVRXLLXE

SEQ ID NO: 444 - protein, 30 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 25: note = 1-methyl tryptophan
HXQGTFTSDVSKXLDSXRAQDFVRXPLLXE

SEQ ID NO: 445 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
  MOD_RES 25: note = 5-Bromo trptophan
HXQGTFTSDVSKXLDSXRAQDFVRXLLXE

SEQ ID NO: 446 - protein, 30 aa, synthetic construct
  MOD_RES 2: note = 2-Aminoisobutyric acid
  MOD_RES 29: note = 2-Aminoisobutyric acid
  MOD_RES 25: note = 2-Aminoisobutyric acid
  MOD_RES 13: note = alpha-methyl-L-phenylalanine
  MOD_RES 17: note = 2-Aminoisobutyric acid Lys, wherein the side chain of Lys is connected to O2Oc-O2Oc-gamma-Glu-C18diacid
HXQGTFTSDVSKXLDTXRARDFVQXLLEXG

SEQ ID NO: 447 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDRXRAQDFVRWLVXE

SEQ ID NO: 448 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLDRXRAQDFVRWLVXE

SEQ ID NO: 449 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDKXRAQDFVRWLEAXG

SEQ ID NO: 450 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDRXRAQDFVRWLLXE

SEQ ID NO: 451 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLDKXRARDFVRWLLXE

SEQ ID NO: 452 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLDKXRAQDFVRWLLXE

SEQ ID NO: 453 - protein, 30 aa, synthetic construct; identical to SEQ ID NO: 94
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid

SEQ ID NO: 454 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDKXRARDFVRWLLXE

SEQ ID NO: 455 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLETXRARDFVQWLLEXG

SEQ ID NO: 456 - protein, 30 aa, synthetic construct; identical to SEQ ID NO: 455
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid

SEQ ID NO: 457 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C18diacid
HXQGTFTSDVSKXLDRXRAQDFVRWLVXE

SEQ ID NO: 458 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C20diacid
HXQGTFTSDVSKXLDRXRAQDFVRWLVXE

SEQ ID NO: 459 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C18diacid
HXQGTFTSDVSKXLDKXRARDFVRWLLXE

SEQ ID NO: 460 - protein, 30 aa, synthetic construct; identical to SEQ ID NO: 100
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C20diacid

SEQ ID NO: 461 - protein, 30 aa, synthetic construct; identical to SEQ ID NO: 94
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C18diacid

SEQ ID NO: 462 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLDKXRARDFVLWLLXE

SEQ ID NO: 463 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLDKXRALDFVRWLLXE

SEQ ID NO: 464 - protein, 30 aa, synthetic construct; identical to SEQ ID NO: 94
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 465 - protein, 30 aa, synthetic construct; identical to SEQ ID NO: 449
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 466 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C20diacid
HXQGTFTSDVSKXLDKXRAQDFVRWLLXE

SEQ ID NO: 467 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLDRXRAQDFVRWLLXE

SEQ ID NO: 468 - protein, 30 aa, synthetic construct; identical to SEQ ID NO: 100
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 469 - protein, 30 aa, synthetic construct; identical to SEQ ID NO: 100
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C20diacid

SEQ ID NO: 470 - protein, 28 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDKXRAQDFVRWLLA

SEQ ID NO: 471 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDKXRAQDFVRWLLAE

SEQ ID NO: 472 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDRXRAQDFVRWLLAE

SEQ ID NO: 473 - protein, 30 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDTXRARDFVQWLLEAG

SEQ ID NO: 474 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C18diacid
HXQGTFTSDVSKXLDSXRAQDFVQWLLXT

SEQ ID NO: 475 - protein, 30 aa, synthetic construct
  MOD_RES 2: note = 2-Aminoisobutyric acid
  METAL 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDTXRAQDFVQWLLEXG

SEQ ID NO: 476 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C18diacid
HXQGTFTSDVSKXLDLXRAQDFVQWLLXT

SEQ ID NO: 477 - protein, 30 aa, synthetic construct; identical to SEQ ID NO: 475
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid

SEQ ID NO: 478 - protein, 30 aa, synthetic construct; identical to SEQ ID NO: 475
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 479 - protein, 30 aa, synthetic construct; identical to SEQ ID NO: 475
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C18diacid

SEQ ID NO: 480 - protein, 30 aa, synthetic construct; identical to SEQ ID NO: 475
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C20diacid

SEQ ID NO: 481 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLDKXRARDFVQWLLXE

SEQ ID NO: 482 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLDRXRARDFVQWLLXE

SEQ ID NO: 483 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDKXRARDFVQWLLXE

SEQ ID NO: 484 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDRXRARDFVQWLLXE

SEQ ID NO: 485 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDTXRARDFVQWLLAXG

SEQ ID NO: 486 - protein, 30 aa, synthetic construct; identical to SEQ ID NO: 485
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 487 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDTXRAQDFVRWLLAXG

SEQ ID NO: 488 - protein, 30 aa, synthetic construct; identical to SEQ ID NO: 487
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 489 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDTXRAQDFVRWLLEXK

SEQ ID NO: 490 - protein, 30 aa, synthetic construct; identical to SEQ ID NO: 489
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 491 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDTXRARDFVQWLLEXK

SEQ ID NO: 492 - protein, 30 aa, synthetic construct; identical to SEQ ID NO: 491
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 493 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLESXRAQDFVQWLLXE

SEQ ID NO: 494 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  METAL 17: note = Lys, wherein the side chain of Lys is connected to O2Oc-O2Oc-gamma-Glu-C18diacid
HXQGTFTSDVSKXLDTXRAQDFVQWLLXE

SEQ ID NO: 495 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLDTXRAQDFVQWLLXE

SEQ ID NO: 496 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLDTXRAQDFVQWLLXE

SEQ ID NO: 497 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLDLXRAQDFVRWLLXE

SEQ ID NO: 498 - protein, 30 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDTXRARDFVQWLLXTE

SEQ ID NO: 499 - protein, 30 aa, synthetic construct; identical to SEQ ID NO: 498
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 500 - protein, 30 aa, synthetic construct; identical to SEQ ID NO: 498
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid

SEQ ID NO: 501 - protein, 30 aa, synthetic construct; identical to SEQ ID NO: 498
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 502 - protein, 30 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDTXRARDFVRWLLXTE

SEQ ID NO: 503 - protein, 30 aa, synthetic construct; identical to SEQ ID NO: 502
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 504 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDTXRAQDFVRWLLRXK

SEQ ID NO: 505 - protein, 30 aa, synthetic construct; identical to SEQ ID NO: 504
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 506 - protein, 30 aa, synthetic construct; identical to SEQ ID NO: 504
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid

SEQ ID NO: 507 - protein, 30 aa, synthetic construct; identical to SEQ ID NO: 504
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 508 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDTXRAQDFVRWLLRXA

SEQ ID NO: 509 - protein, 30 aa, synthetic construct; identical to SEQ ID NO: 508
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 510 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDTXRAQDFVQWLLRXA

SEQ ID NO: 511 - protein, 30 aa, synthetic construct; identical to SEQ ID NO: 510
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 512 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDTXRARDFVQWLLRXA

SEQ ID NO: 513 - protein, 30 aa, synthetic construct; identical to SEQ ID NO: 512
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 514 - protein, 30 aa, synthetic construct; identical to SEQ ID NO: 489
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 515 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLESXRAQDFVRWLLEXK

SEQ ID NO: 516 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLDTXRAQDFVQWLLEXK

SEQ ID NO: 517 - protein, 30 aa, synthetic construct; identical to SEQ ID NO: 112
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 518 - protein, 30 aa, synthetic construct; identical to SEQ ID NO: 112
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 519 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLESXRAQDFVRWLVEXG

SEQ ID NO: 520 - protein, 30 aa, synthetic construct; identical to SEQ ID NO: 84
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 521 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLDTXRARDFVRWLLXE

SEQ ID NO: 522 - protein, 29 aa, synthetic construct
  MOD_RES 2: note = 2-Aminoisobutyric acid
  MOD_RES 28: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLDTXRARDFVRWLLXE

SEQ ID NO: 523 - protein, 30 aa, synthetic construct
  MOD_RES 2: note = 2-Aminoisobutyric acid
  MOD_RES 29: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 17: note = Lys, wherein the side chain of Lys is connected to O2Oc-O2Oc-gamma-Glu-C18diacid
HXQGTXTSDVSKXLDTXRAQDXVRWLLAXG

SEQ ID NO: 524 - protein, 30 aa, synthetic construct
  MOD_RES 2: note = 2-Aminoisobutyric acid
  MOD_RES 29: note = 2-Aminoisobutyric acid
  MOD_RES 6: note = alpha-methyl-L-phenylalanine
  MOD_RES 13: note = alpha-methyl-L-phenylalanine
  MOD_RES 22: note = alpha-methyl-L-phenylalanine
  MOD_RES 17: note = Lys, wherein the side chain of Lys is connected to O2Oc-O2Oc-gamma-Glu-C18diacid
HXQGTXTSDVSKXLDTXRARDXVQWLLAXG

SEQ ID NO: 525 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C18diacid
HXQGTFTSDVSKXLDTXAARDFVQWLLEXG

SEQ ID NO: 526 - protein, 30 aa, synthetic construct; identical to SEQ ID NO: 525
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid

SEQ ID NO: 527 - protein, 29 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-C20diacid
HXQGTFTSDVSKXLESXRAQDFVRWLLXG

SEQ ID NO: 528 - protein, 29 aa, synthetic construct
  MOD_RES 2: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C18diacid
HXQGTFTSDVSKXLEAXAAREFIAWLLET

SEQ ID NO: 529 - protein, 30 aa, synthetic construct; identical to SEQ ID NO: 100
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to O2Oc-O2Oc-gamma-Glu-gamma-Glu-C18diacid

SEQ ID NO: 530 - protein, 30 aa, synthetic construct; identical to SEQ ID NO: 100
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to gamma-Glu-gamma-Glu-O2Oc-O2Oc-gamma-Glu-gamma-Glu-C18diaci d

SEQ ID NO: 531 - protein, 30 aa, synthetic construct; identical to SEQ ID NO: 100
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 17: note = Lys, wherein the side chain of Lys is connected to gamma-Glu-gamma-Glu- O2Oc-O2Oc-gamma-Glu-C20diacid

SEQ ID NO: 532 - protein, 30 aa, synthetic construct
  MOD_RES 2: note = 2-Aminoisobutyric acid
  MOD_RES 13: note = 2-Aminoisobutyric acid
  MOD_RES 20: note = Lys, wherein the side chain of Lys is connected to O2Oc- O2Oc- gamma-Glu -C18diacid
HXQGTFTSDVSKXLDSERAXDFVQWLEAGG

SEQ ID NO: 533 - protein, 30 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to O2Oc- O2Oc- gamma-Glu -C18diacid
HXQGTFTSDVSKXLDSERAXDFVAWLEAGG

SEQ ID NO: 534 - protein, 30 aa, synthetic construct; identical to SEQ ID NO: 533
  MOD_RES 2: note = 2-Aminoisobutyric acid
  MOD_RES 13: note = alpha-methyl-L-phenylalanine
  MOD_RES 20: note = Lys, wherein the side chain of Lys is connected to O2Oc- O2Oc- gamma-Glu -C20diacid

SEQ ID NO: 535 - protein, 30 aa, synthetic construct
  MOD_RES 2: note = 2-Aminoisobutyric acid
  MOD_RES 13: note = alpha-methyl-L-phenylalanine
  MOD_RES 20: note = Lys, wherein the side chain of Lys is connected to O2Oc- O2Oc- gamma-Glu -C18diacid
HXQGTFTSDVSEXLDSERAXDFVRWLEAGG

SEQ ID NO: 536 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 20: note = Lys, wherein the side chain of Lys is connected to O2Oc- O2Oc- gamma-Glu -C18diacid
HXQGTFTSDVSKXLDSERAXDFVQWLEAXG

SEQ ID NO: 537 - protein, 30 aa, synthetic construct
  MOD_RES 2: note = 2-Aminoisobutyric acid
  MOD_RES 29: note = 2-Aminoisobutyric acid
  MOD_RES 13: note = alpha-methyl-L-phenylalanine
  MOD_RES 20: note = Lys, wherein the side chain of Lys is connected to O2Oc- O2Oc- gamma-Glu -C18diacid
HXQGTFTSDVSKXLDSERAXDFVAWLEAXG

SEQ ID NO: 538-541 - skipped (4 entries)